SEQUENCE LISTING (110 sequences)

SEQ ID NO: 1 - DNA, 1479 bp, Artificial Sequence
  Description of Artificial Sequence Synthetic polynucleotide
atggccgcagacagtcttgttgtccttgttctgtgccttagttgccttttgctgctatctctttggagacaatcatcagggagaggtaaacttccgcctggaccaactccactacccgtcatagggaatatattacaaatcggtataaaggacatctccaagtccctgacgaatctttccaaggtgtatggtcctgtgttcacactatacttcggcttgaaacccatcgtggtcttacatggctacgaggcagtgaaagaggccctgattgatttgggggaagagttcagtgggagaggaatctttccccttgctgagagggctaatcgtggttttgggatagtgttttctaacggaaagaagtggaaagaaataaggcgtttcagcctgatgactttgcgtaattttgggatgggaaaaaggtcaattgaagatcgtgttcaagaagaagcccgttgcctggtggaggagttgagaaagacgaaggcttccccgtgcgatccaactttcatactgggatgtgcgccatgcaatgtcatatgtagtataatctttcataagagattcgactataaggatcagcaattcttgaacttgatggagaaattgaacgagaacataaaaattctgtcttccccctggattcaaatatgtaataactttagcccaataatagactacttcccaggtacgcacaataaactgttaaagaacgtcgcttttatgaaatcttacatattggagaaggtgaaagagcaccaagagagcatggacatgaacaatccgcaagacttcattgattgtttcctgatgaagatggaaaaagaaaagcacaaccagccttctgaatttacgattgaaagccttgaaaatactgcagtcgatctattcggagctggcacagagactacctcaaccacgttaagatatgctttgcttttactactgaagcatccagaggtgactgccaaggtgcaagaagagatcgagagggtcatcggaaggaaccgttccccgtgtatgcaggacaggagccatatgccttacacagacgcggttgtccacgaagtccagcgttacatagatctattaccgacgtcactaccccacgcggtcacttgtgacatcaaatttcgtaactacctgatccccaagggcactaccatattaatttcacttacttccgtgctacacgacaataaggaatttccaaatcccgagatgttcgacccgcatcactttctggacgaagggggaaatttcaagaagtcaaagtacttcatgcctttctccgccggaaagagaatctgtgtaggagaagctctggcggggatggaactattcttgtttttaacctcaatattacagaattttaaccttaaatcccttgtagatcctaagaatctggacacaacgcctgtggttaacgggttcgcgtccgttccgccgttttaccagttatgctttattcccgtttaa

SEQ ID NO: 2 - DNA, 1479 bp, Artificial Sequence
  Description of Artificial Sequence Synthetic polynucleotide
atggccgcagactctcttgttgtattggtattatgcctaagctgcttgcttctattaagcctatggagacaaagcagtgggagagggaaacttccgcccggaccaactcctctacctgtaatcgggaacattttacaaatcggcataaaagatatctcaaaaagtttaacaaatttgtccaaggtgtacggcccggtatttactctttacttcggattgaagccgatagtagttttgcacggctatgaggccgtcaaggaggcacttatagacttaggagaggagttttctgggaggggcattttcccgcttgcagagcgtgcaaatagggggtttgggatagtgttctcaaatggtaagaaatggaaagaaatcaggcgtttttctctgatgacccttaggaacttcggaatgggaaagagatctatcgaagacagggtccaggaggaagcccgttgcctagtagaagaacttcgtaagacgaaggcttccccatgtgaccctacctttattctaggctgtgcgccgtgcaatgtcatatgttctattatttttcataagagattcgattataaggatcagcagttcctgaatttaatggagaaattaaacgagaatgttaaaatacttagttcaccttggatacagatatgtaataacttttcacctataatcgattattttcccggaactcataacaagctcttgaagaatgttgcttttatgaagtcttacattttagagaaagttaaagagcatcaggaatccatggacatgaataacccacaggatttcattgactgcttcttaatgaaaatggaaaaggaaaagcataaccagccaagtgagttcactattgaatctcttgaaaacacggctgtggatctgttcggagcaggaaccgagactacgtctacgacgctgcgttatgcgttactgctattactgaaacatccagaagttacagcgaaggtacaagaggagatcgagagggtcatcggaagaaataggagtccctgtatgcaagatcgttctcatatgccctacacagatgcagtcgttcatgaagtgcagagatatatcgacttgttacccacctccctacctcacgcagtaacctgcgatatcaaatttaggaattatttaatacctaaagggacgaccattctgataagcctaacatcagtcttgcacgataacaaggaatttccgaaccccgagatgtttgacccacaccatttcctggacgagggcgggaacttcaagaaatccaattattttatgcctttcagtgctggtaagaggatatgcgtaggagaggctttagccaggatggagcttttcctattcctgacatctatacttcaaaacttcaatctaaagagtttagtcgatccgaaaaatttagatacgacgcctgttgtaaatgggttcgcctccgtacctcccttctaccaattgtgctttattcccgtgtaa

SEQ ID NO: 3 - DNA, 1479 bp, Artificial Sequence
  Description of Artificial Sequence Synthetic polynucleotide
atggccgcagattcctttgtggtgctggtgctgtgtttaagctgcttattgttactatccttatggcgtcaatcatccggacgtggcaaattgccccctggcccaacacccctgcccgttataggaaatatacttcagattgacataaaagatatcagtaaatccctaacgaatctttctaaagtttatgggcccgtctttaccctttatttcggtctgaaaccgattgtcgttttacacggatacgaggcagtgaaagaggctctgatcgacttaggtgaggagttctctggccgtggacattttccattggcagaacgtgctaatagggggttcggtattgtattctccaacgggaaaaagtggaaggaaatcaggcgtttttccttaatgacgctaagaaacttcggcatgggtaagaggagtatagaagaccgtgttcaagaggaagctagatgcttagtagaggagctgaggaagactaaggcctctccctgtgatccaacattcattctgggctgtgctccgtgcaatgtcatctgtagtataatttttcgtaagagatttgactataaggatcaacagttcctgaatcttatggagaaacttaatgaaaatgtcaagatactgtcttctccctggatacaaatttacaacaacttttctcccatcatagattacttccctggaacgcataacaagctgttgaaaaatgtggcttttatgaagtcctatattctggaaaaggtcaaggaacatcaggaaagtatggacatgaacaatccgcaagatttcatcgattgcttcttaatgaagatggagaaagaaaaacataatcaacctagtgagtttacgatagagagtcttgaaaacactgccgcggacctattcggcgccggcacggaaaccacatctaccacccttaggtatgcattacttctactactaaaacatcctgaagttaccgctaaggtacaagaagagatcgagagagtaataggcaggaatagaagtccgtgtatgcaagataggagccacatgccatacacagacgcagtcgtccatgaagttcagcgttatattgaccttcttccgaccagtctgccacatgcagtcacctgtgacattaaattcaggaattatttaattcccaaaggtacaacaatattaatctctctgacgagcgttctacatgacaataaggagttccctaacccagagatgttcgatccgcaccatttcctagacgaaggtggaaactttaagaagagcaattattttatgccattctccgctgggaaaagaatctgtgttggcgaagcattggccagaatggaattgtttttgttcctaacaagcatcttacaaaatttcaatcttaaatctttggttgacccgaagaatctggacaccacacctgtcgtaaatgggtttgcaagcgtaccacctttttatcaattgtgtttcatccccgtctaa

SEQ ID NO: 4 - DNA, 1479 bp, Artificial Sequence
  Description of Artificial Sequence Synthetic polynucleotide
atggccgcagatctggtagtgttcttggccttgaccctaagctgtttaattctactatcattatggcgtcagtcctccggacgtggtaaactaccgccaggaccaactccgctgcccattatcgggaactttcttcaaatcgacgtcaaaaacatatcacaatcatttacaaacttctcaaaagcatacgggccagtttttactctgtacctaggaagcaaacccacagttattttgcatggctacgaagctgtcaaggaggcgttgatagacagaggagaagaatttgctgggaggggaagtttcccgatggccgaaaagatcatcaagggatttggcgtcgtgttttctaacggcaataggtggaaagaaatgaggagattcacattgatgactctgaggaacctgggtatgggaaagagaaacattgaagatagggtccaggaggaggcacaatgtttggttgaagaactaagaaaaacaaaaggaagtccctgtgatccaacgttcattctatcctgcgctccctgcaatgttatctgttctattatattccaaaaccgtttcgattataaagataaagaatttctaatactaatggataaaattaacgagaacgtgaagatcctatcctcaccctggttgcaagtttgcaattcatttccttccttaatagactattgtccaggttctcatcacaaaatagtgaaaaatttcaactatttaaagtcttatttgctggagaaaattaaagagcataaagagagccttgacgttactaaccccagggactttattgactactatttaattaagcagaaacaggttaaccatattgaacagtcagaattttctttagagaatttagcctctacaattaacgacctgttcggggccgggacagaaaccacgagcacaacgctgagatacgcattactactgctacttaaatatccggatgttactgctaaggttcaggaagaaatcgatagggtagtaggacgtcatcgttcaccatgcatgcaagatcgttcacacatgccttatactgatgcaatgatacacgaagttcagcgttttattgacttgttaccaaccagtttaccgcatgcggtcacatgtgacatcaaatttaggaaatatctgatccccaagggtacaactgtcatcactagcctaagctccgtattgcatgacagtaaagagttcccaaatccagagatgttcgacccagggcactttttgaatgcgaatggcaattttaagaagagcgactatttcatgccctttagcactggcaagagaatatgtgccggagagggactagcaaggatggaattattcctgattcttaccacaatactacagaacttcaaattaaaatcattagtccacccaaaagagatagatattactccagtgatgaacggttttgcatcccttccgccaccctaccaactatgttttattccgctttaa

SEQ ID NO: 5 - DNA, 1480 bp, Artificial Sequence
  Description of Artificial Sequence Synthetic polynucleotide
aatggccgcaattttaggcgtattccttggtttgtttttgacgtgtttactattgttaagtttgtggaagcagaatttccaaaggagaaatttacccccaggaccgacaccacttcccattatcggtaacatacttcaaatcgacttaaaggacatttccaagagtttgagaaacttctcaaaagtctacggcccggtatttaccctgtacttggggaggaaacccgcggtcgttctgcatggttacgaggctgttaaagaggcacttatcgatcacggggaagagttcgcaggtaggggtgtgtttcccgtcgcccaaaagtttaacaagaactgcggggtggttttctcatccggccgtacctggaaggaaatgaggagattctccttgatgacacttaggaattttgggatgggcaagagaagtatagaggatagggtacaggaagaggcacgttgtctagtagacgaacttcgtaaaactaacggggtgccttgtgatccaacctttatcctggggtgcgccccgtgtaacgtgatttgctctatcgtattccaaaacagattcgattacaaagaccaggagtttcttgcgctaatagatatactaaatgaaaacgttgagatccttggatcaccgtggattcaaatttgtaataacttcccagctattattgactatttaccgggaagacacaggaaactgttaaagaactttgcttttgcgaaacattacttcttagctaaagtaattcaacaccaggaatcattagatatcaataatccccgtgatttcatcgactgcttccttataaaaatggagcaggagaagcataatcccaaaactgagtttacttgcgagaacttaatcttcactgcttctgaccttttcgcggccggtacggagacaacctctactacacttcgttattccttattattgttgttaaagtaccctgaggttacggcaaaggtgcaagaagagattgaccacgtgataggtcgtcacaggtctccatgtatgcaagaccgtcatcacatgccgtacacagacgctgtactgcacgagatacagcgttacatcgacctattacccacgagcttacctcacgcgcttacctgtgatatgaagtttagggattatttaatcccgaagggaactaccgttatcgcttctttaacttcagtgctttacgatgataaggagttccctaacccagagaaatttgatccaagccacttccttgacgagaacggaaaattcaaaaagtccgattacttcttcccgttctctactggaaaaaggatctgcgtaggagaggggcttgctcgtaccgaattgtttctattcttaactacaattctgcaaaattttaacctgaagagccctgtagatctgaaggagttagacacgaatcctgtggcaaacggttttgtgtcagtaccaccaaaatttcagatctgttttattcctatataa

SEQ ID NO: 6 - DNA, 1518 bp, Artificial Sequence
  Description of Artificial Sequence Synthetic polynucleotide
atggccgcagcattgataccagacttagcgatggaaacctggttgttgcttgcggtgtctttagtcctactgtatctatacggtactcatagccatggtctgttcaaaaagttaggtatccccggtccaacgccgctacccttccttggtaatattctgtcttatcataagggtttttgcatgttcgatatggagtgtcataagaagtacggtaaggtatggggattttatgacggtcagcagccagtcttggcaataacagacccggacatgatcaagacagtccttgtaaaagagtgttatagcgtgtttacgaacaggagaccgttcgggccagtgggcttcatgaagtccgcaatttctattgcggaagatgaggagtggaaaaggcttcgtagtcttttgagccctacatttacgtctggaaaattgaaggaaatggtccctatcattgctcaatacggagatgttctagtgaggaatttaaggagagaggctgagactggaaagccggttacactaaaagacgttttcggcgcgtactctatggatgtcatcacctctacatctttcggggtaaacatcgacagtctgaataacccgcaagacccctttgttgagaacacaaagaaattactgagattcgactttttggacccgttctttctgtccattactgtattcccctttttgattccgattctggaagttttaaatatttgtgttttcccgcgtgaggttacaaatttcctaaggaaaagtgttaaaaggatgaaggagtccagactggaagatactcaaaagcatagggtagatttcctacaattaatgattgactcacagaatagtaaggagaccgagagccacaaggcccttagtgatcttgaattagtcgcacagtcaattattttcatatttgcgggctacgagacaaccagctcagttctatcatttataatgtatgaactggccacccaccctgatgtgcaacaaaaacttcaggaagagatcgatgcagtccttccaaataaagctccacccacctatgataccgttttgcaaatggagtatcttgacatggttgtaaacgaaaccctgcgtttgtttcctatagcaatgagattggaacgtgtatgtaagaaagacgtggagataaatggaatgtttattcctaaaggtgtggtcgttatgattccctcatatgccttacatcgtgatccaaaatattggacggagcctgaaaaatttctgccagagaggttttccaagaaaaacaaagataatatagatccctacatctatacaccctttggcagcggtccgaggaattgcattggcatgcgttttgctttaatgaatatgaagctggccttaattagggttttgcaaaatttctctttcaaaccgtgcaaggaaactcagataccattaaaactttcattaggaggcctacttcaacctgagaaacctgtggttttaaaagttgagagtagagacggtacggtgagtggcgcttaa

SEQ ID NO: 7 - DNA, 1530 bp, Artificial Sequence
  Description of Artificial Sequence Synthetic polynucleotide
atggccgcagatctaatacctaatctagccgtagagacctggcttctgttaaccaaattggagtttgggttctacatatttccgtttatctacggtactcatagccatggtcttttcaagaaactgggcattccaggcccgacgccattgccgttcctgggtaatatcctatcatacagaaaaggcttctgcatgtttgacatggaatgccacaagaagtatgggaaggtatggggcttttacgatggcagacaaccagttctggcaattacagacccggacatgataaaaacggttctagtaaaggaatgttattctgtattcactaataggcgtcctttcggcccagtggggttcatgaaatctgcgatatctatcgcggaagatgaagagtggaagagaataagatctttacttagccctacattcactagtggcaaattgaaggagatggttcctattattgcccagtacggagacgtcttagtacgtaatcttagaagagaagccgataccggtaagcccgttacactgaaggacgtcttcggagcatacagtatggacgtgatcacatctacttctttcggtgtaaacatagactccttgaacaatccccaagatcccttcgttgaaaacactaagaaactactgagatttgactttttggaccctttctttctatctattatagtctttcctttcttgattccaattctggaggtactgaatatctgcgtatttcctcgtgaagtcacaaacttcctaagaaagtcagtcaagaggatgaaggaaagccggctagaagacactcaaaagcatagggttgactttcttcagttaatgattgattctcaaaactccaaagaaactgagagtcacaaagctctatcagatctggagttagtggcgcagtccataatttttatctttgccggttacgagaccacaagttccgtgctgtcatttatcatgtatgagctggctacccacccagatgtgcagcaaaaactacaggaggagatcgatgcagttttacccaataaggcaccgcccacgtatgacacagttctgcaaatggagtacctggacatggtggtcaatgagacgcttcgtttgttcccagttgctatgaggttggagagggtgtgcaagaaggatgttgagataaacggtatgtttatcccaaagggcgttgtcgtgatgataccaagctacgcacttcaccgtgatcctaaatattggactgagcctgagaaatttttacctgaacgttttagtaagaaaaataaagataacattgatccctatatctacacgcctttcggaagcggaccccgtaattgtataggaatgaggttcgctcttatgaatatgaaattagccctaatacgtgtgctacaaaacttcagcttcaagccatgcaaggagacacagattcccctaaagctgcgtcttgggggtttgctacagccggaaaaacctatcgttctaaaagtcgaaagtagggatggaacagtgtccggggcataa

SEQ ID NO: 8 - DNA, 1491 bp, Artificial Sequence
  Description of Artificial Sequence Synthetic polynucleotide
atggccgcagcacttatacccgatttagcgatggagacgtggttactactagcggtgtcactggtgctgctgtacctatatgggacccatagtcatggactgttcaaaaagttgggcattcccggaccgacgccgctaccctttcttggtaatatttggtcttatcgtaaaggattctgtatgttcgacatggaatgccataagaagtatgggaaagtttgggggttctatgatgggagacagccagttctagctatcactgatcccgatatgattaaaacagttcttgtaaaagagtgttatagtgtcttcacaaaccgtaggcctttcggcccagtcggctttatgaagtctgccatatccattgctgaggatgaggaatggaagagactgagatcccttttgtctccgacctttactagcggcaagttgaaggagatggtaccattgatcgcacaatatggcgacgtacttgtccgtaacctgcgtttagaggccgaaacgggcaaaccggttacgatgaaggttattacttctacaagtttcggggtcaatatagactcactgaataacccacaagatcctttcgtagagaatactaaaaagttgctgagattcgatttcctagacccctttttcctgtctattattgtctttcctttcttgacgcctatacttgaagtattgaacattagtgtgttcccgagggccgttacttcattcttgcgtaaaagtgttaagagaatgaaagagtctaggcttgaagatactcagaaacatcgtgtggacttcttacagctaatgattgactcccaaaatagtaaggagactgagagtcataaagcgttaagcgacttggaattggtagcacaaagcataatcttcatctttgctgggtacgagacgacttccagcgtgctgagttttataacatacgaattggcaacgcacccggacgttcagcaaaaacttcaagaggaaatagatgccgtcttgccgaacaaggcacccccgacttatgatacagtgttgcaaatggagtacctagacatggtagtcaacgagacacttaggttatttcctatagccatgaggttagagagagtctgcaaaaaggacgtagagattaatggtatgttcatcccgaaaggagttgtagtaatgatcccttcctacgccctgcaccacgaccctaagtactggaccgaacccgaaaagttcctgcccgagcgtttctctaagaaaaataaagataatatcgatccctatatttatacaccattcggctctggaccaaggaactgcattggcatgcgttttgccctgatgaatatgaagctggcgctaataagggtactgcagaatttttcctttaaaccgtgcaaggaaacccaaatacctctaaagttacgtctgggaggtctgctacaaccggaaaaacccattgtcttgaaagtggaatccagagatggcaccgtttctggggcgtaa

SEQ ID NO: 9 - DNA, 1530 bp, Artificial Sequence
  Description of Artificial Sequence Synthetic polynucleotide
atggccgcagagttaattccgtccttttctatggaaacttgggtacttctagcgaccagtttggtcttgttatacatatacggtacatattcttatggtctatttaaaaagttaggcattccgggcccgcgtcccgtaccctattttgggtctactatggcctatcataaggggattccggagttcgataaccagtgttttaagaagtatggcaaaatgtgggggttttatgaaggccgtcagcctatgctggcaatcacagacccagatataattaaaacggtactggtaaaagagtgttactctgtattcactaacagacgtatcttcgggcctatgggaataatgaaatacgccatttctctagcatgggacgagcaatggaagcgtatcagaaccttattatccccggcgtttactagcggcaagttaaaagaaatgttccctattatcgggcagtacggagatatgttggttaggaaccttcgtaaggaagccgagaaaggtaaccccgttaatatgaaagatatgtttggagcctactcaatggatgttatcacagggacggctttcggggtgaacattgatagtttgaataatccccacgaccccttcgtggagcattccaagaatcttctaaggttcaggcccttcgacccatttatcttgagcattatcttatttccgttcctaaacccggtgttcgaaatattaaacattactctgtttccgaagagcactgtcgatttctttactaaatctgtcaagaagatcaaagaatccagactaaccgataagcagatgaatagggtggatctgttacagttaatgattaactctcagaactcaaaagaaatagataaccacaaagcccttagcgacatcgagctagtggcccaatctaccatctttatctttggaggttatgaaaccacaagctcaacattgagctttattatctacgaactgacaacgcatcctcatgtacaacagaaggtacaggaagaaattgacgcaacatttccaaacaaggcaccacccacctatgatgcgttggtacagatggagtacctagatatggtagtgaacgaaactttgcgtatgtttcctatagctgggcgtctggaaagggtctgcaagaaggacgtcgaaattcacggggtgacgattcctaagggaacgaccgttctagtacctttatttgtcctacacaacaacccagagctttggcctgaacccgaggagttcaggcctgaaaggttttctaaaaacaataaggacagcatcaacccgtatgtgtacctaccatttggcacaggtcctcgtaattgcctgggtatgcgttttgcgataatgaatatcaaattagctctagtccgtattttacagaatttctcatttaaaccatgcaaggagacgcagattcctctgaagttgtatactcaggggttgactcaacccgaacaaccagtgatcttgaaggtggttccgcgtggtcttggcccgcaggttgaacccgacttcctttaa

SEQ ID NO: 10 - DNA, 1485 bp, Artificial Sequence
  Description of Artificial Sequence Synthetic polynucleotide
atggccgcagattcttttccactgctggcggcattgttcttcatcttagctgctacatggtttattagcttccgtagaccgaggaacctacccccaggtccattcccttacccaatagtaggaaacatgttgcaacttggcacacaaccacacgaaacgttcgcaaaactttccaagaagtatgggccactaatgtcaatccacttgggctccttgtacaccgtaatagtcagcagcccagagatggctaaggagattatgcataagtacggccaagtcttctcaggccgtacagtggcgcaggcggtccacgcgtgcgggcatgataagatcagcatgggctttctgccggtagggggtgagtggcgtgatatgagaaagatttgcaaagagcaaatgttctcacaccaatcaatggaggattcacaatggctgcgtaagcagaaattacagcaactactagaatatgctcagaagtgctcagagaggggtagagccatcgacattagggaggcagcgtttatcaccactttgaacttgatgtccgccactttgttctccatgcaggcgaccgaattcgattccaaggtaactatggaatttaaggagattatagaaggagtcgcctccattgtgggtgtaccaaacttcgcagattattttcctattttacgtcccttcgacccccaaggggttaaaaggcgtgccgacgtatacttcggaagacttttagccatcattgaggggttccttaatgaaagggtggagagtaggaggacgaaccccaacgcacctaaaaaggacgacttcctggaaacgctagttgatacccttcagactaatgacaataagctaaagacggatcacttgactcatttaatgctggacttatttgtgggaggttcagaaactagcacaaccgagatagagtggattatgtgggagcttctagcgaacccggaaaagatggcaaaaatgaaagctgagttgaagtcagtgatgggtgaagagaaggttgttgatgaaagtcagatgccacgtttgccatatttacaggcagttgttaaagaaagcatgaggttacatccaccaggtccattgctattacctagaaaggccgagtccgaccaggtcgtaaatggctatctgattccgaaaggggcgcaggtactgatcaatgcctgggcgattggaagggaccactcaatctggaaaaacccggactcctttgaaccggaaagattcttagatcagaaaattgattttaagggcaccgattacgaacttatccccttcgggagtggcaggagagtctgtccaggaatgcctcttgctaataggatccttcacacagtcactgccacgctagtacataatttcgattggaagcttgagcgtccggaggcctcagacgctcataggggcgtgctgttcggctttgctgtaaggagagcagtccctctaaagattgtgccttttaaggtg

SEQ ID NO: 11 - DNA, 1482 bp, Artificial Sequence
  Description of Artificial Sequence Synthetic polynucleotide
atggcagccgatcccttccctctggtagcagcggcattattcatagctgcaacatggttcattaccttcaaaaggagacgtaatcttccgccggggcctttcccttacccgattgtgggcaatatgttgcaactaggttcccaaccacacgagacatttgccaagctatccaaaaagtacgggccattaatgtcaattcaccttggaagtttatataccgtaataatatcctcccccgaaatggccaaagagataatgcacaagtacgggcaagtcttttctgggagaacaatagctcaggctgtgcacgcatgcgatcacgataaaatatctatgggctttttacctgtgggagcagagtggcgtgacatgaggaagatctgcaaggaacagatgttctctcatcaaagcatggaagatagtcagaacttacgtaaacagaaacttcagcaattgctggaatatgctcaaaaatgcagtgaagaaggaagaggaatcgatatacgtgaggcagcttttattactacattaaacctgatgtctgccacgttattcagcatgcaagccactgaattcgatagtaaagtcactatggagttcaaggaaataatcgaaggagtggcgagcatcgtgggcgtcccaaattttgcagattatttccccattctgcgtcctttcgaccctcaaggggttaagcgtcgtgcggatgtctactttggaagattattaggcttgatcgaaggttatcttaacgaaagaattgaattcagaaaagccaaccccaatgccccaaagaaagacgattttttagaaaccctggtggacgcacttgatgcgaaggattacaaactaaagactgaacaccttactcacctgatgctagacctattcgttggggggagcgagacgagcaccactgaaattgagtggatcatgtgggagttactggcatcacctgagaagatggccaaagtcaaagcagaattgaaaagtgtaatggggggcgaaaaggtcgtggacgagtctatgatgcctagattaccttatctgcaagcagtggttaaagagtcaatgaggttacacccgccaggcccattattacttccaagaaaagcggaaagtgaccaggtcgtaaacggttatttgattcctaagggagcgcaagtactgatcaatgcgtgggcgatgggtagagacccaagcctatggaaaaaccctgactcttttgagccagagcgttttttagaccagaagatcgactttaagggtacagattacgaacttatcccgtttggaagtggcagaagggtgtgccctggaatgcccctggcgaacagaattcttcatacggttactgctactcttgtgcataactttgattggaaattggaaagaccggaggcaagcgacgcgcacaagggagtcctttttggtttcgcggtcaggagagctgtacctttgaagatcgtccctatcaaggca

SEQ ID NO: 12 - DNA, 1491 bp, Artificial Sequence
  Description of Artificial Sequence Synthetic polynucleotide
atggcggcagacagcttcccgttactggcagcacttttctttatcgcagcaactataactttcctgtctttcaggcgtagaagaaacttgccgcccggaccatttccctaccctattgtaggtaatatgctacaactgggtgcaaatccacaccaagtcttcgccaaactttcaaaaagatatgggcctctgatgagtatacatctgggaagcttgtatacggttatagtgagttcccctgagatggcgaaggaaatattacataggcatgggcaagtgttctctggtcgtactattgcccaagctgtccacgcttgcgatcatgacaaaatatctatgggtttccttccagtagccagcgaatggagggacatgaggaaaatttgcaaggagcagatgttcagcaatcaaagcatggaggctagccagggacttcgtaggcagaaactacaacaacttttggatcatgtacagaaatgctccgatagtgggagggccgtcgacattagagaagctgctttcataaccaccttgaatcttatgtccgccacactgttcagctcccaggccaccgagttcgattctaaggctaccatggaatttaaggagattattgaaggtgtagccaccatcgtaggcgtacctaacttcgcagattacttcccaattcttaggccctttgatccccagggagtgaaacgtagggccgacgtatttttcggaaaactgttagccaaaattgaaggctatttaaacgagagattagaatccaagagggcaaacccgaatgcgccaaaaaaggacgatttcttggagattgtcgtcgatattatccaggcaaacgaatttaagttaaagactcaccactttactcacttgatgttggacttatttgttggcggctcagacacgaatacaacgtccatcgagtgggcgatgtctgagttagtaatgaaccccgacaagatggcgcgtttgaaggctgaactgaaatctgtggcaggggatgaaaaaatagttgacgagtcagccatgccaaagttgccttacttgcaggccgtcataaaggaagttatgcgtatacaccctccggggccgttgcttttgcctcgtaaagctgaaagcgatcaagaagtgaatggataccttattccgaaaggcacacagatcctgataaatgcatatgcgataggacgtgatccatcaatctggactgaccccgaaacatttgacccggaacgtttcctggataacaagattgatttcaagggacaagattacgagctgttgccgttcggctccggacgtcgtgtatgtccggggatgccgttagctactagaatacttcacatggcaaccgccacgctagtccacaacttcgactggaaattagaagacgatagtacagccgctgcagaccacgcaggcgagctatttggagttgcagtgagaagggcagttccgcttcgtataatcccgatagtgaagtcc

SEQ ID NO: 13 - DNA, 2040 bp, Artificial Sequence
  Description of Artificial Sequence Synthetic polynucleotide
atggccgcaggagattctcatgtggatacgtcgtccactgtgtctgaagctgttgctgaagaggtatctttattcagtatgactgacatgatcctattcagccttattgtggggctactgacctactggttcctattcagaaagaagaaggaagaggtcccagagttcaccaagatccaaacgctgacttccagcgtccgtgaatcatcctttgttgagaaaatgaagaaaacagggcgtaatatcattgtgttttatggtagtcagactggcactgcagaagagttcgccaacaggttgtctaaagatgcgcacagatatggtatgaggggtatgtctgcggacccggaggaatatgacttagcggacttgtcctctctgcctgaaattgataacgcgctagtggtcttctgtatggctacgtacggagaaggcgatccaaccgataatgcccaagacttctacgattggttgcaagagactgacgtggatttgtccggagtaaagttcgcagtatttgggctgggaaacaaaacttatgaacattttaatgcaatgggtaagtacgtagacaaacgtttagaacaactgggtgcacaaagaatattcgagcttggtttgggggatgatgacggtaatttagaggaggatttcatcacttggagggagcaattctggccggcggtgtgcgagcatttcggcgttgaggcgaccggagaagagtcaagcattagacagtatgaacttgttgtgcacacagatatcgacgccgcaaaagtgtatatgggggagatgggtagattaaaatcttatgagaatcaaaaacctcctttcgatgcgaaaaatccattccttgccgctgtgaccacaaacaggaaactaaatcaaggtacagagcgtcatttgatgcacttggagctagacatcagtgattctaaaattaggtacgaatcaggggatcacgtcgccgtttatcccgccaatgatagcgccttggtgaatcagttaggtaagatactgggtgctgatttggatgtagtcatgagcttgaataaccttgatgaagagtccaataagaaacatcctttcccgtgcccaacaagttatagaaccgcccttacgtactacttagatatcaccaatccaccaagaacgaatgtcctatatgagcttgctcaatatgccagtgagccatccgagcaagagctgcttaggaaaatggcgtcctcatccggtgaaggcaaagaattatacctgtcctgggtggtcgaggccaggaggcatattttagctattttgcaagattgtccttcccttaggccgcccatcgatcatctttgtgagctgcttcctcgtttacaagcaaggtattattctatcgcgtcctcctctaaagtccatccaaacagcgtacacatctgtgccgtggtggtcgagtacgagacgaaggccggtagaatcaacaagggcgttgctacaaactggttgagagccaaggagcccgcgggggaaaacggaggtcgtgcattagtaccgatgtttgtccgtaaatctcaattcaggttgccttttaaggcaaccactccggtaatcatggtcgggcctggcactggcgtagccccatttataggattcattcaggaaagggcctggttgaggcaacaaggcaaggaggttggagagactctgctgtactacggatgccgtaggagcgacgaagattacttgtatcgtgaagagcttgcacaatttcaccgtgacggagcacttactcaattaaatgtggcttttagtcgtgaacagtcacataaggtgtatgtacaacatttattgaagcaagaccgtgaacacctttggaagctgattgaaggtggcgcccatatttatgtatgcggcgatgctcgtaatatggcaagggacgttcaaaacactttctatgacatcgtcgcagaacttggggcgatggagcatgctcaagcagtagattacatcaagaaactaatgaccaaaggtagatattcacttgacgtttggtcctaa

SEQ ID NO: 14 - DNA, 2082 bp, Artificial Sequence
  Description of Artificial Sequence Synthetic polynucleotide
atggccgcacctttcggtattgacaatactgacttcactgtcttggcgggcctggtactggcagtactgttgtatgtgaagaggaacagtataaaagaactgttgatgtcagatgatggtgacatcacagctgtttctagtgggaacagagacattgcccaggtcgtcacggaaaataataaaaactatctagtcctgtatgcttcacagaccggcaccgcagaagattatgcgaagaaatttagcaaggagctggtagccaagttcaaccttaatgtgatgtgcgctgacgtagagaattacgacttcgaatccttaaacgatgtaccggttatcgtttctatcttcatttccacctacggagagggcgattttccagacggtgcggttaacttcgaagactttatatgtaatgctgaggctggagctttaagtaacttacgttataatatgtttggtcttgggaactctacttatgagttctttaatggcgctgccaagaaagccgaaaaacacttatctgcggcgggggccatcagactgggcaaacttggagaggccgacgatggtgccgggacaacggacgaggattatatggcttggaaggattctatattggaggttctaaaggatgaactacacttagatgagcaggaagccaaatttacttcccagtttcagtatactgttctgaacgaaataacagactccatgtctttgggcgaaccgtctgcgcattacctacccagccatcaactgaacagaaacgcggacggaatacagctagggccctttgacttatcgcagccttacattgccccaattgtaaaatctagggagctgtttagttctaatgataggaattgcatacatagcgagttcgacttgtccggttctaatattaagtactctacaggtgaccacctggccgtatggccgagcaatccccttgagaaggtagaacaatttttgtcaatcttcaacctagatccagaaacgatattcgatttgaagcccctggacccgactgttaaggtaccgtttcccactcctaccaccataggtgcggcaatcaagcactatttggaaatcacaggcccggtatcacgtcaattgtttagtagcttaattcaattcgccccgaatgctgacgtaaaggagaaactaaccctgctaagtaaggacaaggaccaattcgctgtggaaattaccagtaaatatttcaacatagcggatgctttaaagtatctgagtgatggggctaaatgggacactgtgcccatgcaatttctggtggagtccgtgccccaaatgacccccaggtactacagtatcagttcatccagcctaagtgagaagcagacggtccatgtaacaagcatagtagagaatttcccaaatcccgaattaccggatgcgccccctgtcgtgggagtgacaaccaatcttctaaggaatatccaactagcccaaaacaatgtgaatatcgcggaaacgaacctacccgttcactacgatcttaatggacccaggaaactttttgcaaattacaaacttcccgtccatgttaggagatcaaattttaggctaccttccaatccaagcactccagtgatcatgattggaccgggtactggagttgcgcctttccgtgggttcattagggaaagagtagcctttttggagagtcagaagaaaggcggaaataatgtcagcttgggcaaacacatattgttttacggttcacgtaacaccgacgacttcctttaccaggatgaatggccagagtacgctaagaaactagacgggtcttttgagatggttgtggcccactctaggcttccaaacacgaagaaggtctatgtacaggataagctgaaagactatgaggatcaagtttttgaaatgataaacaacggggcgttcatttatgtttgcggagacgcaaaagggatggctaagggtgtgagcacagccttggtcggtatcttatcaagagggaagtcaataactacagacgaagccactgagctaattaaaatgcttaaaacgagcggaaggtaccaagaggacgtttggtaa

SEQ ID NO: 15 - DNA, 2043 bp, Artificial Sequence
  Description of Artificial Sequence Synthetic polynucleotide
atggccgcaggagacagccacgaagatactagtgcgaccgttccggaggcagtggcggaagaggtgagcctattcagtactaccgatattgtacttttctccctaattgtgggtgtgctgacttactggttcatatttaaaaagaagaaagaggagattcccgaattttccaaaatccaaacgacagctccacccgttaaagaaagtagtttcgtcgagaaaatgaagaagactgggaggaatataatagttttctatggaagccaaacagggaccgcagaggagttcgcgaacagactaagtaaagacgctcatagatacggtatgcgtggtatgtccgctgacccagaggagtacgacctggcagacctaagctcactgccagagattgacaaaagcctagtggtcttctgtatggctacatatggtgaaggtgatccaactgataacgctcaggatttctacgattggttacaagagacagatgtggacctgactggagttaaatttgcagtcttcggcttggggaataaaacatacgaacactttaatgctatggggaaatacgtcgatcaaagattggagcaacttggcgcccagagaattttcgagctaggcttgggagacgacgatgggaatcttgaggaggattttataacttggagagaacagttttggccagccgtgtgcgaatttttcggagtcgaggcgacaggcgaagagtcaagtatcaggcaatatgagctagttgtgcatgaagatatggacacggcgaaagtctacaccggcgagatgggacgtctaaaaagttacgagaaccaaaaaccgccttttgatgcgaagaatccattcttggccgccgtcacgacaaaccgtaagttaaaccaagggactgaaagacatctgatgcacttagagcttgacatctccgatagtaaaataaggtatgaaagtggagatcacgtcgccgtatacccggctaacgattcaactctagttaatcagatcggggaaatattaggggccgacctagacgtcataatgagtttaaacaacctagatgaagaatcaaacaagaaacacccattcccctgtccaaccacttacaggacagcgttgacttattatcttgatatcaccaatcccccgagaaccaacgtgttatatgaacttgctcagtatgccagtgaaccatctgagcaggaacatctgcacaagatggcatcctcatcaggagaaggaaaagaattatatctgtcctgggtcgtggaggctaggagacatatccttgcgatcctgcaggactatcctagcttgcgtccgcctatcgaccatttatgcgaactgctacctcgtttgcaggccaggtactacagcatagcctctagtagtaaagtacatcctaactctgtgcatatatgtgccgtggccgtggagtacgaggctaaatcaggaagagtaaataaaggagtcgcaacgagttggctgaggactaaggaaccagccggcgagaacggtaggagagcactggtgcccatgtttgtgaggaagtcccaatttcgtctgccatttaagcctacaaccccggtaattatggtcggtcccgggacaggtgtagctccgttcatgggatttatccaagagcgtgcctggttgagggaacagggtaaagaagtcggagagaccttattatactatggatgtaggcgttcagacgaggattatttataccgtgaggagttagcccgttttcacaaagacggggccttaacccagcttaatgtagctttttctagggagcaagcgcataaggtctatgttcaacacttgcttaaaagggataaagaacacttgtggaagctaatacacgaaggaggagcccatatctatgtttgcggagatgccaggaacatggccaaggacgtacaaaataccttttatgatattgtcgcagaatttggtcctatggagcacacacaagctgtagactatgttaagaaactaatgacaaaaggcaggtacagtctggatgtctggtcttaa

SEQ ID NO: 16 - DNA, 2043 bp, Artificial Sequence
  Description of Artificial Sequence Synthetic polynucleotide
atggccgcaggtgattcccatgaggacacttccgctactatgccggaggccgtagcggaggaggtctcattgttttccacgactgacatggtcctgttcagcctgatcgtcggcgttttgacgtattggttcatatttagaaagaaaaaggaagaaatccccgagttctccaaaatccaaaccactgcccctccggtaaaagaaagctcttttgttgagaagatgaagaagacaggccgtaatatcatcgtgttttatggtagccagactggtacagccgaggaatttgcaaacagactgagcaaggacgcgcacaggtacggtatgcgtggcatgtccgccgatcccgaagagtatgatctagccgacctgagcagcttaccggaaatcgataaatcccttgttgtcttttgcatggcgacctatggagagggcgatccgaccgataacgcacaggacttctatgattggttgcaagagacggacgtagacctgacaggcgtgaagttcgccgtcttcggactgggcaataaaacatacgagcacttcaacgcaatgggcaagtatgtggatcagcgtttagagcaactaggcgcccaaaggattttcgagttgggtctgggagacgacgatggaaacctagaagaagatttcataacctggcgtgagcaattctggcctgcagtatgcgagttctttggtgttgaggccacgggcgaagaatcatctataaggcagtatgaattggtcgttcacgaagatatggacgccgcgaaggtatacaccggcgaaatggggcgtcttaaatcatacgaaaaccaaaaacctccctttgacgctaaaaatccatttctagctgctgtcaccgcaaatcgtaagttaaaccagggcactgagaggcacctaatgcacctggagctggatatcagcgattccaaaatcagatacgaatcaggagaccacgtcgcggtgtatcccgcaaacgattcagcgttagtaaaccaaatcggggaaatacttggagcggatcttgatgtgataatgtctttaaataacctagacgaggaatccaataagaaacacccatttccctgtcctacgacatacagaaccgcgctgacgtattatttggatataacaaatcctcccagaacgaacgttctatatgagttagcccagtatgcttcagagccgagtgaacaggaacatctgcacaagatggcgagcagttcaggagagggtaaggaattatacctttcctgggtcgttgaggcgcgtagacacatacttgcaattctacaagactaccctagcctaagaccacctatagaccatctgtgcgagttattgccacgtttgcaagccaggtattatagcatcgcaagttcttctaaagttcaccccaactctgtgcacatatgtgcagttgctgtcgaatacgaagcaaaatccgggagggttaataagggagtagctacgagttggctaagagcaaaagaaccagctggtgaaaatggaggtcgtgcccttgtcccgatgtttgtaagaaaatcccaattcagactaccgtttaagagtaccacgcccgtgatcatggttggtccggggactggtatagcaccttttatggggttcatccaggagcgtgcctggctacgtgagcaaggcaaagaggtaggcgagacattgctttactacgggtgtagacgtagcgatgaagattacctgtacagagaagagttggcgagattccacaaagacggcgctttaacccaactaaacgtcgcttttagcagagaacaggctcataaagtgtacgtccagcacttgctgaaaagggacagggagcatttatggaaactgattcatgaaggtggcgcgcacatatacgtatgcggggatgctcgtaatatggctaaggatgtgcagaatacattttacgacattgtagcggagttcggccctatggagcatacgcaagctgtagattatgtcaagaaattaatgaccaaaggtagatactcattggacgtttggagctaa

SEQ ID NO: 17 - DNA, 2049 bp, Artificial Sequence
  Description of Artificial Sequence Synthetic polynucleotide
atggccgcaatcaacatgggtgactctcatgttgacacaagttccaccgtttccgaagctgtagcggaagaagtgagcctattcagcatgacagatatgattctattctcacttattgtcggtttactgacttactggtttctattcagaaagaagaaggaagaggtccccgaatttacgaaaatacaaacacttacttcctcagttagggaatcatcattcgttgagaaaatgaaaaagacggggcgtaacatcatcgtgttttatggttcacaaaccggaactgccgaagagtttgcgaatagattatctaaggacgctcatagatacggaatgcgtgggatgtctgccgatcccgaggaatatgatctggcagatcttagcagtctaccggaaatcgacaatgcacttgtggtgttctgcatggcaacatacggggaaggagatccgacggataatgcacaggacttttatgactggttgcaagagaccgacgtggatctatccggtgtcaagtttgccgtttttgggcttgggaataagacctacgagcacttcaatgcaatgggaaaatatgtggataagagactggagcagctgggagcccaaagaatattcgagttaggattaggtgacgatgatgggaatcttgaggaagacttcatcacctggcgtgaacagttttggccggcagtttgcgaacactttggtgtagaagccactggggaagagtcttccatcaggcaatacgagctggtagtgcatacagatattgatgcagctaaggtatatatgggtgagatgggaaggttaaaaagttatgagaaccagaaaccaccttttgatgccaaaaatccttttctggccgcagttacgacaaacaggaaattaaaccagggtacggaaagacacttaatgcatttagaactggacatctcagacagcaagattaggtacgaaagtggggaccacgtcgcagtgtacccggctaatgacagcgcgctagtgaatcagctgggtaaaattctaggggcggatcttgacatcgtgatgagcctgaataatttggacgaagagagcaacaagaaacatcccttcccctgtcctacttcctataggacggctctaacatattatctggacataacgaatccccctagaacgaacgtcttgtacgaattggcacagtacgcttctgaaccgtcagaacaggaattactacgtaaaatggcgagttctagtggtgaaggaaaagagttatacctaagttgggttgtagaggcaaggagacacattctggctattttacaagattgccctagtttgaggccgcctatagaccacctttgtgagttattaccgaggttacaggctcgttactattctatagcaagcagtagtaaggtccacccgaatagcgtacacatatgcgccgtagtagtagaatacgaaacaaaggcgggtcgtatcaacaaaggagtggcgaccaactggttgcgtgccaaagaaccggcaggagaaaacggtgggagggctctagttccgatgtttgtccgtaaaagtcagtttaggttaccgttcaaggccacgacccccgtaatcatggtaggtcccgggactggagttgcgccgttcataggctttatccaggaaagggcctggttgcgtcagcagggcaaggaggtcggtgaaacactgttatactatggatgcaggaggagtgacgaggactatctgtacagagaagagcttgcccaattccacagggacggcgcattgactcaacttaacgtagccttctcacgtgaacagtctcacaaggtgtacgtccaacacctgctgaagagagacagggagcatctgtggaaattaatagaaggcggagcacatatctatgtatgtggcgatgcaagaaacatggcacgtgatgtccagaataccttttacgatattgtcgcggagcttggggcgatggaacacgctcaagccgtagattacatcaagaaactaatgacaaaggggcgttattctcttgacgtatggtcctaa

SEQ ID NO: 18 - DNA, 2049 bp, Artificial Sequence
  Description of Artificial Sequence Synthetic polynucleotide
atggccgcaattaacatgggagattcccacatggatacatccagcacagtgtccgaagcagttgcggaggaagtgagtttattttccatgacagacatgatccttttctcacttattgtggggttacttacctactggtttttgtttaggaaaaagaaagaggaggtcccggaatttactaaaatacagactttgacatcatcagtacgtgagtcttcctttgtagagaaaatgaagaagacaggcaggaatataatagtattttatggatctcagaccgggaccgcagaggagttcgctaaccgtctatccaaagacgcccaccgttatggtatgaggggcatgagtgcggaccccgaggagtacgatctagccgatctatccagcttacctgagatagagaacgcattggttgtattttgtatggccacgtatggcgaaggtgatccgacagataatgctcaagatttctacgactggttacaagagaccgacgtagacttgtctggtgtaaagttcgctgtatttggattaggaaataagacctatgaacactttaacgccatgggcaagtatgtcgataaacgtctggaacaattaggcgctcagagaattttcgaactgggactaggcgatgacgacggaaacctggaggaggactttattacgtggagagaacaattttggcccgctgtctgtgaacatttcggcgttgaggcaaccggcgaagaaagctcgataaggcaatatgaattggtagtacatacagatatagacgccgctaaagtgtacatgggagaaatgggaaggttgaagagttatgagaaccaaaaacctccgttcgacgctaagaatccgttcctagcggctgtcactacaaatagaaagttgaatcaaggcacagaaaggcatttaatgcatttagagcttgatatttctgacagcaaaatcagatacgaatccggggaccatgttgcggtctacccagcaaacgacagtgccttagtaaatcagctagggaaaatacttggggcggatttggatgtcgtaatgagtcttaataacctagatgaagaatcaaataagaaacatccatttccttgtcccacaagctataggaccgcgctgacttactatctggacatcactaatcccccaagaaccaatgtgttgtatgagttagcccagtatgccagcgagacgagtgagcaagagttattgagaaaaatggcaagtagctcaggagaagggaaggagctgtatctgagctgggttgtagaggcacgtagacacattctggccattttgcaagattgcccttctttgagaccacctatagatcatctttgtgagcttctaccaaggctgcaggctcgttactacagtattgctagttcaagcaaagttcatccaaacagtgtccacatctgcgcggtcgtagttgaatacgagacaaaggccgggagaattaataaaggtgtggccactaattggctaagagccaaagaacctgctggtgagaacggtgggcgggcgcttgtcccgatgtttgtaaggaagagtcaatttcgtctgccatttaaggcgaccacgcctgttattatggtgggtccaggaacaggtgtagccccatttataggattcattcaagaaagggcctggttaaggcaacaaggcaaagaggttggtgaaactcttctttattatgggtgtcgtagatcagatgaagattacctgtatcgtgaagagcttgtacaattccatagagacggcgcgcttacacaattgaatgttgcttttagtcgtgagcaaagtcataaagtctacgttcaacatttattaaaaagggatagagagcacttgtggaaactgatagaaggaggggcgcatatctatgtatgcggagacgcgaggaatatggctagagacgtgcaaaataccttttatgatatcgttgcagaattgggtgcaatggagcatacgcaagccgttgattacataaagaagttgatgaccaaaggacgttacagcctagatgtttggtcttaa

SEQ ID NO: 19 - DNA, 2049 bp, Artificial Sequence
  Description of Artificial Sequence Synthetic polynucleotide
atggccgcaaatatggcagacagtaatatggatgcgggaacgaccacatctgagatggtagcagaggaggtaagcctattttccactacggatgtgatattgttcagtttgatcgtaggtgttatgacttattggtttcttttcaggaaaaagaaagaagaggtgccggagttcacaaagattcagaccactacctcctccgttaaggatagatcatttgttgaaaagatgaagaaaaccggcaggaatatcattgtgttctacggtagccagacaggaacggcggaagagttcgccaaccgtctatccaaagacgctcacagatatggtatgaggggaatggcggccgacccggaggagtacgacctggctgatctgtcttctttgccagaaatagagaaggcgttggctatattctgtatggcaacctatggagaaggggacccaacagataacgctcaggacttttacgattggttgcaagagactgatgtggacctaagtggtgtaaagtatgcggtatttgccctggggaacaagacgtacgagcacttcaatgcaatgggtaagtacgtagataagagactggaacagttgggagcgcaaaggatattcgacttgggattaggtgatgacgacggaaacctagaggaagatttcataacctggagggaacaattctggcccgccgtttgtgagcattttggcgttgaagcaacgggtgaagagagttctatccgtcaatacgagttgatggtacatacggatatggatatggcaaaagtttacaccggtgaaatgggaagactaaagtcatacgaaaaccagaagcccccatttgatgcgaaaaaccctttcctagcagtcgtaacgacgaaccgtaagctgaatcaggggacggagaggcacttaatgcacttagaacttgatatatctgactctaaaattagatatgaatctggggaccatgtagccgtatatccagcaaacgatagcgccttagtaaaccaactgggcgagatattgggggctgatctggatatcataatgagtttgaacaacttggatgaagaaagtaacaagaaacatccgttcccttgtcccacatcatatagaacagcccttacctactacttggacataacaaacccgccaagaactaacgttctatacgagttggctcaatatgcgagcgaaccgaccgaacatgaacaactacgtaaaatggcatcttcatctggtgagggtaaggaactttacttgaggtgggtgcttgaagctagaaggcatatcctggcgattttgcaagactatcctagtctaagaccgccgattgatcacttgtgtgagcttcttcctagacttcaagctaggtactactcaatcgccagcagttcaaaagtgcacccgaactcagtacatatatgtgccgtagccgtcgagtacgagaccaagacgggcaggataaacaaaggagtcgccacaagttggctgagagctaaggaaccagcaggcgaaaatggtgggcgtgccctagtgcctatgtatgtgcgtaaaagtcagttcagattaccgttcaaagctacgacccctgtaataatggtaggcccgggcactggagtggcccccttcattggattcattcaagagcgtgcctggctaaggcaacaggggaaggaggttggggaaacattgttgtattatgggtgcaggagaagcgacgaggattatctatatagggaagagctggcgggcttccataaggacggcgcgctgactcaattaaatgttgcattcagcagggaacaaccccaaaaggtgtacgtgcagcacttacttaaaaaggataaggaacacttatggaaattaattcacgagggtggagcccacatctacgtgtgtggggatgccaggaatatggctagggatgtccagaacacattctatgatattgtagccgaacaaggtgcgatggagcacgcccaagctgttgactatgtcaagaaattgatgaccaaagggagatactctctggacgtctggtcataa

SEQ ID NO: 20 - DNA, 2127 bp, Artificial Sequence
  Description of Artificial Sequence Synthetic polynucleotide
atggccgctgaacctacctcacaaaagcttagtcctcttgacttcattgcggccattctaaaaggtgattattcagatgggcaactggaagctgcatccccggggatggctatgctgctggagaatagagatcttgtaatggttcttacaacaagtgtggctgtattgatagggtgtgtggtggtgttagcctggagacgtacggccggttcagccaccaaaaagcagtttgagcctcccaagcttgtagtaccgaaagccgcagaactggaggaagttgatgacgacaaacctaaggtaagtatcttctttggtacccaaaccggaaccgcggagggctttgcgaaagctttcgccgaggaggccaaggccagatatccccaggctaacttcaaggtgatcgacttagatgattacgcagcagacgacgacgagtatgaggagaaactgaagaaggagacgctggcgttcttctttctggcgtcctacggcgatggagagcccacagacaacgcggctagattctacaagtggttcactgaggggaaggatagaggtgattggttgaaaaatttacaatacggagtgtttggtctaggcaatagacaatatgagcactttaataaaatcgctatcgttgtgaatgacatcattgtcgagcaaggtggaaaaaagctagtgtcagtgggccttggggacgacgatcagtgcatagaagacgacttcgccgcttggagagaattagtatggccagaacttgacaagttgcttcgtaacgaagacgatgccaccgtctctacaccatacaccgcagcagtactgcaatatagggttgtctttcatgatcagacagacggcttaatctccgagaacggcttcttgaacggcagagcgaacgggacgtctgtcttcgatgcccaacacccctgtcgttccaacgtcgcggtcaaaaaagaacttcacacccccgctagtgaccgtagttgtgcccatcttgaatttgatatatctgggactgggttagtctatgaaactggagatcatgttggagtttactgcgaaaacctaattgaaactgtagaggaggccgaaaagttgttaaatattccccctcaaacatatttttccatacatacggataatgaggatgggtcccctcgtagcggcagctctcttccgcctccattcccgccatgcactttaagaacggccttgaccaggtatgcggatttgttgtccgcgcctaaaaaaagcaccttaattgctctagcagagagtgccagcgatcagagtgaagccgacagattacgtcaccttgcgagccccggtggtaaggaggaatacgctcaatatatcaccgcaagccaaaggtccctgctagaggtaatggcggacttccccagtgcgaagccttcattgggcgtctttttcgcagccatagccccccgtttgcagccccgtttttactctatctcaagctcaccgaaaatagcacctagcaggatacatgttacgtgcgcgctggtttatgaaaaaacgcctacgggtcgtgttcataagggtgtctgcagtacatggatgaagaacgctgtgcccctggaggaatctaatgactgtagctgggcgccgatatttgtcaggaactccaacttcaagctacctgcctatcccaaagtgcccataattatgattggccctggaactggtctggccccgttcagaggttttctacaagagcgtcttgcgttaaaagaatcaggtgccgaattgggaccagctatactattcttcgggtgtaggaatagaaaaatggacttcatctatgaagatgagctaaacaacttcgtagaggcgggcgttataagtgaactgatagtagcgtttagtagggagggaccaactaaggagtatgtacaacacaagatgactcagagggcgtcagatgtatggaagatcataagcgatggaggttatgtttatgtgtgcggcgacgcgaaaggaatggcaagggatgttcaccgtacactacatacaatagcacaagagcagggcagcctttcttcatctgaagcagagggaatggtgaaaaatctacagacaaccgggcgttatctgagggacgtatgg

SEQ ID NO: 21 - DNA, 753 bp, Artificial Sequence
  Description of Artificial Sequence Synthetic polynucleotide
atggccgcagacttctcaggtaaaaacgtttgggtcacgggggccggtaaaggtataggttacgcgacagcattggcattcgtagaggcaggggccaaggtcacaggcttcgatcaggcatttacacaggaacagtacccttttgccaccgaggttatggatgtagcggacgccgcccaagtagcacaagtctgtcagcgtctactagctgagacagagagattggatgctctggtgaatgcggcaggtattcttcgtatgggtgccaccgaccaattatctaaggaggactggcaacaaacgttcgctgtaaatgttggaggtgcatttaacctgttccaacagactatgaatcagttcagaaggcagcgtggaggcgctatagtcacagtagccagtgacgccgcgcataccccaaggattggaatgtcagcgtacggagcttccaaggcagccctgaagagcctagctttatcagtcggtctggagctggccgggtcaggggtaaggtgcaacgttgtgtccccgggctccacggatacagacatgcagagaactctgtgggtgtctgacgacgcagaggaacaacggatcagaggtttcggagagcagttcaaactagggattccgctgggcaagatcgctagaccacaagagatagctaatactatacttttcctagcatccgatttagccagtcacatcactttacaagacatcgtagtggatggtggttcaacactaggcgcttaa

SEQ ID NO: 22 - DNA, 819 bp, Artificial Sequence
  Description of Artificial Sequence Synthetic polynucleotide
atggccgcaagcgatctgcataatgagtccattttcattacaggcggaggctctggtcttgggctggccttagtggaaaggtttatagaggaaggggcacaggttgctacacttgagcttagcgcagcaaaagtcgcgtctctacgtcaacgttttggagaacatatattggccgtggaaggcaacgtcacgtgttatgccgactatcaaagagctgtagatcagatactaacccgttctgggaagttagattgctttatagggaatgcaggtatatgggatcataacgcttccctggttaataccccagcagaaacgctagaaacagggtttcatgagctttttaacgtaaacgtcctggggtacttactgggagcaaaagcatgtgctcctgcgcttatcgcgtcagagggttcaatgatatttaccctttcaaacgcggcttggtacccaggtggagggggtcctttatatacggcctccaaacatgcagcaactggcctgatccgtcaactagcctatgaacttgcacccaaggtaagggttaatggagtgggtccctgcggcatggctagtgatcttaggggaccacaagccttagggcaatcagaaacgagtataatgcagtcattgacccccgaaaagattgcggcgatattacctctgcaatttttcccacaaccggcggacttcactggaccatacgtcatgttaacatctaggcgtaataatagggcactgagcggcgttatgattaacgcggacgctgggttggctatcagaggcattaggcacgtggcagcaggacttgacctataa

SEQ ID NO: 23 - DNA, 810 bp, Artificial Sequence
  Description of Artificial Sequence Synthetic polynucleotide
atggccgcaacgggatggttagcgggaaaaagagctttgatcgtcggtgcgggttccggaatcggaagagctacagttgacgcatttctaaacgaggacgcgagagttgcagttctggagtatgactccgataagtgtgcaacacttaggcaccagttaccagacgttcccgtgatagaaggcgatgggaccacaaggaccgctaacgatgaggccgttcaggtcgctgtggacgcattcgggggactagatactctggtcaactgtgttggaatattcgacttctaccgtcgtatccaagacattcccgcagagctgatcgatcaggcatttgacgaaatgtttagaatcaatgtattatcacatatccactctgttaaagcagcggtacctgctctgatgggtcaggacggagcatctattgtgctgacggagagtgcttcttcattctatcccggtaggggcgggttgttgtatgtggcgtcaaaatttgctgttcgtggtgtcgtaaccgcactggcccatgagttggctcccaggattcgtgttaatggagtagctcctggcggaacccttaatacagatctgaggggccttgacagtttggaccttggtgcccgtaggttagatgccgcgcctgacagagctagagaacttgcagcgaggaccccactgggggtcgcattgtccggtgaagaccacgcctggtcttacgttttcctggcctctcataggagtagaggtctaacaggcgaaacgattcaccctgatggcggcttttctttaggaccgccgccacaaaggaattaa

SEQ ID NO: 24 - DNA, 804 bp, Artificial Sequence
  Description of Artificial Sequence Synthetic polynucleotide
atgagtagtatcgagaccaaaatctttcctgggcgttttgatggtaggtgtcttaccataacaggtgccgcccaaggcattgggttgacagtagctacgaggatagcggcagaaggcggtgaagtggtgcttgttgaccgtgcagaccttgtacacgaggtggcagagcagctacgtgaggcaggaggcaaggcgcactcagtaacggctgatttagaaacatttgagggtgctgaggaagcgatctctcatgccgtaaggacgactggcagaatcgatgtactaatcaatgttgtgggcgggactatatgggcaaaaccgtatgagcactacgccccggaggaaatagaaaaagaaattagaagatccttattccctacgctatggacatgtagagctgcggcaccgcatttaatcgaacgtagagcaggaacgatagtgaacgtaagctccgttgctacgaggggcgtaaatcgtgttccctattccgcagcaaagggaggtgttaatgctattactgcgtctctggcgttggaattagccccgtacggggtaagggttgtcgcaacggctccaggcgggaccgtcgcgccagagagaagaatcgccagagggcctagcccacagagtgagcaggagaaagcctggtaccagcagattgtagatcagacagttgactcctcattacttaaaaggtatggtactcttgatgaacaagcagccgcgatctgcttccttgcatcagaagaggcgtcatacatcaccggaactgtcttgccggtggccggaggggacttaggataa

SEQ ID NO: 25 - DNA, 813 bp, Artificial Sequence
  Description of Artificial Sequence Synthetic polynucleotide
atgagtagtaccggctggctagacggcaaaagggccttagttgttgggggaggaagtgggataggtagagctgtcgtagacgctttcttagctgaaggagcttgcgtagccgtcctggaaagggacccgaataagtgtagagtcctaagagaacatctgccgcaggtgcccgtaattgaaggagatgcaacaagggctgcagataatgacgcagcggtagctgcagcagttgctgcatttggaggactagacacgcttgtaaattgtgtgggtatctttgacttctatcagggcatcgaggacattccggcggacacccttgacgtagcattcgatgaaatgttcagaacgaacgtactatcccacatgcatagtgtaaaggcggcagttcccgagttacgtaaacataggggctcttctatcgttctggctgaatccgcctctagcttctatccagggagagggggtgtcctatatgtctcttctaaatttgccgtcagaggtctggtaaccactctagcatacgagttggccccagatatcagggtgaatggggtcgccccaggtggtacgctgaatacggatctgcgtggcttagcgtcactaggaagggatgctgacaggctagatgataaccctaatagggccaatgagttagcagccagaactccgcttaacgtggcccttagtggggaagatcatgcgtggtcttttgtcttcttcgcttccgacagaagcaggggaattacagccggggctactcatccagatggaggctttggaattggtgcgcccaagccctctactagataa

SEQ ID NO: 26 - DNA, 822 bp, Artificial Sequence
  Description of Artificial Sequence Synthetic polynucleotide
atgagtagtgggtttctggatggcaaggttgctcttgtgactggtggcgggagtggtattggaagggccgtcgtcgaattatacgttcagcaaggagctaaagtaggtatcttagaaatctcacccgaaaaagtgaaggacctgaggaatgccctaccagctgacagtgtcgtggtaacagagggagatgctacgagtatggcggataacgagagggcagtcgcggacgttgttgacgcattcggaccccttactacgttagtttgtgtggtgggggtattcgattactttacagagattcctcagctacctaaagataaaatctctgaagcctttgatcaactttttggggtaaatgttaaatccaacctattgtctgtgaaagcggcgttagacgagctaattgagaacgaaggagacataatactgacgctaagtaacgcagctttttatgccggtggaggcggcccactgtatgtttctagtaagtttgctgtaaggggcttggtgactgagttagcatatgagcttgccccaaaagtacgtgtcaacggggtagccccagggggaacgattaccgaacttagaggaatcccggccttggcgaatgaaggacaaaggctgaaagacgttcctgacatcgagggattaatagaaggaattaatccccttggtatcgttgctcagcctgaggaccactcctgggcctacgcgttattagcaagtagagaaaggacatcagcggtaacaggcacgattataaacagcgatggaggattaggagtcaggggcatgactcgtatggccggtctggcacaataa

SEQ ID NO: 27 - DNA, 777 bp, Artificial Sequence
  Description of Artificial Sequence Synthetic polynucleotide
atgagtagtagtaggtctgtgactttggtagtcggcgctgcccaaggaattggcagggctaccgcattgacgcttgcgacggcgggtcacagggtcgtgttggcggatagggacgtagacggcttggccgagactgctgcgcttctacacgtcgctgcaccggttcacggacttgacgtatgtgatgctgctggggtggcggaagcggttgcgagggtggaggtcgagcacggaccggtagatgctctagctcatgtcgcgggggtgtttaccacgggctctgtacttgattcagacttagcagagtggcaacgtatgtttgacgtcaacgtgacggggctaatcaatgtactgcgtgtcgtggggcatggcatgagagaacgtagacgtggagcaatcgtcactgtcggttctaattccgctggtgtaccaagggtggggatgggagcttatggtgcatcaaaatccgcagcacatatgctggtacgtgtattaggattggaattagcaagattcggcgtcagggcgaatgttgttgccccagggtccacggacacagcgatgcaacgttctctttggcccgaccctgctgacgacgctggcgcccgtactgcgatagacggtgacgccgcttcatttaaggtcgggattccactggggaggatcgcagacccagccgacatcgcggacgccgtcgagttcctgctatctgatcgtgctaggcacataacaatgcagactctatatgtagatggtggtgctaccctgagagcataa

SEQ ID NO: 28 - DNA, 834 bp, Artificial Sequence
  Description of Artificial Sequence Synthetic polynucleotide
atgagtagtcaaatgctggatgaccacgtagctctgatactaggtggtgggagtggattgggtctaggaattgcgcgtcattttctcggagaaggggctcaggtggccatctttgagatcagtgaatccaaattattagacctaaaagctgagttcggggacgacgtacttcttttacagggggatgtaacatcaattgacgacctagaggcagcccgtgccgcagtagtggataggttcggaaggttggatgcacttattggtgcgcaagggatttttgatgggaacatcccattgagagacatcccgaccgagagaatcgaaaaggttttcgacgaagtgctacatgttgacgtgctaggttatatattagccgctagggtcttcctggaagagctggagaagacagacggagcaattgtgtttaccagcagtactgcggcttacgcagccgatggaggaggtttgttttacactgccgccaagggtgccgttagaagtgtaatcaatcagcttgcattcgagttcgcgccgaaggtcagagtcaacggagtcgctccatccggcatcgctaattcacagcttcaagggccgcgtgccctaggattagagaacaacaagcagagtgatattcccgttgaggattttacgaaccaatttctgtctctgacgttgacacctaccctgcccactccggaggaatatgcgccactttatgcatatttagcgtccaggaacaataccacaatgacagggcaaacgataattgcagatcagggcctatttaacagagcggtcatatctaacggcgttgcagatagagtaggcaaataa

SEQ ID NO: 29 - DNA, 1281 bp, Artificial Sequence
  Description of Artificial Sequence Synthetic polynucleotide
atgagtagttctggccccgcgcacagcaatttagagcaagtattcgctaacgtggcttcaaattaccgtggggctgatgtagacttgcacgcggtttatagagaaatgcgtgagaagtctcccgtgttgcctgaaaatttcatggccaggcttggtgtgccgtctatagcagggctggacccaaataggccaacttttacgttgtttaaatatgacgatgtgatggctgtaatgagagatgcgactaatttcactagtggttttattgcggaaggtctgggctctttcttcgatggtttaattctaacagcaatggacggtgaagcacacaagaatatacgttcattgttacagccggtctttatgccagaaactgttaataggtggaaagagaccaaaattgacagagtgataagggaagagtatcttagaccaatggtggcttcaaagcgtgccgatatcatggagtttgctttatatttccccattagagttatttactcattgattggattcccagaggaccgtccggaggagatcgaacagtatgcggcttgggccttagcgattctggccggacctcaagtagatcctgaaaaagcagcagcggcacgtggagcagcaatggaagccgcccaagcactgtacgacgttgttaaggtagtcgtagcgcaaaggagggccgaaggggcgacaggcgacgacctgatttgcagactgatcagagcagagtacgaaggacgtagtctggatgaccatgaaataacgacgtttgttagaagccttctgccagcagcttctgaaacgacgacgcgtacgtttggtacattgatgactctgttgctagaacgtcctgaactgttggcacgtatccgtgaggatcgttctttagtcggaaaagctattgatgaggcggtacgttatgaaccagtggctacttttaaggtaaggcaagccgcaaaagacgtggaaattagaggggtggcaattccgaagggcgcgatggtgtcctgcatcgtgactagcgcaaatcgtgacgaggacgcttttgagaatgcggatacattcgatatcgaccgtagggctaagccgtcatttggatttggattcggtccacatatgtgtattggtcagtttgttgctaaaaccgaaataaactgcgccctaaatgccatactggatttgatgccaaacatccgtttagacccagataaacccgcgccagagattataggggcgcagctaagaggaccccatcacgtccacgtgatttgggactaa

SEQ ID NO: 30 - DNA, 1227 bp, Artificial Sequence
  Description of Artificial Sequence Synthetic polynucleotide
atgagtagtaggtcaactgaccttccggacctgaaatctgcggcctttcttgcggaccgttacccaacgtacaggagactacaaagtgatttcccgcacttcgaaatgaatataaatggagaggagtgtatcgtgctgacaagatacagcgacgtcgatgaagtcttacgaaacccgttggccacggttcaacaagctcctggtgtatttccagaaaggataggtcaaggtgctggggcccgtttctatcgtgagtcactacccaatattgatgcccccgatcacacgcgtatcaggcgtatagttacaccggcgttcaacccgaaaacagttgctaacatgagaggttgggttgagaaggtaatagtggagcacctagaccgtcttgaaggattggacgaaattgactttgtctctagctttgccgacccggtgccagcggaaatagcatgtaggttgcttcatgtgcctgtgtctgatgctccagaactttttgctaggcagcatggattgaatgctgtgctatctgttagcgacatcacacctgagagattagccgaagcggacgcatccgctgctttctactatgaatacatggacgacgttttaaacacactgaagggtaaattgccggaagatgatttcgtgggagcgttaatggctgccgaggcgcgtgactctggattaactaggtctgaattggttactacgcttatcggatttctggtagcctcatatcacaccacgaaggtggccatgacaaacactgtcctagctctacttaatcacgatggcgagagagctaggcttgtggcgcagccggatttggcgagaaatgcctgggaagaatcattgagatatgactccccagtgcatttcgtccaccgttatgcatctgaaccactgacaataggtggtcagcccgtggcccaaggtaaaaggctattattgggcttgcatgcagctagtagagacgaaaataggtttgcccaggcagatcactacttgattgacagaccggataaccgtcacctggcgtttgctgggggagggcacttttgcttggggtctcaacttagccgtttggaaggagacgtactgttgcgtacaatttttcaaagattccccgcaatgaggcttacggaaaccagattcgaaagagtaccggacttgacttttccaatgttactaaggatgacagtttcattaagggcggagcaaggttaa

SEQ ID NO: 31 - DNA, 1464 bp, Artificial Sequence
  Description of Artificial Sequence Synthetic polynucleotide
atgagtagtacctctaattcaattaggagcccattgagtccgccccagccgagacgtactccgccgccttgtacctcctcaagggaaccgcccatcgtccgtggtacttggcttttaggcagcacccgtgacttgttaagggacccactggagctagggctgcgtggatacgctgaaggcggggacgtggtaagatatgtagttgggttacctggtcgtagaagagagttcttcacggttaaccatcccgatggggttggggaactgcttaatgctccccgtcacttagactatcgtaaggacagtgaattttaccgtgccatgagggatttatatggaaatgggcttgttaccagtcaagatgaaacttggctgagacagagaaggttcatacagccgttgtttactccacagagtgttgatggttacgtcacaccaatggtcgcggaggctgatagggtagcaataaggtggcacaattgtacctcccgtctggtagatttggacggcgagatgcgtgccctaacattaggcgtggccgccagaatcctattcggagttcaagccccgaggatgcttcctatcctgaggactaccctaccggtacttggtagggccgttctgcaacaaggtgcgtcagctatcagatttcctagctcttggcctaccccgggtaatcgtcgtatcgccagtgcagaatctcgtctggatggtttgtgtgatgctattatagagcgtcgtaggacagtagccgagccaggtacggatttgctgggtcgtttggtcgctgcaagagaggacggtgatacgctgtcaacggaggaaataagagatcaagtcaaggtatttctcttggctggtcacgatacaacggcaacgatgctgacgtttgccttatacctgcttggtaaggacgctggcgttcaggatcaagcgcgtgacgaagcggaacgtgtcttgggggcggggacgccgaccgcaagcgacgtccaccgtctgacatatactacgatggtactggaggaggcggcgagactgtacccaccgtctccctatttaactcgtagagcggtcgaggaaagcgaggtctgcgggtacagaatacccgctggggccgatgtcaacctggctccatgggtgatccatcaccgtgccgatttatggcctgatcctttccgtttcgatcccgacagattcaccccggatagggtaaaagaaagacacaaatacgcgtggttcccgtttggacacggaccaaggggttgtatcggtcagagattcgcaatgctggaagcggcagttactttagcgattcttctaagagaatttgagtttaggtctccgcctggcagcgttccattaacagtagacttactgttgcatcccgccggcgaggttccttgccgtgtgaggaggcgtgtacctgtgcattcagcggttcatcgtactcaccagccaagttaa

SEQ ID NO: 32 - DNA, 1245 bp, Artificial Sequence
  Description of Artificial Sequence Synthetic polynucleotide
atgagtagtgccccggacattctttctcccgagtttctggataacccttatcctcttcaccgtgtgctacgtgaccactaccccgctttacaccacgaggggaccgacagctatctaatatcaaggtacgccgattgcgcagaagcatttcgttcacctaaattctcctcccgtaactatgaatggcagcttgaaccgatacacggtagaacaattttgcaaatggaagggcgtgagcattctacccatagagcattgctaaatccgtttttcagaggcaacggactagagagattcatgcctgccattacacacaacgcagcacaactaataggcgatatagtcgccaggaatgcaggggaattgctgggtgcggttgccagacagggggaagcggaattggtatcacaattcactagtcgttttcctataaacgtaatggtggacatgctgggactgccgaagtccgaccacgaaaggtttagaggctggtatttctccattatggcttatcttaataacctggcaggggaccctgaaattaacgccgcggcggagcgtacacatgttgaactaagggagtacatgcttccaattattagagagcgtaggagtggagatggagacgaccttctatccagattatgtcgtgccgaagttgacggtgagcagatgagtgatgaggagataaaggcctttgtctctctactgctggtcgccggcggagagaccacagataaggcaatagcaagcatgatcagaaatttgatcgaccacccagatcagatgagggcggttagagaagatcgttcacttgctgatagggtaatagcagagacccttcgttattccggacccgtacatatgatcatgagacaaacagaggatgaggttcagatagaggactctaccattccagcgggagcaacctgcataatgatgttagcagccgcgaacagagatgaacgtcatttttcaaacccggacgagttcgatatatttcgtacggacctaaacgtagacagagccttctcaggggcggccaatcatgtccaatttatattgggccgtcatttttgcgtcgggtccatgttggctaaaactgagatgaccattgcacttaatctggtcttggacacaatggatagcatagaataccaagatggttttgttcccagagaggaggggctgtacaccagaagcatcccggagcttagggtaaaatttgaaggtaagttagggtaa

SEQ ID NO: 33 - DNA, 1284 bp, Artificial Sequence
  Description of Artificial Sequence Synthetic polynucleotide
atgagtagtagcactcctgccgctgctacatccttggagagtgcctttgcgggcgtcgcggacaattataaaggttccgacgtggaccttcatgcaatctatagagatatgagacgtaactctcctgtcatcgctgaggatttcatggcacgtctgggtgttccgaatattgcaggcctagacgctaaaaggccaacatttacccttttcaagtacaaggacgtgatgtctgtcttgagggatgctaccaatttcacatcaggctttatcgcggaaggattaggggcgtttttcgacggcttaatcctgactgggatggatggtgaagcacaccgtagaactaggtccctattgcagccggttttcatgcccgacgttgtcaaccgttggagggaaacgaaaatggcaccaatagtcaggaatgaatatattgaaccgatggtcccgaaaaggcgtgctgaccttatggactttggacttcacttccctatacgtctaatctacagtttgatagggttcccagacaataggccggagcagatcgaacagtacgctgcctgggcacttgccatcctggcagggccgcaggtggacgcagagaaagcagcccaggcgcgtaaagctgcgatggaagccgcccaggcgctttacgacgcagttaaacttgaagttacagaggtccgtaaaaatggagcccagggtgacgatctaatctgcaggctaattagagctgagtatgaaggccgtcatcttgatgatcatgaagtcacaacctttgtcaggtcacttctgccagccgctggagagacaactacgagaacgttcggttcactgatggtcgctcttctggaaagacctgaattactggaacgtgttagggctgatagatccttagtgccaaaggcgatcgacgaagcggtgaggttcgaaccagtagctacttttaaggtcaggcaggcggcacaggatacggaaattggcgggttctccataccgaagggagcaatggttcaatgtatagtcagttccgccaacagggacgaagaggtcttcgaaaactctgagagctttgacattgatagaaagctgaaaccgtcattcggcttcgggttcggtccacatatgtgcatagggcagttcattgcaaaggtcgagttatcagtggccgtaaacactattttagatttattgccaaaccttcgtttagatccagacaggccgaaacctagaatagtaggtgctcagctgagaggtccccacgcgcttcatgttatttgggactaa

SEQ ID NO: 34 - DNA, 1278 bp, Artificial Sequence
  Description of Artificial Sequence Synthetic polynucleotide
atgagtagttctccctcagtggcagagttaagccaggagttgggagaagcattccgtctatccagcatggacgatccgtatccgatgttggcagagaggagaagagagactcctgtgatgaaaggggatataatggtggccttaggtgcgccaagctatatgggccaacacgccggcgagactcatactgtattcaggcatgacgacgtaatggctatccttcgtaatcacgaaacgttctcaagcagtatttgggaaatttctcaagggccactaataggtagatccatcctggcaatggacggggcagagcacagacaatggaggggatacttacagtctgtatttggagggaagctattgtcttcatgggatgagtccatattcaggccccttgcggcaaagtatgtcgcagaccttgctagtaagagaggtgcggacctaatagcgatggcgttggagtatccccttagggctatctacgagatcctgggcttggaagattttaaagacaattatgaggaatttcacgctgacgtactgacgattctactagccctatggtctacacccgacccagcgcaagccgaccagttcttgctacgttttcaaaaagctacggaagcatctgctaggagttgggaccgtctactacccatcgtccaaagaaagagggcggcgggtgcgagcaggaacgaccttatttctagtctaattagggcggaatacgagggtggtgttttggatgatgaacaaatcaccagttttcttaggtctctattgcttgcagccaccgatactactacccgtcagtttttgaatactttgaccttgcttttacagaggccagatgagttggatcgtattcgtagggataggagcagattgagattggcattggcggaaggggaaaggttggaaccgcccgccctattcataccccgtatgataacgagggatgttgttattaggggtaccgagttgacggcggggacccccttactacttgccatcgggagcgcgaatcgtgatcctgaagcctacccacccgacccagatgaatttcgtatcgatagaacgggaccacaccacgccacgttcggttttggtactcacatctgctccgggatgaacactactcgtcgtgagatagcagccttgatcgatgcgatgttagacgggctaccgggacttcgtgtcgatcccgacgctcccgcgccacttatatcagggattcattttagaggcccatccgcactgccggttgtatgggattaa

SEQ ID NO: 35 - DNA, 1098 bp, Artificial Sequence
  Description of Artificial Sequence Synthetic polynucleotide
atgagtagtgattactccaggacacccgagtccctgcgtccggctgatagttatgccgcgctatcctactccacagttaatgctgctctgcgtaacgatagagtattctcttcaaagatgtacgactccaccattggagtgtttatgggtcctacaatcttggctatgagtggcactaaacacagggctcacagaaaccttgtatccgctgctttcaagccgcaaagtctgagagtttgggaacctgatattgtaagaccaatttgtaatgcactaattgatgagtttgccgggacaggccacgcagacctggttcgtgacttcacgtttgaatttcctactagagtaatagctagactgctaggcttaccagcggaggatttgccattctttagaaaggccgcagtggcgattatcagttatgcaggaaacgttccgagagcgttggaagcgtccgaggacctgaagaactactttctaggacacatagagcaaagacgtagtcagcctaccgatgatattatatctgatttagttacggcagaagttgaaggagagcaattgaccgatgaggcaatttattcattcctgcgtctgctgttacctgctgggttagagacaacctaccgtagtagtggaaatttgctgtacctattattacgtcacccaaggcaatttgcggccgtgcaaggaaaccatggtcttattcctcaagccgtagaagagggtctgcgttatgagacgcctctaacgtttgtccagcgtttcacaaccgaagacacggagcttgggggcgttcctgttcccgcgggcgcagtagtagatttagtcttgggctctgccaacagggatgaagacagatgggaacgtccgggcgagttcgacatattcagaaaacccgtgccccatataagttttacggcgggagcccatacttgtttaggactgcatttagccaggatggagacgagggttgctgtcgagtgcctactaactcgtctgactaacttcagacttcaggatgaaggagacccccacataaccggacagccattccgtagtccgaatcttcttccagtaactttcgacgtggtttaa

SEQ ID NO: 36 - DNA, 1362 bp, Artificial Sequence
  Description of Artificial Sequence Synthetic polynucleotide
atgagtagtccgacgccaaggtggaggataccggtgctaggcgatcttctttcagttgaccccgcgaagcctgttcaaaaggaaatggctatggcggcggaactaggtccgttattcgagcgtaagattatagggagcagacttacagtcgttagcggcgtggacctagtcgctgaggtcaacgacgagaaacattgggctagagctttggggaggcccatactgaagctaagagatgttgcaggtgatgggttgttcacagcgttcaacagcgagcctgcatgggctagggctcatagcgtgttgggccctggcttctcacaaagcgcattgagaacctaccatggcagtatgactagggtgttggatgatttggtggcgacatgggacgatgcagcggcatcaggtgcccgtgtcgatgtcgctcgtgatatgacgagactgactttcgatgtgattggcagagccggctttggtcgtgacttcggctctttgaggggtgatgatctggacccctttgccgctgccatgggtagagcacttggttatgtgaatcaaacatcaaatgacataccacttctacgtatggtattcggtaggggcgcggccaaaaggtaccagacagacgtcgcatttatgcgtgataccgtagacgagctagttgcgagcagggctgggcgtgccgagaggagcgatgatcttcttgacctaatgttacacagtgctgacccggatactggggagaggttggacatggaaaacattaggaatcaagttcttaccttccttgttgccggtaatgagacaacagctagtacattggcgtttgcactgtattttctggctagagagcccgaagttgtcgaaagagccagggccgagatcgcggatgtagtcggagacggtgagatcgctttcgagcaagtggctaaattacgttatgtcaggagggttgtcgatgagacgttaagactgtggcctgccgctccgggctactttcgtaaagttaggcatgatacggtattaggcggtcgttatcccatgcctaaaggttcatgggttttcgtgctgttaccacagcttcatcgtgaccctgtatggggtgacgatccggaaaggtttgaccccgatagattcgcaccagacgctgtgcgtgcaaggcctaaagatgcttatagaccgtttggcacaggccccagaagttgtatagggaggcagttcgcgttgcacgaggcggtacttgccctggcgacgttgttgagaagatacgacgttgccccagacccagcatatcgtttagatatcgtagaagctgtaacgctaaagcctagaggctttgagcttacactacagaggaggtaa

SEQ ID NO: 37 - DNA, 1281 bp, Artificial Sequence
  Description of Artificial Sequence Synthetic polynucleotide
atgagtagttcagcatcttcccagtctaacctagagcaagtctttgccaacgtagcatcaaattatagaggagcagacatagacttgcacgcagtatatcgtgaaatgagggaaaagtctccggttctgccagagaatttcatggcccgtctaggtgtgccctcaatcgctggtctggaccccgaccgtcctgccttcacgctattcaaatatgacgacgttatggcagtcatgcgtgatgctacaaactttacttcaggctttatagccgagggtttggggtccttctttgatggacttatattgacagcaatggacggtgaggcacataaaaatatacgttccttattgcagcctgtctttatgccagaaaccgttaacagatggaaagagactaagatcgacagagtgataagggaagaatacctgcaaccaatggtggcatccaaaggggcggatattatggagtttgctctgtattttccaattagagttatttattccctgataggattcccagaagatagacccgaggaaatcgaacaatacgcagcatgggcgctcgcaatcctggcgggcccacaagtggaccccgaaaaggcagttgccgcgcgtggagccgctatggaagctgcccaggcgttgtatgacgtggtgaaagtcgtagtcgcgcagaggcgttctcaaggtgccacgggagatgacttgatatccaggctgatacgtgccgagtacgaaggtcgtagcctggatgaccacgagataaccacgttcgtcaggtccctactgcccgcggcatctgagacaacgaccagaacattcgggacattgatgactttactattggaaagaccggagcttctagcacgtattcgtgaagacagaagcctggtgccaaaagcaattgatgaggctgttaggtacgaacctgtagcaacctttaaggtcagacaggccgctaaagacgttgagatacgtggggtagccattcctcaaggagccatggttagctgtattgtaacatctgcaaatagggacgaagacgcgttcgaaaatgctgatactttcaatatcgatagaagagcgaaaccatcattcggtttcggattcggcccacacatgtgcattggacaatttgtagccaagaccgagataaattgcgctctaaatgctattctggacttaatgcccaatatacgtcttgatcccgataaacctgcaccagaaatcataggtgcccagctaaggggtccccaccatgtacacgtcatttgggactaa

SEQ ID NO: 38 - DNA, 1377 bp, Artificial Sequence
  Description of Artificial Sequence Synthetic polynucleotide
atgagtagtactgccacagaattgagggatgcacctgggagtgcgccaggcctacccaggagatccatgttatcccttttacccagaatggcacgtgatagattgtcagttatgacaagtgtagcggcgcgttatggggacgccgtgacgttgcccttgggcttatcaacgttacacttcttcaaccaccccgactatgctaagcacgtactggctgataatagctcaaactaccacaagggcatcggcttaatccacgcgaagcgtgcgttaggtgacggacttcttacgtcagagggtgagttatggagaaaacagaggaaaaccattcagccggcatttgctgttaaaaggttggctggacaagcgggggcaatcgcagaggaagctgataggttggtagagcatctgctggcccgtcaagggagagggccagttgacatcaggcacgagatgactgcccttaccctaggtgtgttaggccgtaccctacttgatgcggacttaggcgctttcggttcagtgggccactggttcgaggctgtacaagaccaggcgatgtttgacatgatgagccttggtactgtaccactatggtctcccttgcccaagcaactgagattcaggagagcgaggagggaattggagtcagtggtggaccgtctagtagctcagcgaggggatagacctagggcagacggcgatgatgttgtgtccaggcttgtcgatagtacaggaagggagcgtgatcctgcactaaggagaaagagaatgcacgatgaattggtgactctgttactggcgggccacgagacaacagcatctacccttagctggacattccatttggccgatgaacaccctgaggtctgggagcgtttacacgccgaagccgtggaggtactaggtgataggcgtccggtctttgaagatttacatcgtttgcgttacacaaatcgtgtactaaatgaagttatgaggttgtaccctccagtttggctgcttcctagaagagctgtcgctgacgacgttgttggaggatatagagtaccggctggatctgatgttttaatctgcccttatacgctacacagacatcctgagttttgggagcttccaagtaggttcgaccctgataggttcgatccggaaaggtctgccaacaggcccagatatgcttacattccttttggtgcgggtccacgtttttgcgttggtaacaacctaggactaatggaggcagccttcgttattgcagctatagcaagaagaatgagactaaggaaggttccgggaggaactgtcgttcctgaaccaatgttgactttacgtgttagaagtgggctgcctatgacggtgcacgcgcttgaccgttaa

SEQ ID NO: 39 - DNA, 1554 bp, Artificial Sequence
  Description of Artificial Sequence Synthetic polynucleotide
atgagtagtcagagaagagatttccttaagtattctgtggcccttggcgttgcctcagctttgcccctgtggtctagggccgtctttgccgcggaaagaccgactcttccgatccccgacttgctgacgaccgatgccagaaatagaattcaactaaccatcggggcaggccagagtaccttcggcggcaaaaccgccacgacttggggttacaatggtaacctgttagggcctgctgtcaaactacaacgtggcaaagcggtcacggtagacatatataaccaactaactgaggaaacaacgttgcactggcatggcctagaagtgcccggcgaagtagatggaggtccccagggcattatccccccagggggtaaaagatcagtcacattgaatgtcgaccagcctgcggctacatgctggttccatccacatcagcacgggaagacggggaggcaagtggcaatggggcttgctggtttagttgtaatagaggatgacgagatcttgaaactaatgcttccaaaacaatgggggatagacgacgtacctgtaatcgttcaagataaaaaatttagcgcagatgggcaaatcgactaccagctggatgtcatgacagcggcagtgggatggtttggggacacactgctaactaacggggctatatacccccagcacgccgctccaaggggttggttacgtctgcgtctattaaacggttgcaacgcccgtagcttaaattttgcgacctcagacaatcgtcccttgtatgtaatcgcgagcgacggtggattattgccggagcccgtaaaagtctccgagttgcctgtgctgatgggagaaagatttgaggttttggtggaggttaacgataacaagccctttgatctagttacccttcctgtaagccaaatggggatggccatcgctccatttgacaaacctcaccccgtcatgagaattcaacccatcgctataagtgcgtctggtgcgcttccagatactctgtctagcctaccagcgctaccgtctcttgaaggtttaacagtaaggaaactgcaactatctatggatccaatgttagatatgatgggaatgcaaatgttaatggagaagtacggtgatcaggcaatggcgggtatggaccactcccagatgatgggccacatgggtcacggcaatatgaatcatatgaaccatgggggcaaattcgacttccatcacgctaacaagattaatggtcaagccttcgacatgaacaagcctatgtttgccgcggctaagggtcagtacgaaagatgggtcatctccggggtaggggacatgatgctgcatccgttccacatccatggcacacaatttaggattcttagtgaaaatggaaaacctcctgctgcacatagggcgggatggaaggatactgtgaaggtggaaggtaacgttagtgaggtgctagtcaaattcaatcacgatgcccccaaagaacatgcctatatggcccactgtcaccttttggagcatgaggatacgggaatgatgctaggtttcacagtc

SEQ ID NO: 40 - DNA, 1566 bp, Artificial Sequence
  Description of Artificial Sequence Synthetic polynucleotide
atgtctagcagactgagcttcttaacgtcattggttacattggcgttggtatctagcacgtatgccggagttgggcccgttgtagatcttacagtttcaaacgccgttatttcacctgatgggtttgacagagacgcgattgtagttaacggcgtgttcccagcgcctcttatcacaggtaagaaaggtgacagattccagctaaatgtgatcgataacatgactaaccatactatgctgaagtcaacaagtattcattggcatgggttttttcaaaaaggtactaactgggccgatggcggggcctttgtcaaccaatgtccaatcgctcctggccactccttcctatacgatttccgtgtaccggaccaagcaggcacattctggtaccactcacacctttctacgcaatattgcgacggtttaagagggcccatcgtggtatatgaccccaacgaccctcatgcggacctgtacgatgtggataatgattccactgtgatcacacttgccgactggtaccacgttgccgcccgtcttgggcccagatttccgctgggagcagattctacggttattaacggtcttgggcgttcccttagcacgcctaacgctgacttagctgtgatctcagtcactcaaggtaaaagatataggttccgtctaatatctctttcatgcgaccccttccatactttttctatcgatggacatgacttgaccattatagaggcggacagcgtgaacacggagcccttggtggtggatgcaattccaatcttcgccggacaacgttattcttttgtcttgagcgccgtcaaggacatagataactattggatacgtgcggacccaaactttggaactacaggctttgcatcaggtatcaactcagcgatccttcgttatgacggggctgcacctattgaaccaaccgctgttttagctccggtaagcgttaatcccttggttgagacggatttgcacccgcttgaggatatgcctgtacccggtagaccaacaaagggtggcgttgataaagcaatcaacctggattttagttttagcttccctaattttttcattaacaatgccacatttacaagccccacagtgcctatcctgctacagataatgtccggcgcgcaagccgcgcaggatttattgccttctggtagcgtgattgaactgccagcgcagtccaccatagaactaactcttcccgcgacggtcaatgcccccggagtgccacatccatttcatttgcatggccacacattcgccgtagtacgttccgccggtagcactgcctacaattacgacaaccctatttggcgtgacgtcgtatccactggcacgcccgccgcaaatgacaacgtcactattagatttacaacggacaatcccggaccttggtttttacattgccacattgacttccaccttgaggctggcttcgccgtggtattcgcggagggtgtgccgcagacccaagtggcgaatccagtacctcaagcgtgggaggaactgtgcccgatttatgacgcattaccggaagatgatcag

SEQ ID NO: 41 - DNA, 1674 bp, Artificial Sequence
  Description of Artificial Sequence Synthetic polynucleotide
atgtctagttttaaagtcagctgtaaggtcactaacaacaatggtgatcagaacgtagaaacgaattccgttgatagaaggaatgttctgctgggcctgggggggctatatggtgtcgctaatgccatcccgctagcagcctcagcggctccaacgccaccaccagacctaaagacttgtgggaaggcgacgataagtgacgggcctctagttggatacacctgttgtcctccccctatgcctacaaattttgacaatataccctactataagttcccaagcatgacaaagcttagaatccgtagtccggcacatgccgttgacgaagaatatatcgctaaatacaatttagcgatttccaggatgaaagatctagataaaaccgaacccttaaaccctctagggttcaagcagcaggctaacatccactgtgcgtactgtaacggtgcgtatgtgttcggcgacaaggtacttcaggtacataactcctggctgttcttcccctttcatcgttggtatttatacttctatgagaggatattgggcaagttaatagatgatcccacgtttgctctgccatattggaattgggatcacccaaaaggcatgcgtttgccgccgatgtttgacagagagggtacatccatctatgatgaaaggaggaatcagcaagtgcgtaatgggaccgtcatggatttgggatcattcggagacaaagtagaaacgacccaactgcaacttatgtccaacaatttgactttgatgtatcgtcaaatggtcacaaatgcgccctgcccactactgttttttggagccccgtatgttcttggaaacaatgtagaagcccctggcacaattgaaaatataccgcacattcccgtgcatatatgggctggcacggtgcgtggctccaccttccctaacggggatacgtcttacggagaagacatgggtaatttttactccgcaggtttagatagcgttttttactgccatcatggaaacgttgatcgtatgtggaacgagtggaaggctataggtggtaagaggcgtgacctgtctgaaaaagattggttgaatagtgaattttttttttatgatgagaacaagaagccgtatagggtcaaagttcgtgattgcctggacgcaaagaagatgggctacgattatgcgcccatgcccactccctggcgtaatttcaaacccaaaacgaaggtgagcgcaggcaaggtcaacacatcatcccttccgcctgtcaacgaggtttttcccttggctaaaatggataaggtgattagtttttcaataaacaggccggctagctcaagaacacagcaagaaaaaaatgaacaggaggagatgttgacatttgataacatcaagtacgacaatcgtggttacattcgttttgacgtcttcttgaacgtcgacaacaacgttaacgcgaacgagctggacaaagttgaattcgctggaagctatacctcattaccacatgtgcatcgtgtcggagaaaatgatcacacggccaccgttaccttccagctagccatcactgaactactggaagatatcggcctagaagatgaagaaaccatagctgtgactctagtacccaagaaggggggtgaaggaattagtattgagaatgtggaaattaaattattagactgt

SEQ ID NO: 42 - DNA, 1677 bp, Artificial Sequence
  Description of Artificial Sequence Synthetic polynucleotide
atgtctggccagaataaaatgggtcttatacttgtatttctgtttctggacgggttgcttgtctgtttagctgcggatgtggatgtacataactacacctttgttctgcaggaaaaaaactttactaaatggtgtagcactaaaagtatgctggtcgtaaacggttcattccctgggccaactattacagccagaaagggggatacgatatttgtcaacgtcataaatcaagggaagtacgggttaaccatccattggcatggtgttaagcaaccaaggaatccctggagcgacggacccgaatatataactcaatgcccgattaaaccgggtacgaacttcatttacgaggtcattctgtcaaccgaggagggaacactgtggtggcatgcacactccgactggacgcgtgccaccgtgcatggtgcgttagtgattttacccgctaacggaaccacatatccttttccacccccgtaccaggagcagacgatagtcttagcgagctggtttaaaggcgatgtgatggaggtaattacatcttctgaagagacgggggtttttcccgccgcggctgacgggtttacaatcaatggcgaactgggagacctgtacaattgcagcaaggaaaccacatacaggctttccgtacagccgaacaaaacatatttactaagaattgtgaatgcagtcctaaacgaggaaaagttttttggtatagcgaaacacacattgacagtagttgctcaggacgcttcatatattaagcctataaatacctcttatataatgatcacgcctggccaaacgatggatgtattattcacgaccgaccaaactccttctcactactatatggttgcgagtccgtttcacgacgcactagacacgtttgcaaattttagcactaatgcaatcatacaatataatgggtcctataaagcaccgaaaagtcccttcgtgaaaccgttgcccgtttataatgacatcaaggcagcagataaattcacggggaaactgcgttctcttgccaatgagaagttcccagtaaacgtccccaaggtcaacgttagaaggatatttatggcagtctcactaaatatcgttaagtgtgcaaataagagctgcaacaataatataggacactctacttcagcctccctaaacaacataagttttgcgctacctcagacagatgtactgcaggcatattatagaaacatcagcggcgtattcggtagagattttcctacagttcagaagaaggctaacttttccttaaatacagcccaaggcactcaagtactaatgatagagtatggcgaggccgttgagatcgtatatcagggtactaatttgggagccgcaaccagtcatccgatgcaccttcatggctttaacttctatctagttggcacgggtgctggaacgttcaacaacgtgactgatcctcccaagtataacctggtcgacccgcctgagttgaatactataaacctaccacgtatcggctgggcagcaattaggtttgtcgcggacaacccaggggtctggttccttcactgtcacttcgagagacatacaacggagggtatggcaacagtcgtgattgtgaaagatggcggaactacaaacacttctatgctaccaagtcccgcgtacatgccaccatgcagc

SEQ ID NO: 43 - DNA, 2004 bp, Artificial Sequence
  Description of Artificial Sequence Synthetic polynucleotide
atgtcttcccgtaagatttgtctagggtgttcacattctttaagctcccaaccctttacatatacaactcagaagactgtaagtagtaggcgtatcggtgactctcagtggcgtcttagccgtggttacacccgtacgctgacctctgcaagtgcaagcgttgctacagctcccgctaagctacttacggtcaatgaaactcaaaaatgcctaaggaacatggtccgtggcggagacgtaattagctacattctttcccattcttcccgtaacgcagaccagaatttgaaagatttagacagcttaatattggagcctgtctgcagtgctacgcacgagatgttcgacgttttcgagatcccagaacacattttgactccgttttgcgataacagaaatgtccccgaggaacaagtcacccgtaatcctaatctgagaaccgactgtctgacgatgaagaggtttgtgctattacagagcctagtcgcggttgcatccgccggaattgggccagtagcagatctgtacgtaggaaatagaatactggctccggatgggtttaacagaagtacagttctaggaggtaccagttcatctgattttggattcccagcgccactaatcaccggcacaaaaggggacaggtttcaactgaacgtcatcaatcaattaaccgacactacgatgttaagatcaacaagcatacattggcacgggttattccaggctggctcatcttgggccgacggccctgtaggagtaaatcaatgccctatagctccaggaaactcatttctgtacgactttaacgtccctgaccaggcgggaactttctggtatcatagtcattatagcacacagtactgtgatggtcttaggggggcttttgtggtaagagatcctaacgatccacatgcgagtctttacgatgtcgataatgatgacacagttataacattggctgattggtatcatacgagcgctaaagagctatcaggctcctttccggcagaagaggcgaccttgatcaatgggctgggtaggtatagcgggggtcctacttccccattagctatcgtcaatgtagaagcgggcaagaggtaccgtttccgtttggtatccataagctgcgatccattctacaccttctccattgatggtcacgatttgaccattatagaggcggacggggagaacactgatccactagtagtggactatctggaaatatacgctgggcaacgttacagcgtggtgttaaacgcgaaccagccagtagacaattactggattagggcaaattcttccaatggtccgagggactttgttggcggcacaaattctgccatactgcgttacgccggtgcatcaaactcagatccgacaacagagctagggccgcgtaataataggcttgttgagaataaccttcatgctctgggatcccctggtgtgccaggcacgcatacgattggagaggccgatgtaaacattaatcttgaaatattgtttacgccaccgaatgtcctaaccgttaatggcgcccaattcattccacctactgctcccgttttattgcagatattgtccgggacaaaacaagcaacggatttgttacccccaggttccgtatatgttctgcctagaaacgcggtagttgagctaacaatcccgggtgggtcaggcggaagtcctcatccgatgcatctgcatggccacgtctttgacgtagttagatcagctggatcagataccataaattgggacaatccggtcagaagagatgtcgtgaacattgggactagcacatctgacaatgccacgattaggttcacgaccgacaacccgggaccatggatttttcattgtcatatcgactggcacttggaggttgggctggcagttgtttttgctgaggatccggatacaattgaaaatagtacacatcccgctgcgtgggatgagctgtgcccaatttacgacaaccttccttccgacgagtta

SEQ ID NO: 44 - DNA, 1545 bp, Artificial Sequence
  Description of Artificial Sequence Synthetic polynucleotide
atgagctccacattggaaaagttcgtagatgccttaccgatcccagatacattaaagccggtacaacaatctaaagaaaaaacgtattacgaggtcacgatggaggaatgtacgcatcaattacatagagatcttccgcccacaaggctatggggatataacggtttatttcctggtccgacgatcgaagtgaagagaaatgaaaacgtatacgtaaagtggatgaataatttaccttcaacacattttcttcctatagatcataccatccaccacagcgactcccaacatgaagagcctgaggtaaagacggtagtgcatcttcatggcggtgttactccggatgactccgacggctatccagaagcatggttcagcaaggatttcgaacaaacgggcccgtacttcaaaagggaagtatatcactacccaaaccagcagcgtggtgccatcctatggtatcatgatcatgcaatggccttgactcgtttgaatgtttatgcaggtctagtcggggcatacattatacacgatcccaaggaaaagagattaaaactgccttcagatgagtacgatgtacccctactgatcacggacaggacaataaacgaggatggttctcttttttaccccagcgcgccagaaaatccatccccctcactgccaaaccctagcattgtcccggcattttgcggggagacaatccttgtgaatggtaaagtatggccgtacttggaggtcgaaccaaggaagtatagatttagggttataaatgcgagcaacacaagaacatataacttatccttagacaatggcggcgacttcattcaaataggatctgatgggggcttgttaccccgttcagtgaagttgaattccttttcattagcacctgcagaaaggtacgatataatcattgactttaccgcatacgaaggtgagagcattatcttagctaatagtgctggctgcgggggggatgtcaatcctgagacggacgcgaatattatgcaatttagagttacaaagcctctggcccaaaaggatgaatccagaaaaccaaagtacttggcatcctatccgtcagttcaacatgagaggattcaaaacataaggacactgaaattagcaggtacgcaagacgaatatggtcgtccggtacttttgctgaataataagcgttggcacgatccagttactgaaacgcctaaggtgggtaccaccgagatttggagcataataaatcccacgagaggcacccatcccattcacctacatcttgtcagtttcagagtcttagaccgtcgtccgttcgatatagctcgttatcaggagtcaggggaactttcctacactggacctgctgtaccgccgccaccgtcagaaaagggttggaaggacacgatccaggcccatgcgggtgaagttctaagaatcgcagctaccttcggtccgtacagcgggaggtatgtgtggcactgtcatatcttggagcacgaagactacgatatgatgaggcctatggatatcactgatccacacaag

SEQ ID NO: 45 - DNA, 1554 bp, Artificial Sequence
  Description of Artificial Sequence Synthetic polynucleotide
atgagctctgtgtttagtgctgcgttttccgcattcgttgccttaggtctaactctgggcgcttttgctgccgttggcccggtcgcggacatccacattaccgatgataccatagcacctgacggatttagtagggctgccgtactggcaggtgggaccttcccagggcccctaatcaccgggaacatgggagacgcctttaagttaaacgtcatcgacgagttgacggatgcctctatgttgaaaagtaccagtatccattggcacggcttctttcagaagggaacgaactgggctgatggcccagctttcgtgaatcaatgccccattacaactggaaactccttcttgtatgacttccaagtgccagaccaagcaggtacttattggtatcactcccacctaagcactcagtactgtgacggactgagaggtgccttcgtggtttacgacccaagtgatccgcacaaagatctgtacgacgtggacgatgaatccaccgtcataaccctagcagactggtaccacacgctggccaggcagattgtgggagtggcgattagcgataccacgcttatcaatggcctggggcgtaatacagacggacccgcagatgctgccttagccgtgatcaatgtagaagctggcaaaagatatagatttcgtttagtaagcatcagttgcgacccgaattgggtgtttagtattgacaatcatgactttacggttattgaggtagacggcgtgaacagccagcctctgaatgttgacagcgtacaaatatttgcagggcagaggtattccctagtgttgaacgcgaaccagcccgtcgataactattggattagggctgatcctaaccttggtaccacagggttcgcgggtggaataaattcagcaattctacgttataagggtgcggccgttgccgagccgactacatcccaaaccacaagcaccaagcccttattggagactgacttgcaccccttggttagtacaccagtcccaggattaccgcaacctggcggcacggatgtagtccaaaaccttattctaggcttcaatgctgggcagttcacaatcaatggcgcatcctttgtgccaccaacagttccagttttgttacaaatcttatctggaacaacgaacgcgcaagacctgctaccgtccggtagtgtatttgagttaccgttgggaaaaacggtcgaattaaccctggcagccggcgttttaggcggaccacacccgtttcacttacacggtcataatttccatgtcgtcaggtccgctggacaggacacgcccaattacgacgatccaattgtccgtgacgttgtctcaaccggagcgtctggggacaatgttacaattaggtttactaccgacaatcctgggccctggttcctacactgtcatatcgattggcacctagaggcaggctttgcggttgtattcgcagaggcagtgaatgagactaaatctggcaatcctacaccggcagcctgggataacctatgcactctttacgatgctctagctgatggtgacaag

SEQ ID NO: 46 - DNA, 1908 bp, Artificial Sequence
  Description of Artificial Sequence Synthetic polynucleotide
atgtcctcctgtttggccgctatatggtcaaggaaaagagcggagcatgccgcgtcaaggcttccagctttacaggagaaaaggtccacactaagctacgcgtatgctaggttagatggcagtcttgcgagtatgtttccaaataggttttggtcaagcgttagtcttggagctaggattaaaccggtggatgggagtagtgaagaacccaccgcaaggcccagcagctgtgccaggcccttcttacactcagcatcatctgaatcagggttcgtctcctcctcacgtccgaccagcttttgcgttacgtgctcccgtcgttggagatgctgtagtcttttggcaatgctgggattcaggttcttacacacaagcgtccttgctgcattgactcttagtctaaagagttatgcggcgataggaccggttacagacttgaccgtcgctaatgcgaatatttcacccgatggttatgaaagagctgcggtgttagccggcggttcatttcccggcccactaattactggcagaaagggggaccactttcagattaatgtagtagatcagctaaccaaccacaccatgcttaaaagcacctctatccattggcacgggctgttccagaaagggactaactgggcagacgggccggcgtttgttaaccagtgtcccatctccactgggaactccttcttatacgacttccatgttcctgatcaagcggggactttttggtatcattcccatctaagcacacagtactgtgatggtctaaggggtgccatggtggtgtatgaccccaatgaccctcacaagaacctttatgacgtagataatgacgataccgtaataaccctagcagattggtatcatgtagcctctaagctggggcctgctgtcccttttgggggggactcaaccttgataaatggcaagggtcgtagcactgcaacaccaaccgccgaccttgctgtcattagtgtaactcaaggtaaaagatataggttccgtctggtgtcactttcatgcgacccgaatttcacgtttagtatagatggtcatgccctgaccgtaatagaggccgatgctgtttcaactcagccattaactgtcgacagtatccaaatattcgcgggtcaaaggtactcctttgtgcttaatgccaatcagtccgttgattcatactggattcgtgcccagccatcccttggtaatgtgggctttgatggagggcttaattctgcgatccttcgttatgacggggctgcgccgaccgaaccatccgcgctagctgttccagtctctactaatcctttggttgagacggcactgaggccgcttaattcaatgcccgtccccggtaaggctgaggtgggcggtgtggataaagcgattaaccttgcgtttagtttcaatggcacgaactttttcatcaatggggcaacgtttgtgccgcccgccgtgcccgttctactacagatcatgagtggcgcccagagtgctagtgatcttcttcctagcggctcagtgtttgtgctacccagtaacgctaccatcgaattaagttttccagcaactgcaaacgccccaggcgctccacatcccttccacctacatggacacacgttcgcggtagtacgttctgctggttccgcggagtataattatgaaaatcctatatggagagacgttgtttcaaccggttctccgggagacaatgtcaccatacgtttcaggaccgataatccgggcccctggtttctgcattgtcatatcgatccccatctggaggccggctttgcggtggttatggcggaggacactagggacgtcaaggccgacaatcccgaacctaaagcctgggacgatctttgccccacatacaatgcgctagcagtggatgaccaa

SEQ ID NO: 47 - DNA, 2325 bp, Artificial Sequence
  Description of Artificial Sequence Synthetic polynucleotide
atgtttccaggggcgcgtattctggccaccctgacgttggcactgcatctgctgcacggtaccaatgccgcaataggacccactggagacatgtacatagttaacgaagacgtgtcccctgacggcttcaccaggtccgcagtagttgctaggagtgatcctaccacaaacgggacatccgaaactctaacgggtgtcttagttcaggggaacaagggagacaacttccaattgaacgtgctgaaccagttatcagacacaactatgctaaaaacgacaagcattcattggcacggcttttttcaatctggatccacctgggccgacggcccagcctttgtaaaccaatgtccaatagctagcggtaattctttcctttacgattttaatgtaccagaccaggccggcacgttctggtaccacagtcatctgtcaacccagtactgtgacggtttgaggggaccatttatcgtttacgacccaagcgatccccacctgtctctatacgacgtcgacaatgcggacactatcataacgctggaagactggtaccacgtagtggcacctcagaacgccgtattgccgacggccgatagcacactaattaacggcaaaggccgtttcgccgggggtcctacaagcgccttagcggtcatcaatgttgagtctaataaacgttacagattccgtcttatatccatgtcctgtgaccctaattttacgtttagtatagacggacatagcctacaagtgattgaggccgatgccgtgaacattgtaccgatagtagtggattccatccagatattcgccggacaaaggtacagctttgtacttaacgctaaccagaccgtggacaactactggatcagagctgatccgaatttgggcagcactggtttcgacggaggtatcaactctgctattttaaggtatgctggagcaacggaagatgaccccacgacgacaagttccacctcaactccattagaagaaacaaacttggtacccctagagaatcctggggcgccaggaccggcggtacccggcggtgcagacataaatatcaatcttgccatggctttcgacgtgacgaatttcgaattaacgattaacggatcccctttcaaagcacctaccgcccccgtacttttacagatattgtcaggagcaacgacggccgcttccttgctgccatcagggtccatttactcactagaagccaataaagtagtggagatttccatacctgctctagctgtggggggtccgcatcctttccacttacacgggcatacatttgacgttatacgttcagcggggagtacgacttacaattttgacactcccgctcgtagggatgtcgttaatacaggaacggacgctaatgataatgtaacaataagatttgttaccgacaacccagggccgtggtttctgcattgccacatagactggcacttagagatagggctggccgtcgttttcgccgaggacgttactagcataaccgcacctccagcggcgtgggatgatttgtgtcccatctatgacgcattatctgatagcgacaaggataatcctaggtttggatttgcaccagcgacaggaggtaaagcaacgggcaggagaaattggttctcaaaggctaggcgtagagcaattttggttccctatttaaaacttttaaaattggggttggtgatggtcttctatataagagcagaaaggaaccatggtagactttcccaatctacacctccgaatcgtagggtagatcagcgtgaattaattactaacacatgggtggagaggtttttcctgcaccgtctaatgtttctgaagctttttgttggaaccgcatgtttcatgcacatcttcaattcagttagctcactagggatgtgtacgttgaggaccagccatgggtcctccgagtcattagcttctccatcagcgaccatgatgttaggaggcggcctaactcttcttagtgctaacataagactttggtgttatgccgagatgagagatttgtacgacttcgaagttaatatcaaaaaggcccaccgtcttgtaacgactgggccgtatagtgtttctatggttatgtttagcaaggatcattggttgtatcaatgcggtctgcgttccatggttggcgttgtgctaagttgtatatggtgcgcggaagttgtactgatcaacggaattatggtcccggcacgtatgaaggtggaagacgacggattgagaaggcacttcgggcgtgagtgggatgaatatgcatcacgtgtggcctataggttggtccccgagatttat

SEQ ID NO: 48 - DNA, 1824 bp, Artificial Sequence
  Description of Artificial Sequence Synthetic polynucleotide
atgtcaagtaagagctttatctctgccgcgaccctactggtcggaatacttacgccgagtgtggcggctgctccacctagcacccctgagcaaagggacctgttggttcccattacagaaagggaggaagctgctgttaaagcgcgtcagcagtcttgcaacactccctcaaaccgtgcatgctggacggatggctatgacatcaatacagactatgaagtagattctcctgatacgggtgttgttcgtccctacactttgacgctgaccgaggttgataactggactgggcctgatggtgtcgtcaaggaaaaggttatgctggtaaacaattcaataatcggacccacaatttttgccgattggggtgataccatccaagtcacggtgattaataaccttgagaccaatggaacgagtattcattggcacggcctacatcagaagggtacgaacttgcacgatggagctaatgggattactgaatgccccatcccgcccaaggggggcagaaaggtttatagattcaaagcacagcaatatggaacgagttggtatcatagtcacttttccgcgcagtacggcaacggtgtggttggcgcgatacagatcaacgggccggccagtttaccatacgatacggacctgggcgtttttcctatcagcgattattattattcctcagcggatgagctagttgaattgaccaaaaacagcggtgcacccttttcagataatgtcctttttaacggaacggcaaagcacccagaaacaggcgagggcgagtacgcaaatgtaacgttaaccccaggaaggaggcatcgtttgcgtctgattaacacgagtgttgaaaaccatttccaagtctctctagttaatcataccatgacgatcattgccgccgatatggttccagtaaatgctatgaccgttgattcactgttcctgggcgtcggacaaaggtacgacgtagtaatagaagctagtagaactccagggaattattggttcaatgtgacattcgggggcggcctgttgtgcggaggcagtaggaatccttacccagctgcaatatttcactatgcaggcgcccctggtggaccgccgactgatgaaggaaaagcgccggtggatcacaactgcttggatctgccgaaccttaaacctgttgttgctcgtgatgtgccattatctggtttcgccaagaggcccgacaacactttagacgtcactttggacacgactggaactccccttttcgtctggaaggtaaacggtagtgctattaacatagactggggccgtccggtcgtggattacgtactaacacaaaacacttctttcccacccggttacaatatagtcgaggtcaacggcgcagatcagtggtcatactggttgattgagaatgacccaggtgcgccattcacgctaccgcacccgatgcacctacatgggcatgacttttatgtactaggtagaagtccggatgaatcacctgctagcaatgaacgtcacgtatttgatcccgcccgtgatgcgggattactgtccggggcgaacccagtgaggcgtgatgttactatgttgcctgcgtttggatgggttgtgctggccttcagggctgacaaccccggggcatggctttttcattgccatatagcatggcacgtatccggcgggctaggtgttgtctacctagagcgtgcagacgacctgaggggagcggtatcagacgcggacgcggatgacttggataggctttgcgctgattggaggagatactggccgacaaatccgtatcccaaatcagactctggtctt

SEQ ID NO: 49 - DNA, 1557 bp, Artificial Sequence
  Description of Artificial Sequence Synthetic polynucleotide
atgtcatcccgtttccagagcctatttttctttgtgctggtaagcttgactgcggtggcgaatgcggctattgggccggtggctgaccttacacttacaaacgcacaagtttccccagatggcttcgctagagaagcggtcgtggttaacggaatcaccccagcaccattgattacggggaacaagggggacagatttcagttaaatgtgatcgaccagcttactaaccacacgatgttgaagacgtcttctatacactggcatggttttttccagcagggtactaactgggcagatggccctgctttcgttaaccagtgtccgattgcgtccggtcatagttttttgtacgactttcaggtccctgatcaagcggggacgttctggtatcactcacacctaagtacccaatactgtgacggactgcgtggaccgttcgtggtgtacgaccctaatgatccccatgcgagcctttatgacatcgacaatgacgatactgtcataactctggcggactggtaccatgtagccgcgaaattaggtccacgtttcccattcggttcagatagcaccctaataaacggccttggcagaactaccggaattgcgccgtctgaccttgcagtcatcaaagtgacacagggcaagcgttaccgtttccgtctggtctctttgtcctgtgacccaaaccacacattctccattgacaatcacaccatgacgatcatcgaggccgactctatcaatacgcagccactagaggtggatagcatccagatattcgctgctcagcgttattctttcgtgctggacgctagccaaccggtggataactactggataagagcaaatccggcgttcggtaacaccgggtttgctggtgggataaactctgccatacttagatacgatggtgcaccagaaatcgagcctacttctgtccaaacaaccccgactaagcctctgaatgaagtggatttgcaccctttgtcaccgatgccagtaccaggatctccagaaccgggaggagtggataagccacttaacctagtgttcaatttcaatgggacaaactttttcattaatgaccacacctttgtgccaccctctgtgcccgtacttttgcaaatattgagtggtgctcaggcggcgcaagacctggtcccggaggggtccgtgttcgttcttcctagtaattctagcattgagatctcctttccagcaaccgctaatgctccaggtttcccgcatccattccatctacacggacacgcatttgcggttgtaaggagtgcggggagttcagtttacaactatgacaaccccatattcagggacgtagtaagcacaggacaaccaggtgacaatgtgactataagattcgagaccaataaccccggtccttggttcttacattgccacatagactttcacttagacgcgggttttgcagtggtcatggccgaggatactcctgatactaaagccgcgaatccagtgcctcaagcctggtctgatttatgtccgatctatgatgcgctggatccttccgattta

SEQ ID NO: 50 - DNA, 1563 bp, Artificial Sequence
  Description of Artificial Sequence Synthetic polynucleotide
atgagctccggacttcaacgtttcagtttcttcgttacgttagcattagtggcccgttcacttgctgcaatcggaccagtggcatccctggtagttgcaaacgctccagtgagtccggacggttttcttagggacgccattgtggtaaacggagtggtaccgagtccactaataactggcaaaaaaggagaccgtttccagctgaatgtcgatgataccctgacaaatcatagtatgcttaagagcacgagcatacactggcacggtttttttcaagcaggaacaaactgggccgacggaccggctttcgtcaatcaatgtcccatcgctagtgggcactccttcctatacgattttcatgttccagaccaagcggggacgttttggtaccatagtcatctaagtacccaatactgcgatgggcttcgtgggcctttcgtagtgtacgacccaaaggatccccatgcgtccaggtacgatgtcgacaatgaaagcacggtgattacgctgacagattggtatcataccgctgcgaggttaggaccgcgttttccccttggagcagacgccactctaatcaatgggttgggacgttctgccagtacaccgaccgccgcgctggctgttataaacgtacaacatgggaaaagatacaggtttaggttagtatctatcagttgtgaccctaattatacattctctatagatggtcataaccttacggtcattgaagttgacggcatcaattcccagcccttactggttgatagtatccagatcttcgctgcccagagatattcttttgtgctgaatgctaatcagacagttggtaactattgggtcagggccaaccctaactttgggacggttggttttgctggggggatcaactcagccatactaaggtaccaaggtgcgcccgtagcagaacctactaccacgcagacaaccagtgtaatccctttgattgagaccaatctgcatccgcttgcacgtatgcccgtaccgggctcaccgacaccaggaggagtggacaaagccttaaatctagcttttaacttcaatggaacaaatttcttcatcaacaacgccacgtttacaccaccaacggtgcctgtattacttcagatcttaagcggcgcccagacggcacaggatttgctgccagcaggatcagtatatcctctacccgcgcactcaaccatagaaataacgcttcctgccacagcacttgctcctggggctccacaccctttccacctacatgggcacgcattcgccgtagtgagatctgcgggatccacgacttacaattacaatgaccccatcttccgtgatgtggtgagcacagggacaccagcagcgggagataatgttactattcgtttccaaactgacaacccggggccatggtttctgcactgccacatagattttcatcttgacgccggctttgcgatcgtgttcgccgaggatgtcgcagacgtgaaggccgccaaccccgttccaaaggcgtggtcagatctatgtccgatatatgacggcttatctgaagccaatcaa

SEQ ID NO: 51 - protein, 492 aa, Artificial Sequence
  Description of Artificial Sequence Synthetic polypeptide
MAADSLVVLVLCLSCLLLLSLWRQSSGRGKLPPGPTPLPVIGNILQIGIKDISKSLTNLSKVYGPVFTLYFGLKPIVVLHGYEAVKEALIDLGEEFSGRGIFPLAERANRGFGIVFSNGKKWKEIRRFSLMTLRNFGMGKRSIEDRVQEEARCLVEELRKTKASPCDPTFILGCAPCNVICSIIFHKRFDYKDQQFLNLMEKLNENIKILSSPWIQICNNFSPIIDYFPGTHNKLLKNVAFMKSYILEKVKEHQESMDMNNPQDFIDCFLMKMEKEKHNQPSEFTIESLENTAVDLFGAGTETTSTTLRYALLLLLKHPEVTAKVQEEIERVIGRNRSPCMQDRSHMPYTDAVVHEVQRYIDLLPTSLPHAVTCDIKFRNYLIPKGTTILISLTSVLHDNKEFPNPEMFDPHHFLDEGGNFKKSKYFMPFSAGKRICVGEALAGMELFLFLTSILQNFNLKSLVDPKNLDTTPVVNGFASVPPFYQLCFIPV

SEQ ID NO: 52 - protein, 492 aa, Artificial Sequence
  Description of Artificial Sequence Synthetic polypeptide
MAADSLVVLVLCLSCLLLLSLWRQSSGRGKLPPGPTPLPVIGNILQIGIKDISKSLTNLSKVYGPVFTLYFGLKPIVVLHGYEAVKEALIDLGEEFSGRGIFPLAERANRGFGIVFSNGKKWKEIRRFSLMTLRNFGMGKRSIEDRVQEEARCLVEELRKTKASPCDPTFILGCAPCNVICSIIFHKRFDYKDQQFLNLMEKLNENVKILSSPWIQICNNFSPIIDYFPGTHNKLLKNVAFMKSYILEKVKEHQESMDMNNPQDFIDCFLMKMEKEKHNQPSEFTIESLENTAVDLFGAGTETTSTTLRYALLLLLKHPEVTAKVQEEIERVIGRNRSPCMQDRSHMPYTDAVVHEVQRYIDLLPTSLPHAVTCDIKFRNYLIPKGTTILISLTSVLHDNKEFPNPEMFDPHHFLDEGGNFKKSNYFMPFSAGKRICVGEALARMELFLFLTSILQNFNLKSLVDPKNLDTTPVVNGFASVPPFYQLCFIPV

SEQ ID NO: 53 - protein, 492 aa, Artificial Sequence
  Description of Artificial Sequence Synthetic polypeptide
MAADSFVVLVLCLSCLLLLSLWRQSSGRGKLPPGPTPLPVIGNILQIDIKDISKSLTNLSKVYGPVFTLYFGLKPIVVLHGYEAVKEALIDLGEEFSGRGHFPLAERANRGFGIVFSNGKKWKEIRRFSLMTLRNFGMGKRSIEDRVQEEARCLVEELRKTKASPCDPTFILGCAPCNVICSIIFRKRFDYKDQQFLNLMEKLNENVKILSSPWIQIYNNFSPIIDYFPGTHNKLLKNVAFMKSYILEKVKEHQESMDMNNPQDFIDCFLMKMEKEKHNQPSEFTIESLENTAADLFGAGTETTSTTLRYALLLLLKHPEVTAKVQEEIERVIGRNRSPCMQDRSHMPYTDAVVHEVQRYIDLLPTSLPHAVTCDIKFRNYLIPKGTTILISLTSVLHDNKEFPNPEMFDPHHFLDEGGNFKKSNYFMPFSAGKRICVGEALARMELFLFLTSILQNFNLKSLVDPKNLDTTPVVNGFASVPPFYQLCFIPV

SEQ ID NO: 54 - protein, 492 aa, Artificial Sequence
  Description of Artificial Sequence Synthetic polypeptide
MAADLVVFLALTLSCLILLSLWRQSSGRGKLPPGPTPLPIIGNFLQIDVKNISQSFTNFSKAYGPVFTLYLGSKPTVILHGYEAVKEALIDRGEEFAGRGSFPMAEKIIKGFGVVFSNGNRWKEMRRFTLMTLRNLGMGKRNIEDRVQEEAQCLVEELRKTKGSPCDPTFILSCAPCNVICSIIFQNRFDYKDKEFLILMDKINENVKILSSPWLQVCNSFPSLIDYCPGSHHKIVKNFNYLKSYLLEKIKEHKESLDVTNPRDFIDYYLIKQKQVNHIEQSEFSLENLASTINDLFGAGTETTSTTLRYALLLLLKYPDVTAKVQEEIDRVVGRHRSPCMQDRSHMPYTDAMIHEVQRFIDLLPTSLPHAVTCDIKFRKYLIPKGTTVITSLSSVLHDSKEFPNPEMFDPGHFLNANGNFKKSDYFMPFSTGKRICAGEGLARMELFLILTTILQNFKLKSLVHPKEIDITPVMNGFASLPPPYQLCFIPL

SEQ ID NO: 55 - protein, 492 aa, Artificial Sequence
  Description of Artificial Sequence Synthetic polypeptide
MAAILGVFLGLFLTCLLLLSLWKQNFQRRNLPPGPTPLPIIGNILQIDLKDISKSLRNFSKVYGPVFTLYLGRKPAVVLHGYEAVKEALIDHGEEFAGRGVFPVAQKFNKNCGVVFSSGRTWKEMRRFSLMTLRNFGMGKRSIEDRVQEEARCLVDELRKTNGVPCDPTFILGCAPCNVICSIVFQNRFDYKDQEFLALIDILNENVEILGSPWIQICNNFPAIIDYLPGRHRKLLKNFAFAKHYFLAKVIQHQESLDINNPRDFIDCFLIKMEQEKHNPKTEFTCENLIFTASDLFAAGTETTSTTLRYSLLLLLKYPEVTAKVQEEIDHVIGRHRSPCMQDRHHMPYTDAVLHEIQRYIDLLPTSLPHALTCDMKFRDYLIPKGTTVIASLTSVLYDDKEFPNPEKFDPSHFLDENGKFKKSDYFFPFSTGKRICVGEGLARTELFLFLTTILQNFNLKSPVDLKELDTNPVANGFVSVPPKFQICFIPI

SEQ ID NO: 56 - protein, 505 aa, Artificial Sequence
  Description of Artificial Sequence Synthetic polypeptide
MAAALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 57 - protein, 509 aa, Artificial Sequence
  Description of Artificial Sequence Synthetic polypeptide
MAADLIPNLAVETWLLLTKLEFGFYIFPFIYGTHSHGLFKKLGIPGPTPLPFLGNILSYRKGFCMFDMECHKKYGKVWGFYDGRQPVLAITDPDMIKTVLVKECYSVFTNRRPFGPVGFMKSAISIAEDEEWKRIRSLLSPTFTSGKLKEMVPIIAQYGDVLVRNLRREADTGKPVTLKDVFGAYSMDVITSTSFGVNIDSLNNPQDPFVENTKKLLRFDFLDPFFLSIIVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPVAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLRLGGLLQPEKPIVLKVESRDGTVSGA

SEQ ID NO: 58 - protein, 496 aa, Artificial Sequence
  Description of Artificial Sequence Synthetic polypeptide
MAAALIPDLAMETWLLLAVSLVLLYLYGTHSHGLFKKLGIPGPTPLPFLGNIWSYRKGFCMFDMECHKKYGKVWGFYDGRQPVLAITDPDMIKTVLVKECYSVFTNRRPFGPVGFMKSAISIAEDEEWKRLRSLLSPTFTSGKLKEMVPLIAQYGDVLVRNLRLEAETGKPVTMKVITSTSFGVNIDSLNNPQDPFVENTKKLLRFDFLDPFFLSIIVFPFLTPILEVLNISVFPRAVTSFLRKSVKRMKESRLEDTQKHRVDFLQLMIDSQNSKETESHKALSDLELVAQSIIFIFAGYETTSSVLSFITYELATHPDVQQKLQEEIDAVLPNKAPPTYDTVLQMEYLDMVVNETLRLFPIAMRLERVCKKDVEINGMFIPKGVVVMIPSYALHHDPKYWTEPEKFLPERFSKKNKDNIDPYIYTPFGSGPRNCIGMRFALMNMKLALIRVLQNFSFKPCKETQIPLKLRLGGLLQPEKPIVLKVESRDGTVSGA

SEQ ID NO: 59 - protein, 509 aa, Artificial Sequence
  Description of Artificial Sequence Synthetic polypeptide
MAAELIPSFSMETWVLLATSLVLLYIYGTYSYGLFKKLGIPGPRPVPYFGSTMAYHKGIPEFDNQCFKKYGKMWGFYEGRQPMLAITDPDIIKTVLVKECYSVFTNRRIFGPMGIMKYAISLAWDEQWKRIRTLLSPAFTSGKLKEMFPIIGQYGDMLVRNLRKEAEKGNPVNMKDMFGAYSMDVITGTAFGVNIDSLNNPHDPFVEHSKNLLRFRPFDPFILSIILFPFLNPVFEILNITLFPKSTVDFFTKSVKKIKESRLTDKQMNRVDLLQLMINSQNSKEIDNHKALSDIELVAQSTIFIFGGYETTSSTLSFIIYELTTHPHVQQKVQEEIDATFPNKAPPTYDALVQMEYLDMVVNETLRMFPIAGRLERVCKKDVEIHGVTIPKGTTVLVPLFVLHNNPELWPEPEEFRPERFSKNNKDSINPYVYLPFGTGPRNCLGMRFAIMNIKLALVRILQNFSFKPCKETQIPLKLYTQGLTQPEQPVILKVVPRGLGPQVEPDFL

SEQ ID NO: 60 - protein, 495 aa, Artificial Sequence
  Description of Artificial Sequence Synthetic polypeptide
MAADSFPLLAALFFILAATWFISFRRPRNLPPGPFPYPIVGNMLQLGTQPHETFAKLSKKYGPLMSIHLGSLYTVIVSSPEMAKEIMHKYGQVFSGRTVAQAVHACGHDKISMGFLPVGGEWRDMRKICKEQMFSHQSMEDSQWLRKQKLQQLLEYAQKCSERGRAIDIREAAFITTLNLMSATLFSMQATEFDSKVTMEFKEIIEGVASIVGVPNFADYFPILRPFDPQGVKRRADVYFGRLLAIIEGFLNERVESRRTNPNAPKKDDFLETLVDTLQTNDNKLKTDHLTHLMLDLFVGGSETSTTEIEWIMWELLANPEKMAKMKAELKSVMGEEKVVDESQMPRLPYLQAVVKESMRLHPPGPLLLPRKAESDQVVNGYLIPKGAQVLINAWAIGRDHSIWKNPDSFEPERFLDQKIDFKGTDYELIPFGSGRRVCPGMPLANRILHTVTATLVHNFDWKLERPEASDAHRGVLFGFAVRRAVPLKIVPFKV

SEQ ID NO: 61 - protein, 494 aa, Artificial Sequence
  Description of Artificial Sequence Synthetic polypeptide
MAADPFPLVAAALFIAATWFITFKRRRNLPPGPFPYPIVGNMLQLGSQPHETFAKLSKKYGPLMSIHLGSLYTVIISSPEMAKEIMHKYGQVFSGRTIAQAVHACDHDKISMGFLPVGAEWRDMRKICKEQMFSHQSMEDSQNLRKQKLQQLLEYAQKCSEEGRGIDIREAAFITTLNLMSATLFSMQATEFDSKVTMEFKEIIEGVASIVGVPNFADYFPILRPFDPQGVKRRADVYFGRLLGLIEGYLNERIEFRKANPNAPKKDDFLETLVDALDAKDYKLKTEHLTHLMLDLFVGGSETSTTEIEWIMWELLASPEKMAKVKAELKSVMGGEKVVDESMMPRLPYLQAVVKESMRLHPPGPLLLPRKAESDQVVNGYLIPKGAQVLINAWAMGRDPSLWKNPDSFEPERFLDQKIDFKGTDYELIPFGSGRRVCPGMPLANRILHTVTATLVHNFDWKLERPEASDAHKGVLFGFAVRRAVPLKIVPIKA

SEQ ID NO: 62 - protein, 497 aa, Artificial Sequence
  Description of Artificial Sequence Synthetic polypeptide
MAADSFPLLAALFFIAATITFLSFRRRRNLPPGPFPYPIVGNMLQLGANPHQVFAKLSKRYGPLMSIHLGSLYTVIVSSPEMAKEILHRHGQVFSGRTIAQAVHACDHDKISMGFLPVASEWRDMRKICKEQMFSNQSMEASQGLRRQKLQQLLDHVQKCSDSGRAVDIREAAFITTLNLMSATLFSSQATEFDSKATMEFKEIIEGVATIVGVPNFADYFPILRPFDPQGVKRRADVFFGKLLAKIEGYLNERLESKRANPNAPKKDDFLEIVVDIIQANEFKLKTHHFTHLMLDLFVGGSDTNTTSIEWAMSELVMNPDKMARLKAELKSVAGDEKIVDESAMPKLPYLQAVIKEVMRIHPPGPLLLPRKAESDQEVNGYLIPKGTQILINAYAIGRDPSIWTDPETFDPERFLDNKIDFKGQDYELLPFGSGRRVCPGMPLATRILHMATATLVHNFDWKLEDDSTAAADHAGELFGVAVRRAVPLRIIPIVKS

SEQ ID NO: 63 - protein, 679 aa, Artificial Sequence
  Description of Artificial Sequence Synthetic polypeptide
MAAGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 64 - protein, 693 aa, Artificial Sequence
  Description of Artificial Sequence Synthetic polypeptide
MAAPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

SEQ ID NO: 65 - protein, 680 aa, Artificial Sequence
  Description of Artificial Sequence Synthetic polypeptide
MAAGDSHEDTSATVPEAVAEEVSLFSTTDIVLFSLIVGVLTYWFIFKKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDTAKVYTGEMGRLKSYENQKPPFDAKNPFLAAVTTNRKLNQGTERHLMHLELDISDSKIRYESGDHVAVYPANDSTLVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRTKEPAGENGRRALVPMFVRKSQFRLPFKPTTPVIMVGPGTGVAPFMGFIQERAWLREQGKEVGETLLYYGCRRSDEDYLYREELARFHKDGALTQLNVAFSREQAHKVYVQHLLKRDKEHLWKLIHEGGAHIYVCGDARNMAKDVQNTFYDIVAEFGPMEHTQAVDYVKKLMTKGRYSLDVWS

SEQ ID NO: 66 - protein, 680 aa, Artificial Sequence
  Description of Artificial Sequence Synthetic polypeptide
MAAGDSHEDTSATMPEAVAEEVSLFSTTDMVLFSLIVGVLTYWFIFRKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDAAKVYTGEMGRLKSYENQKPPFDAKNPFLAAVTANRKLNQGTERHLMHLELDISDSKIRYESGDHVAVYPANDSALVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRAKEPAGENGGRALVPMFVRKSQFRLPFKSTTPVIMVGPGTGIAPFMGFIQERAWLREQGKEVGETLLYYGCRRSDEDYLYREELARFHKDGALTQLNVAFSREQAHKVYVQHLLKRDREHLWKLIHEGGAHIYVCGDARNMAKDVQNTFYDIVAEFGPMEHTQAVDYVKKLMTKGRYSLDVWS

SEQ ID NO: 67 - protein, 682 aa, Artificial Sequence
  Description of Artificial Sequence Synthetic polypeptide
MAAINMGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDIVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKRDREHLWKLIEGGAHIYVCGDARNMARDVQNTFYDIVAELGAMEHAQAVDYIKKLMTKGRYSLDVWS

SEQ ID NO: 68 - protein, 682 aa, Artificial Sequence
  Description of Artificial Sequence Synthetic polypeptide
MAAINMGDSHMDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIENALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASETSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELVQFHRDGALTQLNVAFSREQSHKVYVQHLLKRDREHLWKLIEGGAHIYVCGDARNMARDVQNTFYDIVAELGAMEHTQAVDYIKKLMTKGRYSLDVWS

SEQ ID NO: 69 - protein, 682 aa, Artificial Sequence
  Description of Artificial Sequence Synthetic polypeptide
MAANMADSNMDAGTTTSEMVAEEVSLFSTTDVILFSLIVGVMTYWFLFRKKKEEVPEFTKIQTTTSSVKDRSFVEKMKKTGRNIIVFYGSQTGTAEEFANRLSKDAHRYGMRGMAADPEEYDLADLSSLPEIEKALAIFCMATYGEGDPTDNAQDFYDWLQETDVDLSGVKYAVFALGNKTYEHFNAMGKYVDKRLEQLGAQRIFDLGLGDDDGNLEEDFITWREQFWPAVCEHFGVEATGEESSIRQYELMVHTDMDMAKVYTGEMGRLKSYENQKPPFDAKNPFLAVVTTNRKLNQGTERHLMHLELDISDSKIRYESGDHVAVYPANDSALVNQLGEILGADLDIIMSLNNLDEESNKKHPFPCPTSYRTALTYYLDITNPPRTNVLYELAQYASEPTEHEQLRKMASSSGEGKELYLRWVLEARRHILAILQDYPSLRPPIDHLCELLPRLQARYYSIASSSKVHPNSVHICAVAVEYETKTGRINKGVATSWLRAKEPAGENGGRALVPMYVRKSQFRLPFKATTPVIMVGPGTGVAPFIGFIQERAWLRQQGKEVGETLLYYGCRRSDEDYLYREELAGFHKDGALTQLNVAFSREQPQKVYVQHLLKKDKEHLWKLIHEGGAHIYVCGDARNMARDVQNTFYDIVAEQGAMEHAQAVDYVKKLMTKGRYSLDVWS

SEQ ID NO: 70 - protein, 709 aa, Artificial Sequence
  Description of Artificial Sequence Synthetic polypeptide
MAAEPTSQKLSPLDFIAAILKGDYSDGQLEAASPGMAMLLENRDLVMVLTTSVAVLIGCVVVLAWRRTAGSATKKQFEPPKLVVPKAAELEEVDDDKPKVSIFFGTQTGTAEGFAKAFAEEAKARYPQANFKVIDLDDYAADDDEYEEKLKKETLAFFFLASYGDGEPTDNAARFYKWFTEGKDRGDWLKNLQYGVFGLGNRQYEHFNKIAIVVNDIIVEQGGKKLVSVGLGDDDQCIEDDFAAWRELVWPELDKLLRNEDDATVSTPYTAAVLQYRVVFHDQTDGLISENGFLNGRANGTSVFDAQHPCRSNVAVKKELHTPASDRSCAHLEFDISGTGLVYETGDHVGVYCENLIETVEEAEKLLNIPPQTYFSIHTDNEDGSPRSGSSLPPPFPPCTLRTALTRYADLLSAPKKSTLIALAESASDQSEADRLRHLASPGGKEEYAQYITASQRSLLEVMADFPSAKPSLGVFFAAIAPRLQPRFYSISSSPKIAPSRIHVTCALVYEKTPTGRVHKGVCSTWMKNAVPLEESNDCSWAPIFVRNSNFKLPAYPKVPIIMIGPGTGLAPFRGFLQERLALKESGAELGPAILFFGCRNRKMDFIYEDELNNFVEAGVISELIVAFSREGPTKEYVQHKMTQRASDVWKIISDGGYVYVCGDAKGMARDVHRTLHTIAQEQGSLSSSEAEGMVKNLQTTGRYLRDVW

SEQ ID NO: 71 - protein, 250 aa, Artificial Sequence
  Description of Artificial Sequence Synthetic polypeptide
MAADFSGKNVWVTGAGKGIGYATALAFVEAGAKVTGFDQAFTQEQYPFATEVMDVADAAQVAQVCQRLLAETERLDALVNAAGILRMGATDQLSKEDWQQTFAVNVGGAFNLFQQTMNQFRRQRGGAIVTVASDAAHTPRIGMSAYGASKAALKSLALSVGLELAGSGVRCNVVSPGSTDTDMQRTLWVSDDAEEQRIRGFGEQFKLGIPLGKIARPQEIANTILFLASDLASHITLQDIVVDGGSTLGA

SEQ ID NO: 72 - protein, 272 aa, Artificial Sequence
  Description of Artificial Sequence Synthetic polypeptide
MAASDLHNESIFITGGGSGLGLALVERFIEEGAQVATLELSAAKVASLRQRFGEHILAVEGNVTCYADYQRAVDQILTRSGKLDCFIGNAGIWDHNASLVNTPAETLETGFHELFNVNVLGYLLGAKACAPALIASEGSMIFTLSNAAWYPGGGGPLYTASKHAATGLIRQLAYELAPKVRVNGVGPCGMASDLRGPQALGQSETSIMQSLTPEKIAAILPLQFFPQPADFTGPYVMLTSRRNNRALSGVMINADAGLAIRGIRHVAAGLDL

SEQ ID NO: 73 - protein, 269 aa, Artificial Sequence
  Description of Artificial Sequence Synthetic polypeptide
MAATGWLAGKRALIVGAGSGIGRATVDAFLNEDARVAVLEYDSDKCATLRHQLPDVPVIEGDGTTRTANDEAVQVAVDAFGGLDTLVNCVGIFDFYRRIQDIPAELIDQAFDEMFRINVLSHIHSVKAAVPALMGQDGASIVLTESASSFYPGRGGLLYVASKFAVRGVVTALAHELAPRIRVNGVAPGGTLNTDLRGLDSLDLGARRLDAAPDRARELAARTPLGVALSGEDHAWSYVFLASHRSRGLTGETIHPDGGFSLGPPPQRN

SEQ ID NO: 74 - protein, 267 aa, Artificial Sequence
  Description of Artificial Sequence Synthetic polypeptide
MSSIETKIFPGRFDGRCLTITGAAQGIGLTVATRIAAEGGEVVLVDRADLVHEVAEQLREAGGKAHSVTADLETFEGAEEAISHAVRTTGRIDVLINVVGGTIWAKPYEHYAPEEIEKEIRRSLFPTLWTCRAAAPHLIERRAGTIVNVSSVATRGVNRVPYSAAKGGVNAITASLALELAPYGVRVVATAPGGTVAPERRIARGPSPQSEQEKAWYQQIVDQTVDSSLLKRYGTLDEQAAAICFLASEEASYITGTVLPVAGGDLG

SEQ ID NO: 75 - protein, 270 aa, Artificial Sequence
  Description of Artificial Sequence Synthetic polypeptide
MSSTGWLDGKRALVVGGGSGIGRAVVDAFLAEGACVAVLERDPNKCRVLREHLPQVPVIEGDATRAADNDAAVAAAVAAFGGLDTLVNCVGIFDFYQGIEDIPADTLDVAFDEMFRTNVLSHMHSVKAAVPELRKHRGSSIVLAESASSFYPGRGGVLYVSSKFAVRGLVTTLAYELAPDIRVNGVAPGGTLNTDLRGLASLGRDADRLDDNPNRANELAARTPLNVALSGEDHAWSFVFFASDRSRGITAGATHPDGGFGIGAPKPSTR

SEQ ID NO: 76 - protein, 273 aa, Artificial Sequence
  Description of Artificial Sequence Synthetic polypeptide
MSSGFLDGKVALVTGGGSGIGRAVVELYVQQGAKVGILEISPEKVKDLRNALPADSVVVTEGDATSMADNERAVADVVDAFGPLTTLVCVVGVFDYFTEIPQLPKDKISEAFDQLFGVNVKSNLLSVKAALDELIENEGDIILTLSNAAFYAGGGGPLYVSSKFAVRGLVTELAYELAPKVRVNGVAPGGTITELRGIPALANEGQRLKDVPDIEGLIEGINPLGIVAQPEDHSWAYALLASRERTSAVTGTIINSDGGLGVRGMTRMAGLAQ

SEQ ID NO: 77 - protein, 258 aa, Artificial Sequence
  Description of Artificial Sequence Synthetic polypeptide
MSSSRSVTLVVGAAQGIGRATALTLATAGHRVVLADRDVDGLAETAALLHVAAPVHGLDVCDAAGVAEAVARVEVEHGPVDALAHVAGVFTTGSVLDSDLAEWQRMFDVNVTGLINVLRVVGHGMRERRRGAIVTVGSNSAGVPRVGMGAYGASKSAAHMLVRVLGLELARFGVRANVVAPGSTDTAMQRSLWPDPADDAGARTAIDGDAASFKVGIPLGRIADPADIADAVEFLLSDRARHITMQTLYVDGGATLRA

SEQ ID NO: 78 - protein, 277 aa, Artificial Sequence
  Description of Artificial Sequence Synthetic polypeptide
MSSQMLDDHVALILGGGSGLGLGIARHFLGEGAQVAIFEISESKLLDLKAEFGDDVLLLQGDVTSIDDLEAARAAVVDRFGRLDALIGAQGIFDGNIPLRDIPTERIEKVFDEVLHVDVLGYILAARVFLEELEKTDGAIVFTSSTAAYAADGGGLFYTAAKGAVRSVINQLAFEFAPKVRVNGVAPSGIANSQLQGPRALGLENNKQSDIPVEDFTNQFLSLTLTPTLPTPEEYAPLYAYLASRNNTTMTGQTIIADQGLFNRAVISNGVADRVGK

SEQ ID NO: 79 - protein, 426 aa, Artificial Sequence
  Description of Artificial Sequence Synthetic polypeptide
MSSSGPAHSNLEQVFANVASNYRGADVDLHAVYREMREKSPVLPENFMARLGVPSIAGLDPNRPTFTLFKYDDVMAVMRDATNFTSGFIAEGLGSFFDGLILTAMDGEAHKNIRSLLQPVFMPETVNRWKETKIDRVIREEYLRPMVASKRADIMEFALYFPIRVIYSLIGFPEDRPEEIEQYAAWALAILAGPQVDPEKAAAARGAAMEAAQALYDVVKVVVAQRRAEGATGDDLICRLIRAEYEGRSLDDHEITTFVRSLLPAASETTTRTFGTLMTLLLERPELLARIREDRSLVGKAIDEAVRYEPVATFKVRQAAKDVEIRGVAIPKGAMVSCIVTSANRDEDAFENADTFDIDRRAKPSFGFGFGPHMCIGQFVAKTEINCALNAILDLMPNIRLDPDKPAPEIIGAQLRGPHHVHVIWD

SEQ ID NO: 80 - protein, 408 aa, Artificial Sequence
  Description of Artificial Sequence Synthetic polypeptide
MSSRSTDLPDLKSAAFLADRYPTYRRLQSDFPHFEMNINGEECIVLTRYSDVDEVLRNPLATVQQAPGVFPERIGQGAGARFYRESLPNIDAPDHTRIRRIVTPAFNPKTVANMRGWVEKVIVEHLDRLEGLDEIDFVSSFADPVPAEIACRLLHVPVSDAPELFARQHGLNAVLSVSDITPERLAEADASAAFYYEYMDDVLNTLKGKLPEDDFVGALMAAEARDSGLTRSELVTTLIGFLVASYHTTKVAMTNTVLALLNHDGERARLVAQPDLARNAWEESLRYDSPVHFVHRYASEPLTIGGQPVAQGKRLLLGLHAASRDENRFAQADHYLIDRPDNRHLAFAGGGHFCLGSQLSRLEGDVLLRTIFQRFPAMRLTETRFERVPDLTFPMLLRMTVSLRAEQG

SEQ ID NO: 81 - protein, 487 aa, Artificial Sequence
  Description of Artificial Sequence Synthetic polypeptide
MSSTSNSIRSPLSPPQPRRTPPPCTSSREPPIVRGTWLLGSTRDLLRDPLELGLRGYAEGGDVVRYVVGLPGRRREFFTVNHPDGVGELLNAPRHLDYRKDSEFYRAMRDLYGNGLVTSQDETWLRQRRFIQPLFTPQSVDGYVTPMVAEADRVAIRWHNCTSRLVDLDGEMRALTLGVAARILFGVQAPRMLPILRTTLPVLGRAVLQQGASAIRFPSSWPTPGNRRIASAESRLDGLCDAIIERRRTVAEPGTDLLGRLVAAREDGDTLSTEEIRDQVKVFLLAGHDTTATMLTFALYLLGKDAGVQDQARDEAERVLGAGTPTASDVHRLTYTTMVLEEAARLYPPSPYLTRRAVEESEVCGYRIPAGADVNLAPWVIHHRADLWPDPFRFDPDRFTPDRVKERHKYAWFPFGHGPRGCIGQRFAMLEAAVTLAILLREFEFRSPPGSVPLTVDLLLHPAGEVPCRVRRRVPVHSAVHRTHQPS

SEQ ID NO: 82 - protein, 414 aa, Artificial Sequence
  Description of Artificial Sequence Synthetic polypeptide
MSSAPDILSPEFLDNPYPLHRVLRDHYPALHHEGTDSYLISRYADCAEAFRSPKFSSRNYEWQLEPIHGRTILQMEGREHSTHRALLNPFFRGNGLERFMPAITHNAAQLIGDIVARNAGELLGAVARQGEAELVSQFTSRFPINVMVDMLGLPKSDHERFRGWYFSIMAYLNNLAGDPEINAAAERTHVELREYMLPIIRERRSGDGDDLLSRLCRAEVDGEQMSDEEIKAFVSLLLVAGGETTDKAIASMIRNLIDHPDQMRAVREDRSLADRVIAETLRYSGPVHMIMRQTEDEVQIEDSTIPAGATCIMMLAAANRDERHFSNPDEFDIFRTDLNVDRAFSGAANHVQFILGRHFCVGSMLAKTEMTIALNLVLDTMDSIEYQDGFVPREEGLYTRSIPELRVKFEGKLG

SEQ ID NO: 83 - protein, 427 aa, Artificial Sequence
  Description of Artificial Sequence Synthetic polypeptide
MSSSTPAAATSLESAFAGVADNYKGSDVDLHAIYRDMRRNSPVIAEDFMARLGVPNIAGLDAKRPTFTLFKYKDVMSVLRDATNFTSGFIAEGLGAFFDGLILTGMDGEAHRRTRSLLQPVFMPDVVNRWRETKMAPIVRNEYIEPMVPKRRADLMDFGLHFPIRLIYSLIGFPDNRPEQIEQYAAWALAILAGPQVDAEKAAQARKAAMEAAQALYDAVKLEVTEVRKNGAQGDDLICRLIRAEYEGRHLDDHEVTTFVRSLLPAAGETTTRTFGSLMVALLERPELLERVRADRSLVPKAIDEAVRFEPVATFKVRQAAQDTEIGGFSIPKGAMVQCIVSSANRDEEVFENSESFDIDRKLKPSFGFGFGPHMCIGQFIAKVELSVAVNTILDLLPNLRLDPDRPKPRIVGAQLRGPHALHVIWD

SEQ ID NO: 84 - protein, 425 aa, Artificial Sequence
  Description of Artificial Sequence Synthetic polypeptide
MSSSPSVAELSQELGEAFRLSSMDDPYPMLAERRRETPVMKGDIMVALGAPSYMGQHAGETHTVFRHDDVMAILRNHETFSSSIWEISQGPLIGRSILAMDGAEHRQWRGYLQSVFGGKLLSSWDESIFRPLAAKYVADLASKRGADLIAMALEYPLRAIYEILGLEDFKDNYEEFHADVLTILLALWSTPDPAQADQFLLRFQKATEASARSWDRLLPIVQRKRAAGASRNDLISSLIRAEYEGGVLDDEQITSFLRSLLLAATDTTTRQFLNTLTLLLQRPDELDRIRRDRSRLRLALAEGERLEPPALFIPRMITRDVVIRGTELTAGTPLLLAIGSANRDPEAYPPDPDEFRIDRTGPHHATFGFGTHICSGMNTTRREIAALIDAMLDGLPGLRVDPDAPAPLISGIHFRGPSALPVVWD

SEQ ID NO: 85 - protein, 365 aa, Artificial Sequence
  Description of Artificial Sequence Synthetic polypeptide
MSSDYSRTPESLRPADSYAALSYSTVNAALRNDRVFSSKMYDSTIGVFMGPTILAMSGTKHRAHRNLVSAAFKPQSLRVWEPDIVRPICNALIDEFAGTGHADLVRDFTFEFPTRVIARLLGLPAEDLPFFRKAAVAIISYAGNVPRALEASEDLKNYFLGHIEQRRSQPTDDIISDLVTAEVEGEQLTDEAIYSFLRLLLPAGLETTYRSSGNLLYLLLRHPRQFAAVQGNHGLIPQAVEEGLRYETPLTFVQRFTTEDTELGGVPVPAGAVVDLVLGSANRDEDRWERPGEFDIFRKPVPHISFTAGAHTCLGLHLARMETRVAVECLLTRLTNFRLQDEGDPHITGQPFRSPNLLPVTFDVV

SEQ ID NO: 86 - protein, 453 aa, Artificial Sequence
  Description of Artificial Sequence Synthetic polypeptide
MSSPTPRWRIPVLGDLLSVDPAKPVQKEMAMAAELGPLFERKIIGSRLTVVSGVDLVAEVNDEKHWARALGRPILKLRDVAGDGLFTAFNSEPAWARAHSVLGPGFSQSALRTYHGSMTRVLDDLVATWDDAAASGARVDVARDMTRLTFDVIGRAGFGRDFGSLRGDDLDPFAAAMGRALGYVNQTSNDIPLLRMVFGRGAAKRYQTDVAFMRDTVDELVASRAGRAERSDDLLDLMLHSADPDTGERLDMENIRNQVLTFLVAGNETTASTLAFALYFLAREPEVVERARAEIADVVGDGEIAFEQVAKLRYVRRVVDETLRLWPAAPGYFRKVRHDTVLGGRYPMPKGSWVFVLLPQLHRDPVWGDDPERFDPDRFAPDAVRARPKDAYRPFGTGPRSCIGRQFALHEAVLALATLLRRYDVAPDPAYRLDIVEAVTLKPRGFELTLQRR

SEQ ID NO: 87 - protein, 426 aa, Artificial Sequence
  Description of Artificial Sequence Synthetic polypeptide
MSSSASSQSNLEQVFANVASNYRGADIDLHAVYREMREKSPVLPENFMARLGVPSIAGLDPDRPAFTLFKYDDVMAVMRDATNFTSGFIAEGLGSFFDGLILTAMDGEAHKNIRSLLQPVFMPETVNRWKETKIDRVIREEYLQPMVASKGADIMEFALYFPIRVIYSLIGFPEDRPEEIEQYAAWALAILAGPQVDPEKAVAARGAAMEAAQALYDVVKVVVAQRRSQGATGDDLISRLIRAEYEGRSLDDHEITTFVRSLLPAASETTTRTFGTLMTLLLERPELLARIREDRSLVPKAIDEAVRYEPVATFKVRQAAKDVEIRGVAIPQGAMVSCIVTSANRDEDAFENADTFNIDRRAKPSFGFGFGPHMCIGQFVAKTEINCALNAILDLMPNIRLDPDKPAPEIIGAQLRGPHHVHVIWD

SEQ ID NO: 88 - protein, 458 aa, Artificial Sequence
  Description of Artificial Sequence Synthetic polypeptide
MSSTATELRDAPGSAPGLPRRSMLSLLPRMARDRLSVMTSVAARYGDAVTLPLGLSTLHFFNHPDYAKHVLADNSSNYHKGIGLIHAKRALGDGLLTSEGELWRKQRKTIQPAFAVKRLAGQAGAIAEEADRLVEHLLARQGRGPVDIRHEMTALTLGVLGRTLLDADLGAFGSVGHWFEAVQDQAMFDMMSLGTVPLWSPLPKQLRFRRARRELESVVDRLVAQRGDRPRADGDDVVSRLVDSTGRERDPALRRKRMHDELVTLLLAGHETTASTLSWTFHLADEHPEVWERLHAEAVEVLGDRRPVFEDLHRLRYTNRVLNEVMRLYPPVWLLPRRAVADDVVGGYRVPAGSDVLICPYTLHRHPEFWELPSRFDPDRFDPERSANRPRYAYIPFGAGPRFCVGNNLGLMEAAFVIAAIARRMRLRKVPGGTVVPEPMLTLRVRSGLPMTVHALDR

SEQ ID NO: 89 - protein, 518 aa, Artificial Sequence
  Description of Artificial Sequence Synthetic polypeptide
MSSQRRDFLKYSVALGVASALPLWSRAVFAAERPTLPIPDLLTTDARNRIQLTIGAGQSTFGGKTATTWGYNGNLLGPAVKLQRGKAVTVDIYNQLTEETTLHWHGLEVPGEVDGGPQGIIPPGGKRSVTLNVDQPAATCWFHPHQHGKTGRQVAMGLAGLVVIEDDEILKLMLPKQWGIDDVPVIVQDKKFSADGQIDYQLDVMTAAVGWFGDTLLTNGAIYPQHAAPRGWLRLRLLNGCNARSLNFATSDNRPLYVIASDGGLLPEPVKVSELPVLMGERFEVLVEVNDNKPFDLVTLPVSQMGMAIAPFDKPHPVMRIQPIAISASGALPDTLSSLPALPSLEGLTVRKLQLSMDPMLDMMGMQMLMEKYGDQAMAGMDHSQMMGHMGHGNMNHMNHGGKFDFHHANKINGQAFDMNKPMFAAAKGQYERWVISGVGDMMLHPFHIHGTQFRILSENGKPPAAHRAGWKDTVKVEGNVSEVLVKFNHDAPKEHAYMAHCHLLEHEDTGMMLGFTV

SEQ ID NO: 90 - protein, 522 aa, Artificial Sequence
  Description of Artificial Sequence Synthetic polypeptide
MSSRLSFLTSLVTLALVSSTYAGVGPVVDLTVSNAVISPDGFDRDAIVVNGVFPAPLITGKKGDRFQLNVIDNMTNHTMLKSTSIHWHGFFQKGTNWADGGAFVNQCPIAPGHSFLYDFRVPDQAGTFWYHSHLSTQYCDGLRGPIVVYDPNDPHADLYDVDNDSTVITLADWYHVAARLGPRFPLGADSTVINGLGRSLSTPNADLAVISVTQGKRYRFRLISLSCDPFHTFSIDGHDLTIIEADSVNTEPLVVDAIPIFAGQRYSFVLSAVKDIDNYWIRADPNFGTTGFASGINSAILRYDGAAPIEPTAVLAPVSVNPLVETDLHPLEDMPVPGRPTKGGVDKAINLDFSFSFPNFFINNATFTSPTVPILLQIMSGAQAAQDLLPSGSVIELPAQSTIELTLPATVNAPGVPHPFHLHGHTFAVVRSAGSTAYNYDNPIWRDVVSTGTPAANDNVTIRFTTDNPGPWFLHCHIDFHLEAGFAVVFAEGVPQTQVANPVPQAWEELCPIYDALPEDDQ

SEQ ID NO: 91 - protein, 558 aa, Artificial Sequence
  Description of Artificial Sequence Synthetic polypeptide
MSSFKVSCKVTNNNGDQNVETNSVDRRNVLLGLGGLYGVANAIPLAASAAPTPPPDLKTCGKATISDGPLVGYTCCPPPMPTNFDNIPYYKFPSMTKLRIRSPAHAVDEEYIAKYNLAISRMKDLDKTEPLNPLGFKQQANIHCAYCNGAYVFGDKVLQVHNSWLFFPFHRWYLYFYERILGKLIDDPTFALPYWNWDHPKGMRLPPMFDREGTSIYDERRNQQVRNGTVMDLGSFGDKVETTQLQLMSNNLTLMYRQMVTNAPCPLLFFGAPYVLGNNVEAPGTIENIPHIPVHIWAGTVRGSTFPNGDTSYGEDMGNFYSAGLDSVFYCHHGNVDRMWNEWKAIGGKRRDLSEKDWLNSEFFFYDENKKPYRVKVRDCLDAKKMGYDYAPMPTPWRNFKPKTKVSAGKVNTSSLPPVNEVFPLAKMDKVISFSINRPASSRTQQEKNEQEEMLTFDNIKYDNRGYIRFDVFLNVDNNVNANELDKVEFAGSYTSLPHVHRVGENDHTATVTFQLAITELLEDIGLEDEETIAVTLVPKKGGEGISIENVEIKLLDC

SEQ ID NO: 92 - protein, 559 aa, Artificial Sequence
  Description of Artificial Sequence Synthetic polypeptide
MSGQNKMGLILVFLFLDGLLVCLAADVDVHNYTFVLQEKNFTKWCSTKSMLVVNGSFPGPTITARKGDTIFVNVINQGKYGLTIHWHGVKQPRNPWSDGPEYITQCPIKPGTNFIYEVILSTEEGTLWWHAHSDWTRATVHGALVILPANGTTYPFPPPYQEQTIVLASWFKGDVMEVITSSEETGVFPAAADGFTINGELGDLYNCSKETTYRLSVQPNKTYLLRIVNAVLNEEKFFGIAKHTLTVVAQDASYIKPINTSYIMITPGQTMDVLFTTDQTPSHYYMVASPFHDALDTFANFSTNAIIQYNGSYKAPKSPFVKPLPVYNDIKAADKFTGKLRSLANEKFPVNVPKVNVRRIFMAVSLNIVKCANKSCNNNIGHSTSASLNNISFALPQTDVLQAYYRNISGVFGRDFPTVQKKANFSLNTAQGTQVLMIEYGEAVEIVYQGTNLGAATSHPMHLHGFNFYLVGTGAGTFNNVTDPPKYNLVDPPELNTINLPRIGWAAIRFVADNPGVWFLHCHFERHTTEGMATVVIVKDGGTTNTSMLPSPAYMPPCS

SEQ ID NO: 93 - protein, 668 aa, Artificial Sequence
  Description of Artificial Sequence Synthetic polypeptide
MSSRKICLGCSHSLSSQPFTYTTQKTVSSRRIGDSQWRLSRGYTRTLTSASASVATAPAKLLTVNETQKCLRNMVRGGDVISYILSHSSRNADQNLKDLDSLILEPVCSATHEMFDVFEIPEHILTPFCDNRNVPEEQVTRNPNLRTDCLTMKRFVLLQSLVAVASAGIGPVADLYVGNRILAPDGFNRSTVLGGTSSSDFGFPAPLITGTKGDRFQLNVINQLTDTTMLRSTSIHWHGLFQAGSSWADGPVGVNQCPIAPGNSFLYDFNVPDQAGTFWYHSHYSTQYCDGLRGAFVVRDPNDPHASLYDVDNDDTVITLADWYHTSAKELSGSFPAEEATLINGLGRYSGGPTSPLAIVNVEAGKRYRFRLVSISCDPFYTFSIDGHDLTIIEADGENTDPLVVDYLEIYAGQRYSVVLNANQPVDNYWIRANSSNGPRDFVGGTNSAILRYAGASNSDPTTELGPRNNRLVENNLHALGSPGVPGTHTIGEADVNINLEILFTPPNVLTVNGAQFIPPTAPVLLQILSGTKQATDLLPPGSVYVLPRNAVVELTIPGGSGGSPHPMHLHGHVFDVVRSAGSDTINWDNPVRRDVVNIGTSTSDNATIRFTTDNPGPWIFHCHIDWHLEVGLAVVFAEDPDTIENSTHPAAWDELCPIYDNLPSDEL

SEQ ID NO: 94 - protein, 515 aa, Artificial Sequence
  Description of Artificial Sequence Synthetic polypeptide
MSSTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQDEYGRPVLLLNNKRWHDPVTETPKVGTTEIWSIINPTRGTHPIHLHLVSFRVLDRRPFDIARYQESGELSYTGPAVPPPPSEKGWKDTIQAHAGEVLRIAATFGPYSGRYVWHCHILEHEDYDMMRPMDITDPHK

SEQ ID NO: 95 - protein, 518 aa, Artificial Sequence
  Description of Artificial Sequence Synthetic polypeptide
MSSVFSAAFSAFVALGLTLGAFAAVGPVADIHITDDTIAPDGFSRAAVLAGGTFPGPLITGNMGDAFKLNVIDELTDASMLKSTSIHWHGFFQKGTNWADGPAFVNQCPITTGNSFLYDFQVPDQAGTYWYHSHLSTQYCDGLRGAFVVYDPSDPHKDLYDVDDESTVITLADWYHTLARQIVGVAISDTTLINGLGRNTDGPADAALAVINVEAGKRYRFRLVSISCDPNWVFSIDNHDFTVIEVDGVNSQPLNVDSVQIFAGQRYSLVLNANQPVDNYWIRADPNLGTTGFAGGINSAILRYKGAAVAEPTTSQTTSTKPLLETDLHPLVSTPVPGLPQPGGTDVVQNLILGFNAGQFTINGASFVPPTVPVLLQILSGTTNAQDLLPSGSVFELPLGKTVELTLAAGVLGGPHPFHLHGHNFHVVRSAGQDTPNYDDPIVRDVVSTGASGDNVTIRFTTDNPGPWFLHCHIDWHLEAGFAVVFAEAVNETKSGNPTPAAWDNLCTLYDALADGDK

SEQ ID NO: 96 - protein, 636 aa, Artificial Sequence
  Description of Artificial Sequence Synthetic polypeptide
MSSCLAAIWSRKRAEHAASRLPALQEKRSTLSYAYARLDGSLASMFPNRFWSSVSLGARIKPVDGSSEEPTARPSSCARPFLHSASSESGFVSSSRPTSFCVTCSRRWRCCSLLAMLGFRFLHTSVLAALTLSLKSYAAIGPVTDLTVANANISPDGYERAAVLAGGSFPGPLITGRKGDHFQINVVDQLTNHTMLKSTSIHWHGLFQKGTNWADGPAFVNQCPISTGNSFLYDFHVPDQAGTFWYHSHLSTQYCDGLRGAMVVYDPNDPHKNLYDVDNDDTVITLADWYHVASKLGPAVPFGGDSTLINGKGRSTATPTADLAVISVTQGKRYRFRLVSLSCDPNFTFSIDGHALTVIEADAVSTQPLTVDSIQIFAGQRYSFVLNANQSVDSYWIRAQPSLGNVGFDGGLNSAILRYDGAAPTEPSALAVPVSTNPLVETALRPLNSMPVPGKAEVGGVDKAINLAFSFNGTNFFINGATFVPPAVPVLLQIMSGAQSASDLLPSGSVFVLPSNATIELSFPATANAPGAPHPFHLHGHTFAVVRSAGSAEYNYENPIWRDVVSTGSPGDNVTIRFRTDNPGPWFLHCHIDPHLEAGFAVVMAEDTRDVKADNPEPKAWDDLCPTYNALAVDDQ

SEQ ID NO: 97 - protein, 775 aa, Artificial Sequence
  Description of Artificial Sequence Synthetic polypeptide
MFPGARILATLTLALHLLHGTNAAIGPTGDMYIVNEDVSPDGFTRSAVVARSDPTTNGTSETLTGVLVQGNKGDNFQLNVLNQLSDTTMLKTTSIHWHGFFQSGSTWADGPAFVNQCPIASGNSFLYDFNVPDQAGTFWYHSHLSTQYCDGLRGPFIVYDPSDPHLSLYDVDNADTIITLEDWYHVVAPQNAVLPTADSTLINGKGRFAGGPTSALAVINVESNKRYRFRLISMSCDPNFTFSIDGHSLQVIEADAVNIVPIVVDSIQIFAGQRYSFVLNANQTVDNYWIRADPNLGSTGFDGGINSAILRYAGATEDDPTTTSSTSTPLEETNLVPLENPGAPGPAVPGGADININLAMAFDVTNFELTINGSPFKAPTAPVLLQILSGATTAASLLPSGSIYSLEANKVVEISIPALAVGGPHPFHLHGHTFDVIRSAGSTTYNFDTPARRDVVNTGTDANDNVTIRFVTDNPGPWFLHCHIDWHLEIGLAVVFAEDVTSITAPPAAWDDLCPIYDALSDSDKDNPRFGFAPATGGKATGRRNWFSKARRRAILVPYLKLLKLGLVMVFYIRAERNHGRLSQSTPPNRRVDQRELITNTWVERFFLHRLMFLKLFVGTACFMHIFNSVSSLGMCTLRTSHGSSESLASPSATMMLGGGLTLLSANIRLWCYAEMRDLYDFEVNIKKAHRLVTTGPYSVSMVMFSKDHWLYQCGLRSMVGVVLSCIWCAEVVLINGIMVPARMKVEDDGLRRHFGREWDEYASRVAYRLVPEIY

SEQ ID NO: 98 - protein, 608 aa, Artificial Sequence
  Description of Artificial Sequence Synthetic polypeptide
MSSKSFISAATLLVGILTPSVAAAPPSTPEQRDLLVPITEREEAAVKARQQSCNTPSNRACWTDGYDINTDYEVDSPDTGVVRPYTLTLTEVDNWTGPDGVVKEKVMLVNNSIIGPTIFADWGDTIQVTVINNLETNGTSIHWHGLHQKGTNLHDGANGITECPIPPKGGRKVYRFKAQQYGTSWYHSHFSAQYGNGVVGAIQINGPASLPYDTDLGVFPISDYYYSSADELVELTKNSGAPFSDNVLFNGTAKHPETGEGEYANVTLTPGRRHRLRLINTSVENHFQVSLVNHTMTIIAADMVPVNAMTVDSLFLGVGQRYDVVIEASRTPGNYWFNVTFGGGLLCGGSRNPYPAAIFHYAGAPGGPPTDEGKAPVDHNCLDLPNLKPVVARDVPLSGFAKRPDNTLDVTLDTTGTPLFVWKVNGSAINIDWGRPVVDYVLTQNTSFPPGYNIVEVNGADQWSYWLIENDPGAPFTLPHPMHLHGHDFYVLGRSPDESPASNERHVFDPARDAGLLSGANPVRRDVTMLPAFGWVVLAFRADNPGAWLFHCHIAWHVSGGLGVVYLERADDLRGAVSDADADDLDRLCADWRRYWPTNPYPKSDSGL

SEQ ID NO: 99 - protein, 519 aa, Artificial Sequence
  Description of Artificial Sequence Synthetic polypeptide
MSSRFQSLFFFVLVSLTAVANAAIGPVADLTLTNAQVSPDGFAREAVVVNGITPAPLITGNKGDRFQLNVIDQLTNHTMLKTSSIHWHGFFQQGTNWADGPAFVNQCPIASGHSFLYDFQVPDQAGTFWYHSHLSTQYCDGLRGPFVVYDPNDPHASLYDIDNDDTVITLADWYHVAAKLGPRFPFGSDSTLINGLGRTTGIAPSDLAVIKVTQGKRYRFRLVSLSCDPNHTFSIDNHTMTIIEADSINTQPLEVDSIQIFAAQRYSFVLDASQPVDNYWIRANPAFGNTGFAGGINSAILRYDGAPEIEPTSVQTTPTKPLNEVDLHPLSPMPVPGSPEPGGVDKPLNLVFNFNGTNFFINDHTFVPPSVPVLLQILSGAQAAQDLVPEGSVFVLPSNSSIEISFPATANAPGFPHPFHLHGHAFAVVRSAGSSVYNYDNPIFRDVVSTGQPGDNVTIRFETNNPGPWFLHCHIDFHLDAGFAVVMAEDTPDTKAANPVPQAWSDLCPIYDALDPSDL

SEQ ID NO: 100 - protein, 521 aa, Artificial Sequence
  Description of Artificial Sequence Synthetic polypeptide
MSSGLQRFSFFVTLALVARSLAAIGPVASLVVANAPVSPDGFLRDAIVVNGVVPSPLITGKKGDRFQLNVDDTLTNHSMLKSTSIHWHGFFQAGTNWADGPAFVNQCPIASGHSFLYDFHVPDQAGTFWYHSHLSTQYCDGLRGPFVVYDPKDPHASRYDVDNESTVITLTDWYHTAARLGPRFPLGADATLINGLGRSASTPTAALAVINVQHGKRYRFRLVSISCDPNYTFSIDGHNLTVIEVDGINSQPLLVDSIQIFAAQRYSFVLNANQTVGNYWVRANPNFGTVGFAGGINSAILRYQGAPVAEPTTTQTTSVIPLIETNLHPLARMPVPGSPTPGGVDKALNLAFNFNGTNFFINNATFTPPTVPVLLQILSGAQTAQDLLPAGSVYPLPAHSTIEITLPATALAPGAPHPFHLHGHAFAVVRSAGSTTYNYNDPIFRDVVSTGTPAAGDNVTIRFQTDNPGPWFLHCHIDFHLDAGFAIVFAEDVADVKAANPVPKAWSDLCPIYDGLSEANQ

SEQ ID NO: 101 - DNA, 1164 bp, Artificial Sequence
  Description of Artificial Sequence Synthetic polynucleotide
atgaagattgaggagggaaaacttgtcatatggattaatggcgacaaaggctataatgggttagcagaagtcggtaaaaagtttgagaaagacactgggattaaggtaacggtcgagcacccagataagctggaagagaaattcccacaggttgccgcgactggggatggccccgacatcatattctgggcgcacgacagatttggcggttatgcacaaagtgggttactagctgaaattaccccagataaggcatttcaagacaaactatatcctttcacttgggatgcggttagatataacggaaaattgatagcctatcctattgccgtggaggctttatcactaatctataacaaggacctattgccgaacccgcccaaaacatgggaagaaatccctgccttagacaaagaacttaaagcgaaaggcaagagtgctctaatgttcaatcttcaagagccttattttacttggcccttgatagcggccgatggcggctacgccttcaagtacgagaacgggaagtatgatattaaagacgttggagtggataacgcgggtgcgaaggctggcctgacgttcttagtggacttgattaaaaataagcacatgaacgcggacacggactacagcatcgcggaggcggcttttaataagggcgaaactgctatgacgatcaatggaccttgggcttggtcaaatatagatacaagtaaggtaaattatggagtaactgtgctgccgacctttaagggccaacctagtaaaccgtttgtcggcgtgttgtccgccgggataaacgccgcctcccccaacaaagaattagcaaaggaatttttggagaattacttactgaccgatgagggcttggaggcagtcaataaggataagcccctgggcgctgtcgcattgaagtcatatgaagaagaacttgcaaaagatccccgtattgctgccacaatggagaatgcacagaaaggtgaaataatgcccaacataccgcagatgagtgcgttctggtatgcggtaagaacagctgttatcaacgctgcgtccgggaggcaaacagttgatgaggctttgaaagacgctcagaccaattcctccagcaacaacaataataataacaataacaacaacttaggtatagaaggtagataa

SEQ ID NO: 102 - DNA, 720 bp, Artificial Sequence
  Description of Artificial Sequence Synthetic polynucleotide
atggttagtaaaggagaagagttattcactggcgttgtacctattctggttgagctagacggagatgttaatggccacaaattctccgtatccggggagggggagggcgatgcaacatatggaaaacttacgctaaaactaatctgtacgactgggaaactacccgttccgtggcccacattggttacgacacttggctatggcctacagtgtttcgctagataccctgatcatatgaagcaacatgatttctttaagagtgcaatgccggagggttacgttcaggaaagaacaattttcttcaaggatgacggcaattacaagacgagggccgaggtaaaattcgagggggatacgctggttaacaggatagaattaaaaggtatagattttaaagaagacgggaacattctaggtcataaacttgagtacaattacaactcccataatgtctacataacagcggacaagcagaagaatggtataaaggcaaattttaagatcagacataacattgaagacgggggagtccagttggctgaccactatcaacaaaatacccccattggggacggtccggtgttgcttccagataaccactatctttcttaccagtcagccctatccaaagacccaaacgagaagagggatcatatggttcttctggagtttgtcaccgcagcagggattactttggggatggacgagctatacaagtaa

SEQ ID NO: 103 - DNA, 354 bp, Artificial Sequence
  Description of Artificial Sequence Synthetic polynucleotide
atggctatgttctgcactttcttcgaaaaacatcatcgtaaatgggacattttactagagaaatccaccggtgtgatggaggcgatgaaagtgacatccgaagaaaaggaacaactgagcacagctattgaccgtatgaacgagggcctggatgcttttatccagctatataacgagtccgaaatagacgagccccttatccagcttgacgatgacacagccgaattaatgaaacaagctagagatatgtacggtcaggagaagttaaatgaaaaactaaatacaatcattaagcaaattttgtcaatctctgtatccgaggagggagagaaagaaggcagcggatcaggataa

SEQ ID NO: 104 - DNA, 285 bp, Artificial Sequence
  Description of Artificial Sequence Synthetic polynucleotide
atgtatcttctagggattgggcttattttagcactgattgcctgcaagcaaaacgtttcttcactagacgagaaaaactcagtgtcagtagaccttcctggtgagatgaaggttttggtcagcaaggaaaagaacaaagatggcaagtacgatttaattgctaccgtggataagttggagctgaaaggaacatccgacaagaacaacggctctggggtacttgaaggagtcaaggccgataaaagcaaagtcaagctaacaatttccgacgacgggtctggataa

SEQ ID NO: 105 - DNA, 528 bp, Artificial Sequence
  Description of Artificial Sequence Synthetic polynucleotide
atggcagacagagataggtcaggtatctatggcggtgctcatgcgacgtatggccaacagcaacagcaaggaggaggcgggagacctatgggcgaacaagtcaagggcatgctgcatgacaagggccctacggcgtcccaggccttgacagttgctaccttatttcctttaggagggctgcttttggtgcttagtggattagcccttactgcttccgtagtcggtctagcagtcgcaacgccggtctttttgatcttcagtccggtgctagtcccagcggcattgctaatcggcactgccgtcatggggttcttgacctccggtgctctgggtctgggtggtttgtcctctctgacctgtctagcaaacactgcgcgtcaggcttttcagcgtacccctgactacgtggaagaagctcatagaaggatggctgaggcagcagcgcatgccggacataagacggctcaggctgggcaagctattcaaggcagggctcaagaggctggcgctggtggtggggccgggtaa

SEQ ID NO: 106 - protein, 388 aa, Artificial Sequence
  Description of Artificial Sequence Synthetic polypeptide
MSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGR

SEQ ID NO: 107 - protein, 239 aa, Artificial Sequence
  Description of Artificial Sequence Synthetic polypeptide
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 108 - protein, 118 aa, Artificial Sequence
  Description of Artificial Sequence Synthetic polypeptide
MSAMFCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKEGSGSG

SEQ ID NO: 109 - protein, 96 aa, Artificial Sequence
  Description of Artificial Sequence Synthetic polypeptide
MSSYLLGIGLILALIACKQNVSSLDEKNSVSVDLPGEMKVLVSKEKNKDGKYDLIATVDKLELKGTSDKNNGSGVLEGVKADKSKVKLTISDDGSG

SEQ ID NO: 110 - protein, 177 aa, Artificial Sequence
  Description of Artificial Sequence Synthetic polypeptide
MSSADRDRSGIYGGAHATYGQQQQQGGGGRPMGEQVKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEAHRRMAEAAAHAGHKTAQAGQAIQGRAQEAGAGGGAG

(19) United States
(12) Patent Application Publication
(10) Pub. No.: US 2022/0298533 A1
(43) Pub. Date: Sep. 22, 2022

(54) BIOSYNTHETIC METHODS FOR THE MODIFICATION OF CANNABINOIDS

(71) Applicant: CB Therapeutics, Inc., Carlsbad, CA (US)

(72) Inventors: Erin Marie Scott, Encinitas, CA (US); Jacob Michael Vogan, San Diego, CA (US)

(73) Assignee: CB Therapeutics, Inc., Carlsbad, CA (US)

(21) Appl. No.: 17/701,625

(22) Filed: Mar. 22, 2022

Related U.S. Application Data
(60) Provisional application No. 63/164,126, filed on Mar. 22, 2021.

(51) Int. Cl. C12P 7/22; C12N 15/52; C12N 15/81; C12P 17/06; C12N 9/02

(52) U.S. Cl. CPC C12P 7/22; C12N 15/52; C12N 15/81; C12P 17/06; C12N 9/0042; C12R 2001/865

(57) ABSTRACT

Provided is a method of modifying a first cannabinoid into a second cannabinoid or a non-cannabinoid. The method comprises combining the first cannabinoid with an enzyme that can modify the first cannabinoid into the second cannabinoid or non-cannabinoid under conditions where the first cannabinoid is modified into the second cannabinoid or non-cannabinoid. Also provided is a non-naturally occurring enzyme that can modify a first cannabinoid into a second cannabinoid or a non-cannabinoid. A nucleic acid encoding that enzyme is additionally provided. Further provided is a non-naturally occurring nucleic acid that encodes an enzyme having the enzymatic activity of the above non-naturally occurring enzyme. An expression cassette comprising that nucleic acid is additionally provided. A cell comprising the above expression cassette is further provided. Also provided is a plant expression cassette comprising the above-identified nucleic acid.

### CROSS-REFERENCE TO RELATED APPLICATIONS

This application claims the benefit of U.S. Provisional Application No. 63/164,126, filed Mar. 22, 2021, and incorporated by reference herein in its entirety.

### SEQUENCE LISTING

The instant application contains a Sequence Listing which has been submitted electronically in ASCII format and is hereby incorporated by reference in its entirety. Said ASCII copy, created on Mar. 22, 2022, is named CBTH-12-US_SL.txt and is 339,457 bytes in size.

### BACKGROUND OF THE INVENTION

### (1) Field of the Invention

The present application generally relates to manipulation of cannabinoids. More specifically, the application provides methods and compositions for the enzymatic modification or degradation of cannabinoids.

### (2) Description of the Related Art

Cannabinoids are a class of organic small molecules of meroterpenoid structures found in the plant genus Cannabis. The small molecules are currently under investigation as therapeutic agents for a wide variety of health issues, including epilepsy, pain, and other neurological problems, and mental health conditions such as depression, PTSD, opioid addiction, and alcoholism (Committee on the Health Effects of Marijuana, 2017).

Numerous cannabinoids of varying structure are produced in Cannabis spp., each with their own therapeutic profile. However, since some cannabinoids are made in very small quantities in Cannabis spp. and are challenging to separate from other cannabinoids in Cannabis extracts, it is difficult to evaluate the therapeutic and psychotropic effect of each particular cannabinoid.

Rare cannabinoids from Cannabis spp. or from microbial bioproduction are gaining intense interest in the nutraceutical and clinical markets.

In one example, conversion of the abundant cannabinoid, tetrahydrocannabinol (THC) to a rare cannabinoid, cannabinol (CBN) is desirable for many reasons. THC is lower value, has intoxicating psychoactive side effects and is illegal in many jurisdictions. CBN is a high value, legal molecule that shows great clinical promise in treating sleep and skin disorders, and it has shown potential as a therapeutic for amyotrophic lateral sclerosis (Lou Gehrig's disease) (Carter, 2010; reviewed in Giacoppo, 2016). CBN is naturally formed by slow and inefficient non-enzymatic oxidation of THC in Cannabis spp. However, there is no known enzymatic route to produce CBN from THC. CBN can also be synthesized in small batches using organic chemistry (Caprioglio, 2019). Other approaches to make CBN include non-enzymatic oxidation methods applied to purified plant derived cannabinoids, such as heating and exposure to UV light or sunlight (PCT Patent Application Publication WO2014/159688A1 and US Patent Application Publication 2017/0020943A1) These routes are expensive, slow and environmentally unfriendly. An enzymatic route to CBN would greatly aid efforts to produce larger, cheaper and more consistent batches of this highly valuable compound.

There is thus a need to (a) synthesize individual cannabinoids, (b) convert one cannabinoid into another cannabinoid, or (c) convert a particular cannabinoid into a non-cannabinoid. The present invention addresses that need.

### BRIEF SUMMARY OF THE INVENTION

The present invention provides enzymes and methods using those enzymes to modify or degrade cannabinoids. Thus, in some embodiments, a method of modifying a first cannabinoid into a second cannabinoid or a non-cannabinoid is provided. The method comprises combining the first cannabinoid with an enzyme that can modify the first cannabinoid into the second cannabinoid or non-cannabinoid under conditions where the first cannabinoid is modified into the second cannabinoid or non-cannabinoid.

Also provided is a non-naturally occurring enzyme that can modify a first cannabinoid into a second cannabinoid or a non-cannabinoid. A nucleic acid encoding that enzyme is additionally provided.

Further provided is a non-naturally occurring nucleic acid that encodes an enzyme having the enzymatic activity of the above non-naturally occurring enzyme. An expression cassette comprising that nucleic acid is additionally provided.

In other embodiments, a cell comprising the above expression cassette is provided. In these embodiments, the cell is capable of expressing the enzyme provided above, or a naturally occurring equivalent thereof.

Also provided is a plant expression cassette comprising the above-identified nucleic acid, as is a plant comprising the expression cassette, where the plant is capable of expressing the above-identified enzyme, or a naturally occurring equivalent thereof.

### BRIEF DESCRIPTION OF THE SEVERAL VIEWS OF THE DRAWINGS

FIG. 1 depicts cannabinoid synthase substrates, the structures of various cannabinoids, and cannabinoid decarboxylation reactions. Panel A shows the alkylresorcylic acid prenyl acceptor and the polyprenol diphosphate prenyl donor in cannabinoid synthase reactions; Panel B shows various cannabinoid compounds; and Panel C shows cannabinoid decarboxylation reactions.

FIG. 2A depicts the CBN biosynthesis pathway and structures of variants cannabinoids.

FIG. 2B depicts the 11-hydroxylation of THC and CBN by cytochrome P450 CYP2C19.

FIG. 2C depicts oxidases acting on representative cannabinoids, THC and CBN, to form homopolymers and heteropolymers.

FIGS. 3A, 3B and 3C depict different mechanisms by which different classes of enzymes might form an aromatic ring during CBN biosynthesis. FIG. 3A depicts a ring desaturation mechanism carried out by an aromatase. FIG. 3B depicts a ring desaturation mechanism carried out by a dehydrogenase. FIG. 3C depicts a ring desaturation mechanism carried out by a desaturase.

FIG. 4A depicts methods for making CBN biosynthetically using this technology where the entire CBN biosynthesis pathway is contained within one microbial host. Also depicted is a complete biosynthesis pathway to 11-OH CBN where the entire 11-OH CBN biosynthesis pathway is contained within one microbial host.

FIG. 4B depicts a bioconversion strategy where one microbe makes THC, and a second microbe converts THC to CBN.

FIG. 4C depicts bioconversion of crude plant or microbial material by microbe with CBN synthase.

FIG. 4D depicts bioconversion of purified cannabinoids by a microbe containing CBN synthase.

FIG. 4E depicts enzymatic conversion of purified cannabinoids using purified recombinant CBN synthase.

FIG. 4F depicts enzymatic conversion of crude plant or microbial material using purified recombinant CBN synthase.

FIG. 4G depicts a cannabinoid producing plant that is not modified and a plant that is modified to express a CBN synthase

FIG. 5A depicts methods for selective THC degradation where the entire pathway producing THC and CBD is contained within one microbial host.

FIG. 5B depicts a bioconversion strategy where one microbe makes THC, and a second microbe degrades THC.

FIG. 5C depicts elimination of THC from crude plant or microbial material by a microbe expressing THC degradase.

FIG. 5D depicts elimination of THC from purified cannabinoids by a microbe expressing a THC degradase.

FIG. 5E depicts selective enzymatic degradation of THC in purified cannabinoids using purified recombinant THC degradase.

FIG. 5F depicts selective enzymatic degradation of THC in crude plant or microbial material using purified recombinant THC degradase.

FIG. 5G depicts a cannabinoid producing plant that is not modified and a plant that is modified to express a THC degradase.

FIG. 6A depicts HPLC data showing selective degradation of THC and bioconversion of THC into CBN by a microbe possessing CBN synthase activity relative to THC incubated with a microbe that does not have this activity.

FIG. 6B depicts HPLC data showing selective degradation of THC by a microbe possessing THC degradase activity relative to THC incubated with a microbe that does not have this activity.

### DETAILED DESCRIPTION OF THE INVENTION

### Abbreviations and Definitions

To facilitate understanding of the invention, a number of terms and abbreviations as used herein are defined below as follows:

Conservative amino acid substitutions: As used herein, when referring to mutations in a protein, “conservative amino acid substitutions” are those in which at least one amino acid of the polypeptide encoded by the nucleic acid sequence is substituted with another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Functional variant: The term “functional variant,” as used herein, refers to a recombinant enzyme such as a CBN synthase that comprises a nucleotide and/or amino acid sequence that is altered by one or more nucleotides and/or amino acids compared to the nucleotide and/or amino acid sequences of the parent protein and that is still capable of performing an enzymatic function (e.g., synthesis of CBN) of the parent enzyme. In other words, the modifications in the amino acid and/or nucleotide sequence of the parent enzyme may cause desirable changes in reaction parameters without altering fundamental enzymatic function encoded by the nucleotide sequence or containing the amino acid sequence. The functional variant may have conservative change including nucleotide and amino acid substitutions, additions and deletions. These modifications can be introduced by standard techniques known in the art, such as site-directed mutagenesis and random PCR-mediated mutagenesis, and may comprise natural as well as non-natural nucleotides and amino acids. Also envisioned is the use of amino acid analogs, e.g. amino acids not DNA or RNA encoded in biological systems, and labels such as fluorescent dyes, radioactive elements, electron dense agents, or any other protein modification, now known or later discovered.

Recombinant nucleic acid and recombinant protein: As used herein, a recombinant nucleic acid or protein is a nucleic acid or protein produced by recombinant DNA technology, e.g., as described in Green and Sambrook (2012).

Polypeptide, protein, and peptide: The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, and the like. Modifications also include intra-molecular crosslinking and covalent attachment of various moieties such as lipids, flavin, biotin, polyethylene glycol or derivatives thereof, and the like. In addition, modifications may also include protein cyclization, branching of the amino acid chain, and cross-linking of the protein. Further, amino acids other than the conventional twenty amino acids encoded by genes may also be included in a polypeptide.

The term “protein” or “polypeptide” may also encompass a “purified” polypeptide that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

Primer, probe and oligonucleotide: The terms “primer,” “probe,” and “oligonucleotide” may be used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated apart by denaturation. In certain aspects, they are of a length of from about 8 nucleotides to about 200 nucleotides. In other aspects, they are from about 12 nucleotides to about 100 nucleotides. In additional aspects, they are about 18 to about 50 nucleotides. They can be labeled with detectable markers or modified in any conventional manners for various molecular biological applications.

Vector: As used herein, the term “vector” refers to a nucleic acid molecule capable of transporting another nucleic acid to which it has been linked. One type of vector is an episome, i.e., a nucleic acid capable of extra-chromosomal replication. Various vectors are those capable of autonomous replication and/expression of nucleic acids to which they are linked. Vectors capable of directing the expression of genes to which they are operatively linked are referred to herein as “expression vectors.”

Linker: The term “linker” refers to a short amino acid sequence that separates multiple domains of a polypeptide. In some embodiments, the linker prohibits energetically or structurally unfavorable interactions between the discrete domains.

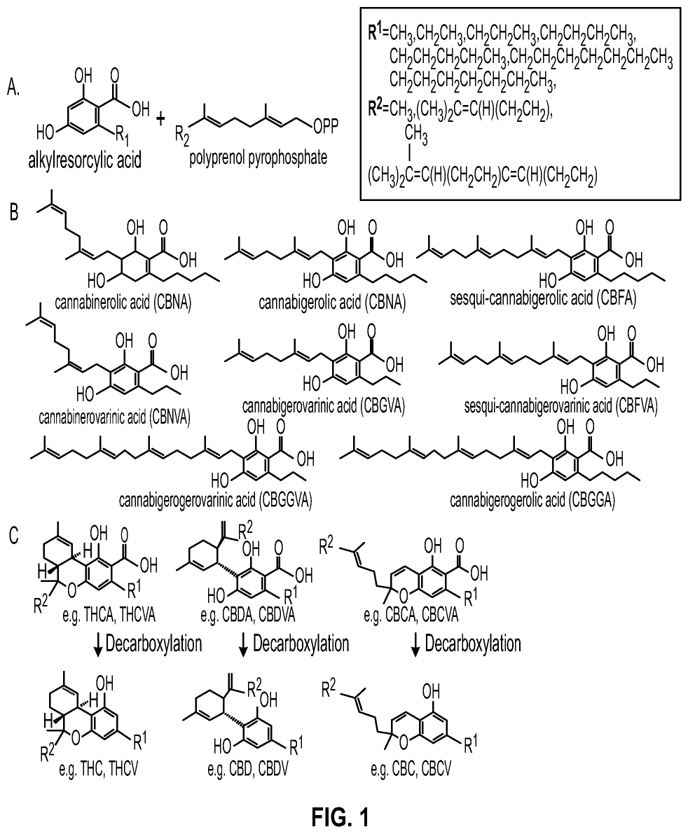

Cannabinoid: As used herein, the term “cannabinoid” refers to a family of structurally related aromatic meroterpenoid molecules. Cannabinoids are generally formed by the enzymatic fusion, by a cannabinoid synthase (having geranylpyrophosphate:olivetolate geranyltransferase activity), of an alkylresorcylic acid

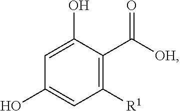

where R¹═CH₃, (CH₂)₂CH₃ (divarinolic acid), (CH₂)₄CH₃ (olivetolic acid), or (CH₂)₆CH₃, with a polyprenyl pyrophosphate such as geranyl pyrophosphate, neryl pyrophosphate, geranylgeranyl pyrophosphate, of farnesyl pyrophosphate (FIG. 1; see also Luo et al., 2019; Carvalho et al., 2017; and Gülck and Møller, 2020 and references cited therein). The polyprenyl pyrophosphate is synthesized by geranyl pyrophosphate synthase (GPPS) (U.S. Provisional Patent Application 63/141,486).

Codon optimized: As used herein, a recombinant gene is “codon optimized” when its nucleotide sequence is modified to accommodate codon bias of the host organism to improve gene expression and increase translational efficiency of the gene.

Expression cassette: As used herein, an “expression cassette” is a nucleic acid that comprises a gene and a regulatory sequence operatively coupled to the gene such that the promoter drives the expression of the gene in a cell. An example is a gene for an enzyme with a promoter functional in yeast, where the promoter is situated such that the promoter drives the expression of the enzyme in a yeast cell.

The present invention is directed to methods and compositions for modifying a first cannabinoid into a second cannabinoid or a non-cannabinoid using recombinant enzymes in microorganisms.

### Methods of Modifying or Degrading Cannabinoids

In some embodiments, a method of modifying a first cannabinoid into a second cannabinoid or a non-cannabinoid is provided. The method comprises combining the first cannabinoid with an enzyme that can modify the first cannabinoid into the second cannabinoid or non-cannabinoid under conditions where the first cannabinoid is modified into the second cannabinoid or non-cannabinoid.

In these embodiments, the first cannabinoid and the second cannabinoid can be any cannabinoid now known or later discovered. In some of these embodiments, the first and/or second cannabinoid comprises the structure

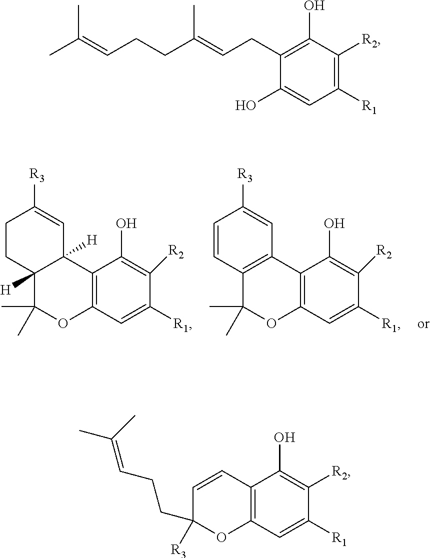

wherein R¹═CH₃, CH₂CH₃, (CH₂)₂CH₃, (CH₂)₃CH₃, (CH₂)₃CH₃, (CH₂)₄CH₃, (CH₂)₅CH₃, or (CH₂)₆CH₃; R₂═H or COOH; and R₃═CH₃ or CH₂OH.

Non-limiting examples of the first cannabinoid or the second cannabinoid are cannabigerolic acid (CBGA), cannabidiolic acid (CBDA), cannabichromene (CBC), cannabidivarin (CBCV), cannabichromenic acid (CBCA), cannabichromevarinic acid (CBCVA) cannabinol (CBN), cannabinerolic acid (CBNA), cannabivarin (CBV), cannabigerolic acid (CBGA), cannabinerovarinic acid (CBNVA), cannabigerophorolic acid (CBGPA), cannabigerovarinic acid (CBGVA), cannabigerogerovarinic acid (CB GGVA), tetrahydrocannabinol (THC), tetrahydrocannabinolic acid (THCA), tetrahydrocannabivarin (THCV), tetrahydrocannabivarin acid (THCVA), cannabinerovarinic acid (CBNVA), sesquicannabigerol (CBF), cannabigerogerol (CBGG), sesqui-cannabigerolic acid (CBFA), cannabigerogerolic acid (CBGGA), sesquicannabigerolic acid (CBFA), sesquicannabidiolic acid (CBDFA), sesquiTHCA (THCFA), sesqui-cannabigerovarinic acid (CBFVA), sesquiCBCA (CBCFA), sesquiCBGPA (CBFPA), tetrahydrocannabivarin (THCV), cannabidiol (CBD), cannabidiolic acid (CBDA), cannabidivarinic acid (CBDVA), or cannabidivarin (CBDV) (FIG. 1). The decarboxylation reactions shown in FIG. 1C can be carried out by heat (e.g., combustion) or a-decarboxylase.

These methods can use any enzyme, now known or later discovered, that can carry out the conversion of the first cannabinoid into the second cannabinoid or degrade the first cannabinoid. In some embodiments, the enzyme is an aromatase, a dehydrogenase, an oxidase or a desaturase.

In some embodiments, the first cannabinoid is tetrahydrocannabinol (THC) or tetrahydrocannabinolic acid (THCA) and the second cannabinoid is cannabinol (CBN) or cannabinolic acid (CBNA). In other embodiments, the first cannabinoid is tetrahydrocannabivarinic acid (THCVA), tetrahydrocannabiphorolic acid (TCHPA), tetrahydrocannabiorcinic acid (THCOA) or sesquiTHCA (THCFA) and the second cannabinoid is cannabinerolic acid (CBNA), cannabinerovarinic acid (CBNVA), cannabiphorolic acid (CBNPA), cannabinorcinic acid (CBNOA) or sesqui cannabinerolic acid (sesqui-CBNA), respectively. In additional embodiments, the first cannabinoid is tetrahydrocannabivarinol (THCV), tetrahydrocannabiphorol (TCHP), tetrahydrocannabiorcinol (THCO) or sesquitetrahydrocannabinolic acid (sesquiTHCA) and the second cannabinoid is cannabinerovarinol (CBNV), cannabiphorol (CBNP), cannabinorcinol (CBNO) or sesqui cannabinerol (sesqui-CBN), respectively.

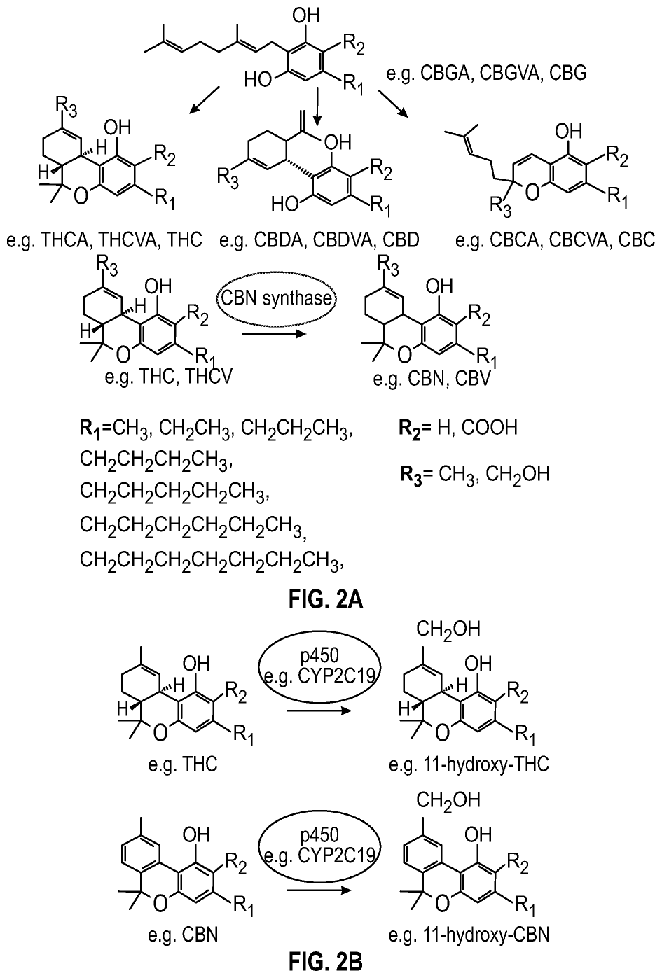

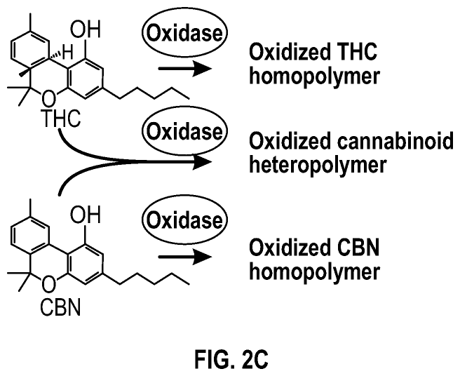

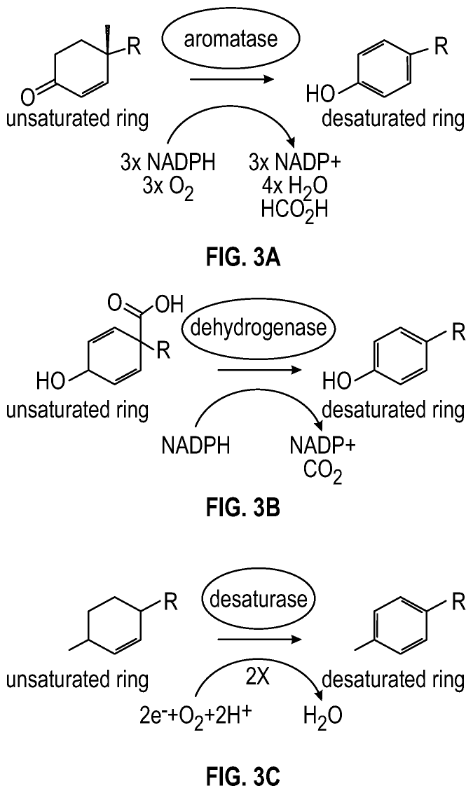

Exemplary enzymatic reactions are shown in FIGS. 2A, 2B, 2C, 3A, 3B, and 3C. FIG. 2A shows the enzymatic conversion of the initial products of cannabinoid synthase, e.g., CBGA, CBGVA and CBG, into THCA, THCVA, THC, CBDA, CBDVA, CBD, CBCA, CBCVA or CBC. FIG. 2A also shows the conversion of THC or THCV into CBN or CBV by CBN synthase. In various embodiments, the CBN synthase is a desaturase, an aromatase, a dehydrogenase, or an oxidase.

In various embodiments, the first cannabinoid is converted into a second cannabinoid that is an 11-hydroxy derivative of the first cannabinoid. In some of these embodiments, the conversion is carried out by the combination of a cytochrome P450 (CYP-450) and a cytochrome P450 reductase (CPR). FIG. 2B shows a nonlimiting example of the conversion of THC and CBN into 11-hydroxy-THC and 11-hydroxy-CBN, respectively, by a CYP-450, for example CYP2C19, and a P450 reductase.

In various embodiments, a cannabinoid is oxidized by an oxidase into a polymeric state, such as a dimer of cannabinoids. This can occur between oxidized cannabinoids of the same species, such as THC or CBN, respectively, to form homopolymers, or a mixture of cannabinoid species, such as THC and CBN, which are oxidized to a heteropolymer of cannabinoids, as show in FIG. 2C.

The enzyme utilized in these methods can have any activity that can modify the first cannabinoid into the second cannabinoid. For example, FIG. 3A shows a generalized aromatase activity that can be utilized to convert, e.g., THC or THCV into CBN or CBV, and FIG. 3B and FIG. 3C show generalized dehydrogenase and desaturase activities, respectively, that, as discussed above, can also serve to create the aromatic ring.

In some embodiments, the enzymes utilized in these methods additionally enable reduction of cannabinoid, e.g., THC, levels in pure cannabinoid preparations while not affecting other cannabinoid molecules. Cannabidiol (CBD) products often contain unwanted THC. Federal law bans any product containing more than 0.3% THC, so even small reductions in THC are critical to maintenance of cannabis products under this legal limit. Enzymes that destroy THC completely or convert THC to a molecule besides CBN are useful for certain applications and are commercially valuable.

The invention methods can be part of a complete biosynthesis pathway for cannabinoids such as CBN, including production of its acidic cannabinoid variant, cannabinolic acid (CBNA). The complete biosynthesis pathway for any cannabinoid is amenable to integration in a cannabinoid producing host cell. If the pathway includes a functional CBN synthase, accumulation of THC during an industrial fermentation is avoided.

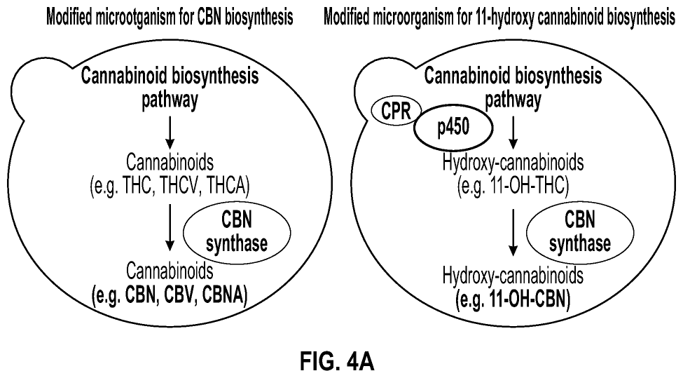

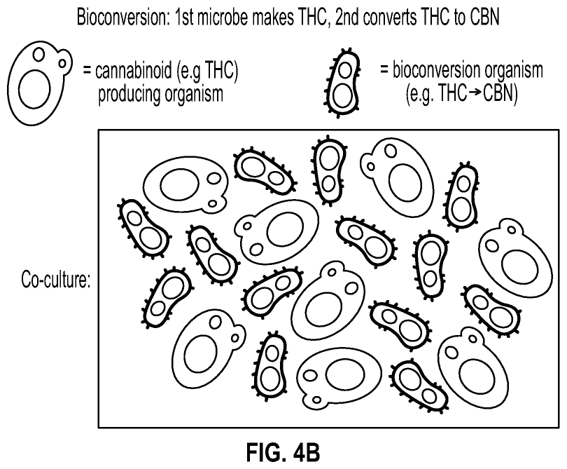

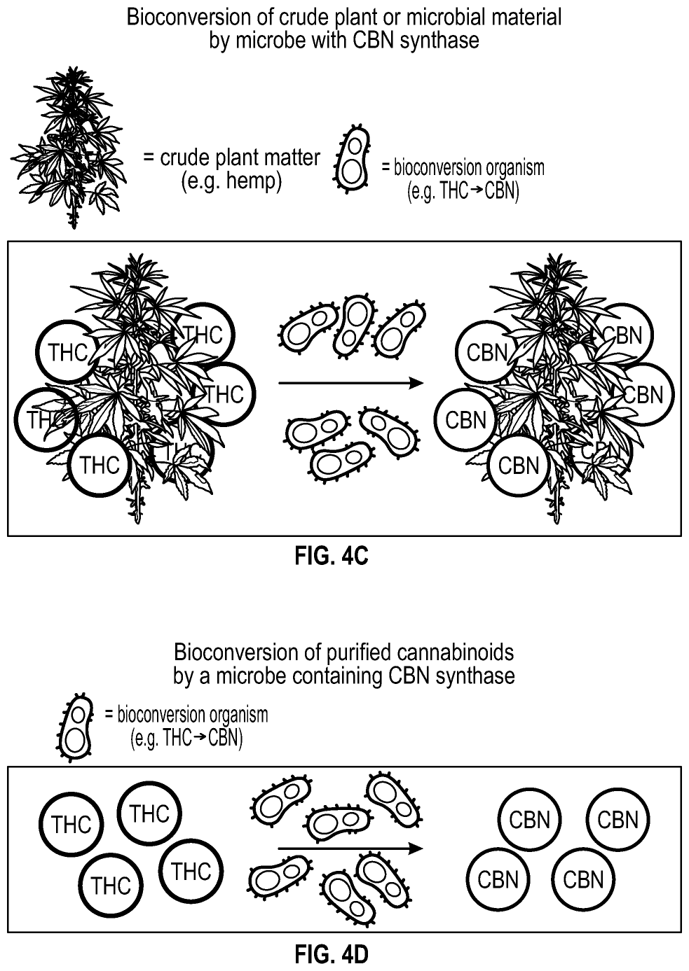

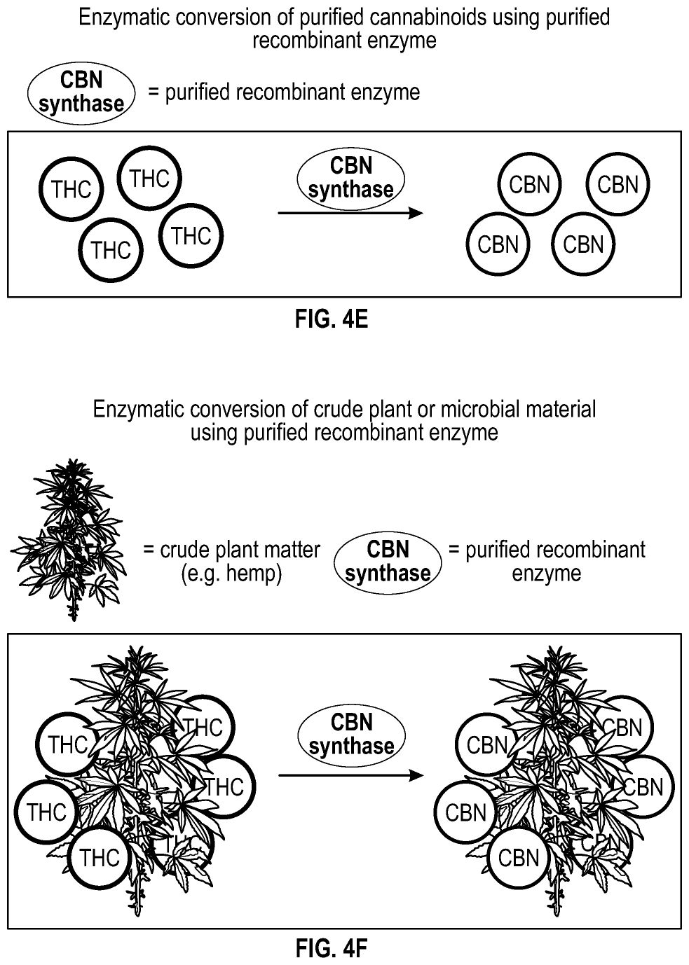

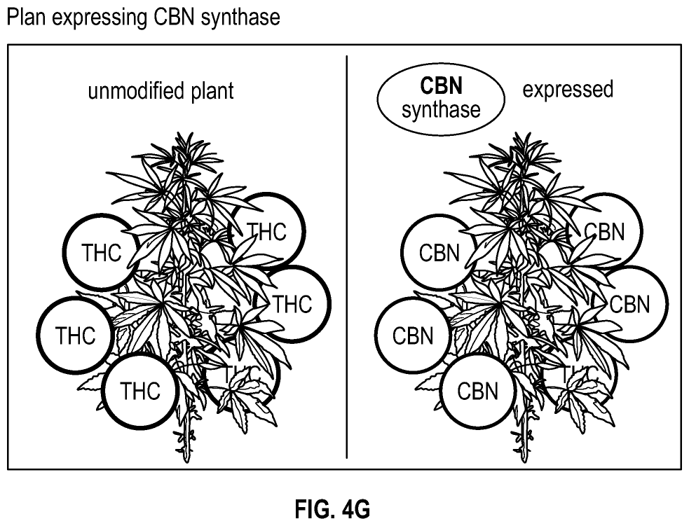

The microorganism, e.g., yeast or bacterium, in which the methods are carried out can further comprise other enzymes, e.g., recombinantly transformed enzymes, that can affect the cannabinoid pathway, for example an enzyme that synthesizes the first cannabinoid from a non-cannabinoid or from another cannabinoid. This is illustrated in FIG. 2B and the right panel of FIG. 4A, showing an illustration of a microorganism that is transformed with a CYP-450 and a CPR that converts a cannabinoid (e.g., THC) into an 11-hydroxy cannabinoid (e.g., 11-OH-THC), then converting that 11-hydroxy THC into 11-OH-CBN with CBN synthase. See also Watanabe, 2007.

To execute a CYP reaction, a CPR (cytochrome P450 reductase) is necessary to supply the P450 enzyme with reducing equivalents in the form of NADPH. The combination of the recombinant P450 and CPR genes and enzymes results in an 11-OH hydroxylase capable of acting on various cannabinoid substrates. In some embodiments, the hydroxyl group at the 11-position is added by recombinant CYP-450+CPR before the conversion of tetrahydrocannabinol or tetrahydrocannabinolic acid (THC/A) to CBN/A, yielding a conversion from 11-hydroxy tetrahydrocannabinol (11-OH THC) to 11-OH CBN.

The recombinant hydroxylation enzymes herein described may also hydroxylate other cannabinoid substrates, such as CBD, when expressed in a recombinant host capable of cannabinoid bioproduction. Additional reactions, substrates, and products for the above reconstituted biosynthetic pathways in a modified organism are depicted in FIG. 2A, where cannabinoid variants such as cannabivarinol (CBV) can also be produced via CBN synthases and bioconversion organisms herein described.

The enzymes used in these methods can be recombinantly expressed in a microorganism such as a yeast or bacterium, or a plant such as a Cannabis sp. In those systems, the gene for those enzymes can be modified, e.g., by codon optimizing the gene for the recombinant microorganism or plant.

In other embodiments, the enzyme is not naturally occurring. Such enzymes can be modified from a naturally occurring enzyme by, e.g., having conservative amino acid substitutions or substitutions that alter the enzymatic activity. Those enzymes can also be derived from a naturally occurring gene that has been codon optimized for expression in a recombinant host such as bacteria, yeast or plants.

In some of these methods, the first cannabinoid is converted (degraded) into a non-cannabinoid, for example by eliminating the cannabinoid aromatic ring that is derived from an alkylresorcylic acid in the naturally occurring cannabinoid pathway in Cannabis spp. Acetyl-CoA can also be produced as a result of this conversion.

These methods can be carried out in vivo or in vitro. When in vitro, the enzyme can be synthesized in a recombinant microorganism or plant and extracts of the microorganism or plant can be combined with the first cannabinoid. In various embodiments, the enzyme can be at least partially purified from the extract.

In these in vitro methods, the first cannabinoid can be present in a crude extract of a Cannabis sp. plant or a microorganism from which the first cannabinoid was synthesized. Alternatively, the first cannabinoid can be substantially purified when combined with the enzyme.

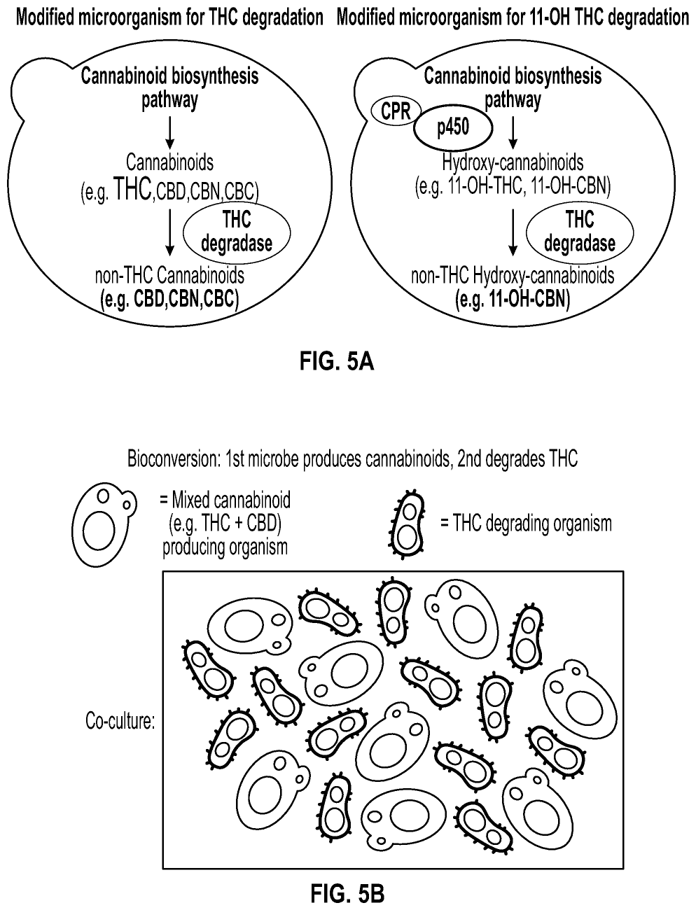

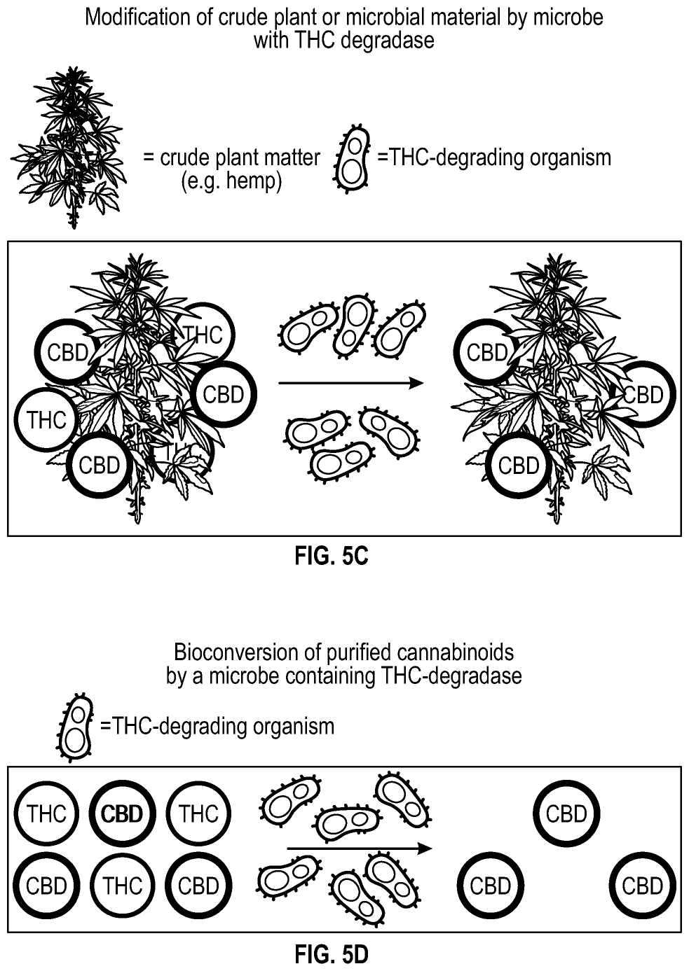

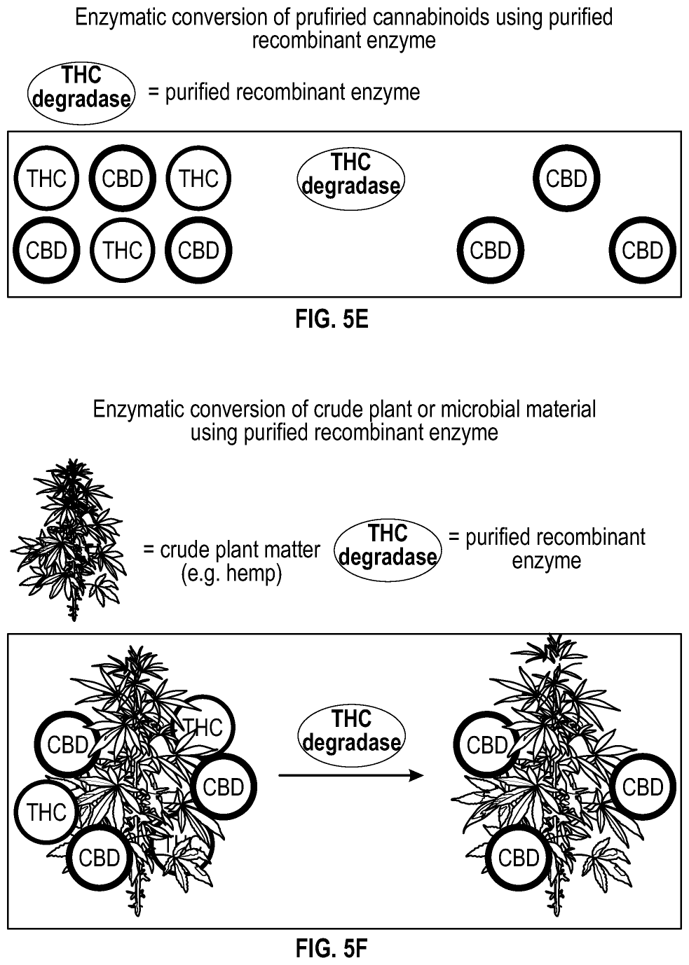

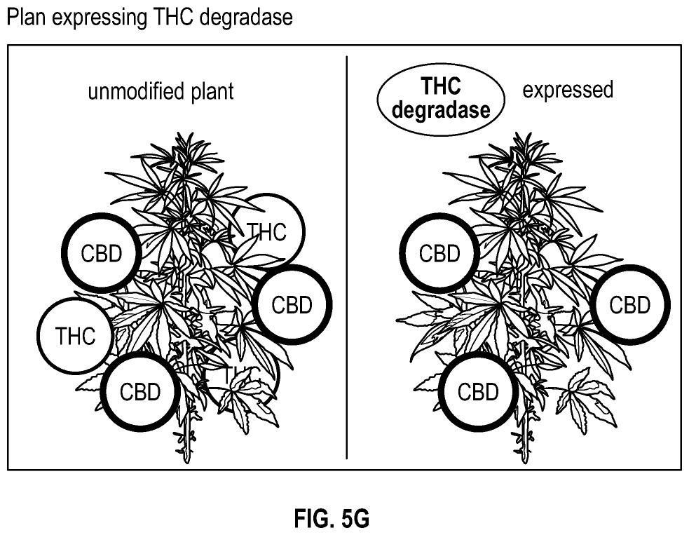

Exemplary in vitro methods are illustrated in FIGS. 4E, 4F, 5D and 5E. In FIG. 4E, THC is incubated with purified CBN synthase, converting the THC to CBN. In FIG. 4F, purified CBN synthase is incubated with a crude Cannabis sp. (hemp) preparation, converting THC therein into CBN. FIG. 5D illustrates utilizing a THC degradase inside an organism to degrade THC in a purified mixture of THC and CBD, leaving the CBD. FIG. 5E illustrates the same reaction, where the degradase degrades the THC in a crude Cannabis sp. (hemp) preparation, leaving the CBD.

In other embodiments, bioconversion of THC to CBN takes place using lysate of a microbe containing the CBN synthase while the THC precursor is produced in a second microorganism. The first microbe could express the CBN synthase natively or recombinantly.

In additional embodiments, bioconversion of THC to CBN takes place using lysate of a microorganism containing the CBN synthase while the THC precursor is supplied as lysate from a second, cannabinoid producing microorganism. The first microbe could express the THC-to-CBN synthase natively or recombinantly.

In further embodiments, the CBN synthase is expressed recombinantly in a microbial host and the enzyme purified. The purified enzyme can then be used on purified plant derived THC to do an enzymatic conversion of THC to CBN in vitro.

The methods provided herein can facilitate development of industrial processes to eliminate THC and/or produce CBN in crude cannabinoid preparations, including plant material and microbial cell mass.

In the above exemplary embodiments, THC/A can be selectively degraded instead of being converted to CBN.

When the method is carried out in vivo, the method can be carried out by a living organism that synthesizes the enzyme. Any living organism can be utilized to carry out the method. In some embodiments, the method is carried out in a plant, e.g., a tobacco or Cannabis sp. plant.

In other embodiments, the method is carried out in a microorganism, as illustrated in FIG. 4A. The left panel of FIG. 4A shows an illustration of a microorganism transformed with a CBN synthase gene, that can convert THC, THCV or THCA to CBN, CBV or CBNA. Any microorganism capable of being transformed with a recombinant form of the enzyme can be utilized here. In some of these embodiments, the first microorganism is a yeast, e.g., a yeast that is a species of Saccharomyces, Candida, Pichia, Schizosaccharomyces, Scheffersomyces, Blakeslea, Rhodotorula, or Yarrowia. In other embodiments, the first microorganism is a bacterium, e.g., a bacterium of the genus Rhodococcus, Gordonia, Dietzia, Streptomyces, Escherichia, Nocardia or Mycobacterium.

The microorganism can also comprise a recombinant enzyme “upstream” from cannabinoid synthase, e.g., a recombinant geranyl pyrophosphate synthase (GPPS) (see U.S. Provisional Patent Application 63/141,486). In various embodiments, the microorganism further comprises a recombinant GPPS and cannabinoid synthase, where the cannabinoid synthase can combine a polyprenyl pyrophosphate with alkylresorcylic acid to create a cannabinoid.

In some in vivo embodiments of these methods where the enzyme is in a first microorganism (yeast or bacteria), the first cannabinoid is synthesized in a second microorganism, wherein the method further comprises incubating the first microorganism, or an extract thereof, with the second microorganism. This is illustrated in FIG. 4B, which shows a transgenic microorganism that produces a first cannabinoid (e.g., THC) in co-culture with a transgenic microorganism that converts the first cannabinoid into a second cannabinoid (e.g., CBN). In that example, bioconversion of THC to CBN takes place using a microbe containing CBN synthase while the THC precursor is produced in a second microorganism. The first microbe could express the CBN synthase natively or recombinantly. This bioconversion strategy would follow that outlined by Abbott (1977), but incorporate a recombinant THC producing microbe as well as use on crude plant material or microbial biomass.

In other embodiments, the first cannabinoid is synthesized in a Cannabis sp. plant and matter from the Cannabis sp. plant is incubated with the first microorganism. This is illustrated in FIG. 4C, where THC is produced in a Cannabis sp. (i.e., hemp) plant, and crude plant matter is incubated with the first microorganism (e.g., a yeast or bacterium) that converts the THC into CBN.

In embodiments described above where the first cannabinoid is extracted from the second microorganism or a plant (e.g., a Cannabis sp. plant or tobacco), the first cannabinoid can be in a crude extract or can be partially or substantially purified from the second microorganism.

Various additional in vivo scenarios are illustrated in FIGS. 4D, 5A, 5B and 5C. FIG. 4D illustrates the bioconversion of purified THC into CBN by a microorganism (e.g., a yeast or bacterium) that expresses a recombinant CBN synthase. In FIG. 5B, a first microorganism that produces both THC and CBD is co-cultured with a second microorganism that produces a THC degradase, thus degrading the THC, but not the CBD produced by the first microorganism. Similarly, FIG. 5C illustrates the incubation of a crude preparation of Cannabis sp. (hemp) with a microorganism that produces a THC degradase, thus degrading the THC, but not the CBD in the hemp preparation. In another similar scenario, FIG. 5D illustrates the incubation of a purified cannabinoid preparation comprising THC and CBD with a microorganism that produces a THC degradase, thus eliminating the THC from the preparation.

Nonlimiting examples of enzymes that can be utilized in these reactions are provided in Table 1, where SEQ ID NOs:1-50 provide nucleic acid sequences for the enzymes, codon optimized for expression in yeast, and SEQ ID NOs:51-100 provide corresponding amino acid sequences. SEQ ID NOs:1-12 and 51-62 are P450 nucleic acid and amino acid sequences, respectively; SEQ ID NOs:13-20 and 63-70 are CPR nucleic acid and amino acid sequences, respectively; SEQ ID NOs:21-28 and 71-78 are CBN synthase nucleic acid and amino acid sequences, respectively; SEQ ID NOs:29-38 and 79-88 are THC degradase nucleic acid and amino acid sequences, respectively; and SEQ ID NOs:39-50 and 89-100 are oxidase nucleic acid and amino acid sequences, respectively. Of the oxidase enzymes provided, those comprising nucleic acid sequences SEQ ID NOs:42-50 and amino acid sequences SEQ ID NO:92-100 are laccases.

TABLE 1
Summary of codon optimized sequences provided herewith.

| | Codon Optimized | Amino Acid |
|---|---|---|
| | Nucleic Acid | Sequence for |
| Shorthand | Sequence | Isolated Protein |
| p450_1 | SEQ ID NO: 1 | SEQ ID NO: 51 |
| p450_2 | SEQ ID NO: 2 | SEQ ID NO: 52 |
| p450_3 | SEQ ID NO: 3 | SEQ ID NO: 53 |
| p450_4 | SEQ ID NO: 4 | SEQ ID NO: 54 |
| p450_5 | SEQ ID NO: 5 | SEQ ID NO: 55 |
| p450_6 | SEQ ID NO: 6 | SEQ ID NO: 56 |
| p450_7 | SEQ ID NO: 7 | SEQ ID NO: 57 |
| p450_8 | SEQ ID NO: 8 | SEQ ID NO: 58 |
| p450_9 | SEQ ID NO: 9 | SEQ ID NO: 59 |
| p450_10 | SEQ ID NO: 10 | SEQ ID NO: 60 |
| p450_11 | SEQ ID NO: 11 | SEQ ID NO: 61 |
| p450_12 | SEQ ID NO: 12 | SEQ ID NO: 62 |
| CPR_1 | SEQ ID NO: 13 | SEQ ID NO: 63 |
| CPR_2 | SEQ ID NO: 14 | SEQ ID NO: 64 |
| CPR_3 | SEQ ID NO: 15 | SEQ ID NO: 65 |
| CPR_4 | SEQ ID NO: 16 | SEQ ID NO: 66 |
| CPR_5 | SEQ ID NO: 17 | SEQ ID NO: 67 |
| CPR_6 | SEQ ID NO: 18 | SEQ ID NO: 68 |
| CPR_7 | SEQ ID NO: 19 | SEQ ID NO: 69 |
| CPR_8 | SEQ ID NO: 20 | SEQ ID NO: 70 |
| CBNsyn_1 | SEQ ID NO: 21 | SEQ ID NO: 71 |
| CBNsyn_2 | SEQ ID NO: 22 | SEQ ID NO: 72 |
| CBNsyn_3 | SEQ ID NO: 23 | SEQ ID NO: 73 |
| CBNsyn_4 | SEQ ID NO: 24 | SEQ ID NO: 74 |
| CBNsyn_5 | SEQ ID NO: 25 | SEQ ID NO: 75 |
| CBNsyn_6 | SEQ ID NO: 26 | SEQ ID NO: 76 |
| CBNsyn_7 | SEQ ID NO: 27 | SEQ ID NO: 77 |
| CBNsyn_8 | SEQ ID NO: 28 | SEQ ID NO: 78 |
| THCdeg_1 | SEQ ID NO: 29 | SEQ ID NO: 79 |
| THCdeg_2 | SEQ ID NO: 30 | SEQ ID NO: 80 |
| THCdeg_3 | SEQ ID NO: 31 | SEQ ID NO: 81 |
| THCdeg_4 | SEQ ID NO: 32 | SEQ ID NO: 82 |
| THCdeg_5 | SEQ ID NO: 33 | SEQ ID NO: 83 |
| THCdeg_6 | SEQ ID NO: 34 | SEQ ID NO: 84 |
| THCdeg_7 | SEQ ID NO: 35 | SEQ ID NO: 85 |
| THCdeg_8 | SEQ ID NO: 36 | SEQ ID NO: 86 |
| THCdeg_9 | SEQ ID NO: 37 | SEQ ID NO: 87 |
| THCdeg_10 | SEQ ID NO: 38 | SEQ ID NO: 88 |
| Oxid_1 | SEQ ID NO: 39 | SEQ ID NO: 89 |
| Oxid_2 | SEQ ID NO: 40 | SEQ ID NO: 90 |
| Oxid_3 | SEQ ID NO: 41 | SEQ ID NO: 91 |
| Oxid_4 | SEQ ID NO: 42 | SEQ ID NO: 92 |
| Oxid_5 | SEQ ID NO: 43 | SEQ ID NO: 93 |
| Oxid_6 | SEQ ID NO: 44 | SEQ ID NO: 94 |
| Oxid_7 | SEQ ID NO: 45 | SEQ ID NO: 95 |
| Oxid_8 | SEQ ID NO: 46 | SEQ ID NO: 96 |
| Oxid_9 | SEQ ID NO: 47 | SEQ ID NO: 97 |
| Oxid_10 | SEQ ID NO: 48 | SEQ ID NO: 98 |
| Oxid_11 | SEQ ID NO: 49 | SEQ ID NO: 99 |
| Oxid_12 | SEQ ID NO: 50 | SEQ ID NO: 100 |

### Enzymes

Also provided is a non-naturally occurring enzyme that can modify a first cannabinoid into a second cannabinoid or a non-cannabinoid.

The non-naturally occurring enzyme in these embodiments can have any alterations from a naturally occurring counterpart. In some embodiments, the enzyme comprises at least one amino acid that is not in a naturally occurring enzyme that has the same enzymatic activity. In some of those embodiments, the enzyme comprises a conservative substitution of an amino acid in a naturally occurring enzyme that has the same enzymatic activity. In various embodiments, the naturally occurring enzyme comprises any of SEQ ID NOs:51-100.

These enzymes can be utilized in the above-described methods. As such, in some embodiments, the first and/or second cannabinoid comprises the structure

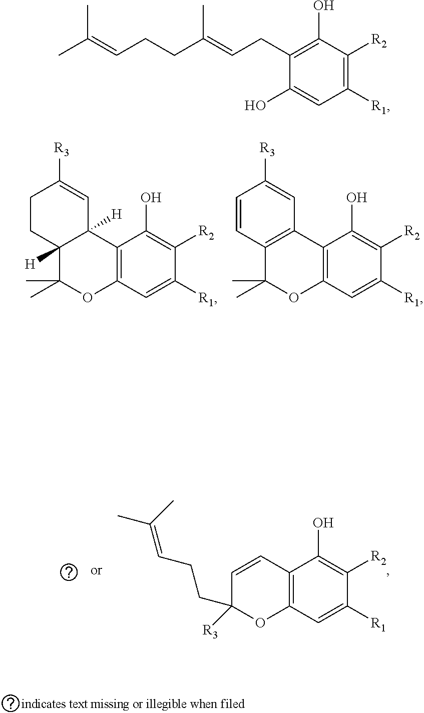

where R₁═CH₃, CH₂CH₃, (CH₂)₂CH₃, (CH₂)₃CH₃, (CH₂)₃CH₃, (CH₂)₄CH₃, (CH₂)₅CH₃, or (CH₂)₆CH₃; R₂═H or COOH; and R₃═CH₃ or CH₂OH. In other embodiments, the enzyme is an aromatase, a dehydrogenase, an oxidase or a desaturase. In additional embodiments, the first cannabinoid is tetrahydrocannabinol (THC) or tetrahydrocannabinolic acid (THCA) and the second cannabinoid is cannabinol (CBN) or cannabinolic acid (CBNA) and the enzyme is an aromatase, a dehydrogenase, an oxidase or a desaturase. In further embodiments, the first cannabinoid is tetrahydrocannabivarinic acid (THCVA), tetrahydrocannabiphorolic acid (TCHPA), tetrahydrocannabiorcinic acid (THCOA) or sesquiTHCA (THCFA) and the second cannabinoid is cannabinerolic acid (CBNA), cannabinerovarinic acid (CBNVA), cannabiphorolic acid (CBNPA), cannabinorcinic acid (CBNOA) or sesqui cannabinerolic acid (sesqui-CBNA), respectively. Also, the first cannabinoid can be tetrahydrocannabivarinol (THCV), tetrahydrocannabiphorol (TCHP), tetrahydrocannabiorcinol (THCO) or sesquitetrahydrocannabinolic acid (sesquiTHCA) and the second cannabinoid can be cannabinerovarinol (CBNV), cannabiphorol (CBNP), cannabinorcinol (CBNO) or sesqui cannabinerol (sesqui-CBN), respectively.

Where the enzyme activity is the conversion of the first cannabinoid, e.g., THC, THCA, CBN or CBNA, into a 11-hydroxy analog, the enzyme can be a combination of a cytochrome P450 (CYP-450) and a cytochrome P450 reductase (CPR). In some of these embodiments, the CYP-450 is a CYP2C9 or a CYP3A4 or a CYP76AH22-24 or a CYP76AH1 (ferruginol synthases).

In various embodiments, the enzyme is expressed from a codon optimized gene sequence in a yeast or a bacterium, e.g. E. coli.

The enzyme can be in vivo (e.g., in a yeast, bacterium or plant), or in vitro. Nonlimiting examples of transgenic plants in which the enzyme can be expressed are a Cannabis sp. or a tobacco plant. Nonlimiting examples of transgenic yeast in which the enzyme can be expressed are species of Saccharomyces, Candida, Pichia, Schizosaccharomyces, Scheffersomyces, Blakeslea, Rhodotorula, or Yarrowia. In some embodiments, the enzyme is in a yeast that further comprises enzymes to synthesize the first cannabinoid.

### Chemistry of the CBN Synthase Reaction

Chemically, the conversion of THC to CBN requires creation of 2 double bonds in a cyclohexene ring resulting in formation of an aromatic ring. See FIG. 2A. This is an oxidation reaction. Enzyme families catalyzing similar reactions include aromatases, dehydrogenases, desaturases, and oxidases (FIGS. 3A, 3B and 3C).

Classes of enzymes that are capable of derivatizing cannabinoids and species that contain such enzymes are provided herewith. Multiple CBN synthase enzymes and enzymes specific for THC catabolism without production of CBN can be provided. Different enzymatic specificity is also envisioned, e.g. conversion of the acid derivative of THC (THCA) to CBNA. Derivatives of THC can also be converted to the appropriate derivatives of CBN, e.g. THCVA to CBVA. See FIG. 2A.

Also envisioned are enzymes of these classes that selectively degrade THC by converting it to molecules other than CBN but leave other cannabinoids untouched.

### Enzyme Classes

The conversion of THC/A to CBN/A is an oxidation reaction, so it may be catalyzed by oxidases. CYP-450s are examples of enzymes of this reaction. Some oxygenases may add hydroxyl or ketone groups to the structure as they form the aromatic ring of CBN/A. This would generate a hydroxylated variant of CBN/A, a novel molecule. Oxidases may also include non P450s such as flavin-dependent monooxygenases, copper-dependent monooxygenases, bacterial polysaccharide monooxygenases, non-heme iron-dependent monooxygenases, pterin-dependent monooxygenases, diiron hydroxylases, alpha-ketoglutarate-dependent hydroxylases, other cofactor-dependent monooxygenases, cofactor-independent monooxygenases, and/or laccases (reviewed in Tones Pazmino, 2010).

An aromatic ring is formed by the CBN synthase, so it may also be catalyzed by aromatases (FIG. 3A). An example would be CYP19, an aromatase responsible for adding 2 double bonds to testosterone to create the aromatic ring in estradiol. The reaction is described here: https://www.uniprot.org/uniprot/Q16449.

As hydrogen atoms are abstracted to make the double bonds in CBN/A, a dehydrogenase may be able to catalyze the reaction. An example of a dehydrogenase that catalyzes a similar reaction would be arogenate dehydrogenase, as described here: https://www.uniprot.org/uniprot/Q944B6. Since double bonds are formed in creation of CBN/A, a desaturase may be responsible. An example of a desaturase that catalyzes a similar reaction would be arogenate dehydratase/prephenate dehydratase, as described at https://www.uniprot.org/uniprot/Q9LMR3

Some enzymes of these classes will also degrade THC by converting it to molecules other than CBN. A non-limiting example is reversing THCA synthase to generate CBGA.

In some embodiments, the CBN synthase can use any variant of tetrahydrocannabinolic acid THCA, as starting material, including: tetrahydrocannabivarinic acid (THCVA), tetrahydrocannabiphorolic acid (TCHPA), tetrahydrocannabiorcinic acid (THCOA), sesquiTHCA (THCFA) and produce, respectively, cannabinerolic acid (CBNA), cannabinerovarinic acid (CBNVA), cannabiphorolic acid (CBNPA), cannabinorcinic acid (CBNOA) sesqui cannabinerolic acid (sesqui-CBNA). Decarboxylation of any of these products, either enzymatically or by non-enzymatic methods such as heat, will produce the respective decarboxylated derivatives and is an optional last step of the pathway.

### Organisms Originating the Enzymes

The enzyme can be a naturally occurring enzyme, or an enzyme derived from a naturally occurring enzyme, now known or later discovered, that occurs in any living organism, for example a bacterium, an archaeon, a protist, a fungus, an algae, an animal or a plant.

Many microbial enzymes catalyze reactions of these classes using similar substrates, but have never been tested for activity on cannabinoids. To determine a source of a CBN synthase, microbes can be screened for bioconversion activity of appropriate cannabinoids, after the methods of Abbott (1977). Microbes possessing this activity should have their genomes sequenced if there is no publicly available genome. Enzymes from the above listed enzyme classes should be found from the sequenced genomes and thereby identified as good candidates for the CBN synthase activity. Organisms that make molecules similar to desired cannabinoids can be identified from literature and those genomes searched as well to identify additional candidate enzymes. Bioinformatics methods to do this are in U.S. Pat. No. 10,671,632

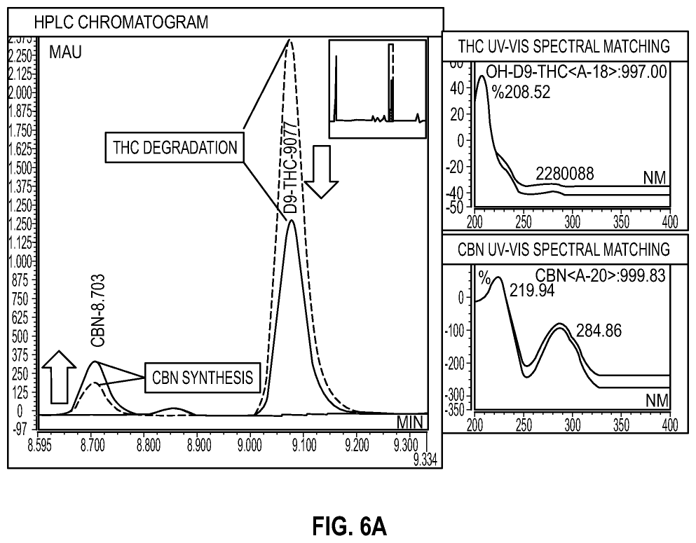

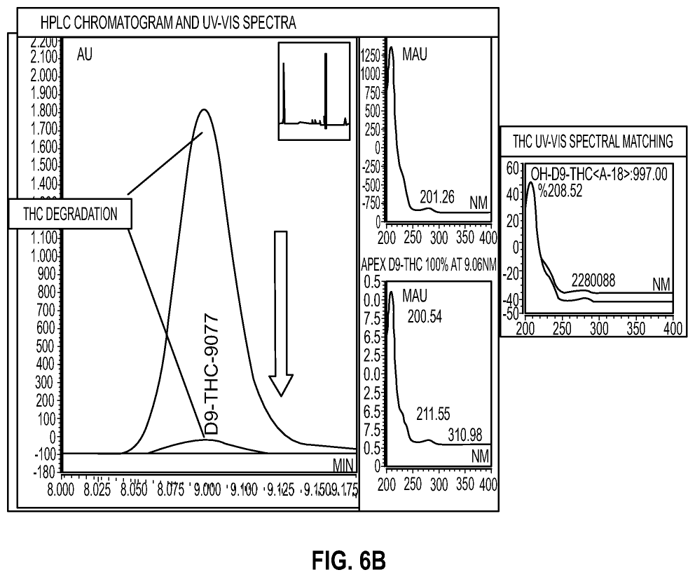

Some microbes screened will contain a THC degradase instead of a CBN synthase. This is detectable as a reduction in a THC containing starting material relative to a negative control (FIGS. 6A and 6B).

In some embodiments, the gene for the enzyme is derived from a bacterium. It is envisioned that an enzyme derived from any bacterium now known or later discovered can be utilized in the present invention. For example, the bacterium can be from phylum Abditibacteriota, including class Abditibacteria, including order Abditibacteriales; phylum Abyssubacteria or Acidobacteria, including class Acidobacteriia, Blastocatellia, Holophagae, Thermoanaerobaculia, or Vicinamibacteria, including order Acidobacteriales, Bryobacterales, Blastocatellales, Acanthopleuribacterales, Holophagales, Thermotomaculales, Thermoanaerobaculales, or Vicinamibacteraceae; phylum Actinobacteria, including class Acidimicrobiia, Actinobacteria, Actinomarinidae, Coriobacteriia, Nitriliruptoria, Rubrobacteria, or Thermoleophilia, including orders Acidimicrobiales, Acidothermales, Actinomycetales, Actinopolysporales, Bifidobacteriales, Nanopelagicales, Catenulisporales, Corunebacteriales, Cryptosporangiales, Frankiales, Geodermatophilales, Glycomycetales, Jiangellales, Micrococcales, Micromonosporales, Nakamurellales, Propionibacteriales, Pseudonocardiales, Sporichthyales, Streptomycetales, Streptosporangiales, Actinomarinales, Coriobacteriales, Eggerthellales, Egibacterales, Egicoccales, Euzebyales, Nitriliruptorales, Gaiellales, Rubrobacterales, Solirubrobacterales, or Thermoleophilales; phylum Aquificae, including class Aquificae, including order Aquificales or Desulfurobacteriales; phylum Armatimonadetes, including class Armatimonadia, including order Armatimonadales, Capsulimonadales, Chthonomonadetes, Chthonomonadales, Fimbriimonadia, or Fimbriimonadales; phylum Aureabacteria or Bacteroidetes, including class Armatimonadia, Bacteroidia, Chitinophagia, Cytophagia, Flavobacteria, Saprospiria or Sphingobacteriia, including order Bacteroidales, Marinilabiliales, Chitinophagales, Cytophagales, Flavobacteriales, Saprospirales, or Sphingopacteriales; phylum Balneolaeota, Caldiserica, Calditrichaeota, or Chlamydiae, including class Balneolia, Caldisericia, Calditrichae, or Chlamydia, including order Balneolales, Caldisericales, Calditrichales, Anoxychlamydiales, Chlamydiales, or Parachlamydiales; phylum Chlorobi or Chloroflexi, including class Chlorobia, Anaerolineae, Ardenticatenia, Caldilineae, Thermofonsia, Chloroflexia, Dehalococcoidia, Ktedonobacteria, Tepidiformia, Thermoflexia, Thermomicrobia, or Sphaerobacteridae, including order Chlorobiales, Anaerolineales, Ardenticatenales, Caldilineales, Chloroflexales, Herpetosiphonales, Kallotenuales, Dehalococcoidales, Dehalogenimonas, Kte donob acteral es, Thermogemmatisporales, Tepidiformales, Thermoflexales, Thermomicrobiales, or Sphaerobacterales; phylum Chrysiogenetes, Cloacimonetes, Coprothermobacterota, Cryosericota, or Cyanobacteria, including class Chrysiogenetes, Coprothermobacteria, Gloeobacteria, or Oscillatoriophycideae, including order Chrysiogenales, Coprothermobacterales, Chroococcidiopsidales, Gloeoemargaritales, Nostocales, Pleurocapsales, Spirulinales, Synechococcales, Gloeobacterales, Chroococcales, or Oscillatoriales; phyla: Eferribacteres, Deinococcus-thermus, Dictyoglomi, Dormibacteraeota, Elusimicrobia, Eremiobacteraeota, Fermentibacteria, or Fibrobacteres, including class Deferribacteres, Deinococci, Dictyoglomia, Elusimicrobia, Endomicrobia, Chitinispirillia, Chitinivibrionia, or Fibrobacteria, including order Deferribacterales, Deinococcales, Thermales, Dictyoglomales, Elusimicrobiales, Endomicrobiales, Chitinspirillales, Chitinvibrionales, Fibrobacterales, or Fibromonadales; phylum Firmicutes, Fusobacteria, Gemmatimonadetes, or Hydrogenedentes, including class Bacilli, Clostridia, Erysipelotrichia, Limnochordia, Negativicutes, Thermolithobacteria, Tissierellia, Fusobacteriia, Gemmatimonadetes, Longimicrobia, including order Bacillales, Lactobacillales, Borkfalkiales, Clostridiales, Halanaerobiales, Natranaerobiales, Thermoanaerobacterales, Erysipelotrichales, Limnochordales, Acidaminococcales, Selenomonadales, Veillonellales, Thermolithobacterales, Tissierellales, Fusobacteriales, Gemmatimonadales, or Longimicrobia; phylum Hydrogenedentes, Ignavibacteriae, Kapabacteria, Kiritimatiellaeota, Krumholzibacteriota, Kryptonia, Latescibacteria, LCP-89, Lentisphaerae, Margulisbacteria, Marinimicrobia, Melainabacteria, Nitrospinae, or Omnitrophica, including class Ignavibacteria, Kiritimatiellae, Krumholzibacteria, Lentisphaeria, Oligosphaeria, or Nitrospinae, including order Ignavibacteriales, Kiritimatiellales, Krumholzibacteriales, Lentisphaerales, Victivallales, Oligosphaerales, or Nitrospinia; phylum Omnitrophica or Planctomycetes, including class Brocadiae, Phycisphaerae, Planctomycetia, or Phycisphaerales, including order Sedimentisphaerales, Tepidisphaerales, Gemmatales, Isosphaerales, Pirellulales, or Planctomycetales; phylum Proteobacteria including class Acidithiobacillia, Alphaproteobacteria, Betaproteobacteria, Lambdaproteobacteria, Muproteobacteria, Deltaproteobacteria, Epsilonproteobacteria, Gammaproteobacteria, Hydrogenophilalia, Oligoflexia, or Zetaproteobacteria, including order Acidithiobacillales, Caulobacterales, Emcibacterales, Holosporales, Iodidimonadales, Kiloniellales, Kopriimonadales, Kordiimonadales, Magnetococcales, Micropepsales, Minwuiales, Parvularculales, Pelagibacterales, Rhizobiales, Rhodobacterales, Rhodospirillales, Rhodothalassiales, Rickettsiales, Sneathiellales, Sphingomonadales, Burkholderiales, Ferritrophicales, Ferrovales, Neisseriales, Nitrosomonadales, Procabacteriales, Rhodocyclales, Bradymonadales, Acidulodesulfobacterales, Desulfarculales, Desulfobacterales, Desulfovibrionales, Desulfurellales, Desulfuromonadales, Myxococcales, Syntrophobacterales, Campylobacterales, Nautiliales, Acidiferrobacterales, Aeromonadales, Alteromonadales, Arenicellales, Cardiobacteriales, Cellvibrionales, Chromatiales, Enterobacterales, Immundisolibacterales, Legionellales, Methylococcales, Nevskiales, Oceanospirillales, Orbales, Pasteurellales Pseudomonadales, Salinisphaerales, Thiotrichales, Vibrionales, Xanthomonadales, Hydrogenophilales, Bacteriovoracales, Bdellovibrionales, Oligoflexales, Silvanigrellales, or Mariprofundales; phylum Rhodothermaeota, Saganbacteria, Sericytochromatia, Spirochaetes, Synergistetes, Tectomicrobia, or Tenericutes, including class Rhodothermia, Spirochaetia, Synergistia, Izimaplasma, or Mollicutes, including order Rhodothermales, Brachyspirales, Brevinematales, Leptospirales, Spirochaetales, Synergistales, Acholeplasmatales, Anaeroplasmatales, Entomoplasmatales, or Mycoplasmatales; phylum Thermodesulfobacteria, Thermotogae, Verrucomicrobia, or Zixibacteria, including class Thermodesulfobacteria, Thermotogae, Methylacidiphilae, Opitutae, Spartobacteria, or Verrucomicrobiae, including order Thermodesulfobacteriales, Kosmotogales, Mesoaciditogales, Petrotogales, Thermotogales, Methylacidiphilales, Opitutales, Puniceicoccales, Xiphinematobacter, Chthoniobacterales, Terrimicrobium, or Verrucomicrobiales.

In other embodiments, the gene for the enzyme is derived from an archaeon. It is envisioned that an enzyme derived from any archaeon now known or later discovered can be utilized in the present invention. For example, the archaeon can be from phylum Euryarchaeota, including class Archaeoglobi, Hadesarchaea, Halobacteria, Methanobacteria, Methanococci, Methanofastidiosa, Methanomicrobia, Methanopyri, Nanohaloarchaea, Theionarchaea, Thermococci, or Thermoplasmata, including order Archaeoglobales, Hadesarchaeales, Halobacteriales, Methanobacteriales, Methanococcales, Methanocellales, Methanomicrobiales, Methanophagales, Methanosarcinales, Methanopyrales, Thermococcales, Methanomassiliicoccales, Thermoplasmatales, or Nanoarchaeales; DPANN superphylum, including subphyla Aenigmarcheota, Altiarchaeota, Diapherotrites, Micrarchaeota, Nanoarchaeota, Pacearchaeota, Parvarchaeota, or Woesearchaeota; TACK superphylum, including subphylum Korarchaeota, Crenarchaeota, Aigarchaeota, Geoarchaeota, Thaumarchaeota, or Bathyarchaeota; Asgard superphylum including subphylium Odinarchaeota, Thorarchaeota, Lokiarchaeota, Helarchaeota, or Heimdallarchaeota.

In additional embodiments, the gene for the enzyme is derived from a fungus. It is envisioned that a CBN synthase or THC degradase from any fungus now known or later discovered can be utilized in the present invention. This includes but is not limited to the phyla Chytridiomycota, Basidiomycota, Ascomycota, Blastocladiomycota, Ascomycota, Microsporidia, Basidiomycota, Glomeromycota, Symbiomycota, and Neocallimastigomycota. For example, the fungus can be from the phylum Ascomycota, including classes and orders Pezizomycotina, Arthoniomycetes, Coniocybomycetes, Dothideomycetes, Eurotiomycetes, Geoglossomycetes, Laboulbeniomycetes, Lecanoromycetes, Leotiomycetes, Lichinomycetes, Orbiliomycetes, Pezizomycetes, Sordariomycetes, Xylonomycetes, Lahmiales, Itchiclahmadion, Triblidiales, Saccharomycotina, Saccharomycetes, Taphrinomycotina, Archaeorhizomyces, Neolectomycetes, Pneumocystidomycetes, Schizosaccharomycetes, Taphrinomycetes; phylum Basidiomycota including subphyla or classes Pucciniomycotina, Ustilaginomycotina, Wallemiomycetes, and Entorrhizomycetes; subphylum Agaricomycotina including classes Tremellomycetes, Dacrymycetes, and Agaricomycetes; phylum Symbiomycota, including class Entorrhizomycota; subphylum Ustilaginomycotina including classes Ustilaginomycetes and Exobasidiomycetes; phylum Glomeromycota including classes Archaeosporomycetes, Glomeromycetes, and Paraglomeromycetes; subphylum Pucciniomycotina including orders and classes: Pucciniomycotina, Cystobasidiomycetes, Agaricostilbomycetes, Microbotryomycetes, Atractiellomycetes, Classiculomycetes, Mixiomycetes, and Cryptomycocolacomycetes; subphylum incertae sedis Mucoromyceta including orders Calcarisporiellomycota and Mucoromycota; phylum Mortierellomyceta including class Mortierellomycota; subphylum incertae sedis Entomophthoromycotina including order Entomophthorales; phylum Zoopagomyceta including classes Basidiobolomycota, Entomophthoromycota, Kickxellomycota, and Zoopagomycotina; subphylum incertae sedis Mucoromycotina including orders Mucorales, Endogonales, and Mortierellales; phylum Neocallimastigomycota including class Neocallimastigomycetes; phylum Blastocladiomycota including classes Physodermatomycetes and Blastocladiomycetes; phylum Rozellomyceta including classes Rozellomycota and Microsporidia; phylum Aphelidiomyceta including class Aphelidiomycota; Chytridiomyceta including classes Chytridiomycetes and Monoblepharidomycetes; and phylum Oomycota including classes or orders Leptomitales, Myzocytiopsidales, Olpidiopsidales, Peronosporales, Pythiales, Rhipidiales, Salilagenidiales, Saprolegniales, Sclerosporales, Anisolpidiales, Lagenismatales, Rozellopsidales, and Haptoglossales.

### Nucleic Acids

The present invention is additionally directed to nucleic acids encoding any of the above-identified enzymes. In some embodiments, the nucleic acids are codon optimized to improve expression, e.g., using techniques as disclosed in U.S. Pat. No. 10,435,727. In some of these embodiments, the codon optimized nucleic acids comprise any of SEQ ID NOs:1-50.

More specifically, optimized nucleotide sequences are generated based on a number of considerations: (1) For each amino acid of the recombinant polypeptide to be expressed, a codon (triplet of nucleotide bases) is selected based on the frequency of each codon in the Saccharomyces cerevisiae genome; the codon can be chosen to be the most frequent codon or can be selected probabilistically based on the frequencies of all possible codons. (2) In order to prevent DNA cleavage due to a restriction enzyme, certain restriction sites are removed by changing codons that cover those sites. (3) To prevent low-complexity regions, long repeats (sequences of any single base longer than five bases) are modified. (2) and (3) are performed recursively to ensure that codon modification does not lead to additional undesirable sequences. (4) A ribosome binding site is added to the N-terminus. (5) A stop codon is added.

In various embodiments, the nucleic acids further comprise additional nucleic acids encoding amino acids that are not part of the enzyme. In some of these embodiments, the additional sequences encode additional amino acids present when the nucleic acid is translated, encoding, for example, an additional protein domain, with or without a linker sequence, creating a fusion protein. Other examples are localization sequences, i.e., signals directing the localization of the folded protein to a specific subcellular compartment or membrane.

In some embodiments, the nucleic acids have, at the 5′ end, a nucleic acid encoding codon optimized cofolding peptides to create a fusion protein, e.g., having SEQ ID NOs:69-73 (Table 2), joining the sequences together to form a fusion polypeptide, e.g., having the amino acid sequence of SEQ ID NO:74-78 fused at the N terminus of the enzyme polypeptide, generating recombinant fusion polypeptides.

TABLE 2

| | Codon Optimized | Amino Acid |
|---|---|---|
| | Nucleic Acid | Sequence for |
| NAME | Sequence | Isolated Protein |
| MBP | Seq. ID NO: 101 | Seq. ID NO: 106 |
| VEN | Seq. ID NO: 102 | Seq. ID NO: 107 |
| MST | Seq. ID NO: 103 | Seq. ID NO: 108 |
| OSP | Seq. ID NO: 104 | Seq. ID NO: 109 |
| OLE | Seq. ID NO: 105 | Seq. ID NO: 110 |

Further provided is a non-naturally occurring nucleic acids that encode an enzyme having the enzymatic activity of any of the non-naturally occurring enzymes described above, or a naturally occurring enzyme having any of the enzyme activities described above. The nucleic acids may be codon optimized, e.g., for production in yeast.

In some embodiments, the nucleic acid comprises additional nucleotide sequences that are not translated. Examples include promoters, terminators, barcodes, Kozak sequences, targeting sequences, and enhancer elements. Particularly useful here are promoters that are functional in yeast.

Expression of a gene encoding an enzyme is determined by the promoter controlling the gene. In order for a gene to be expressed, a promoter must be present within 1,000 nucleotides upstream of the gene. A gene is generally cloned under the control of a desired promoter. The promoter regulates the amount of enzyme expressed in the cell and also the timing of expression, or expression in response to external factors such as sugar source.

Any promoter now known or later discovered can be utilized to drive the expression of the various genes (e.g., 11-OH hydroxylase, CBN synthase, THC degradase) described herein. See e.g. http://parts.igem.org/Yeast for a listing of various yeast promoters. Exemplary promoters listed in Table 3 below drive strong expression, constant gene expression, medium or weak gene expression, or inducible gene expression. Inducible or repressible gene expression is dependent on the presence or absence of a certain molecule. For example, the GAL1, GAL 7, and GAL10 promoters are activated by the presence of the sugar galactose and repressed by the presence of the sugar glucose. The HO promoter is active and drives gene expression only in the presence of the alpha factor peptide. The HXT1 promoter is activated by the presence of glucose while the ADH2 promoter is repressed by the presence of glucose.

TABLE 3
Exemplary yeast promoters

| Strong | Medium and weak | Inducible/ |
|---|---|---|
| constitutive | constitutive | repressible |
| promoters | promoters | promoters |
| TEF1 | STE2 | GAL1 |
| PGK1 | TPI1 | GAL7 |
| PGI1 | PYK1 | GAL10 |
| TDH3 | | HO |
| | | HXT1 |
| | | ADH2 |

In various embodiments, the nucleic acid is in a yeast expression cassette. Any yeast expression cassette capable of expressing the enzyme in a yeast cell can be utilized. In some embodiments, the expression cassette consists of a nucleic acid encoding a CBN synthase or THC degradase with a promoter.

Additional regulatory elements can also be present in the expression cassette, including restriction enzyme cleavage sites, antibiotic resistance genes, integration sites, auxotrophic selection markers, origins of replication, and degrons.

The expression cassette can be present in a vector that, when transformed into a host cell, either integrates into chromosomal DNA or remains episomal in the host cell. Such vectors are well-known in the art. See e.g. http://parts.igem.org/Yeast for a listing of various yeast vectors.

A nonlimiting example of a yeast vector is a yeast episomal plasmid (YEp) that contains the pBluescript II SK(+) phagemid backbone, an auxotrophic selectable marker, yeast and bacterial origins of replication and multiple cloning sites enabling gene cloning under a suitable promoter (see Table 3). Other exemplary vectors include pRS series plasmids.

### Host Cells

The present invention is also directed to genetically engineered host cells that comprise the above-described nucleic acids. Such cells may be, e.g., any species of filamentous fungus, including but not limited to any species of Aspergillus, which have been genetically altered to produce precursor molecules, intermediate molecules, or cannabinoid molecules. Host cells may also be any species of bacteria, including but not limited to Escherichia, Corynebacterium, Caulobacter, Pseudomonas, Streptomyces, Bacillus, or Lactobacillus.

In some embodiments, the genetically engineered host cell is a yeast cell, which may comprise any of the above-described expression cassettes, and capable of expressing the recombinant enzyme encoded therein.

Any yeast cell capable of being genetically engineered can be utilized in these embodiments. Nonlimiting examples of such yeast cells include species of Saccharomyces, Candida, Pichia, Schizosaccharomyces, Scheffersomyces, Blakeslea, Rhodotorula, or Yarrowia.

These cells can achieve gene expression controlled by inducible promoter systems; natural or induced mutagenesis, recombination, and/or shuffling of genes, pathways, and whole cells performed sequentially or in cycles; overexpression and/or deletion of single or multiple genes and reducing or eliminating parasitic side pathways that reduce precursor concentration.

The host cells of the recombinant organism may also be engineered to produce any or all precursor molecules necessary for the biosynthesis of cannabinoids, including but not limited to olivetolic acid (OA), olivetol (OL), FPP and GPP, hexanoic acid and hexanoyl-CoA, malonic acid and malonyl-CoA, dimethylallylpyrophosphate (DMAPP) and isopentenylpyrophosphate (IPP) as disclosed in U.S. Pat. No. 10,435,727.

Construction of Saccharomyces cerevisiae strains expressing a cannabinoid modifying or degrading enzyme such as CBN synthase or THC degradase is carried out via expression of a gene which encodes for the enzyme. The gene encoding the enzyme can be cloned into vectors with the proper regulatory elements for gene expression (e.g. promoter, terminator) and the derived plasmid can be confirmed by DNA sequencing. As an alternative to expression from an episomal plasmid, the gene encoding the enzyme may be inserted into the recombinant host genome. Integration may be achieved by a single or double cross-over insertion event of a plasmid, or by nuclease-based genome editing methods, as are known in the art e.g. CRISPR, TALEN and ZFR. Strains with the integrated gene can be screened by rescue of auxotrophy and genome sequencing. See, e.g., Green and Sambrook (2012).

To produce the desired cannabinoid, each candidate polypeptide may be introduced into a host cell genetically modified to contain all necessary components for cannabinoid biosynthesis using standard yeast cell transformation techniques (Green and Sambrook, 2012). Cells are subjected to fermentation under conditions that activate the promoter controlling the candidate polypeptide (see, e.g., Table 3). The broth may be subsequently subjected to HPLC analysis (FIGS. 6A and 6B).

In some embodiments, for recombinant enzyme purification, the gene encoding the enzyme is cloned into an expression vector such as the pET expression vectors from Novagen, transformed into a protease deficient strain of E. coli such as BL21 and expressed by induction with IPTG. The protein of interest may be tagged with a common tag to facilitate purification, e.g. hexahistidine, GST, calmodulin, TAP, AP, CAT, HA, FLAG, MBP etc. Coexpression of a bacterial chaperone such as dnaK, GroES/GroEL or SecY may help facilitate protein folding. See Green and Sambrook (2012).

Any of the enzymes described above can also be produced in transgenic plants, using techniques known in the art (see, e.g., Keshavareddy et al., 2018). In these embodiments, the above-described nucleic acid encoding the enzyme further comprises a promoter functional in a plant. In various embodiments, the nucleic acid is in a plant expression cassette. Any plant capable of being transformed with the nucleic acid can be utilized here. In some embodiments, the plant is a tobacco or a Cannabis sp. plant. Cannabis sp. that are transformed with a THC degradase are particularly useful, since such an enzyme expressed in Cannabis sp. plants grown for fiber could reduce the THC content to below the 0.3% current legal THC limit.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

Various methods and compositions provided in U.S. patent applications Ser. Nos. 16/553,103, 16/553,120, 16/558,973, 17/068,636 and 63/053,539; U.S. Pat. No. 10,435,727; and US Patent Publications 2020/0063170 and 2020/0063171 are utilized in the examples.

### EXAMPLE 1

### Expression of a Recombinant Fusion Polypeptides for THC/A Conversion, Degradation, and 11-Hydroxy Cannabinoid Variant Production in a Modified Host Organism

Construction of Saccharomyces cerevisiae strains expressing CBN synthase, THC degradase, P450, and/or CPR enzymes fused with N terminal cofolding peptides from Table 1, having SEQ ID NOs:106-110 to produce CBN/A from THC/A, and 11-hydroxy variants such as 11-OH CBN, is carried out via expression of a fusion gene of any codon optimized nucleic acid sequence SEQ ID NOs:101-105 combined at the 5′ end of a nucleic acid sequence encoding an enzyme that modifies a first cannabinoid into a second cannabinoid or non-cannabinoid. The fusion genes were cloned into vectors with the proper regulatory elements for gene expression (e.g. promoter, terminator) and the derived plasmid was confirmed by DNA sequencing. The fusion genes were also inserted into the recombinant host genome. Integration was achieved by a single or double cross-over insertion event of the plasmid. Strains with the integrated gene were screened by rescue of auxotrophy and genome sequencing.

### EXAMPLE 2

### Method of Growth of Host Cells

Modified host cells which yield cannabinoids such as THC/A, express recombinant (i) CBN synthase for THC/A conversion to CBN/A, (ii) p450 and CPR protein combinations (11-OH hydroxylases) for 11-OH hydroxy variants of cannabinoids such as 11-OH-THC, or (iii) a combination of CBN synthase and 11-OH hydroxylases for production of cannabinoids such as 11-OH-CBN. More specifically, the cannabinoid-producing strain expressing CBN synthases and/or 11-OH hydroxylases herein is grown in a feedstock as described in U.S. patent application Ser. No. 17/068,636. An example feedstock used for a modified host expressing the recombinant CBN synthase is growing the strain in a minimal-complete or rich culture media containing yeast nitrogen base, amino acids, vitamins, ammonium sulfate, and a carbon source, such as glucose or molasses. The feedstock is consumed by the modified host which expresses the recombinant CBN synthase with a cannabinoid biosynthesis pathway to convert the feedstock into (i) biomass, (ii) THC/A and 11-OH-THC variants thereof, (iii) CBN/A and 11-OH CBN, and variants thereof, or (iv) biomass and the cannabinoids products in (ii) and (iii). Strains expressing the recombinant CBN synthase genes can be grown on feedstock for 12 to 160 hours at 25-37° C. for isolation of products.

### EXAMPLE 3

### Removal of THC/A by Formation of a Homopolymer or Heteropolymer

Cells are genetically engineered to contain one or more laccase enzymes. Integration is achieved by a single or double cross-over insertion event of the plasmid. Strains with the integrated gene are screened by rescue of auxotrophy and genome sequencing. The laccase gene can be under the control of an inducible promoter. When polymerization of THC/A is desired, inducer is added to the culture along with supplemental copper at a final concentration of 100 μM-100 mM. Polymerized cannabinoids can be separated from the culture by filtration, centrifugation or dialysis. Membranes for filtration and dialysis should be selected such that molecules corresponding to the size of a monomeric cannabinoid pass through the pores of the membrane, but larger molecules such as polymers are retained on the other side of the membrane.

### EXAMPLE 4

### Purification of Recombinant CBN Synthase and THC Degradase Enzymes for THC/A Conversion or Degradation

The CBN synthase or THC degradase enzyme is cloned into a high-copy vector with key features that allow 1) tight induction by the lactose analog, β-D-thiogalactoside (IPTG), 2) an N-terminal secretory signal peptide (e.g., MKKTAIAIAVALAGFATVAQA), and 3) C-terminal fusion to a HIS tag for purification. E. coli cells harboring the CBN synthase or THC degradase expression vector are grown in M9 minimal media with 1% glucose for 18 h at 37° C. and shaking at 300 rpm. Concentrated cell culture is diluted to an OD₆₀₀=1 in fresh M9 minimal media with 1% glucose and 0.2 mM IPTG and grown for 48 h.

The supernatant containing the recombinant proteins is equilibrated in binding buffer (50 mM sodium phosphate, 0.5 M NaCl, 20 mM imidazole, 1 mM MgCl₂, 10% glycerol, 10 mM 2-mercaptoethanol, 1 mM PMSF, Complete EDTA-free (1 tablet/100 ml), 20 mM 1-phenyl-2-thiourea; pH 7.4) and centrifuged at 2,500 g for 5 min to remove insoluble matter. Then the supernatant is filtered through a 0.45 μm filter (Millipore, MA, USA) and applied onto a HisTrap HP column (GE Healthcare Bioscience). The recombinant proteins are eluted with a step gradient of imidazole (concentrations of 5, 20, 40 and 300 mM). Fractions are analyzed by SDS-PAGE.

Purified CBN synthase or THC degradase protein is resuspended in activity buffer [100 mM sodium phosphate buffer, pH 6.55, 1 mM PMSF, EDTA-free protease inhibitor cocktail at working concentration (Roche, Meylan, France)] for use in converting or degrading THC/A in crude plant matter or THC/A in cannabinoid isolate via incubation and continuous shaking for 6-12 hrs at 30° C.

### EXAMPLE 5

### Preparation of Cell Lysate from a Host Expression Recombinant CBN Synthase and THC Degradase for Conversion and Degradation of THC/A

Host cells expressing recombinant CBN synthase or THC degradase are resuspended in lysis buffer consisting of 50 mM Tris-HCl pH7.5, 200 mM NaCl, 1 mM MgCl₂, 5 mM DTT, 1 mM PMSF, and DNAse. Resuspended host cells are then lysed by sonication/French press/homogenization or enzymatic lysis such as zymolyase or lysozyme. Lysate is cleared by centrifugation at 16000 rpm for 15 min at 4° C. Cleared lysate is added to crude or purified cannabinoid preparations at concentrations ranging from 1 mg/gram to 1 g/g. The mixture is incubated with continuous shaking for 6-12 hrs at 30° C. Cannabinoids are then extracted.

### EXAMPLE 6

### Detection of Isolated Product

To identify cannabinoid conversion products from CBN synthase, the degradation of THC via THC degradase, 11-hydroxy variants of cannabinoids, and all other products of converted plant matter, cannabinoid isolate, or from a host cell expressing an engineered biosynthetic pathway for cannabinoids, an Agilent 1100 series liquid chromatography (LC) system equipped with a reverse phase C18 column (Agilent Eclipse Plus C18, Santa Clara, Calif., USA) was used. A gradient was used of mobile phase A (ultraviolet (UV) grade H₂O+0.1% formic acid) and mobile phase B (UV grade acetonitrile+0.1% formic acid). Column temperature was set at 30° C. Compound absorbance was measured at 210 nm and 305 nm using a diode array detector (DAD) and spectral analysis from 200 nm to 400 nm wavelengths. A 0.1 milligram (mg)/milliliter (mL) analytical standard was made from certified reference material for each terpene and cannabinoid (Cayman Chemical Company, USA). Each sample was prepared by diluting 1) fermentation biomass from a recombinant host expressing the engineered cannabinoid and CBN synthase biosynthesis pathway or 2) a conversion or degradation reaction containing CBN synthase or THC degradase by 1:3 or 1:20 in 100% acetonitrile and filtered in 0.2 um nanofilter vials. The retention time and UV-visible absorption spectrum (i.e., spectral fingerprint) of the samples were compared to the analytical standard retention time and UV-visible spectra (i.e. spectral fingerprint) when identifying the terpene and cannabinoid compounds.

FIG. 6A depicts the detection of CBN and THC isolated from fermentation broth with a recombinant CBN synthase host and from fermentation broth with a control microorganism. Detection and isolation of product are depicted by retention time matching of post-fermentation conversion and degradation of THC into CBN with CBN and THC analytical standards, along with a matching UV-vis spectral fingerprint of the post-fermentation conversion and degradation of THC with the THC analytical standard and CBN with the CBN analytical standard. This also corroborates that the recombinant host is able to successfully convert and degrade THC, which further validates that the systems and methods herein enzymatically target THC/A molecules for conversion and degradation.

FIG. 6B depicts the detection of THC isolated from fermentation broth with a recombinant THC degrading host and from fermentation broth with a control microorganism. Detection and isolation of product are depicted by retention time matching of post-fermentation conversion and degradation of THC with a THC analytical standard, along with a matching UV-vis spectral fingerprint of the post-fermentation conversion and degradation of THC with the THC analytical standard. This also corroborates that the recombinant host is able to successfully convert and degrade THC, which further validates that the systems and methods herein enzymatically target THC/A molecules for conversion and degradation.

### REFERENCES

Abbott et al., 1977, Experientia 33:718-720.

Carter et al., 2010, Am J Hosp Palliat Care 27:347-56.

Caprioglio et al., 2019, Org. Lett. 21:6122-6125.

Carvalho et al., 2017, FEMS Yeast Res. 17:fox037.

Committee on the Health Effects of Marijuana, 2017, The Health Effects of Cannabis and Cannabinoids: The Current State of Evidence and Recommendations for Research, National Academies Press.

Giacoppo S. and Mazzon E. 2016, Neural Regeneration Res. 11:1896-4899.

Green and Sambrook (2012) Molecular Cloning: A Laboratory Manual (Fourth Edition), Cold Spring Harbor Laboratory Press, Cold Spring Harbor, N.Y.

Gülck and Møller, 2020, Trends in Plant Science 25:985-1004.

Keshavareddy et al., 2018, Int. J. Curr. Microbiol.App. Sci 7:2656-2668.

Luo et al., 2019, Nature 567:123-126.

Torres Pazmino, Daniel & Winkler, Margit & Glieder, Anton & Fraaije, Marco. (2010). Monooxygenases as biocatalysts: Classification, mechanistic aspects and biotechnological applications. Journal of biotechnology. 146. 9-24. 10.1016/j.jbiotec.2010.01.021.

Watanabe et al., 2007, Life Sciences 80:1415-1419.

http://parts.igem.org/Yeast.

https://www.uniprot.org/uniprot/Q16449.

PCT Patent Application Publication WO 2014/159688 A1.

US Patent Application Publication 2017/0020943 A1.

U.S. Pat. No. 10,435,727.

U.S. Pat. No. 10,671,632.

U.S. patent application Ser. No. 16/553,103.

U.S. patent application Ser. No. 16/553,120.

U.S. patent application Ser. No. 16/558,973.

U.S. patent application Ser. No. 17/068,636.

U.S. Provisional Patent Application 63/035,692.

U.S. Provisional Patent Application 63/053,539.

U.S. Provisional Patent Application 63/141,486.

US Patent Application Publication 2020/0063170.

US Patent Application Publication 2020/0063171.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc

SEQ ID NOs
>p450_1
Seq. ID NO: 1
ATGGCCGCAGACAGTCTTGTTGTCCTTGTTCTGTGCCTTAGTTGCCTTTTGCTGCTAT
CTCTTTGGAGACAATCATCAGGGAGAGGTAAACTTCCGCCTGGACCAACTCCACTAC
CCGTCATAGGGAATATATTACAAATCGGTATAAAGGACATCTCCAAGTCCCTGACGA
ATCTTTCCAAGGTGTATGGTCCTGTGTTCACACTATACTTCGGCTTGAAACCCATCGT
GGTCTTACATGGCTACGAGGCAGTGAAAGAGGCCCTGATTGATTTGGGGGAAGAGT
TCAGTGGGAGAGGAATCTTTCCCCTTGCTGAGAGGGCTAATCGTGGTTTTGGGATAG
TGTTTTCTAACGGAAAGAAGTGGAAAGAAATAAGGCGTTTCAGCCTGATGACTTTGC
GTAATTTTGGGATGGGAAAAAGGTCAATTGAAGATCGTGTTCAAGAAGAAGCCCGT
TGCCTGGTGGAGGAGTTGAGAAAGACGAAGGCTTCCCCGTGCGATCCAACTTTCATA
CTGGGATGTGCGCCATGCAATGTCATATGTAGTATAATCTTTCATAAGAGATTCGAC
TATAAGGATCAGCAATTCTTGAACTTGATGGAGAAATTGAACGAGAACATAAAAAT
TCTGTCTTCCCCCTGGATTCAAATATGTAATAACTTTAGCCCAATAATAGACTACTTC
CCAGGTACGCACAATAAACTGTTAAAGAACGTCGCTTTTATGAAATCTTACATATTG
GAGAAGGTGAAAGAGCACCAAGAGAGCATGGACATGAACAATCCGCAAGACTTCA
TTGATTGTTTCCTGATGAAGATGGAAAAAGAAAAGCACAACCAGCCTTCTGAATTTA
CGATTGAAAGCCTTGAAAATACTGCAGTCGATCTATTCGGAGCTGGCACAGAGACT
ACCTCAACCACGTTAAGATATGCTTTGCTTTTACTACTGAAGCATCCAGAGGTGACT
GCCAAGGTGCAAGAAGAGATCGAGAGGGTCATCGGAAGGAACCGTTCCCCGTGTAT
GCAGGACAGGAGCCATATGCCTTACACAGACGCGGTTGTCCACGAAGTCCAGCGTT
ACATAGATCTATTACCGACGTCACTACCCCACGCGGTCACTTGTGACATCAAATTTC
GTAACTACCTGATCCCCAAGGGCACTACCATATTAATTTCACTTACTTCCGTGCTACA
CGACAATAAGGAATTTCCAAATCCCGAGATGTTCGACCCGCATCACTTTCTGGACGA
AGGGGGAAATTTCAAGAAGTCAAAGTACTTCATGCCTTTCTCCGCCGGAAAGAGAA
TCTGTGTAGGAGAAGCTCTGGCGGGGATGGAACTATTCTTGTTTTTAACCTCAATAT
TACAGAATTTTAACCTTAAATCCCTTGTAGATCCTAAGAATCTGGACACAACGCCTG
TGGTTAACGGGTTCGCGTCCGTTCCGCCGTTTTACCAGTTATGCTTTATTCCCGTTTA
A
>p450_2
Seq. ID NO: 2
ATGGCCGCAGACTCTCTTGTTGTATTGGTATTATGCCTAAGCTGCTTGCTTCTATTAA
GCCTATGGAGACAAAGCAGTGGGAGAGGGAAACTTCCGCCCGGACCAACTCCTCTA
CCTGTAATCGGGAACATTTTACAAATCGGCATAAAAGATATCTCAAAAAGTTTAACA
AATTTGTCCAAGGTGTACGGCCCGGTATTTACTCTTTACTTCGGATTGAAGCCGATA
GTAGTTTTGCACGGCTATGAGGCCGTCAAGGAGGCACTTATAGACTTAGGAGAGGA
GTTTTCTGGGAGGGGCATTTTCCCGCTTGCAGAGCGTGCAAATAGGGGGTTTGGGAT
AGTGTTCTCAAATGGTAAGAAATGGAAAGAAATCAGGCGTTTTTCTCTGATGACCCT
TAGGAACTTCGGAATGGGAAAGAGATCTATCGAAGACAGGGTCCAGGAGGAAGCCC
GTTGCCTAGTAGAAGAACTTCGTAAGACGAAGGCTTCCCCATGTGACCCTACCTTTA
TTCTAGGCTGTGCGCCGTGCAATGTCATATGTTCTATTATTTTTCATAAGAGATTCGA
TTATAAGGATCAGCAGTTCCTGAATTTAATGGAGAAATTAAACGAGAATGTTAAAAT
ACTTAGTTCACCTTGGATACAGATATGTAATAACTTTTCACCTATAATCGATTATTTT
CCCGGAACTCATAACAAGCTCTTGAAGAATGTTGCTTTTATGAAGTCTTACATTTTA
GAGAAAGTTAAAGAGCATCAGGAATCCATGGACATGAATAACCCACAGGATTTCAT
TGACTGCTTCTTAATGAAAATGGAAAAGGAAAAGCATAACCAGCCAAGTGAGTTCA
CTATTGAATCTCTTGAAAACACGGCTGTGGATCTGTTCGGAGCAGGAACCGAGACTA
CGTCTACGACGCTGCGTTATGCGTTACTGCTATTACTGAAACATCCAGAAGTTACAG
CGAAGGTACAAGAGGAGATCGAGAGGGTCATCGGAAGAAATAGGAGTCCCTGTATG
CAAGATCGTTCTCATATGCCCTACACAGATGCAGTCGTTCATGAAGTGCAGAGATAT
ATCGACTTGTTACCCACCTCCCTACCTCACGCAGTAACCTGCGATATCAAATTTAGG
AATTATTTAATACCTAAAGGGACGACCATTCTGATAAGCCTAACATCAGTCTTGCAC
GATAACAAGGAATTTCCGAACCCCGAGATGTTTGACCCACACCATTTCCTGGACGAG
GGCGGGAACTTCAAGAAATCCAATTATTTTATGCCTTTCAGTGCTGGTAAGAGGATA
TGCGTAGGAGAGGCTTTAGCCAGGATGGAGCTTTTCCTATTCCTGACATCTATACTT
CAAAACTTCAATCTAAAGAGTTTAGTCGATCCGAAAAATTTAGATACGACGCCTGTT
GTAAATGGGTTCGCCTCCGTACCTCCCTTCTACCAATTGTGCTTTATTCCCGTGTAA
>p450_3
Seq. ID NO: 3
ATGGCCGCAGATTCCTTTGTGGTGCTGGTGCTGTGTTTAAGCTGCTTATTGTTACTAT
CCTTATGGCGTCAATCATCCGGACGTGGCAAATTGCCCCCTGGCCCAACACCCCTGC
CCGTTATAGGAAATATACTTCAGATTGACATAAAAGATATCAGTAAATCCCTAACGA
ATCTTTCTAAAGTTTATGGGCCCGTCTTTACCCTTTATTTCGGTCTGAAACCGATTGT
CGTTTTACACGGATACGAGGCAGTGAAAGAGGCTCTGATCGACTTAGGTGAGGAGT
TCTCTGGCCGTGGACATTTTCCATTGGCAGAACGTGCTAATAGGGGGTTCGGTATTG
TATTCTCCAACGGGAAAAAGTGGAAGGAAATCAGGCGTTTTTCCTTAATGACGCTAA
GAAACTTCGGCATGGGTAAGAGGAGTATAGAAGACCGTGTTCAAGAGGAAGCTAGA
TGCTTAGTAGAGGAGCTGAGGAAGACTAAGGCCTCTCCCTGTGATCCAACATTCATT
CTGGGCTGTGCTCCGTGCAATGTCATCTGTAGTATAATTTTTCGTAAGAGATTTGACT
ATAAGGATCAACAGTTCCTGAATCTTATGGAGAAACTTAATGAAAATGTCAAGATA
CTGTCTTCTCCCTGGATACAAATTTACAACAACTTTTCTCCCATCATAGATTACTTCC
CTGGAACGCATAACAAGCTGTTGAAAAATGTGGCTTTTATGAAGTCCTATATTCTGG
AAAAGGTCAAGGAACATCAGGAAAGTATGGACATGAACAATCCGCAAGATTTCATC
GATTGCTTCTTAATGAAGATGGAGAAAGAAAAACATAATCAACCTAGTGAGTTTAC
GATAGAGAGTCTTGAAAACACTGCCGCGGACCTATTCGGCGCCGGCACGGAAACCA
CATCTACCACCCTTAGGTATGCATTACTTCTACTACTAAAACATCCTGAAGTTACCGC
TAAGGTACAAGAAGAGATCGAGAGAGTAATAGGCAGGAATAGAAGTCCGTGTATGC
AAGATAGGAGCCACATGCCATACACAGACGCAGTCGTCCATGAAGTTCAGCGTTAT
ATTGACCTTCTTCCGACCAGTCTGCCACATGCAGTCACCTGTGACATTAAATTCAGG
AATTATTTAATTCCCAAAGGTACAACAATATTAATCTCTCTGACGAGCGTTCTACAT
GACAATAAGGAGTTCCCTAACCCAGAGATGTTCGATCCGCACCATTTCCTAGACGAA
GGTGGAAACTTTAAGAAGAGCAATTATTTTATGCCATTCTCCGCTGGGAAAAGAATC
TGTGTTGGCGAAGCATTGGCCAGAATGGAATTGTTTTTGTTCCTAACAAGCATCTTA
CAAAATTTCAATCTTAAATCTTTGGTTGACCCGAAGAATCTGGACACCACACCTGTC
GTAAATGGGTTTGCAAGCGTACCACCTTTTTATCAATTGTGTTTCATCCCCGTCTAA
>p450_4
Seq. ID NO: 4
ATGGCCGCAGATCTGGTAGTGTTCTTGGCCTTGACCCTAAGCTGTTTAATTCTACTAT
CATTATGGCGTCAGTCCTCCGGACGTGGTAAACTACCGCCAGGACCAACTCCGCTGC
CCATTATCGGGAACTTTCTTCAAATCGACGTCAAAAACATATCACAATCATTTACAA
ACTTCTCAAAAGCATACGGGCCAGTTTTTACTCTGTACCTAGGAAGCAAACCCACAG
TTATTTTGCATGGCTACGAAGCTGTCAAGGAGGCGTTGATAGACAGAGGAGAAGAA
TTTGCTGGGAGGGGAAGTTTCCCGATGGCCGAAAAGATCATCAAGGGATTTGGCGT
CGTGTTTTCTAACGGCAATAGGTGGAAAGAAATGAGGAGATTCACATTGATGACTCT
GAGGAACCTGGGTATGGGAAAGAGAAACATTGAAGATAGGGTCCAGGAGGAGGCA
CAATGTTTGGTTGAAGAACTAAGAAAAACAAAAGGAAGTCCCTGTGATCCAACGTT
CATTCTATCCTGCGCTCCCTGCAATGTTATCTGTTCTATTATATTCCAAAACCGTTTC
GATTATAAAGATAAAGAATTTCTAATACTAATGGATAAAATTAACGAGAACGTGAA
GATCCTATCCTCACCCTGGTTGCAAGTTTGCAATTCATTTCCTTCCTTAATAGACTAT
TGTCCAGGTTCTCATCACAAAATAGTGAAAAATTTCAACTATTTAAAGTCTTATTTGC
TGGAGAAAATTAAAGAGCATAAAGAGAGCCTTGACGTTACTAACCCCAGGGACTTT
ATTGACTACTATTTAATTAAGCAGAAACAGGTTAACCATATTGAACAGTCAGAATTT
TCTTTAGAGAATTTAGCCTCTACAATTAACGACCTGTTCGGGGCCGGGACAGAAACC
ACGAGCACAACGCTGAGATACGCATTACTACTGCTACTTAAATATCCGGATGTTACT
GCTAAGGTTCAGGAAGAAATCGATAGGGTAGTAGGACGTCATCGTTCACCATGCAT
GCAAGATCGTTCACACATGCCTTATACTGATGCAATGATACACGAAGTTCAGCGTTT
TATTGACTTGTTACCAACCAGTTTACCGCATGCGGTCACATGTGACATCAAATTTAG
GAAATATCTGATCCCCAAGGGTACAACTGTCATCACTAGCCTAAGCTCCGTATTGCA
TGACAGTAAAGAGTTCCCAAATCCAGAGATGTTCGACCCAGGGCACTTTTTGAATGC
GAATGGCAATTTTAAGAAGAGCGACTATTTCATGCCCTTTAGCACTGGCAAGAGAAT
ATGTGCCGGAGAGGGACTAGCAAGGATGGAATTATTCCTGATTCTTACCACAATACT
ACAGAACTTCAAATTAAAATCATTAGTCCACCCAAAAGAGATAGATATTACTCCAGT
GATGAACGGTTTTGCATCCCTTCCGCCACCCTACCAACTATGTTTTATTCCGCTTTAA
>p450_5
Seq. ID NO: 5
AATGGCCGCAATTTTAGGCGTATTCCTTGGTTTGTTTTTGACGTGTTTACTATTGTTA
AGTTTGTGGAAGCAGAATTTCCAAAGGAGAAATTTACCCCCAGGACCGACACCACT
TCCCATTATCGGTAACATACTTCAAATCGACTTAAAGGACATTTCCAAGAGTTTGAG
AAACTTCTCAAAAGTCTACGGCCCGGTATTTACCCTGTACTTGGGGAGGAAACCCGC
GGTCGTTCTGCATGGTTACGAGGCTGTTAAAGAGGCACTTATCGATCACGGGGAAG
AGTTCGCAGGTAGGGGTGTGTTTCCCGTCGCCCAAAAGTTTAACAAGAACTGCGGG
GTGGTTTTCTCATCCGGCCGTACCTGGAAGGAAATGAGGAGATTCTCCTTGATGACA
CTTAGGAATTTTGGGATGGGCAAGAGAAGTATAGAGGATAGGGTACAGGAAGAGGC
ACGTTGTCTAGTAGACGAACTTCGTAAAACTAACGGGGTGCCTTGTGATCCAACCTT
TATCCTGGGGTGCGCCCCGTGTAACGTGATTTGCTCTATCGTATTCCAAAACAGATT
CGATTACAAAGACCAGGAGTTTCTTGCGCTAATAGATATACTAAATGAAAACGTTGA
GATCCTTGGATCACCGTGGATTCAAATTTGTAATAACTTCCCAGCTATTATTGACTAT
TTACCGGGAAGACACAGGAAACTGTTAAAGAACTTTGCTTTTGCGAAACATTACTTC
TTAGCTAAAGTAATTCAACACCAGGAATCATTAGATATCAATAATCCCCGTGATTTC
ATCGACTGCTTCCTTATAAAAATGGAGCAGGAGAAGCATAATCCCAAAACTGAGTTT
ACTTGCGAGAACTTAATCTTCACTGCTTCTGACCTTTTCGCGGCCGGTACGGAGACA
ACCTCTACTACACTTCGTTATTCCTTATTATTGTTGTTAAAGTACCCTGAGGTTACGG
CAAAGGTGCAAGAAGAGATTGACCACGTGATAGGTCGTCACAGGTCTCCATGTATG
CAAGACCGTCATCACATGCCGTACACAGACGCTGTACTGCACGAGATACAGCGTTA
CATCGACCTATTACCCACGAGCTTACCTCACGCGCTTACCTGTGATATGAAGTTTAG
GGATTATTTAATCCCGAAGGGAACTACCGTTATCGCTTCTTTAACTTCAGTGCTTTAC
GATGATAAGGAGTTCCCTAACCCAGAGAAATTTGATCCAAGCCACTTCCTTGACGAG
AACGGAAAATTCAAAAAGTCCGATTACTTCTTCCCGTTCTCTACTGGAAAAAGGATC
TGCGTAGGAGAGGGGCTTGCTCGTACCGAATTGTTTCTATTCTTAACTACAATTCTGC
AAAATTTTAACCTGAAGAGCCCTGTAGATCTGAAGGAGTTAGACACGAATCCTGTG
GCAAACGGTTTTGTGTCAGTACCACCAAAATTTCAGATCTGTTTTATTCCTATATAA
>p450_6
Seq. ID NO: 6
ATGGCCGCAGCATTGATACCAGACTTAGCGATGGAAACCTGGTTGTTGCTTGCGGTG
TCTTTAGTCCTACTGTATCTATACGGTACTCATAGCCATGGTCTGTTCAAAAAGTTAG
GTATCCCCGGTCCAACGCCGCTACCCTTCCTTGGTAATATTCTGTCTTATCATAAGGG
TTTTTGCATGTTCGATATGGAGTGTCATAAGAAGTACGGTAAGGTATGGGGATTTTA
TGACGGTCAGCAGCCAGTCTTGGCAATAACAGACCCGGACATGATCAAGACAGTCC
TTGTAAAAGAGTGTTATAGCGTGTTTACGAACAGGAGACCGTTCGGGCCAGTGGGCT
TCATGAAGTCCGCAATTTCTATTGCGGAAGATGAGGAGTGGAAAAGGCTTCGTAGTC
TTTTGAGCCCTACATTTACGTCTGGAAAATTGAAGGAAATGGTCCCTATCATTGCTC
AATACGGAGATGTTCTAGTGAGGAATTTAAGGAGAGAGGCTGAGACTGGAAAGCCG
GTTACACTAAAAGACGTTTTCGGCGCGTACTCTATGGATGTCATCACCTCTACATCTT
TCGGGGTAAACATCGACAGTCTGAATAACCCGCAAGACCCCTTTGTTGAGAACACA
AAGAAATTACTGAGATTCGACTTTTTGGACCCGTTCTTTCTGTCCATTACTGTATTCC
CCTTTTTGATTCCGATTCTGGAAGTTTTAAATATTTGTGTTTTCCCGCGTGAGGTTAC
AAATTTCCTAAGGAAAAGTGTTAAAAGGATGAAGGAGTCCAGACTGGAAGATACTC
AAAAGCATAGGGTAGATTTCCTACAATTAATGATTGACTCACAGAATAGTAAGGAG
ACCGAGAGCCACAAGGCCCTTAGTGATCTTGAATTAGTCGCACAGTCAATTATTTTC
ATATTTGCGGGCTACGAGACAACCAGCTCAGTTCTATCATTTATAATGTATGAACTG
GCCACCCACCCTGATGTGCAACAAAAACTTCAGGAAGAGATCGATGCAGTCCTTCC
AAATAAAGCTCCACCCACCTATGATACCGTTTTGCAAATGGAGTATCTTGACATGGT
TGTAAACGAAACCCTGCGTTTGTTTCCTATAGCAATGAGATTGGAACGTGTATGTAA
GAAAGACGTGGAGATAAATGGAATGTTTATTCCTAAAGGTGTGGTCGTTATGATTCC
CTCATATGCCTTACATCGTGATCCAAAATATTGGACGGAGCCTGAAAAATTTCTGCC
AGAGAGGTTTTCCAAGAAAAACAAAGATAATATAGATCCCTACATCTATACACCCTT
TGGCAGCGGTCCGAGGAATTGCATTGGCATGCGTTTTGCTTTAATGAATATGAAGCT
GGCCTTAATTAGGGTTTTGCAAAATTTCTCTTTCAAACCGTGCAAGGAAACTCAGAT
ACCATTAAAACTTTCATTAGGAGGCCTACTTCAACCTGAGAAACCTGTGGTTTTAAA
AGTTGAGAGTAGAGACGGTACGGTGAGTGGCGCTTAA
>p450_7
Seq. ID NO: 7
ATGGCCGCAGATCTAATACCTAATCTAGCCGTAGAGACCTGGCTTCTGTTAACCAAA
TTGGAGTTTGGGTTCTACATATTTCCGTTTATCTACGGTACTCATAGCCATGGTCTTT
TCAAGAAACTGGGCATTCCAGGCCCGACGCCATTGCCGTTCCTGGGTAATATCCTAT
CATACAGAAAAGGCTTCTGCATGTTTGACATGGAATGCCACAAGAAGTATGGGAAG
GTATGGGGCTTTTACGATGGCAGACAACCAGTTCTGGCAATTACAGACCCGGACATG
ATAAAAACGGTTCTAGTAAAGGAATGTTATTCTGTATTCACTAATAGGCGTCCTTTC
GGCCCAGTGGGGTTCATGAAATCTGCGATATCTATCGCGGAAGATGAAGAGTGGAA
GAGAATAAGATCTTTACTTAGCCCTACATTCACTAGTGGCAAATTGAAGGAGATGGT
TCCTATTATTGCCCAGTACGGAGACGTCTTAGTACGTAATCTTAGAAGAGAAGCCGA
TACCGGTAAGCCCGTTACACTGAAGGACGTCTTCGGAGCATACAGTATGGACGTGAT
CACATCTACTTCTTTCGGTGTAAACATAGACTCCTTGAACAATCCCCAAGATCCCTTC
GTTGAAAACACTAAGAAACTACTGAGATTTGACTTTTTGGACCCTTTCTTTCTATCTA
TTATAGTCTTTCCTTTCTTGATTCCAATTCTGGAGGTACTGAATATCTGCGTATTTCCT
CGTGAAGTCACAAACTTCCTAAGAAAGTCAGTCAAGAGGATGAAGGAAAGCCGGCT
AGAAGACACTCAAAAGCATAGGGTTGACTTTCTTCAGTTAATGATTGATTCTCAAAA
CTCCAAAGAAACTGAGAGTCACAAAGCTCTATCAGATCTGGAGTTAGTGGCGCAGT
CCATAATTTTTATCTTTGCCGGTTACGAGACCACAAGTTCCGTGCTGTCATTTATCAT
GTATGAGCTGGCTACCCACCCAGATGTGCAGCAAAAACTACAGGAGGAGATCGATG
CAGTTTTACCCAATAAGGCACCGCCCACGTATGACACAGTTCTGCAAATGGAGTACC
TGGACATGGTGGTCAATGAGACGCTTCGTTTGTTCCCAGTTGCTATGAGGTTGGAGA
GGGTGTGCAAGAAGGATGTTGAGATAAACGGTATGTTTATCCCAAAGGGCGTTGTC
GTGATGATACCAAGCTACGCACTTCACCGTGATCCTAAATATTGGACTGAGCCTGAG
AAATTTTTACCTGAACGTTTTAGTAAGAAAAATAAAGATAACATTGATCCCTATATC
TACACGCCTTTCGGAAGCGGACCCCGTAATTGTATAGGAATGAGGTTCGCTCTTATG
AATATGAAATTAGCCCTAATACGTGTGCTACAAAACTTCAGCTTCAAGCCATGCAAG
GAGACACAGATTCCCCTAAAGCTGCGTCTTGGGGGTTTGCTACAGCCGGAAAAACCT
ATCGTTCTAAAAGTCGAAAGTAGGGATGGAACAGTGTCCGGGGCATAA
>p450_8
Seq. ID NO: 8
ATGGCCGCAGCACTTATACCCGATTTAGCGATGGAGACGTGGTTACTACTAGCGGTG
TCACTGGTGCTGCTGTACCTATATGGGACCCATAGTCATGGACTGTTCAAAAAGTTG
GGCATTCCCGGACCGACGCCGCTACCCTTTCTTGGTAATATTTGGTCTTATCGTAAAG
GATTCTGTATGTTCGACATGGAATGCCATAAGAAGTATGGGAAAGTTTGGGGGTTCT
ATGATGGGAGACAGCCAGTTCTAGCTATCACTGATCCCGATATGATTAAAACAGTTC
TTGTAAAAGAGTGTTATAGTGTCTTCACAAACCGTAGGCCTTTCGGCCCAGTCGGCT
TTATGAAGTCTGCCATATCCATTGCTGAGGATGAGGAATGGAAGAGACTGAGATCC
CTTTTGTCTCCGACCTTTACTAGCGGCAAGTTGAAGGAGATGGTACCATTGATCGCA
CAATATGGCGACGTACTTGTCCGTAACCTGCGTTTAGAGGCCGAAACGGGCAAACC
GGTTACGATGAAGGTTATTACTTCTACAAGTTTCGGGGTCAATATAGACTCACTGAA
TAACCCACAAGATCCTTTCGTAGAGAATACTAAAAAGTTGCTGAGATTCGATTTCCT
AGACCCCTTTTTCCTGTCTATTATTGTCTTTCCTTTCTTGACGCCTATACTTGAAGTAT
TGAACATTAGTGTGTTCCCGAGGGCCGTTACTTCATTCTTGCGTAAAAGTGTTAAGA
GAATGAAAGAGTCTAGGCTTGAAGATACTCAGAAACATCGTGTGGACTTCTTACAG
CTAATGATTGACTCCCAAAATAGTAAGGAGACTGAGAGTCATAAAGCGTTAAGCGA
CTTGGAATTGGTAGCACAAAGCATAATCTTCATCTTTGCTGGGTACGAGACGACTTC
CAGCGTGCTGAGTTTTATAACATACGAATTGGCAACGCACCCGGACGTTCAGCAAA
AACTTCAAGAGGAAATAGATGCCGTCTTGCCGAACAAGGCACCCCCGACTTATGAT
ACAGTGTTGCAAATGGAGTACCTAGACATGGTAGTCAACGAGACACTTAGGTTATTT
CCTATAGCCATGAGGTTAGAGAGAGTCTGCAAAAAGGACGTAGAGATTAATGGTAT
GTTCATCCCGAAAGGAGTTGTAGTAATGATCCCTTCCTACGCCCTGCACCACGACCC
TAAGTACTGGACCGAACCCGAAAAGTTCCTGCCCGAGCGTTTCTCTAAGAAAAATA
AAGATAATATCGATCCCTATATTTATACACCATTCGGCTCTGGACCAAGGAACTGCA
TTGGCATGCGTTTTGCCCTGATGAATATGAAGCTGGCGCTAATAAGGGTACTGCAGA
ATTTTTCCTTTAAACCGTGCAAGGAAACCCAAATACCTCTAAAGTTACGTCTGGGAG
GTCTGCTACAACCGGAAAAACCCATTGTCTTGAAAGTGGAATCCAGAGATGGCACC
GTTTCTGGGGCGTAA
>p450_9
Seq. ID NO: 9
ATGGCCGCAGAGTTAATTCCGTCCTTTTCTATGGAAACTTGGGTACTTCTAGCGACC
AGTTTGGTCTTGTTATACATATACGGTACATATTCTTATGGTCTATTTAAAAAGTTAG
GCATTCCGGGCCCGCGTCCCGTACCCTATTTTGGGTCTACTATGGCCTATCATAAGG
GGATTCCGGAGTTCGATAACCAGTGTTTTAAGAAGTATGGCAAAATGTGGGGGTTTT
ATGAAGGCCGTCAGCCTATGCTGGCAATCACAGACCCAGATATAATTAAAACGGTA
CTGGTAAAAGAGTGTTACTCTGTATTCACTAACAGACGTATCTTCGGGCCTATGGGA
ATAATGAAATACGCCATTTCTCTAGCATGGGACGAGCAATGGAAGCGTATCAGAAC
CTTATTATCCCCGGCGTTTACTAGCGGCAAGTTAAAAGAAATGTTCCCTATTATCGG
GCAGTACGGAGATATGTTGGTTAGGAACCTTCGTAAGGAAGCCGAGAAAGGTAACC
CCGTTAATATGAAAGATATGTTTGGAGCCTACTCAATGGATGTTATCACAGGGACGG
CTTTCGGGGTGAACATTGATAGTTTGAATAATCCCCACGACCCCTTCGTGGAGCATT
CCAAGAATCTTCTAAGGTTCAGGCCCTTCGACCCATTTATCTTGAGCATTATCTTATT
TCCGTTCCTAAACCCGGTGTTCGAAATATTAAACATTACTCTGTTTCCGAAGAGCAC
TGTCGATTTCTTTACTAAATCTGTCAAGAAGATCAAAGAATCCAGACTAACCGATAA
GCAGATGAATAGGGTGGATCTGTTACAGTTAATGATTAACTCTCAGAACTCAAAAG
AAATAGATAACCACAAAGCCCTTAGCGACATCGAGCTAGTGGCCCAATCTACCATC
TTTATCTTTGGAGGTTATGAAACCACAAGCTCAACATTGAGCTTTATTATCTACGAA
CTGACAACGCATCCTCATGTACAACAGAAGGTACAGGAAGAAATTGACGCAACATT
TCCAAACAAGGCACCACCCACCTATGATGCGTTGGTACAGATGGAGTACCTAGATAT
GGTAGTGAACGAAACTTTGCGTATGTTTCCTATAGCTGGGCGTCTGGAAAGGGTCTG
CAAGAAGGACGTCGAAATTCACGGGGTGACGATTCCTAAGGGAACGACCGTTCTAG
TACCTTTATTTGTCCTACACAACAACCCAGAGCTTTGGCCTGAACCCGAGGAGTTCA
GGCCTGAAAGGTTTTCTAAAAACAATAAGGACAGCATCAACCCGTATGTGTACCTAC
CATTTGGCACAGGTCCTCGTAATTGCCTGGGTATGCGTTTTGCGATAATGAATATCA
AATTAGCTCTAGTCCGTATTTTACAGAATTTCTCATTTAAACCATGCAAGGAGACGC
AGATTCCTCTGAAGTTGTATACTCAGGGGTTGACTCAACCCGAACAACCAGTGATCT
TGAAGGTGGTTCCGCGTGGTCTTGGCCCGCAGGTTGAACCCGACTTCCTTTAA
>p450_10
Seq. ID NO: 10
ATGGCCGCAGATTCTTTTCCACTGCTGGCGGCATTGTTCTTCATCTTAGCTGCTACAT
GGTTTATTAGCTTCCGTAGACCGAGGAACCTACCCCCAGGTCCATTCCCTTACCCAA
TAGTAGGAAACATGTTGCAACTTGGCACACAACCACACGAAACGTTCGCAAAACTT
TCCAAGAAGTATGGGCCACTAATGTCAATCCACTTGGGCTCCTTGTACACCGTAATA
GTCAGCAGCCCAGAGATGGCTAAGGAGATTATGCATAAGTACGGCCAAGTCTTCTC
AGGCCGTACAGTGGCGCAGGCGGTCCACGCGTGCGGGCATGATAAGATCAGCATGG
GCTTTCTGCCGGTAGGGGGTGAGTGGCGTGATATGAGAAAGATTTGCAAAGAGCAA
ATGTTCTCACACCAATCAATGGAGGATTCACAATGGCTGCGTAAGCAGAAATTACA
GCAACTACTAGAATATGCTCAGAAGTGCTCAGAGAGGGGTAGAGCCATCGACATTA
GGGAGGCAGCGTTTATCACCACTTTGAACTTGATGTCCGCCACTTTGTTCTCCATGCA
GGCGACCGAATTCGATTCCAAGGTAACTATGGAATTTAAGGAGATTATAGAAGGAG
TCGCCTCCATTGTGGGTGTACCAAACTTCGCAGATTATTTTCCTATTTTACGTCCCTT
CGACCCCCAAGGGGTTAAAAGGCGTGCCGACGTATACTTCGGAAGACTTTTAGCCAT
CATTGAGGGGTTCCTTAATGAAAGGGTGGAGAGTAGGAGGACGAACCCCAACGCAC
CTAAAAAGGACGACTTCCTGGAAACGCTAGTTGATACCCTTCAGACTAATGACAATA
AGCTAAAGACGGATCACTTGACTCATTTAATGCTGGACTTATTTGTGGGAGGTTCAG
AAACTAGCACAACCGAGATAGAGTGGATTATGTGGGAGCTTCTAGCGAACCCGGAA
AAGATGGCAAAAATGAAAGCTGAGTTGAAGTCAGTGATGGGTGAAGAGAAGGTTGT
TGATGAAAGTCAGATGCCACGTTTGCCATATTTACAGGCAGTTGTTAAAGAAAGCAT
GAGGTTACATCCACCAGGTCCATTGCTATTACCTAGAAAGGCCGAGTCCGACCAGGT
CGTAAATGGCTATCTGATTCCGAAAGGGGCGCAGGTACTGATCAATGCCTGGGCGA
TTGGAAGGGACCACTCAATCTGGAAAAACCCGGACTCCTTTGAACCGGAAAGATTC
TTAGATCAGAAAATTGATTTTAAGGGCACCGATTACGAACTTATCCCCTTCGGGAGT
GGCAGGAGAGTCTGTCCAGGAATGCCTCTTGCTAATAGGATCCTTCACACAGTCACT
GCCACGCTAGTACATAATTTCGATTGGAAGCTTGAGCGTCCGGAGGCCTCAGACGCT
CATAGGGGCGTGCTGTTCGGCTTTGCTGTAAGGAGAGCAGTCCCTCTAAAGATTGTG
CCTTTTAAGGTG
>p450_11
Seq. ID NO: 11
ATGGCAGCCGATCCCTTCCCTCTGGTAGCAGCGGCATTATTCATAGCTGCAACATGG
TTCATTACCTTCAAAAGGAGACGTAATCTTCCGCCGGGGCCTTTCCCTTACCCGATTG
TGGGCAATATGTTGCAACTAGGTTCCCAACCACACGAGACATTTGCCAAGCTATCCA
AAAAGTACGGGCCATTAATGTCAATTCACCTTGGAAGTTTATATACCGTAATAATAT
CCTCCCCCGAAATGGCCAAAGAGATAATGCACAAGTACGGGCAAGTCTTTTCTGGG
AGAACAATAGCTCAGGCTGTGCACGCATGCGATCACGATAAAATATCTATGGGCTTT
TTACCTGTGGGAGCAGAGTGGCGTGACATGAGGAAGATCTGCAAGGAACAGATGTT
CTCTCATCAAAGCATGGAAGATAGTCAGAACTTACGTAAACAGAAACTTCAGCAAT
TGCTGGAATATGCTCAAAAATGCAGTGAAGAAGGAAGAGGAATCGATATACGTGAG
GCAGCTTTTATTACTACATTAAACCTGATGTCTGCCACGTTATTCAGCATGCAAGCC
ACTGAATTCGATAGTAAAGTCACTATGGAGTTCAAGGAAATAATCGAAGGAGTGGC
GAGCATCGTGGGCGTCCCAAATTTTGCAGATTATTTCCCCATTCTGCGTCCTTTCGAC
CCTCAAGGGGTTAAGCGTCGTGCGGATGTCTACTTTGGAAGATTATTAGGCTTGATC
GAAGGTTATCTTAACGAAAGAATTGAATTCAGAAAAGCCAACCCCAATGCCCCAAA
GAAAGACGATTTTTTAGAAACCCTGGTGGACGCACTTGATGCGAAGGATTACAAAC
TAAAGACTGAACACCTTACTCACCTGATGCTAGACCTATTCGTTGGGGGGAGCGAGA
CGAGCACCACTGAAATTGAGTGGATCATGTGGGAGTTACTGGCATCACCTGAGAAG
ATGGCCAAAGTCAAAGCAGAATTGAAAAGTGTAATGGGGGGCGAAAAGGTCGTGG
ACGAGTCTATGATGCCTAGATTACCTTATCTGCAAGCAGTGGTTAAAGAGTCAATGA
GGTTACACCCGCCAGGCCCATTATTACTTCCAAGAAAAGCGGAAAGTGACCAGGTC
GTAAACGGTTATTTGATTCCTAAGGGAGCGCAAGTACTGATCAATGCGTGGGCGATG
GGTAGAGACCCAAGCCTATGGAAAAACCCTGACTCTTTTGAGCCAGAGCGTTTTTTA
GACCAGAAGATCGACTTTAAGGGTACAGATTACGAACTTATCCCGTTTGGAAGTGGC
AGAAGGGTGTGCCCTGGAATGCCCCTGGCGAACAGAATTCTTCATACGGTTACTGCT
ACTCTTGTGCATAACTTTGATTGGAAATTGGAAAGACCGGAGGCAAGCGACGCGCA
CAAGGGAGTCCTTTTTGGTTTCGCGGTCAGGAGAGCTGTACCTTTGAAGATCGTCCC
TATCAAGGCA
>p450_12
Seq. ID NO: 12
ATGGCGGCAGACAGCTTCCCGTTACTGGCAGCACTTTTCTTTATCGCAGCAACTATA
ACTTTCCTGTCTTTCAGGCGTAGAAGAAACTTGCCGCCCGGACCATTTCCCTACCCT
ATTGTAGGTAATATGCTACAACTGGGTGCAAATCCACACCAAGTCTTCGCCAAACTT
TCAAAAAGATATGGGCCTCTGATGAGTATACATCTGGGAAGCTTGTATACGGTTATA
GTGAGTTCCCCTGAGATGGCGAAGGAAATATTACATAGGCATGGGCAAGTGTTCTCT
GGTCGTACTATTGCCCAAGCTGTCCACGCTTGCGATCATGACAAAATATCTATGGGT
TTCCTTCCAGTAGCCAGCGAATGGAGGGACATGAGGAAAATTTGCAAGGAGCAGAT
GTTCAGCAATCAAAGCATGGAGGCTAGCCAGGGACTTCGTAGGCAGAAACTACAAC
AACTTTTGGATCATGTACAGAAATGCTCCGATAGTGGGAGGGCCGTCGACATTAGA
GAAGCTGCTTTCATAACCACCTTGAATCTTATGTCCGCCACACTGTTCAGCTCCCAG
GCCACCGAGTTCGATTCTAAGGCTACCATGGAATTTAAGGAGATTATTGAAGGTGTA
GCCACCATCGTAGGCGTACCTAACTTCGCAGATTACTTCCCAATTCTTAGGCCCTTTG
ATCCCCAGGGAGTGAAACGTAGGGCCGACGTATTTTTCGGAAAACTGTTAGCCAAA
ATTGAAGGCTATTTAAACGAGAGATTAGAATCCAAGAGGGCAAACCCGAATGCGCC
AAAAAAGGACGATTTCTTGGAGATTGTCGTCGATATTATCCAGGCAAACGAATTTAA
GTTAAAGACTCACCACTTTACTCACTTGATGTTGGACTTATTTGTTGGCGGCTCAGAC
ACGAATACAACGTCCATCGAGTGGGCGATGTCTGAGTTAGTAATGAACCCCGACAA
GATGGCGCGTTTGAAGGCTGAACTGAAATCTGTGGCAGGGGATGAAAAAATAGTTG
ACGAGTCAGCCATGCCAAAGTTGCCTTACTTGCAGGCCGTCATAAAGGAAGTTATGC
GTATACACCCTCCGGGGCCGTTGCTTTTGCCTCGTAAAGCTGAAAGCGATCAAGAAG
TGAATGGATACCTTATTCCGAAAGGCACACAGATCCTGATAAATGCATATGCGATAG
GACGTGATCCATCAATCTGGACTGACCCCGAAACATTTGACCCGGAACGTTTCCTGG
ATAACAAGATTGATTTCAAGGGACAAGATTACGAGCTGTTGCCGTTCGGCTCCGGAC
GTCGTGTATGTCCGGGGATGCCGTTAGCTACTAGAATACTTCACATGGCAACCGCCA
CGCTAGTCCACAACTTCGACTGGAAATTAGAAGACGATAGTACAGCCGCTGCAGAC
CACGCAGGCGAGCTATTTGGAGTTGCAGTGAGAAGGGCAGTTCCGCTTCGTATAATC
CCGATAGTGAAGTCC
>CPR_1
Seq. ID NO: 13
ATGGCCGCAGGAGATTCTCATGTGGATACGTCGTCCACTGTGTCTGAAGCTGTTGCT
GAAGAGGTATCTTTATTCAGTATGACTGACATGATCCTATTCAGCCTTATTGTGGGG
CTACTGACCTACTGGTTCCTATTCAGAAAGAAGAAGGAAGAGGTCCCAGAGTTCAC
CAAGATCCAAACGCTGACTTCCAGCGTCCGTGAATCATCCTTTGTTGAGAAAATGAA
GAAAACAGGGCGTAATATCATTGTGTTTTATGGTAGTCAGACTGGCACTGCAGAAG
AGTTCGCCAACAGGTTGTCTAAAGATGCGCACAGATATGGTATGAGGGGTATGTCTG
CGGACCCGGAGGAATATGACTTAGCGGACTTGTCCTCTCTGCCTGAAATTGATAACG
CGCTAGTGGTCTTCTGTATGGCTACGTACGGAGAAGGCGATCCAACCGATAATGCCC
AAGACTTCTACGATTGGTTGCAAGAGACTGACGTGGATTTGTCCGGAGTAAAGTTCG
CAGTATTTGGGCTGGGAAACAAAACTTATGAACATTTTAATGCAATGGGTAAGTACG
TAGACAAACGTTTAGAACAACTGGGTGCACAAAGAATATTCGAGCTTGGTTTGGGG
GATGATGACGGTAATTTAGAGGAGGATTTCATCACTTGGAGGGAGCAATTCTGGCC
GGCGGTGTGCGAGCATTTCGGCGTTGAGGCGACCGGAGAAGAGTCAAGCATTAGAC
AGTATGAACTTGTTGTGCACACAGATATCGACGCCGCAAAAGTGTATATGGGGGAG
ATGGGTAGATTAAAATCTTATGAGAATCAAAAACCTCCTTTCGATGCGAAAAATCCA
TTCCTTGCCGCTGTGACCACAAACAGGAAACTAAATCAAGGTACAGAGCGTCATTTG
ATGCACTTGGAGCTAGACATCAGTGATTCTAAAATTAGGTACGAATCAGGGGATCA
CGTCGCCGTTTATCCCGCCAATGATAGCGCCTTGGTGAATCAGTTAGGTAAGATACT
GGGTGCTGATTTGGATGTAGTCATGAGCTTGAATAACCTTGATGAAGAGTCCAATAA
GAAACATCCTTTCCCGTGCCCAACAAGTTATAGAACCGCCCTTACGTACTACTTAGA
TATCACCAATCCACCAAGAACGAATGTCCTATATGAGCTTGCTCAATATGCCAGTGA
GCCATCCGAGCAAGAGCTGCTTAGGAAAATGGCGTCCTCATCCGGTGAAGGCAAAG
AATTATACCTGTCCTGGGTGGTCGAGGCCAGGAGGCATATTTTAGCTATTTTGCAAG
ATTGTCCTTCCCTTAGGCCGCCCATCGATCATCTTTGTGAGCTGCTTCCTCGTTTACA
AGCAAGGTATTATTCTATCGCGTCCTCCTCTAAAGTCCATCCAAACAGCGTACACAT
CTGTGCCGTGGTGGTCGAGTACGAGACGAAGGCCGGTAGAATCAACAAGGGCGTTG
CTACAAACTGGTTGAGAGCCAAGGAGCCCGCGGGGGAAAACGGAGGTCGTGCATTA
GTACCGATGTTTGTCCGTAAATCTCAATTCAGGTTGCCTTTTAAGGCAACCACTCCG
GTAATCATGGTCGGGCCTGGCACTGGCGTAGCCCCATTTATAGGATTCATTCAGGAA
AGGGCCTGGTTGAGGCAACAAGGCAAGGAGGTTGGAGAGACTCTGCTGTACTACGG
ATGCCGTAGGAGCGACGAAGATTACTTGTATCGTGAAGAGCTTGCACAATTTCACCG
TGACGGAGCACTTACTCAATTAAATGTGGCTTTTAGTCGTGAACAGTCACATAAGGT
GTATGTACAACATTTATTGAAGCAAGACCGTGAACACCTTTGGAAGCTGATTGAAGG
TGGCGCCCATATTTATGTATGCGGCGATGCTCGTAATATGGCAAGGGACGTTCAAAA
CACTTTCTATGACATCGTCGCAGAACTTGGGGCGATGGAGCATGCTCAAGCAGTAGA
TTACATCAAGAAACTAATGACCAAAGGTAGATATTCACTTGACGTTTGGTCCTAA
>CPR_2
Seq. ID NO: 14
ATGGCCGCACCTTTCGGTATTGACAATACTGACTTCACTGTCTTGGCGGGCCTGGTA
CTGGCAGTACTGTTGTATGTGAAGAGGAACAGTATAAAAGAACTGTTGATGTCAGA
TGATGGTGACATCACAGCTGTTTCTAGTGGGAACAGAGACATTGCCCAGGTCGTCAC
GGAAAATAATAAAAACTATCTAGTCCTGTATGCTTCACAGACCGGCACCGCAGAAG
ATTATGCGAAGAAATTTAGCAAGGAGCTGGTAGCCAAGTTCAACCTTAATGTGATGT
GCGCTGACGTAGAGAATTACGACTTCGAATCCTTAAACGATGTACCGGTTATCGTTT
CTATCTTCATTTCCACCTACGGAGAGGGCGATTTTCCAGACGGTGCGGTTAACTTCG
AAGACTTTATATGTAATGCTGAGGCTGGAGCTTTAAGTAACTTACGTTATAATATGT
TTGGTCTTGGGAACTCTACTTATGAGTTCTTTAATGGCGCTGCCAAGAAAGCCGAAA
AACACTTATCTGCGGCGGGGGCCATCAGACTGGGCAAACTTGGAGAGGCCGACGAT
GGTGCCGGGACAACGGACGAGGATTATATGGCTTGGAAGGATTCTATATTGGAGGT
TCTAAAGGATGAACTACACTTAGATGAGCAGGAAGCCAAATTTACTTCCCAGTTTCA
GTATACTGTTCTGAACGAAATAACAGACTCCATGTCTTTGGGCGAACCGTCTGCGCA
TTACCTACCCAGCCATCAACTGAACAGAAACGCGGACGGAATACAGCTAGGGCCCT
TTGACTTATCGCAGCCTTACATTGCCCCAATTGTAAAATCTAGGGAGCTGTTTAGTTC
TAATGATAGGAATTGCATACATAGCGAGTTCGACTTGTCCGGTTCTAATATTAAGTA
CTCTACAGGTGACCACCTGGCCGTATGGCCGAGCAATCCCCTTGAGAAGGTAGAAC
AATTTTTGTCAATCTTCAACCTAGATCCAGAAACGATATTCGATTTGAAGCCCCTGG
ACCCGACTGTTAAGGTACCGTTTCCCACTCCTACCACCATAGGTGCGGCAATCAAGC
ACTATTTGGAAATCACAGGCCCGGTATCACGTCAATTGTTTAGTAGCTTAATTCAAT
TCGCCCCGAATGCTGACGTAAAGGAGAAACTAACCCTGCTAAGTAAGGACAAGGAC
CAATTCGCTGTGGAAATTACCAGTAAATATTTCAACATAGCGGATGCTTTAAAGTAT
CTGAGTGATGGGGCTAAATGGGACACTGTGCCCATGCAATTTCTGGTGGAGTCCGTG
CCCCAAATGACCCCCAGGTACTACAGTATCAGTTCATCCAGCCTAAGTGAGAAGCA
GACGGTCCATGTAACAAGCATAGTAGAGAATTTCCCAAATCCCGAATTACCGGATG
CGCCCCCTGTCGTGGGAGTGACAACCAATCTTCTAAGGAATATCCAACTAGCCCAAA
ACAATGTGAATATCGCGGAAACGAACCTACCCGTTCACTACGATCTTAATGGACCCA
GGAAACTTTTTGCAAATTACAAACTTCCCGTCCATGTTAGGAGATCAAATTTTAGGC
TACCTTCCAATCCAAGCACTCCAGTGATCATGATTGGACCGGGTACTGGAGTTGCGC
CTTTCCGTGGGTTCATTAGGGAAAGAGTAGCCTTTTTGGAGAGTCAGAAGAAAGGC
GGAAATAATGTCAGCTTGGGCAAACACATATTGTTTTACGGTTCACGTAACACCGAC
GACTTCCTTTACCAGGATGAATGGCCAGAGTACGCTAAGAAACTAGACGGGTCTTTT
GAGATGGTTGTGGCCCACTCTAGGCTTCCAAACACGAAGAAGGTCTATGTACAGGA
TAAGCTGAAAGACTATGAGGATCAAGTTTTTGAAATGATAAACAACGGGGCGTTCA
TTTATGTTTGCGGAGACGCAAAAGGGATGGCTAAGGGTGTGAGCACAGCCTTGGTC
GGTATCTTATCAAGAGGGAAGTCAATAACTACAGACGAAGCCACTGAGCTAATTAA
AATGCTTAAAACGAGCGGAAGGTACCAAGAGGACGTTTGGTAA
>CPR_3
Seq. ID NO: 15
ATGGCCGCAGGAGACAGCCACGAAGATACTAGTGCGACCGTTCCGGAGGCAGTGGC
GGAAGAGGTGAGCCTATTCAGTACTACCGATATTGTACTTTTCTCCCTAATTGTGGG
TGTGCTGACTTACTGGTTCATATTTAAAAAGAAGAAAGAGGAGATTCCCGAATTTTC
CAAAATCCAAACGACAGCTCCACCCGTTAAAGAAAGTAGTTTCGTCGAGAAAATGA
AGAAGACTGGGAGGAATATAATAGTTTTCTATGGAAGCCAAACAGGGACCGCAGAG
GAGTTCGCGAACAGACTAAGTAAAGACGCTCATAGATACGGTATGCGTGGTATGTC
CGCTGACCCAGAGGAGTACGACCTGGCAGACCTAAGCTCACTGCCAGAGATTGACA
AAAGCCTAGTGGTCTTCTGTATGGCTACATATGGTGAAGGTGATCCAACTGATAACG
CTCAGGATTTCTACGATTGGTTACAAGAGACAGATGTGGACCTGACTGGAGTTAAAT
TTGCAGTCTTCGGCTTGGGGAATAAAACATACGAACACTTTAATGCTATGGGGAAAT
ACGTCGATCAAAGATTGGAGCAACTTGGCGCCCAGAGAATTTTCGAGCTAGGCTTG
GGAGACGACGATGGGAATCTTGAGGAGGATTTTATAACTTGGAGAGAACAGTTTTG
GCCAGCCGTGTGCGAATTTTTCGGAGTCGAGGCGACAGGCGAAGAGTCAAGTATCA
GGCAATATGAGCTAGTTGTGCATGAAGATATGGACACGGCGAAAGTCTACACCGGC
GAGATGGGACGTCTAAAAAGTTACGAGAACCAAAAACCGCCTTTTGATGCGAAGAA
TCCATTCTTGGCCGCCGTCACGACAAACCGTAAGTTAAACCAAGGGACTGAAAGAC
ATCTGATGCACTTAGAGCTTGACATCTCCGATAGTAAAATAAGGTATGAAAGTGGA
GATCACGTCGCCGTATACCCGGCTAACGATTCAACTCTAGTTAATCAGATCGGGGAA
ATATTAGGGGCCGACCTAGACGTCATAATGAGTTTAAACAACCTAGATGAAGAATC
AAACAAGAAACACCCATTCCCCTGTCCAACCACTTACAGGACAGCGTTGACTTATTA
TCTTGATATCACCAATCCCCCGAGAACCAACGTGTTATATGAACTTGCTCAGTATGC
CAGTGAACCATCTGAGCAGGAACATCTGCACAAGATGGCATCCTCATCAGGAGAAG
GAAAAGAATTATATCTGTCCTGGGTCGTGGAGGCTAGGAGACATATCCTTGCGATCC
TGCAGGACTATCCTAGCTTGCGTCCGCCTATCGACCATTTATGCGAACTGCTACCTC
GTTTGCAGGCCAGGTACTACAGCATAGCCTCTAGTAGTAAAGTACATCCTAACTCTG
TGCATATATGTGCCGTGGCCGTGGAGTACGAGGCTAAATCAGGAAGAGTAAATAAA
GGAGTCGCAACGAGTTGGCTGAGGACTAAGGAACCAGCCGGCGAGAACGGTAGGA
GAGCACTGGTGCCCATGTTTGTGAGGAAGTCCCAATTTCGTCTGCCATTTAAGCCTA
CAACCCCGGTAATTATGGTCGGTCCCGGGACAGGTGTAGCTCCGTTCATGGGATTTA
TCCAAGAGCGTGCCTGGTTGAGGGAACAGGGTAAAGAAGTCGGAGAGACCTTATTA
TACTATGGATGTAGGCGTTCAGACGAGGATTATTTATACCGTGAGGAGTTAGCCCGT
TTTCACAAAGACGGGGCCTTAACCCAGCTTAATGTAGCTTTTTCTAGGGAGCAAGCG
CATAAGGTCTATGTTCAACACTTGCTTAAAAGGGATAAAGAACACTTGTGGAAGCTA
ATACACGAAGGAGGAGCCCATATCTATGTTTGCGGAGATGCCAGGAACATGGCCAA
GGACGTACAAAATACCTTTTATGATATTGTCGCAGAATTTGGTCCTATGGAGCACAC
ACAAGCTGTAGACTATGTTAAGAAACTAATGACAAAAGGCAGGTACAGTCTGGATG
TCTGGTCTTAA
>CPR_4
Seq. ID NO: 16
ATGGCCGCAGGTGATTCCCATGAGGACACTTCCGCTACTATGCCGGAGGCCGTAGC
GGAGGAGGTCTCATTGTTTTCCACGACTGACATGGTCCTGTTCAGCCTGATCGTCGG
CGTTTTGACGTATTGGTTCATATTTAGAAAGAAAAAGGAAGAAATCCCCGAGTTCTC
CAAAATCCAAACCACTGCCCCTCCGGTAAAAGAAAGCTCTTTTGTTGAGAAGATGA
AGAAGACAGGCCGTAATATCATCGTGTTTTATGGTAGCCAGACTGGTACAGCCGAG
GAATTTGCAAACAGACTGAGCAAGGACGCGCACAGGTACGGTATGCGTGGCATGTC
CGCCGATCCCGAAGAGTATGATCTAGCCGACCTGAGCAGCTTACCGGAAATCGATA
AATCCCTTGTTGTCTTTTGCATGGCGACCTATGGAGAGGGCGATCCGACCGATAACG
CACAGGACTTCTATGATTGGTTGCAAGAGACGGACGTAGACCTGACAGGCGTGAAG
TTCGCCGTCTTCGGACTGGGCAATAAAACATACGAGCACTTCAACGCAATGGGCAA
GTATGTGGATCAGCGTTTAGAGCAACTAGGCGCCCAAAGGATTTTCGAGTTGGGTCT
GGGAGACGACGATGGAAACCTAGAAGAAGATTTCATAACCTGGCGTGAGCAATTCT
GGCCTGCAGTATGCGAGTTCTTTGGTGTTGAGGCCACGGGCGAAGAATCATCTATAA
GGCAGTATGAATTGGTCGTTCACGAAGATATGGACGCCGCGAAGGTATACACCGGC
GAAATGGGGCGTCTTAAATCATACGAAAACCAAAAACCTCCCTTTGACGCTAAAAA
TCCATTTCTAGCTGCTGTCACCGCAAATCGTAAGTTAAACCAGGGCACTGAGAGGCA
CCTAATGCACCTGGAGCTGGATATCAGCGATTCCAAAATCAGATACGAATCAGGAG
ACCACGTCGCGGTGTATCCCGCAAACGATTCAGCGTTAGTAAACCAAATCGGGGAA
ATACTTGGAGCGGATCTTGATGTGATAATGTCTTTAAATAACCTAGACGAGGAATCC
AATAAGAAACACCCATTTCCCTGTCCTACGACATACAGAACCGCGCTGACGTATTAT
TTGGATATAACAAATCCTCCCAGAACGAACGTTCTATATGAGTTAGCCCAGTATGCT
TCAGAGCCGAGTGAACAGGAACATCTGCACAAGATGGCGAGCAGTTCAGGAGAGG
GTAAGGAATTATACCTTTCCTGGGTCGTTGAGGCGCGTAGACACATACTTGCAATTC
TACAAGACTACCCTAGCCTAAGACCACCTATAGACCATCTGTGCGAGTTATTGCCAC
GTTTGCAAGCCAGGTATTATAGCATCGCAAGTTCTTCTAAAGTTCACCCCAACTCTG
TGCACATATGTGCAGTTGCTGTCGAATACGAAGCAAAATCCGGGAGGGTTAATAAG
GGAGTAGCTACGAGTTGGCTAAGAGCAAAAGAACCAGCTGGTGAAAATGGAGGTCG
TGCCCTTGTCCCGATGTTTGTAAGAAAATCCCAATTCAGACTACCGTTTAAGAGTAC
CACGCCCGTGATCATGGTTGGTCCGGGGACTGGTATAGCACCTTTTATGGGGTTCAT
CCAGGAGCGTGCCTGGCTACGTGAGCAAGGCAAAGAGGTAGGCGAGACATTGCTTT
ACTACGGGTGTAGACGTAGCGATGAAGATTACCTGTACAGAGAAGAGTTGGCGAGA
TTCCACAAAGACGGCGCTTTAACCCAACTAAACGTCGCTTTTAGCAGAGAACAGGCT
CATAAAGTGTACGTCCAGCACTTGCTGAAAAGGGACAGGGAGCATTTATGGAAACT
GATTCATGAAGGTGGCGCGCACATATACGTATGCGGGGATGCTCGTAATATGGCTA
AGGATGTGCAGAATACATTTTACGACATTGTAGCGGAGTTCGGCCCTATGGAGCATA
CGCAAGCTGTAGATTATGTCAAGAAATTAATGACCAAAGGTAGATACTCATTGGAC
GTTTGGAGCTAA
>CPR_5
Seq. ID NO: 17
ATGGCCGCAATCAACATGGGTGACTCTCATGTTGACACAAGTTCCACCGTTTCCGAA
GCTGTAGCGGAAGAAGTGAGCCTATTCAGCATGACAGATATGATTCTATTCTCACTT
ATTGTCGGTTTACTGACTTACTGGTTTCTATTCAGAAAGAAGAAGGAAGAGGTCCCC
GAATTTACGAAAATACAAACACTTACTTCCTCAGTTAGGGAATCATCATTCGTTGAG
AAAATGAAAAAGACGGGGCGTAACATCATCGTGTTTTATGGTTCACAAACCGGAAC
TGCCGAAGAGTTTGCGAATAGATTATCTAAGGACGCTCATAGATACGGAATGCGTG
GGATGTCTGCCGATCCCGAGGAATATGATCTGGCAGATCTTAGCAGTCTACCGGAAA
TCGACAATGCACTTGTGGTGTTCTGCATGGCAACATACGGGGAAGGAGATCCGACG
GATAATGCACAGGACTTTTATGACTGGTTGCAAGAGACCGACGTGGATCTATCCGGT
GTCAAGTTTGCCGTTTTTGGGCTTGGGAATAAGACCTACGAGCACTTCAATGCAATG
GGAAAATATGTGGATAAGAGACTGGAGCAGCTGGGAGCCCAAAGAATATTCGAGTT
AGGATTAGGTGACGATGATGGGAATCTTGAGGAAGACTTCATCACCTGGCGTGAAC
AGTTTTGGCCGGCAGTTTGCGAACACTTTGGTGTAGAAGCCACTGGGGAAGAGTCTT
CCATCAGGCAATACGAGCTGGTAGTGCATACAGATATTGATGCAGCTAAGGTATAT
ATGGGTGAGATGGGAAGGTTAAAAAGTTATGAGAACCAGAAACCACCTTTTGATGC
CAAAAATCCTTTTCTGGCCGCAGTTACGACAAACAGGAAATTAAACCAGGGTACGG
AAAGACACTTAATGCATTTAGAACTGGACATCTCAGACAGCAAGATTAGGTACGAA
AGTGGGGACCACGTCGCAGTGTACCCGGCTAATGACAGCGCGCTAGTGAATCAGCT
GGGTAAAATTCTAGGGGCGGATCTTGACATCGTGATGAGCCTGAATAATTTGGACG
AAGAGAGCAACAAGAAACATCCCTTCCCCTGTCCTACTTCCTATAGGACGGCTCTAA
CATATTATCTGGACATAACGAATCCCCCTAGAACGAACGTCTTGTACGAATTGGCAC
AGTACGCTTCTGAACCGTCAGAACAGGAATTACTACGTAAAATGGCGAGTTCTAGTG
GTGAAGGAAAAGAGTTATACCTAAGTTGGGTTGTAGAGGCAAGGAGACACATTCTG
GCTATTTTACAAGATTGCCCTAGTTTGAGGCCGCCTATAGACCACCTTTGTGAGTTAT
TACCGAGGTTACAGGCTCGTTACTATTCTATAGCAAGCAGTAGTAAGGTCCACCCGA
ATAGCGTACACATATGCGCCGTAGTAGTAGAATACGAAACAAAGGCGGGTCGTATC
AACAAAGGAGTGGCGACCAACTGGTTGCGTGCCAAAGAACCGGCAGGAGAAAACG
GTGGGAGGGCTCTAGTTCCGATGTTTGTCCGTAAAAGTCAGTTTAGGTTACCGTTCA
AGGCCACGACCCCCGTAATCATGGTAGGTCCCGGGACTGGAGTTGCGCCGTTCATA
GGCTTTATCCAGGAAAGGGCCTGGTTGCGTCAGCAGGGCAAGGAGGTCGGTGAAAC
ACTGTTATACTATGGATGCAGGAGGAGTGACGAGGACTATCTGTACAGAGAAGAGC
TTGCCCAATTCCACAGGGACGGCGCATTGACTCAACTTAACGTAGCCTTCTCACGTG
AACAGTCTCACAAGGTGTACGTCCAACACCTGCTGAAGAGAGACAGGGAGCATCTG
TGGAAATTAATAGAAGGCGGAGCACATATCTATGTATGTGGCGATGCAAGAAACAT
GGCACGTGATGTCCAGAATACCTTTTACGATATTGTCGCGGAGCTTGGGGCGATGGA
ACACGCTCAAGCCGTAGATTACATCAAGAAACTAATGACAAAGGGGCGTTATTCTCT
TGACGTATGGTCCTAA
>CPR_6
Seq. ID NO: 18
ATGGCCGCAATTAACATGGGAGATTCCCACATGGATACATCCAGCACAGTGTCCGA
AGCAGTTGCGGAGGAAGTGAGTTTATTTTCCATGACAGACATGATCCTTTTCTCACT
TATTGTGGGGTTACTTACCTACTGGTTTTTGTTTAGGAAAAAGAAAGAGGAGGTCCC
GGAATTTACTAAAATACAGACTTTGACATCATCAGTACGTGAGTCTTCCTTTGTAGA
GAAAATGAAGAAGACAGGCAGGAATATAATAGTATTTTATGGATCTCAGACCGGGA
CCGCAGAGGAGTTCGCTAACCGTCTATCCAAAGACGCCCACCGTTATGGTATGAGG
GGCATGAGTGCGGACCCCGAGGAGTACGATCTAGCCGATCTATCCAGCTTACCTGA
GATAGAGAACGCATTGGTTGTATTTTGTATGGCCACGTATGGCGAAGGTGATCCGAC
AGATAATGCTCAAGATTTCTACGACTGGTTACAAGAGACCGACGTAGACTTGTCTGG
TGTAAAGTTCGCTGTATTTGGATTAGGAAATAAGACCTATGAACACTTTAACGCCAT
GGGCAAGTATGTCGATAAACGTCTGGAACAATTAGGCGCTCAGAGAATTTTCGAAC
TGGGACTAGGCGATGACGACGGAAACCTGGAGGAGGACTTTATTACGTGGAGAGAA
CAATTTTGGCCCGCTGTCTGTGAACATTTCGGCGTTGAGGCAACCGGCGAAGAAAGC
TCGATAAGGCAATATGAATTGGTAGTACATACAGATATAGACGCCGCTAAAGTGTA
CATGGGAGAAATGGGAAGGTTGAAGAGTTATGAGAACCAAAAACCTCCGTTCGACG
CTAAGAATCCGTTCCTAGCGGCTGTCACTACAAATAGAAAGTTGAATCAAGGCACA
GAAAGGCATTTAATGCATTTAGAGCTTGATATTTCTGACAGCAAAATCAGATACGAA
TCCGGGGACCATGTTGCGGTCTACCCAGCAAACGACAGTGCCTTAGTAAATCAGCTA
GGGAAAATACTTGGGGCGGATTTGGATGTCGTAATGAGTCTTAATAACCTAGATGA
AGAATCAAATAAGAAACATCCATTTCCTTGTCCCACAAGCTATAGGACCGCGCTGAC
TTACTATCTGGACATCACTAATCCCCCAAGAACCAATGTGTTGTATGAGTTAGCCCA
GTATGCCAGCGAGACGAGTGAGCAAGAGTTATTGAGAAAAATGGCAAGTAGCTCAG
GAGAAGGGAAGGAGCTGTATCTGAGCTGGGTTGTAGAGGCACGTAGACACATTCTG
GCCATTTTGCAAGATTGCCCTTCTTTGAGACCACCTATAGATCATCTTTGTGAGCTTC
TACCAAGGCTGCAGGCTCGTTACTACAGTATTGCTAGTTCAAGCAAAGTTCATCCAA
ACAGTGTCCACATCTGCGCGGTCGTAGTTGAATACGAGACAAAGGCCGGGAGAATT
AATAAAGGTGTGGCCACTAATTGGCTAAGAGCCAAAGAACCTGCTGGTGAGAACGG
TGGGCGGGCGCTTGTCCCGATGTTTGTAAGGAAGAGTCAATTTCGTCTGCCATTTAA
GGCGACCACGCCTGTTATTATGGTGGGTCCAGGAACAGGTGTAGCCCCATTTATAGG
ATTCATTCAAGAAAGGGCCTGGTTAAGGCAACAAGGCAAAGAGGTTGGTGAAACTC
TTCTTTATTATGGGTGTCGTAGATCAGATGAAGATTACCTGTATCGTGAAGAGCTTG
TACAATTCCATAGAGACGGCGCGCTTACACAATTGAATGTTGCTTTTAGTCGTGAGC
AAAGTCATAAAGTCTACGTTCAACATTTATTAAAAAGGGATAGAGAGCACTTGTGG
AAACTGATAGAAGGAGGGGCGCATATCTATGTATGCGGAGACGCGAGGAATATGGC
TAGAGACGTGCAAAATACCTTTTATGATATCGTTGCAGAATTGGGTGCAATGGAGCA
TACGCAAGCCGTTGATTACATAAAGAAGTTGATGACCAAAGGACGTTACAGCCTAG
ATGTTTGGTCTTAA
>CPR_7
Seq. ID NO: 19
ATGGCCGCAAATATGGCAGACAGTAATATGGATGCGGGAACGACCACATCTGAGAT
GGTAGCAGAGGAGGTAAGCCTATTTTCCACTACGGATGTGATATTGTTCAGTTTGAT
CGTAGGTGTTATGACTTATTGGTTTCTTTTCAGGAAAAAGAAAGAAGAGGTGCCGGA
GTTCACAAAGATTCAGACCACTACCTCCTCCGTTAAGGATAGATCATTTGTTGAAAA
GATGAAGAAAACCGGCAGGAATATCATTGTGTTCTACGGTAGCCAGACAGGAACGG
CGGAAGAGTTCGCCAACCGTCTATCCAAAGACGCTCACAGATATGGTATGAGGGGA
ATGGCGGCCGACCCGGAGGAGTACGACCTGGCTGATCTGTCTTCTTTGCCAGAAATA
GAGAAGGCGTTGGCTATATTCTGTATGGCAACCTATGGAGAAGGGGACCCAACAGA
TAACGCTCAGGACTTTTACGATTGGTTGCAAGAGACTGATGTGGACCTAAGTGGTGT
AAAGTATGCGGTATTTGCCCTGGGGAACAAGACGTACGAGCACTTCAATGCAATGG
GTAAGTACGTAGATAAGAGACTGGAACAGTTGGGAGCGCAAAGGATATTCGACTTG
GGATTAGGTGATGACGACGGAAACCTAGAGGAAGATTTCATAACCTGGAGGGAACA
ATTCTGGCCCGCCGTTTGTGAGCATTTTGGCGTTGAAGCAACGGGTGAAGAGAGTTC
TATCCGTCAATACGAGTTGATGGTACATACGGATATGGATATGGCAAAAGTTTACAC
CGGTGAAATGGGAAGACTAAAGTCATACGAAAACCAGAAGCCCCCATTTGATGCGA
AAAACCCTTTCCTAGCAGTCGTAACGACGAACCGTAAGCTGAATCAGGGGACGGAG
AGGCACTTAATGCACTTAGAACTTGATATATCTGACTCTAAAATTAGATATGAATCT
GGGGACCATGTAGCCGTATATCCAGCAAACGATAGCGCCTTAGTAAACCAACTGGG
CGAGATATTGGGGGCTGATCTGGATATCATAATGAGTTTGAACAACTTGGATGAAG
AAAGTAACAAGAAACATCCGTTCCCTTGTCCCACATCATATAGAACAGCCCTTACCT
ACTACTTGGACATAACAAACCCGCCAAGAACTAACGTTCTATACGAGTTGGCTCAAT
ATGCGAGCGAACCGACCGAACATGAACAACTACGTAAAATGGCATCTTCATCTGGT
GAGGGTAAGGAACTTTACTTGAGGTGGGTGCTTGAAGCTAGAAGGCATATCCTGGC
GATTTTGCAAGACTATCCTAGTCTAAGACCGCCGATTGATCACTTGTGTGAGCTTCTT
CCTAGACTTCAAGCTAGGTACTACTCAATCGCCAGCAGTTCAAAAGTGCACCCGAAC
TCAGTACATATATGTGCCGTAGCCGTCGAGTACGAGACCAAGACGGGCAGGATAAA
CAAAGGAGTCGCCACAAGTTGGCTGAGAGCTAAGGAACCAGCAGGCGAAAATGGT
GGGCGTGCCCTAGTGCCTATGTATGTGCGTAAAAGTCAGTTCAGATTACCGTTCAAA
GCTACGACCCCTGTAATAATGGTAGGCCCGGGCACTGGAGTGGCCCCCTTCATTGGA
TTCATTCAAGAGCGTGCCTGGCTAAGGCAACAGGGGAAGGAGGTTGGGGAAACATT
GTTGTATTATGGGTGCAGGAGAAGCGACGAGGATTATCTATATAGGGAAGAGCTGG
CGGGCTTCCATAAGGACGGCGCGCTGACTCAATTAAATGTTGCATTCAGCAGGGAA
CAACCCCAAAAGGTGTACGTGCAGCACTTACTTAAAAAGGATAAGGAACACTTATG
GAAATTAATTCACGAGGGTGGAGCCCACATCTACGTGTGTGGGGATGCCAGGAATA
TGGCTAGGGATGTCCAGAACACATTCTATGATATTGTAGCCGAACAAGGTGCGATG
GAGCACGCCCAAGCTGTTGACTATGTCAAGAAATTGATGACCAAAGGGAGATACTC
TCTGGACGTCTGGTCATAA
>CPR_8
Seq. ID NO: 20
ATGGCCGCTGAACCTACCTCACAAAAGCTTAGTCCTCTTGACTTCATTGCGGCCATT
CTAAAAGGTGATTATTCAGATGGGCAACTGGAAGCTGCATCCCCGGGGATGGCTAT
GCTGCTGGAGAATAGAGATCTTGTAATGGTTCTTACAACAAGTGTGGCTGTATTGAT
AGGGTGTGTGGTGGTGTTAGCCTGGAGACGTACGGCCGGTTCAGCCACCAAAAAGC
AGTTTGAGCCTCCCAAGCTTGTAGTACCGAAAGCCGCAGAACTGGAGGAAGTTGAT
GACGACAAACCTAAGGTAAGTATCTTCTTTGGTACCCAAACCGGAACCGCGGAGGG
CTTTGCGAAAGCTTTCGCCGAGGAGGCCAAGGCCAGATATCCCCAGGCTAACTTCA
AGGTGATCGACTTAGATGATTACGCAGCAGACGACGACGAGTATGAGGAGAAACTG
AAGAAGGAGACGCTGGCGTTCTTCTTTCTGGCGTCCTACGGCGATGGAGAGCCCACA
GACAACGCGGCTAGATTCTACAAGTGGTTCACTGAGGGGAAGGATAGAGGTGATTG
GTTGAAAAATTTACAATACGGAGTGTTTGGTCTAGGCAATAGACAATATGAGCACTT
TAATAAAATCGCTATCGTTGTGAATGACATCATTGTCGAGCAAGGTGGAAAAAAGC
TAGTGTCAGTGGGCCTTGGGGACGACGATCAGTGCATAGAAGACGACTTCGCCGCTT
GGAGAGAATTAGTATGGCCAGAACTTGACAAGTTGCTTCGTAACGAAGACGATGCC
ACCGTCTCTACACCATACACCGCAGCAGTACTGCAATATAGGGTTGTCTTTCATGAT
CAGACAGACGGCTTAATCTCCGAGAACGGCTTCTTGAACGGCAGAGCGAACGGGAC
GTCTGTCTTCGATGCCCAACACCCCTGTCGTTCCAACGTCGCGGTCAAAAAAGAACT
TCACACCCCCGCTAGTGACCGTAGTTGTGCCCATCTTGAATTTGATATATCTGGGACT
GGGTTAGTCTATGAAACTGGAGATCATGTTGGAGTTTACTGCGAAAACCTAATTGAA
ACTGTAGAGGAGGCCGAAAAGTTGTTAAATATTCCCCCTCAAACATATTTTTCCATA
CATACGGATAATGAGGATGGGTCCCCTCGTAGCGGCAGCTCTCTTCCGCCTCCATTC
CCGCCATGCACTTTAAGAACGGCCTTGACCAGGTATGCGGATTTGTTGTCCGCGCCT
AAAAAAAGCACCTTAATTGCTCTAGCAGAGAGTGCCAGCGATCAGAGTGAAGCCGA
CAGATTACGTCACCTTGCGAGCCCCGGTGGTAAGGAGGAATACGCTCAATATATCAC
CGCAAGCCAAAGGTCCCTGCTAGAGGTAATGGCGGACTTCCCCAGTGCGAAGCCTT
CATTGGGCGTCTTTTTCGCAGCCATAGCCCCCCGTTTGCAGCCCCGTTTTTACTCTAT
CTCAAGCTCACCGAAAATAGCACCTAGCAGGATACATGTTACGTGCGCGCTGGTTTA
TGAAAAAACGCCTACGGGTCGTGTTCATAAGGGTGTCTGCAGTACATGGATGAAGA
ACGCTGTGCCCCTGGAGGAATCTAATGACTGTAGCTGGGCGCCGATATTTGTCAGGA
ACTCCAACTTCAAGCTACCTGCCTATCCCAAAGTGCCCATAATTATGATTGGCCCTG
GAACTGGTCTGGCCCCGTTCAGAGGTTTTCTACAAGAGCGTCTTGCGTTAAAAGAAT
CAGGTGCCGAATTGGGACCAGCTATACTATTCTTCGGGTGTAGGAATAGAAAAATG
GACTTCATCTATGAAGATGAGCTAAACAACTTCGTAGAGGCGGGCGTTATAAGTGA
ACTGATAGTAGCGTTTAGTAGGGAGGGACCAACTAAGGAGTATGTACAACACAAGA
TGACTCAGAGGGCGTCAGATGTATGGAAGATCATAAGCGATGGAGGTTATGTTTAT
GTGTGCGGCGACGCGAAAGGAATGGCAAGGGATGTTCACCGTACACTACATACAAT
AGCACAAGAGCAGGGCAGCCTTTCTTCATCTGAAGCAGAGGGAATGGTGAAAAATC
TACAGACAACCGGGCGTTATCTGAGGGACGTATGG
>CBNsyn_1
Seq. ID NO: 21
ATGGCCGCAGACTTCTCAGGTAAAAACGTTTGGGTCACGGGGGCCGGTAAAGGTAT
AGGTTACGCGACAGCATTGGCATTCGTAGAGGCAGGGGCCAAGGTCACAGGCTTCG
ATCAGGCATTTACACAGGAACAGTACCCTTTTGCCACCGAGGTTATGGATGTAGCGG
ACGCCGCCCAAGTAGCACAAGTCTGTCAGCGTCTACTAGCTGAGACAGAGAGATTG
GATGCTCTGGTGAATGCGGCAGGTATTCTTCGTATGGGTGCCACCGACCAATTATCT
AAGGAGGACTGGCAACAAACGTTCGCTGTAAATGTTGGAGGTGCATTTAACCTGTTC
CAACAGACTATGAATCAGTTCAGAAGGCAGCGTGGAGGCGCTATAGTCACAGTAGC
CAGTGACGCCGCGCATACCCCAAGGATTGGAATGTCAGCGTACGGAGCTTCCAAGG
CAGCCCTGAAGAGCCTAGCTTTATCAGTCGGTCTGGAGCTGGCCGGGTCAGGGGTA
AGGTGCAACGTTGTGTCCCCGGGCTCCACGGATACAGACATGCAGAGAACTCTGTG
GGTGTCTGACGACGCAGAGGAACAACGGATCAGAGGTTTCGGAGAGCAGTTCAAAC
TAGGGATTCCGCTGGGCAAGATCGCTAGACCACAAGAGATAGCTAATACTATACTTT
TCCTAGCATCCGATTTAGCCAGTCACATCACTTTACAAGACATCGTAGTGGATGGTG
GTTCAACACTAGGCGCTTAA
>CBNsyn_2
Seq. ID NO: 22
ATGGCCGCAAGCGATCTGCATAATGAGTCCATTTTCATTACAGGCGGAGGCTCTGGT
CTTGGGCTGGCCTTAGTGGAAAGGTTTATAGAGGAAGGGGCACAGGTTGCTACACTT
GAGCTTAGCGCAGCAAAAGTCGCGTCTCTACGTCAACGTTTTGGAGAACATATATTG
GCCGTGGAAGGCAACGTCACGTGTTATGCCGACTATCAAAGAGCTGTAGATCAGAT
ACTAACCCGTTCTGGGAAGTTAGATTGCTTTATAGGGAATGCAGGTATATGGGATCA
TAACGCTTCCCTGGTTAATACCCCAGCAGAAACGCTAGAAACAGGGTTTCATGAGCT
TTTTAACGTAAACGTCCTGGGGTACTTACTGGGAGCAAAAGCATGTGCTCCTGCGCT
TATCGCGTCAGAGGGTTCAATGATATTTACCCTTTCAAACGCGGCTTGGTACCCAGG
TGGAGGGGGTCCTTTATATACGGCCTCCAAACATGCAGCAACTGGCCTGATCCGTCA
ACTAGCCTATGAACTTGCACCCAAGGTAAGGGTTAATGGAGTGGGTCCCTGCGGCAT
GGCTAGTGATCTTAGGGGACCACAAGCCTTAGGGCAATCAGAAACGAGTATAATGC
AGTCATTGACCCCCGAAAAGATTGCGGCGATATTACCTCTGCAATTTTTCCCACAAC
CGGCGGACTTCACTGGACCATACGTCATGTTAACATCTAGGCGTAATAATAGGGCAC
TGAGCGGCGTTATGATTAACGCGGACGCTGGGTTGGCTATCAGAGGCATTAGGCAC
GTGGCAGCAGGACTTGACCTATAA
>CBNsyn_3
Seq. ID NO: 23
ATGGCCGCAACGGGATGGTTAGCGGGAAAAAGAGCTTTGATCGTCGGTGCGGGTTC
CGGAATCGGAAGAGCTACAGTTGACGCATTTCTAAACGAGGACGCGAGAGTTGCAG
TTCTGGAGTATGACTCCGATAAGTGTGCAACACTTAGGCACCAGTTACCAGACGTTC
CCGTGATAGAAGGCGATGGGACCACAAGGACCGCTAACGATGAGGCCGTTCAGGTC
GCTGTGGACGCATTCGGGGGACTAGATACTCTGGTCAACTGTGTTGGAATATTCGAC
TTCTACCGTCGTATCCAAGACATTCCCGCAGAGCTGATCGATCAGGCATTTGACGAA
ATGTTTAGAATCAATGTATTATCACATATCCACTCTGTTAAAGCAGCGGTACCTGCT
CTGATGGGTCAGGACGGAGCATCTATTGTGCTGACGGAGAGTGCTTCTTCATTCTAT
CCCGGTAGGGGCGGGTTGTTGTATGTGGCGTCAAAATTTGCTGTTCGTGGTGTCGTA
ACCGCACTGGCCCATGAGTTGGCTCCCAGGATTCGTGTTAATGGAGTAGCTCCTGGC
GGAACCCTTAATACAGATCTGAGGGGCCTTGACAGTTTGGACCTTGGTGCCCGTAGG
TTAGATGCCGCGCCTGACAGAGCTAGAGAACTTGCAGCGAGGACCCCACTGGGGGT
CGCATTGTCCGGTGAAGACCACGCCTGGTCTTACGTTTTCCTGGCCTCTCATAGGAG
TAGAGGTCTAACAGGCGAAACGATTCACCCTGATGGCGGCTTTTCTTTAGGACCGCC
GCCACAAAGGAATTAA
>CBNsyn_4
Seq. ID NO: 24
ATGAGTAGTATCGAGACCAAAATCTTTCCTGGGCGTTTTGATGGTAGGTGTCTTACC
ATAACAGGTGCCGCCCAAGGCATTGGGTTGACAGTAGCTACGAGGATAGCGGCAGA
AGGCGGTGAAGTGGTGCTTGTTGACCGTGCAGACCTTGTACACGAGGTGGCAGAGC
AGCTACGTGAGGCAGGAGGCAAGGCGCACTCAGTAACGGCTGATTTAGAAACATTT
GAGGGTGCTGAGGAAGCGATCTCTCATGCCGTAAGGACGACTGGCAGAATCGATGT
ACTAATCAATGTTGTGGGCGGGACTATATGGGCAAAACCGTATGAGCACTACGCCC
CGGAGGAAATAGAAAAAGAAATTAGAAGATCCTTATTCCCTACGCTATGGACATGT
AGAGCTGCGGCACCGCATTTAATCGAACGTAGAGCAGGAACGATAGTGAACGTAAG
CTCCGTTGCTACGAGGGGCGTAAATCGTGTTCCCTATTCCGCAGCAAAGGGAGGTGT
TAATGCTATTACTGCGTCTCTGGCGTTGGAATTAGCCCCGTACGGGGTAAGGGTTGT
CGCAACGGCTCCAGGCGGGACCGTCGCGCCAGAGAGAAGAATCGCCAGAGGGCCT
AGCCCACAGAGTGAGCAGGAGAAAGCCTGGTACCAGCAGATTGTAGATCAGACAGT
TGACTCCTCATTACTTAAAAGGTATGGTACTCTTGATGAACAAGCAGCCGCGATCTG
CTTCCTTGCATCAGAAGAGGCGTCATACATCACCGGAACTGTCTTGCCGGTGGCCGG
AGGGGACTTAGGATAA
>CBNsyn_5
Seq. ID NO: 25
ATGAGTAGTACCGGCTGGCTAGACGGCAAAAGGGCCTTAGTTGTTGGGGGAGGAAG
TGGGATAGGTAGAGCTGTCGTAGACGCTTTCTTAGCTGAAGGAGCTTGCGTAGCCGT
CCTGGAAAGGGACCCGAATAAGTGTAGAGTCCTAAGAGAACATCTGCCGCAGGTGC
CCGTAATTGAAGGAGATGCAACAAGGGCTGCAGATAATGACGCAGCGGTAGCTGCA
GCAGTTGCTGCATTTGGAGGACTAGACACGCTTGTAAATTGTGTGGGTATCTTTGAC
TTCTATCAGGGCATCGAGGACATTCCGGCGGACACCCTTGACGTAGCATTCGATGAA
ATGTTCAGAACGAACGTACTATCCCACATGCATAGTGTAAAGGCGGCAGTTCCCGA
GTTACGTAAACATAGGGGCTCTTCTATCGTTCTGGCTGAATCCGCCTCTAGCTTCTAT
CCAGGGAGAGGGGGTGTCCTATATGTCTCTTCTAAATTTGCCGTCAGAGGTCTGGTA
ACCACTCTAGCATACGAGTTGGCCCCAGATATCAGGGTGAATGGGGTCGCCCCAGG
TGGTACGCTGAATACGGATCTGCGTGGCTTAGCGTCACTAGGAAGGGATGCTGACA
GGCTAGATGATAACCCTAATAGGGCCAATGAGTTAGCAGCCAGAACTCCGCTTAAC
GTGGCCCTTAGTGGGGAAGATCATGCGTGGTCTTTTGTCTTCTTCGCTTCCGACAGA
AGCAGGGGAATTACAGCCGGGGCTACTCATCCAGATGGAGGCTTTGGAATTGGTGC
GCCCAAGCCCTCTACTAGATAA
>CBNsyn_6
Seq. ID NO: 26
ATGAGTAGTGGGTTTCTGGATGGCAAGGTTGCTCTTGTGACTGGTGGCGGGAGTGGT
ATTGGAAGGGCCGTCGTCGAATTATACGTTCAGCAAGGAGCTAAAGTAGGTATCTTA
GAAATCTCACCCGAAAAAGTGAAGGACCTGAGGAATGCCCTACCAGCTGACAGTGT
CGTGGTAACAGAGGGAGATGCTACGAGTATGGCGGATAACGAGAGGGCAGTCGCG
GACGTTGTTGACGCATTCGGACCCCTTACTACGTTAGTTTGTGTGGTGGGGGTATTC
GATTACTTTACAGAGATTCCTCAGCTACCTAAAGATAAAATCTCTGAAGCCTTTGAT
CAACTTTTTGGGGTAAATGTTAAATCCAACCTATTGTCTGTGAAAGCGGCGTTAGAC
GAGCTAATTGAGAACGAAGGAGACATAATACTGACGCTAAGTAACGCAGCTTTTTA
TGCCGGTGGAGGCGGCCCACTGTATGTTTCTAGTAAGTTTGCTGTAAGGGGCTTGGT
GACTGAGTTAGCATATGAGCTTGCCCCAAAAGTACGTGTCAACGGGGTAGCCCCAG
GGGGAACGATTACCGAACTTAGAGGAATCCCGGCCTTGGCGAATGAAGGACAAAGG
CTGAAAGACGTTCCTGACATCGAGGGATTAATAGAAGGAATTAATCCCCTTGGTATC
GTTGCTCAGCCTGAGGACCACTCCTGGGCCTACGCGTTATTAGCAAGTAGAGAAAG
GACATCAGCGGTAACAGGCACGATTATAAACAGCGATGGAGGATTAGGAGTCAGGG
GCATGACTCGTATGGCCGGTCTGGCACAATAA
>CBNsyn_7
Seq. ID NO: 27
ATGAGTAGTAGTAGGTCTGTGACTTTGGTAGTCGGCGCTGCCCAAGGAATTGGCAGG
GCTACCGCATTGACGCTTGCGACGGCGGGTCACAGGGTCGTGTTGGCGGATAGGGA
CGTAGACGGCTTGGCCGAGACTGCTGCGCTTCTACACGTCGCTGCACCGGTTCACGG
ACTTGACGTATGTGATGCTGCTGGGGTGGCGGAAGCGGTTGCGAGGGTGGAGGTCG
AGCACGGACCGGTAGATGCTCTAGCTCATGTCGCGGGGGTGTTTACCACGGGCTCTG
TACTTGATTCAGACTTAGCAGAGTGGCAACGTATGTTTGACGTCAACGTGACGGGGC
TAATCAATGTACTGCGTGTCGTGGGGCATGGCATGAGAGAACGTAGACGTGGAGCA
ATCGTCACTGTCGGTTCTAATTCCGCTGGTGTACCAAGGGTGGGGATGGGAGCTTAT
GGTGCATCAAAATCCGCAGCACATATGCTGGTACGTGTATTAGGATTGGAATTAGCA
AGATTCGGCGTCAGGGCGAATGTTGTTGCCCCAGGGTCCACGGACACAGCGATGCA
ACGTTCTCTTTGGCCCGACCCTGCTGACGACGCTGGCGCCCGTACTGCGATAGACGG
TGACGCCGCTTCATTTAAGGTCGGGATTCCACTGGGGAGGATCGCAGACCCAGCCG
ACATCGCGGACGCCGTCGAGTTCCTGCTATCTGATCGTGCTAGGCACATAACAATGC
AGACTCTATATGTAGATGGTGGTGCTACCCTGAGAGCATAA
>CBNsyn_8
Seq. ID NO: 28
ATGAGTAGTCAAATGCTGGATGACCACGTAGCTCTGATACTAGGTGGTGGGAGTGG
ATTGGGTCTAGGAATTGCGCGTCATTTTCTCGGAGAAGGGGCTCAGGTGGCCATCTT
TGAGATCAGTGAATCCAAATTATTAGACCTAAAAGCTGAGTTCGGGGACGACGTAC
TTCTTTTACAGGGGGATGTAACATCAATTGACGACCTAGAGGCAGCCCGTGCCGCAG
TAGTGGATAGGTTCGGAAGGTTGGATGCACTTATTGGTGCGCAAGGGATTTTTGATG
GGAACATCCCATTGAGAGACATCCCGACCGAGAGAATCGAAAAGGTTTTCGACGAA
GTGCTACATGTTGACGTGCTAGGTTATATATTAGCCGCTAGGGTCTTCCTGGAAGAG
CTGGAGAAGACAGACGGAGCAATTGTGTTTACCAGCAGTACTGCGGCTTACGCAGC
CGATGGAGGAGGTTTGTTTTACACTGCCGCCAAGGGTGCCGTTAGAAGTGTAATCAA
TCAGCTTGCATTCGAGTTCGCGCCGAAGGTCAGAGTCAACGGAGTCGCTCCATCCGG
CATCGCTAATTCACAGCTTCAAGGGCCGCGTGCCCTAGGATTAGAGAACAACAAGC
AGAGTGATATTCCCGTTGAGGATTTTACGAACCAATTTCTGTCTCTGACGTTGACAC
CTACCCTGCCCACTCCGGAGGAATATGCGCCACTTTATGCATATTTAGCGTCCAGGA
ACAATACCACAATGACAGGGCAAACGATAATTGCAGATCAGGGCCTATTTAACAGA
GCGGTCATATCTAACGGCGTTGCAGATAGAGTAGGCAAATAA
>THCdeg_1
Seq. ID NO: 29
ATGAGTAGTTCTGGCCCCGCGCACAGCAATTTAGAGCAAGTATTCGCTAACGTGGCT
TCAAATTACCGTGGGGCTGATGTAGACTTGCACGCGGTTTATAGAGAAATGCGTGAG
AAGTCTCCCGTGTTGCCTGAAAATTTCATGGCCAGGCTTGGTGTGCCGTCTATAGCA
GGGCTGGACCCAAATAGGCCAACTTTTACGTTGTTTAAATATGACGATGTGATGGCT
GTAATGAGAGATGCGACTAATTTCACTAGTGGTTTTATTGCGGAAGGTCTGGGCTCT
TTCTTCGATGGTTTAATTCTAACAGCAATGGACGGTGAAGCACACAAGAATATACGT
TCATTGTTACAGCCGGTCTTTATGCCAGAAACTGTTAATAGGTGGAAAGAGACCAAA
ATTGACAGAGTGATAAGGGAAGAGTATCTTAGACCAATGGTGGCTTCAAAGCGTGC
CGATATCATGGAGTTTGCTTTATATTTCCCCATTAGAGTTATTTACTCATTGATTGGA
TTCCCAGAGGACCGTCCGGAGGAGATCGAACAGTATGCGGCTTGGGCCTTAGCGAT
TCTGGCCGGACCTCAAGTAGATCCTGAAAAAGCAGCAGCGGCACGTGGAGCAGCAA
TGGAAGCCGCCCAAGCACTGTACGACGTTGTTAAGGTAGTCGTAGCGCAAAGGAGG
GCCGAAGGGGCGACAGGCGACGACCTGATTTGCAGACTGATCAGAGCAGAGTACGA
AGGACGTAGTCTGGATGACCATGAAATAACGACGTTTGTTAGAAGCCTTCTGCCAGC
AGCTTCTGAAACGACGACGCGTACGTTTGGTACATTGATGACTCTGTTGCTAGAACG
TCCTGAACTGTTGGCACGTATCCGTGAGGATCGTTCTTTAGTCGGAAAAGCTATTGA
TGAGGCGGTACGTTATGAACCAGTGGCTACTTTTAAGGTAAGGCAAGCCGCAAAAG
ACGTGGAAATTAGAGGGGTGGCAATTCCGAAGGGCGCGATGGTGTCCTGCATCGTG
ACTAGCGCAAATCGTGACGAGGACGCTTTTGAGAATGCGGATACATTCGATATCGA
CCGTAGGGCTAAGCCGTCATTTGGATTTGGATTCGGTCCACATATGTGTATTGGTCA
GTTTGTTGCTAAAACCGAAATAAACTGCGCCCTAAATGCCATACTGGATTTGATGCC
AAACATCCGTTTAGACCCAGATAAACCCGCGCCAGAGATTATAGGGGCGCAGCTAA
GAGGACCCCATCACGTCCACGTGATTTGGGACTAA
>THCdeg_2
Seq. ID NO: 30
ATGAGTAGTAGGTCAACTGACCTTCCGGACCTGAAATCTGCGGCCTTTCTTGCGGAC
CGTTACCCAACGTACAGGAGACTACAAAGTGATTTCCCGCACTTCGAAATGAATATA
AATGGAGAGGAGTGTATCGTGCTGACAAGATACAGCGACGTCGATGAAGTCTTACG
AAACCCGTTGGCCACGGTTCAACAAGCTCCTGGTGTATTTCCAGAAAGGATAGGTCA
AGGTGCTGGGGCCCGTTTCTATCGTGAGTCACTACCCAATATTGATGCCCCCGATCA
CACGCGTATCAGGCGTATAGTTACACCGGCGTTCAACCCGAAAACAGTTGCTAACAT
GAGAGGTTGGGTTGAGAAGGTAATAGTGGAGCACCTAGACCGTCTTGAAGGATTGG
ACGAAATTGACTTTGTCTCTAGCTTTGCCGACCCGGTGCCAGCGGAAATAGCATGTA
GGTTGCTTCATGTGCCTGTGTCTGATGCTCCAGAACTTTTTGCTAGGCAGCATGGATT
GAATGCTGTGCTATCTGTTAGCGACATCACACCTGAGAGATTAGCCGAAGCGGACG
CATCCGCTGCTTTCTACTATGAATACATGGACGACGTTTTAAACACACTGAAGGGTA
AATTGCCGGAAGATGATTTCGTGGGAGCGTTAATGGCTGCCGAGGCGCGTGACTCTG
GATTAACTAGGTCTGAATTGGTTACTACGCTTATCGGATTTCTGGTAGCCTCATATCA
CACCACGAAGGTGGCCATGACAAACACTGTCCTAGCTCTACTTAATCACGATGGCG
AGAGAGCTAGGCTTGTGGCGCAGCCGGATTTGGCGAGAAATGCCTGGGAAGAATCA
TTGAGATATGACTCCCCAGTGCATTTCGTCCACCGTTATGCATCTGAACCACTGACA
ATAGGTGGTCAGCCCGTGGCCCAAGGTAAAAGGCTATTATTGGGCTTGCATGCAGCT
AGTAGAGACGAAAATAGGTTTGCCCAGGCAGATCACTACTTGATTGACAGACCGGA
TAACCGTCACCTGGCGTTTGCTGGGGGAGGGCACTTTTGCTTGGGGTCTCAACTTAG
CCGTTTGGAAGGAGACGTACTGTTGCGTACAATTTTTCAAAGATTCCCCGCAATGAG
GCTTACGGAAACCAGATTCGAAAGAGTACCGGACTTGACTTTTCCAATGTTACTAAG
GATGACAGTTTCATTAAGGGCGGAGCAAGGTTAA
>THCdeg_3
Seq. ID NO: 31
ATGAGTAGTACCTCTAATTCAATTAGGAGCCCATTGAGTCCGCCCCAGCCGAGACGT
ACTCCGCCGCCTTGTACCTCCTCAAGGGAACCGCCCATCGTCCGTGGTACTTGGCTT
TTAGGCAGCACCCGTGACTTGTTAAGGGACCCACTGGAGCTAGGGCTGCGTGGATA
CGCTGAAGGCGGGGACGTGGTAAGATATGTAGTTGGGTTACCTGGTCGTAGAAGAG
AGTTCTTCACGGTTAACCATCCCGATGGGGTTGGGGAACTGCTTAATGCTCCCCGTC
ACTTAGACTATCGTAAGGACAGTGAATTTTACCGTGCCATGAGGGATTTATATGGAA
ATGGGCTTGTTACCAGTCAAGATGAAACTTGGCTGAGACAGAGAAGGTTCATACAG
CCGTTGTTTACTCCACAGAGTGTTGATGGTTACGTCACACCAATGGTCGCGGAGGCT
GATAGGGTAGCAATAAGGTGGCACAATTGTACCTCCCGTCTGGTAGATTTGGACGGC
GAGATGCGTGCCCTAACATTAGGCGTGGCCGCCAGAATCCTATTCGGAGTTCAAGCC
CCGAGGATGCTTCCTATCCTGAGGACTACCCTACCGGTACTTGGTAGGGCCGTTCTG
CAACAAGGTGCGTCAGCTATCAGATTTCCTAGCTCTTGGCCTACCCCGGGTAATCGT
CGTATCGCCAGTGCAGAATCTCGTCTGGATGGTTTGTGTGATGCTATTATAGAGCGT
CGTAGGACAGTAGCCGAGCCAGGTACGGATTTGCTGGGTCGTTTGGTCGCTGCAAG
AGAGGACGGTGATACGCTGTCAACGGAGGAAATAAGAGATCAAGTCAAGGTATTTC
TCTTGGCTGGTCACGATACAACGGCAACGATGCTGACGTTTGCCTTATACCTGCTTG
GTAAGGACGCTGGCGTTCAGGATCAAGCGCGTGACGAAGCGGAACGTGTCTTGGGG
GCGGGGACGCCGACCGCAAGCGACGTCCACCGTCTGACATATACTACGATGGTACT
GGAGGAGGCGGCGAGACTGTACCCACCGTCTCCCTATTTAACTCGTAGAGCGGTCG
AGGAAAGCGAGGTCTGCGGGTACAGAATACCCGCTGGGGCCGATGTCAACCTGGCT
CCATGGGTGATCCATCACCGTGCCGATTTATGGCCTGATCCTTTCCGTTTCGATCCCG
ACAGATTCACCCCGGATAGGGTAAAAGAAAGACACAAATACGCGTGGTTCCCGTTT
GGACACGGACCAAGGGGTTGTATCGGTCAGAGATTCGCAATGCTGGAAGCGGCAGT
TACTTTAGCGATTCTTCTAAGAGAATTTGAGTTTAGGTCTCCGCCTGGCAGCGTTCCA
TTAACAGTAGACTTACTGTTGCATCCCGCCGGCGAGGTTCCTTGCCGTGTGAGGAGG
CGTGTACCTGTGCATTCAGCGGTTCATCGTACTCACCAGCCAAGTTAA
>THCdeg_4
Seq. ID NO: 32
ATGAGTAGTGCCCCGGACATTCTTTCTCCCGAGTTTCTGGATAACCCTTATCCTCTTC
ACCGTGTGCTACGTGACCACTACCCCGCTTTACACCACGAGGGGACCGACAGCTATC
TAATATCAAGGTACGCCGATTGCGCAGAAGCATTTCGTTCACCTAAATTCTCCTCCC
GTAACTATGAATGGCAGCTTGAACCGATACACGGTAGAACAATTTTGCAAATGGAA
GGGCGTGAGCATTCTACCCATAGAGCATTGCTAAATCCGTTTTTCAGAGGCAACGGA
CTAGAGAGATTCATGCCTGCCATTACACACAACGCAGCACAACTAATAGGCGATAT
AGTCGCCAGGAATGCAGGGGAATTGCTGGGTGCGGTTGCCAGACAGGGGGAAGCGG
AATTGGTATCACAATTCACTAGTCGTTTTCCTATAAACGTAATGGTGGACATGCTGG
GACTGCCGAAGTCCGACCACGAAAGGTTTAGAGGCTGGTATTTCTCCATTATGGCTT
ATCTTAATAACCTGGCAGGGGACCCTGAAATTAACGCCGCGGCGGAGCGTACACAT
GTTGAACTAAGGGAGTACATGCTTCCAATTATTAGAGAGCGTAGGAGTGGAGATGG
AGACGACCTTCTATCCAGATTATGTCGTGCCGAAGTTGACGGTGAGCAGATGAGTGA
TGAGGAGATAAAGGCCTTTGTCTCTCTACTGCTGGTCGCCGGCGGAGAGACCACAG
ATAAGGCAATAGCAAGCATGATCAGAAATTTGATCGACCACCCAGATCAGATGAGG
GCGGTTAGAGAAGATCGTTCACTTGCTGATAGGGTAATAGCAGAGACCCTTCGTTAT
TCCGGACCCGTACATATGATCATGAGACAAACAGAGGATGAGGTTCAGATAGAGGA
CTCTACCATTCCAGCGGGAGCAACCTGCATAATGATGTTAGCAGCCGCGAACAGAG
ATGAACGTCATTTTTCAAACCCGGACGAGTTCGATATATTTCGTACGGACCTAAACG
TAGACAGAGCCTTCTCAGGGGCGGCCAATCATGTCCAATTTATATTGGGCCGTCATT
TTTGCGTCGGGTCCATGTTGGCTAAAACTGAGATGACCATTGCACTTAATCTGGTCTT
GGACACAATGGATAGCATAGAATACCAAGATGGTTTTGTTCCCAGAGAGGAGGGGC
TGTACACCAGAAGCATCCCGGAGCTTAGGGTAAAATTTGAAGGTAAGTTAGGGTAA
>THCdeg_5
Seq. ID NO: 33
ATGAGTAGTAGCACTCCTGCCGCTGCTACATCCTTGGAGAGTGCCTTTGCGGGCGTC
GCGGACAATTATAAAGGTTCCGACGTGGACCTTCATGCAATCTATAGAGATATGAG
ACGTAACTCTCCTGTCATCGCTGAGGATTTCATGGCACGTCTGGGTGTTCCGAATATT
GCAGGCCTAGACGCTAAAAGGCCAACATTTACCCTTTTCAAGTACAAGGACGTGAT
GTCTGTCTTGAGGGATGCTACCAATTTCACATCAGGCTTTATCGCGGAAGGATTAGG
GGCGTTTTTCGACGGCTTAATCCTGACTGGGATGGATGGTGAAGCACACCGTAGAAC
TAGGTCCCTATTGCAGCCGGTTTTCATGCCCGACGTTGTCAACCGTTGGAGGGAAAC
GAAAATGGCACCAATAGTCAGGAATGAATATATTGAACCGATGGTCCCGAAAAGGC
GTGCTGACCTTATGGACTTTGGACTTCACTTCCCTATACGTCTAATCTACAGTTTGAT
AGGGTTCCCAGACAATAGGCCGGAGCAGATCGAACAGTACGCTGCCTGGGCACTTG
CCATCCTGGCAGGGCCGCAGGTGGACGCAGAGAAAGCAGCCCAGGCGCGTAAAGCT
GCGATGGAAGCCGCCCAGGCGCTTTACGACGCAGTTAAACTTGAAGTTACAGAGGT
CCGTAAAAATGGAGCCCAGGGTGACGATCTAATCTGCAGGCTAATTAGAGCTGAGT
ATGAAGGCCGTCATCTTGATGATCATGAAGTCACAACCTTTGTCAGGTCACTTCTGC
CAGCCGCTGGAGAGACAACTACGAGAACGTTCGGTTCACTGATGGTCGCTCTTCTGG
AAAGACCTGAATTACTGGAACGTGTTAGGGCTGATAGATCCTTAGTGCCAAAGGCG
ATCGACGAAGCGGTGAGGTTCGAACCAGTAGCTACTTTTAAGGTCAGGCAGGCGGC
ACAGGATACGGAAATTGGCGGGTTCTCCATACCGAAGGGAGCAATGGTTCAATGTA
TAGTCAGTTCCGCCAACAGGGACGAAGAGGTCTTCGAAAACTCTGAGAGCTTTGAC
ATTGATAGAAAGCTGAAACCGTCATTCGGCTTCGGGTTCGGTCCACATATGTGCATA
GGGCAGTTCATTGCAAAGGTCGAGTTATCAGTGGCCGTAAACACTATTTTAGATTTA
TTGCCAAACCTTCGTTTAGATCCAGACAGGCCGAAACCTAGAATAGTAGGTGCTCAG
CTGAGAGGTCCCCACGCGCTTCATGTTATTTGGGACTAA
>THCdeg_6
Seq. ID NO: 34
ATGAGTAGTTCTCCCTCAGTGGCAGAGTTAAGCCAGGAGTTGGGAGAAGCATTCCGT
CTATCCAGCATGGACGATCCGTATCCGATGTTGGCAGAGAGGAGAAGAGAGACTCC
TGTGATGAAAGGGGATATAATGGTGGCCTTAGGTGCGCCAAGCTATATGGGCCAAC
ACGCCGGCGAGACTCATACTGTATTCAGGCATGACGACGTAATGGCTATCCTTCGTA
ATCACGAAACGTTCTCAAGCAGTATTTGGGAAATTTCTCAAGGGCCACTAATAGGTA
GATCCATCCTGGCAATGGACGGGGCAGAGCACAGACAATGGAGGGGATACTTACAG
TCTGTATTTGGAGGGAAGCTATTGTCTTCATGGGATGAGTCCATATTCAGGCCCCTT
GCGGCAAAGTATGTCGCAGACCTTGCTAGTAAGAGAGGTGCGGACCTAATAGCGAT
GGCGTTGGAGTATCCCCTTAGGGCTATCTACGAGATCCTGGGCTTGGAAGATTTTAA
AGACAATTATGAGGAATTTCACGCTGACGTACTGACGATTCTACTAGCCCTATGGTC
TACACCCGACCCAGCGCAAGCCGACCAGTTCTTGCTACGTTTTCAAAAAGCTACGGA
AGCATCTGCTAGGAGTTGGGACCGTCTACTACCCATCGTCCAAAGAAAGAGGGCGG
CGGGTGCGAGCAGGAACGACCTTATTTCTAGTCTAATTAGGGCGGAATACGAGGGT
GGTGTTTTGGATGATGAACAAATCACCAGTTTTCTTAGGTCTCTATTGCTTGCAGCCA
CCGATACTACTACCCGTCAGTTTTTGAATACTTTGACCTTGCTTTTACAGAGGCCAGA
TGAGTTGGATCGTATTCGTAGGGATAGGAGCAGATTGAGATTGGCATTGGCGGAAG
GGGAAAGGTTGGAACCGCCCGCCCTATTCATACCCCGTATGATAACGAGGGATGTT
GTTATTAGGGGTACCGAGTTGACGGCGGGGACCCCCTTACTACTTGCCATCGGGAGC
GCGAATCGTGATCCTGAAGCCTACCCACCCGACCCAGATGAATTTCGTATCGATAGA
ACGGGACCACACCACGCCACGTTCGGTTTTGGTACTCACATCTGCTCCGGGATGAAC
ACTACTCGTCGTGAGATAGCAGCCTTGATCGATGCGATGTTAGACGGGCTACCGGGA
CTTCGTGTCGATCCCGACGCTCCCGCGCCACTTATATCAGGGATTCATTTTAGAGGC
CCATCCGCACTGCCGGTTGTATGGGATTAA
>THCdeg_7
Seq. ID NO: 35
ATGAGTAGTGATTACTCCAGGACACCCGAGTCCCTGCGTCCGGCTGATAGTTATGCC
GCGCTATCCTACTCCACAGTTAATGCTGCTCTGCGTAACGATAGAGTATTCTCTTCAA
AGATGTACGACTCCACCATTGGAGTGTTTATGGGTCCTACAATCTTGGCTATGAGTG
GCACTAAACACAGGGCTCACAGAAACCTTGTATCCGCTGCTTTCAAGCCGCAAAGTC
TGAGAGTTTGGGAACCTGATATTGTAAGACCAATTTGTAATGCACTAATTGATGAGT
TTGCCGGGACAGGCCACGCAGACCTGGTTCGTGACTTCACGTTTGAATTTCCTACTA
GAGTAATAGCTAGACTGCTAGGCTTACCAGCGGAGGATTTGCCATTCTTTAGAAAGG
CCGCAGTGGCGATTATCAGTTATGCAGGAAACGTTCCGAGAGCGTTGGAAGCGTCC
GAGGACCTGAAGAACTACTTTCTAGGACACATAGAGCAAAGACGTAGTCAGCCTAC
CGATGATATTATATCTGATTTAGTTACGGCAGAAGTTGAAGGAGAGCAATTGACCGA
TGAGGCAATTTATTCATTCCTGCGTCTGCTGTTACCTGCTGGGTTAGAGACAACCTAC
CGTAGTAGTGGAAATTTGCTGTACCTATTATTACGTCACCCAAGGCAATTTGCGGCC
GTGCAAGGAAACCATGGTCTTATTCCTCAAGCCGTAGAAGAGGGTCTGCGTTATGAG
ACGCCTCTAACGTTTGTCCAGCGTTTCACAACCGAAGACACGGAGCTTGGGGGCGTT
CCTGTTCCCGCGGGCGCAGTAGTAGATTTAGTCTTGGGCTCTGCCAACAGGGATGAA
GACAGATGGGAACGTCCGGGCGAGTTCGACATATTCAGAAAACCCGTGCCCCATAT
AAGTTTTACGGCGGGAGCCCATACTTGTTTAGGACTGCATTTAGCCAGGATGGAGAC
GAGGGTTGCTGTCGAGTGCCTACTAACTCGTCTGACTAACTTCAGACTTCAGGATGA
AGGAGACCCCCACATAACCGGACAGCCATTCCGTAGTCCGAATCTTCTTCCAGTAAC
TTTCGACGTGGTTTAA
>THCdeg_8
Seq. ID NO: 36
ATGAGTAGTCCGACGCCAAGGTGGAGGATACCGGTGCTAGGCGATCTTCTTTCAGTT
GACCCCGCGAAGCCTGTTCAAAAGGAAATGGCTATGGCGGCGGAACTAGGTCCGTT
ATTCGAGCGTAAGATTATAGGGAGCAGACTTACAGTCGTTAGCGGCGTGGACCTAG
TCGCTGAGGTCAACGACGAGAAACATTGGGCTAGAGCTTTGGGGAGGCCCATACTG
AAGCTAAGAGATGTTGCAGGTGATGGGTTGTTCACAGCGTTCAACAGCGAGCCTGC
ATGGGCTAGGGCTCATAGCGTGTTGGGCCCTGGCTTCTCACAAAGCGCATTGAGAAC
CTACCATGGCAGTATGACTAGGGTGTTGGATGATTTGGTGGCGACATGGGACGATGC
AGCGGCATCAGGTGCCCGTGTCGATGTCGCTCGTGATATGACGAGACTGACTTTCGA
TGTGATTGGCAGAGCCGGCTTTGGTCGTGACTTCGGCTCTTTGAGGGGTGATGATCT
GGACCCCTTTGCCGCTGCCATGGGTAGAGCACTTGGTTATGTGAATCAAACATCAAA
TGACATACCACTTCTACGTATGGTATTCGGTAGGGGCGCGGCCAAAAGGTACCAGA
CAGACGTCGCATTTATGCGTGATACCGTAGACGAGCTAGTTGCGAGCAGGGCTGGG
CGTGCCGAGAGGAGCGATGATCTTCTTGACCTAATGTTACACAGTGCTGACCCGGAT
ACTGGGGAGAGGTTGGACATGGAAAACATTAGGAATCAAGTTCTTACCTTCCTTGTT
GCCGGTAATGAGACAACAGCTAGTACATTGGCGTTTGCACTGTATTTTCTGGCTAGA
GAGCCCGAAGTTGTCGAAAGAGCCAGGGCCGAGATCGCGGATGTAGTCGGAGACGG
TGAGATCGCTTTCGAGCAAGTGGCTAAATTACGTTATGTCAGGAGGGTTGTCGATGA
GACGTTAAGACTGTGGCCTGCCGCTCCGGGCTACTTTCGTAAAGTTAGGCATGATAC
GGTATTAGGCGGTCGTTATCCCATGCCTAAAGGTTCATGGGTTTTCGTGCTGTTACCA
CAGCTTCATCGTGACCCTGTATGGGGTGACGATCCGGAAAGGTTTGACCCCGATAGA
TTCGCACCAGACGCTGTGCGTGCAAGGCCTAAAGATGCTTATAGACCGTTTGGCACA
GGCCCCAGAAGTTGTATAGGGAGGCAGTTCGCGTTGCACGAGGCGGTACTTGCCCT
GGCGACGTTGTTGAGAAGATACGACGTTGCCCCAGACCCAGCATATCGTTTAGATAT
CGTAGAAGCTGTAACGCTAAAGCCTAGAGGCTTTGAGCTTACACTACAGAGGAGGT
AA
>THCdeg_9
Seq. ID NO: 37
ATGAGTAGTTCAGCATCTTCCCAGTCTAACCTAGAGCAAGTCTTTGCCAACGTAGCA
TCAAATTATAGAGGAGCAGACATAGACTTGCACGCAGTATATCGTGAAATGAGGGA
AAAGTCTCCGGTTCTGCCAGAGAATTTCATGGCCCGTCTAGGTGTGCCCTCAATCGC
TGGTCTGGACCCCGACCGTCCTGCCTTCACGCTATTCAAATATGACGACGTTATGGC
AGTCATGCGTGATGCTACAAACTTTACTTCAGGCTTTATAGCCGAGGGTTTGGGGTC
CTTCTTTGATGGACTTATATTGACAGCAATGGACGGTGAGGCACATAAAAATATACG
TTCCTTATTGCAGCCTGTCTTTATGCCAGAAACCGTTAACAGATGGAAAGAGACTAA
GATCGACAGAGTGATAAGGGAAGAATACCTGCAACCAATGGTGGCATCCAAAGGGG
CGGATATTATGGAGTTTGCTCTGTATTTTCCAATTAGAGTTATTTATTCCCTGATAGG
ATTCCCAGAAGATAGACCCGAGGAAATCGAACAATACGCAGCATGGGCGCTCGCAA
TCCTGGCGGGCCCACAAGTGGACCCCGAAAAGGCAGTTGCCGCGCGTGGAGCCGCT
ATGGAAGCTGCCCAGGCGTTGTATGACGTGGTGAAAGTCGTAGTCGCGCAGAGGCG
TTCTCAAGGTGCCACGGGAGATGACTTGATATCCAGGCTGATACGTGCCGAGTACGA
AGGTCGTAGCCTGGATGACCACGAGATAACCACGTTCGTCAGGTCCCTACTGCCCGC
GGCATCTGAGACAACGACCAGAACATTCGGGACATTGATGACTTTACTATTGGAAA
GACCGGAGCTTCTAGCACGTATTCGTGAAGACAGAAGCCTGGTGCCAAAAGCAATT
GATGAGGCTGTTAGGTACGAACCTGTAGCAACCTTTAAGGTCAGACAGGCCGCTAA
AGACGTTGAGATACGTGGGGTAGCCATTCCTCAAGGAGCCATGGTTAGCTGTATTGT
AACATCTGCAAATAGGGACGAAGACGCGTTCGAAAATGCTGATACTTTCAATATCG
ATAGAAGAGCGAAACCATCATTCGGTTTCGGATTCGGCCCACACATGTGCATTGGAC
AATTTGTAGCCAAGACCGAGATAAATTGCGCTCTAAATGCTATTCTGGACTTAATGC
CCAATATACGTCTTGATCCCGATAAACCTGCACCAGAAATCATAGGTGCCCAGCTAA
GGGGTCCCCACCATGTACACGTCATTTGGGACTAA
>THCdeg_10
Seq. ID NO: 38
ATGAGTAGTACTGCCACAGAATTGAGGGATGCACCTGGGAGTGCGCCAGGCCTACC
CAGGAGATCCATGTTATCCCTTTTACCCAGAATGGCACGTGATAGATTGTCAGTTAT
GACAAGTGTAGCGGCGCGTTATGGGGACGCCGTGACGTTGCCCTTGGGCTTATCAAC
GTTACACTTCTTCAACCACCCCGACTATGCTAAGCACGTACTGGCTGATAATAGCTC
AAACTACCACAAGGGCATCGGCTTAATCCACGCGAAGCGTGCGTTAGGTGACGGAC
TTCTTACGTCAGAGGGTGAGTTATGGAGAAAACAGAGGAAAACCATTCAGCCGGCA
TTTGCTGTTAAAAGGTTGGCTGGACAAGCGGGGGCAATCGCAGAGGAAGCTGATAG
GTTGGTAGAGCATCTGCTGGCCCGTCAAGGGAGAGGGCCAGTTGACATCAGGCACG
AGATGACTGCCCTTACCCTAGGTGTGTTAGGCCGTACCCTACTTGATGCGGACTTAG
GCGCTTTCGGTTCAGTGGGCCACTGGTTCGAGGCTGTACAAGACCAGGCGATGTTTG
ACATGATGAGCCTTGGTACTGTACCACTATGGTCTCCCTTGCCCAAGCAACTGAGAT
TCAGGAGAGCGAGGAGGGAATTGGAGTCAGTGGTGGACCGTCTAGTAGCTCAGCGA
GGGGATAGACCTAGGGCAGACGGCGATGATGTTGTGTCCAGGCTTGTCGATAGTAC
AGGAAGGGAGCGTGATCCTGCACTAAGGAGAAAGAGAATGCACGATGAATTGGTG
ACTCTGTTACTGGCGGGCCACGAGACAACAGCATCTACCCTTAGCTGGACATTCCAT
TTGGCCGATGAACACCCTGAGGTCTGGGAGCGTTTACACGCCGAAGCCGTGGAGGT
ACTAGGTGATAGGCGTCCGGTCTTTGAAGATTTACATCGTTTGCGTTACACAAATCG
TGTACTAAATGAAGTTATGAGGTTGTACCCTCCAGTTTGGCTGCTTCCTAGAAGAGC
TGTCGCTGACGACGTTGTTGGAGGATATAGAGTACCGGCTGGATCTGATGTTTTAAT
CTGCCCTTATACGCTACACAGACATCCTGAGTTTTGGGAGCTTCCAAGTAGGTTCGA
CCCTGATAGGTTCGATCCGGAAAGGTCTGCCAACAGGCCCAGATATGCTTACATTCC
TTTTGGTGCGGGTCCACGTTTTTGCGTTGGTAACAACCTAGGACTAATGGAGGCAGC
CTTCGTTATTGCAGCTATAGCAAGAAGAATGAGACTAAGGAAGGTTCCGGGAGGAA
CTGTCGTTCCTGAACCAATGTTGACTTTACGTGTTAGAAGTGGGCTGCCTATGACGG
TGCACGCGCTTGACCGTTAA
>Oxid_1
Seq. ID NO: 39
ATGAGTAGTCAGAGAAGAGATTTCCTTAAGTATTCTGTGGCCCTTGGCGTTGCCTCA
GCTTTGCCCCTGTGGTCTAGGGCCGTCTTTGCCGCGGAAAGACCGACTCTTCCGATC
CCCGACTTGCTGACGACCGATGCCAGAAATAGAATTCAACTAACCATCGGGGCAGG
CCAGAGTACCTTCGGCGGCAAAACCGCCACGACTTGGGGTTACAATGGTAACCTGTT
AGGGCCTGCTGTCAAACTACAACGTGGCAAAGCGGTCACGGTAGACATATATAACC
AACTAACTGAGGAAACAACGTTGCACTGGCATGGCCTAGAAGTGCCCGGCGAAGTA
GATGGAGGTCCCCAGGGCATTATCCCCCCAGGGGGTAAAAGATCAGTCACATTGAA
TGTCGACCAGCCTGCGGCTACATGCTGGTTCCATCCACATCAGCACGGGAAGACGG
GGAGGCAAGTGGCAATGGGGCTTGCTGGTTTAGTTGTAATAGAGGATGACGAGATC
TTGAAACTAATGCTTCCAAAACAATGGGGGATAGACGACGTACCTGTAATCGTTCAA
GATAAAAAATTTAGCGCAGATGGGCAAATCGACTACCAGCTGGATGTCATGACAGC
GGCAGTGGGATGGTTTGGGGACACACTGCTAACTAACGGGGCTATATACCCCCAGC
ACGCCGCTCCAAGGGGTTGGTTACGTCTGCGTCTATTAAACGGTTGCAACGCCCGTA
GCTTAAATTTTGCGACCTCAGACAATCGTCCCTTGTATGTAATCGCGAGCGACGGTG
GATTATTGCCGGAGCCCGTAAAAGTCTCCGAGTTGCCTGTGCTGATGGGAGAAAGAT
TTGAGGTTTTGGTGGAGGTTAACGATAACAAGCCCTTTGATCTAGTTACCCTTCCTGT
AAGCCAAATGGGGATGGCCATCGCTCCATTTGACAAACCTCACCCCGTCATGAGAA
TTCAACCCATCGCTATAAGTGCGTCTGGTGCGCTTCCAGATACTCTGTCTAGCCTACC
AGCGCTACCGTCTCTTGAAGGTTTAACAGTAAGGAAACTGCAACTATCTATGGATCC
AATGTTAGATATGATGGGAATGCAAATGTTAATGGAGAAGTACGGTGATCAGGCAA
TGGCGGGTATGGACCACTCCCAGATGATGGGCCACATGGGTCACGGCAATATGAAT
CATATGAACCATGGGGGCAAATTCGACTTCCATCACGCTAACAAGATTAATGGTCAA
GCCTTCGACATGAACAAGCCTATGTTTGCCGCGGCTAAGGGTCAGTACGAAAGATG
GGTCATCTCCGGGGTAGGGGACATGATGCTGCATCCGTTCCACATCCATGGCACACA
ATTTAGGATTCTTAGTGAAAATGGAAAACCTCCTGCTGCACATAGGGCGGGATGGA
AGGATACTGTGAAGGTGGAAGGTAACGTTAGTGAGGTGCTAGTCAAATTCAATCAC
GATGCCCCCAAAGAACATGCCTATATGGCCCACTGTCACCTTTTGGAGCATGAGGAT
ACGGGAATGATGCTAGGTTTCACAGTC
>Oxid_2
Seq. ID NO: 40
ATGTCTAGCAGACTGAGCTTCTTAACGTCATTGGTTACATTGGCGTTGGTATCTAGC
ACGTATGCCGGAGTTGGGCCCGTTGTAGATCTTACAGTTTCAAACGCCGTTATTTCA
CCTGATGGGTTTGACAGAGACGCGATTGTAGTTAACGGCGTGTTCCCAGCGCCTCTT
ATCACAGGTAAGAAAGGTGACAGATTCCAGCTAAATGTGATCGATAACATGACTAA
CCATACTATGCTGAAGTCAACAAGTATTCATTGGCATGGGTTTTTTCAAAAAGGTAC
TAACTGGGCCGATGGCGGGGCCTTTGTCAACCAATGTCCAATCGCTCCTGGCCACTC
CTTCCTATACGATTTCCGTGTACCGGACCAAGCAGGCACATTCTGGTACCACTCACA
CCTTTCTACGCAATATTGCGACGGTTTAAGAGGGCCCATCGTGGTATATGACCCCAA
CGACCCTCATGCGGACCTGTACGATGTGGATAATGATTCCACTGTGATCACACTTGC
CGACTGGTACCACGTTGCCGCCCGTCTTGGGCCCAGATTTCCGCTGGGAGCAGATTC
TACGGTTATTAACGGTCTTGGGCGTTCCCTTAGCACGCCTAACGCTGACTTAGCTGT
GATCTCAGTCACTCAAGGTAAAAGATATAGGTTCCGTCTAATATCTCTTTCATGCGA
CCCCTTCCATACTTTTTCTATCGATGGACATGACTTGACCATTATAGAGGCGGACAG
CGTGAACACGGAGCCCTTGGTGGTGGATGCAATTCCAATCTTCGCCGGACAACGTTA
TTCTTTTGTCTTGAGCGCCGTCAAGGACATAGATAACTATTGGATACGTGCGGACCC
AAACTTTGGAACTACAGGCTTTGCATCAGGTATCAACTCAGCGATCCTTCGTTATGA
CGGGGCTGCACCTATTGAACCAACCGCTGTTTTAGCTCCGGTAAGCGTTAATCCCTT
GGTTGAGACGGATTTGCACCCGCTTGAGGATATGCCTGTACCCGGTAGACCAACAA
AGGGTGGCGTTGATAAAGCAATCAACCTGGATTTTAGTTTTAGCTTCCCTAATTTTTT
CATTAACAATGCCACATTTACAAGCCCCACAGTGCCTATCCTGCTACAGATAATGTC
CGGCGCGCAAGCCGCGCAGGATTTATTGCCTTCTGGTAGCGTGATTGAACTGCCAGC
GCAGTCCACCATAGAACTAACTCTTCCCGCGACGGTCAATGCCCCCGGAGTGCCACA
TCCATTTCATTTGCATGGCCACACATTCGCCGTAGTACGTTCCGCCGGTAGCACTGC
CTACAATTACGACAACCCTATTTGGCGTGACGTCGTATCCACTGGCACGCCCGCCGC
AAATGACAACGTCACTATTAGATTTACAACGGACAATCCCGGACCTTGGTTTTTACA
TTGCCACATTGACTTCCACCTTGAGGCTGGCTTCGCCGTGGTATTCGCGGAGGGTGT
GCCGCAGACCCAAGTGGCGAATCCAGTACCTCAAGCGTGGGAGGAACTGTGCCCGA
TTTATGACGCATTACCGGAAGATGATCAG
>Oxid_3
Seq. ID NO: 41
ATGTCTAGTTTTAAAGTCAGCTGTAAGGTCACTAACAACAATGGTGATCAGAACGTA
GAAACGAATTCCGTTGATAGAAGGAATGTTCTGCTGGGCCTGGGGGGGCTATATGG
TGTCGCTAATGCCATCCCGCTAGCAGCCTCAGCGGCTCCAACGCCACCACCAGACCT
AAAGACTTGTGGGAAGGCGACGATAAGTGACGGGCCTCTAGTTGGATACACCTGTT
GTCCTCCCCCTATGCCTACAAATTTTGACAATATACCCTACTATAAGTTCCCAAGCAT
GACAAAGCTTAGAATCCGTAGTCCGGCACATGCCGTTGACGAAGAATATATCGCTA
AATACAATTTAGCGATTTCCAGGATGAAAGATCTAGATAAAACCGAACCCTTAAAC
CCTCTAGGGTTCAAGCAGCAGGCTAACATCCACTGTGCGTACTGTAACGGTGCGTAT
GTGTTCGGCGACAAGGTACTTCAGGTACATAACTCCTGGCTGTTCTTCCCCTTTCATC
GTTGGTATTTATACTTCTATGAGAGGATATTGGGCAAGTTAATAGATGATCCCACGT
TTGCTCTGCCATATTGGAATTGGGATCACCCAAAAGGCATGCGTTTGCCGCCGATGT
TTGACAGAGAGGGTACATCCATCTATGATGAAAGGAGGAATCAGCAAGTGCGTAAT
GGGACCGTCATGGATTTGGGATCATTCGGAGACAAAGTAGAAACGACCCAACTGCA
ACTTATGTCCAACAATTTGACTTTGATGTATCGTCAAATGGTCACAAATGCGCCCTG
CCCACTACTGTTTTTTGGAGCCCCGTATGTTCTTGGAAACAATGTAGAAGCCCCTGG
CACAATTGAAAATATACCGCACATTCCCGTGCATATATGGGCTGGCACGGTGCGTGG
CTCCACCTTCCCTAACGGGGATACGTCTTACGGAGAAGACATGGGTAATTTTTACTC
CGCAGGTTTAGATAGCGTTTTTTACTGCCATCATGGAAACGTTGATCGTATGTGGAA
CGAGTGGAAGGCTATAGGTGGTAAGAGGCGTGACCTGTCTGAAAAAGATTGGTTGA
ATAGTGAATTTTTTTTTTATGATGAGAACAAGAAGCCGTATAGGGTCAAAGTTCGTG
ATTGCCTGGACGCAAAGAAGATGGGCTACGATTATGCGCCCATGCCCACTCCCTGGC
GTAATTTCAAACCCAAAACGAAGGTGAGCGCAGGCAAGGTCAACACATCATCCCTT
CCGCCTGTCAACGAGGTTTTTCCCTTGGCTAAAATGGATAAGGTGATTAGTTTTTCA
ATAAACAGGCCGGCTAGCTCAAGAACACAGCAAGAAAAAAATGAACAGGAGGAGA
TGTTGACATTTGATAACATCAAGTACGACAATCGTGGTTACATTCGTTTTGACGTCTT
CTTGAACGTCGACAACAACGTTAACGCGAACGAGCTGGACAAAGTTGAATTCGCTG
GAAGCTATACCTCATTACCACATGTGCATCGTGTCGGAGAAAATGATCACACGGCCA
CCGTTACCTTCCAGCTAGCCATCACTGAACTACTGGAAGATATCGGCCTAGAAGATG
AAGAAACCATAGCTGTGACTCTAGTACCCAAGAAGGGGGGTGAAGGAATTAGTATT
GAGAATGTGGAAATTAAATTATTAGACTGT
>Oxid_4
Seq. ID NO: 42
ATGTCTGGCCAGAATAAAATGGGTCTTATACTTGTATTTCTGTTTCTGGACGGGTTGC
TTGTCTGTTTAGCTGCGGATGTGGATGTACATAACTACACCTTTGTTCTGCAGGAAA
AAAACTTTACTAAATGGTGTAGCACTAAAAGTATGCTGGTCGTAAACGGTTCATTCC
CTGGGCCAACTATTACAGCCAGAAAGGGGGATACGATATTTGTCAACGTCATAAAT
CAAGGGAAGTACGGGTTAACCATCCATTGGCATGGTGTTAAGCAACCAAGGAATCC
CTGGAGCGACGGACCCGAATATATAACTCAATGCCCGATTAAACCGGGTACGAACT
TCATTTACGAGGTCATTCTGTCAACCGAGGAGGGAACACTGTGGTGGCATGCACACT
CCGACTGGACGCGTGCCACCGTGCATGGTGCGTTAGTGATTTTACCCGCTAACGGAA
CCACATATCCTTTTCCACCCCCGTACCAGGAGCAGACGATAGTCTTAGCGAGCTGGT
TTAAAGGCGATGTGATGGAGGTAATTACATCTTCTGAAGAGACGGGGGTTTTTCCCG
CCGCGGCTGACGGGTTTACAATCAATGGCGAACTGGGAGACCTGTACAATTGCAGC
AAGGAAACCACATACAGGCTTTCCGTACAGCCGAACAAAACATATTTACTAAGAAT
TGTGAATGCAGTCCTAAACGAGGAAAAGTTTTTTGGTATAGCGAAACACACATTGAC
AGTAGTTGCTCAGGACGCTTCATATATTAAGCCTATAAATACCTCTTATATAATGAT
CACGCCTGGCCAAACGATGGATGTATTATTCACGACCGACCAAACTCCTTCTCACTA
CTATATGGTTGCGAGTCCGTTTCACGACGCACTAGACACGTTTGCAAATTTTAGCAC
TAATGCAATCATACAATATAATGGGTCCTATAAAGCACCGAAAAGTCCCTTCGTGAA
ACCGTTGCCCGTTTATAATGACATCAAGGCAGCAGATAAATTCACGGGGAAACTGC
GTTCTCTTGCCAATGAGAAGTTCCCAGTAAACGTCCCCAAGGTCAACGTTAGAAGGA
TATTTATGGCAGTCTCACTAAATATCGTTAAGTGTGCAAATAAGAGCTGCAACAATA
ATATAGGACACTCTACTTCAGCCTCCCTAAACAACATAAGTTTTGCGCTACCTCAGA
CAGATGTACTGCAGGCATATTATAGAAACATCAGCGGCGTATTCGGTAGAGATTTTC
CTACAGTTCAGAAGAAGGCTAACTTTTCCTTAAATACAGCCCAAGGCACTCAAGTAC
TAATGATAGAGTATGGCGAGGCCGTTGAGATCGTATATCAGGGTACTAATTTGGGA
GCCGCAACCAGTCATCCGATGCACCTTCATGGCTTTAACTTCTATCTAGTTGGCACG
GGTGCTGGAACGTTCAACAACGTGACTGATCCTCCCAAGTATAACCTGGTCGACCCG
CCTGAGTTGAATACTATAAACCTACCACGTATCGGCTGGGCAGCAATTAGGTTTGTC
GCGGACAACCCAGGGGTCTGGTTCCTTCACTGTCACTTCGAGAGACATACAACGGA
GGGTATGGCAACAGTCGTGATTGTGAAAGATGGCGGAACTACAAACACTTCTATGC
TACCAAGTCCCGCGTACATGCCACCATGCAGC
>Oxid_5
Seq. ID NO: 43
ATGTCTTCCCGTAAGATTTGTCTAGGGTGTTCACATTCTTTAAGCTCCCAACCCTTTA
CATATACAACTCAGAAGACTGTAAGTAGTAGGCGTATCGGTGACTCTCAGTGGCGTC
TTAGCCGTGGTTACACCCGTACGCTGACCTCTGCAAGTGCAAGCGTTGCTACAGCTC
CCGCTAAGCTACTTACGGTCAATGAAACTCAAAAATGCCTAAGGAACATGGTCCGT
GGCGGAGACGTAATTAGCTACATTCTTTCCCATTCTTCCCGTAACGCAGACCAGAAT
TTGAAAGATTTAGACAGCTTAATATTGGAGCCTGTCTGCAGTGCTACGCACGAGATG
TTCGACGTTTTCGAGATCCCAGAACACATTTTGACTCCGTTTTGCGATAACAGAAAT
GTCCCCGAGGAACAAGTCACCCGTAATCCTAATCTGAGAACCGACTGTCTGACGAT
GAAGAGGTTTGTGCTATTACAGAGCCTAGTCGCGGTTGCATCCGCCGGAATTGGGCC
AGTAGCAGATCTGTACGTAGGAAATAGAATACTGGCTCCGGATGGGTTTAACAGAA
GTACAGTTCTAGGAGGTACCAGTTCATCTGATTTTGGATTCCCAGCGCCACTAATCA
CCGGCACAAAAGGGGACAGGTTTCAACTGAACGTCATCAATCAATTAACCGACACT
ACGATGTTAAGATCAACAAGCATACATTGGCACGGGTTATTCCAGGCTGGCTCATCT
TGGGCCGACGGCCCTGTAGGAGTAAATCAATGCCCTATAGCTCCAGGAAACTCATTT
CTGTACGACTTTAACGTCCCTGACCAGGCGGGAACTTTCTGGTATCATAGTCATTAT
AGCACACAGTACTGTGATGGTCTTAGGGGGGCTTTTGTGGTAAGAGATCCTAACGAT
CCACATGCGAGTCTTTACGATGTCGATAATGATGACACAGTTATAACATTGGCTGAT
TGGTATCATACGAGCGCTAAAGAGCTATCAGGCTCCTTTCCGGCAGAAGAGGCGAC
CTTGATCAATGGGCTGGGTAGGTATAGCGGGGGTCCTACTTCCCCATTAGCTATCGT
CAATGTAGAAGCGGGCAAGAGGTACCGTTTCCGTTTGGTATCCATAAGCTGCGATCC
ATTCTACACCTTCTCCATTGATGGTCACGATTTGACCATTATAGAGGCGGACGGGGA
GAACACTGATCCACTAGTAGTGGACTATCTGGAAATATACGCTGGGCAACGTTACA
GCGTGGTGTTAAACGCGAACCAGCCAGTAGACAATTACTGGATTAGGGCAAATTCTT
CCAATGGTCCGAGGGACTTTGTTGGCGGCACAAATTCTGCCATACTGCGTTACGCCG
GTGCATCAAACTCAGATCCGACAACAGAGCTAGGGCCGCGTAATAATAGGCTTGTT
GAGAATAACCTTCATGCTCTGGGATCCCCTGGTGTGCCAGGCACGCATACGATTGGA
GAGGCCGATGTAAACATTAATCTTGAAATATTGTTTACGCCACCGAATGTCCTAACC
GTTAATGGCGCCCAATTCATTCCACCTACTGCTCCCGTTTTATTGCAGATATTGTCCG
GGACAAAACAAGCAACGGATTTGTTACCCCCAGGTTCCGTATATGTTCTGCCTAGAA
ACGCGGTAGTTGAGCTAACAATCCCGGGTGGGTCAGGCGGAAGTCCTCATCCGATG
CATCTGCATGGCCACGTCTTTGACGTAGTTAGATCAGCTGGATCAGATACCATAAAT
TGGGACAATCCGGTCAGAAGAGATGTCGTGAACATTGGGACTAGCACATCTGACAA
TGCCACGATTAGGTTCACGACCGACAACCCGGGACCATGGATTTTTCATTGTCATAT
CGACTGGCACTTGGAGGTTGGGCTGGCAGTTGTTTTTGCTGAGGATCCGGATACAAT
TGAAAATAGTACACATCCCGCTGCGTGGGATGAGCTGTGCCCAATTTACGACAACCT
TCCTTCCGACGAGTTA
>Oxid_6
Seq. ID NO: 44
ATGAGCTCCACATTGGAAAAGTTCGTAGATGCCTTACCGATCCCAGATACATTAAAG
CCGGTACAACAATCTAAAGAAAAAACGTATTACGAGGTCACGATGGAGGAATGTAC
GCATCAATTACATAGAGATCTTCCGCCCACAAGGCTATGGGGATATAACGGTTTATT
TCCTGGTCCGACGATCGAAGTGAAGAGAAATGAAAACGTATACGTAAAGTGGATGA
ATAATTTACCTTCAACACATTTTCTTCCTATAGATCATACCATCCACCACAGCGACTC
CCAACATGAAGAGCCTGAGGTAAAGACGGTAGTGCATCTTCATGGCGGTGTTACTCC
GGATGACTCCGACGGCTATCCAGAAGCATGGTTCAGCAAGGATTTCGAACAAACGG
GCCCGTACTTCAAAAGGGAAGTATATCACTACCCAAACCAGCAGCGTGGTGCCATC
CTATGGTATCATGATCATGCAATGGCCTTGACTCGTTTGAATGTTTATGCAGGTCTAG
TCGGGGCATACATTATACACGATCCCAAGGAAAAGAGATTAAAACTGCCTTCAGAT
GAGTACGATGTACCCCTACTGATCACGGACAGGACAATAAACGAGGATGGTTCTCTT
TTTTACCCCAGCGCGCCAGAAAATCCATCCCCCTCACTGCCAAACCCTAGCATTGTC
CCGGCATTTTGCGGGGAGACAATCCTTGTGAATGGTAAAGTATGGCCGTACTTGGAG
GTCGAACCAAGGAAGTATAGATTTAGGGTTATAAATGCGAGCAACACAAGAACATA
TAACTTATCCTTAGACAATGGCGGCGACTTCATTCAAATAGGATCTGATGGGGGCTT
GTTACCCCGTTCAGTGAAGTTGAATTCCTTTTCATTAGCACCTGCAGAAAGGTACGA
TATAATCATTGACTTTACCGCATACGAAGGTGAGAGCATTATCTTAGCTAATAGTGC
TGGCTGCGGGGGGGATGTCAATCCTGAGACGGACGCGAATATTATGCAATTTAGAG
TTACAAAGCCTCTGGCCCAAAAGGATGAATCCAGAAAACCAAAGTACTTGGCATCC
TATCCGTCAGTTCAACATGAGAGGATTCAAAACATAAGGACACTGAAATTAGCAGG
TACGCAAGACGAATATGGTCGTCCGGTACTTTTGCTGAATAATAAGCGTTGGCACGA
TCCAGTTACTGAAACGCCTAAGGTGGGTACCACCGAGATTTGGAGCATAATAAATCC
CACGAGAGGCACCCATCCCATTCACCTACATCTTGTCAGTTTCAGAGTCTTAGACCG
TCGTCCGTTCGATATAGCTCGTTATCAGGAGTCAGGGGAACTTTCCTACACTGGACC
TGCTGTACCGCCGCCACCGTCAGAAAAGGGTTGGAAGGACACGATCCAGGCCCATG
CGGGTGAAGTTCTAAGAATCGCAGCTACCTTCGGTCCGTACAGCGGGAGGTATGTGT
GGCACTGTCATATCTTGGAGCACGAAGACTACGATATGATGAGGCCTATGGATATCA
CTGATCCACACAAG
>Oxid_7
Seq. ID NO: 45
ATGAGCTCTGTGTTTAGTGCTGCGTTTTCCGCATTCGTTGCCTTAGGTCTAACTCTGG
GCGCTTTTGCTGCCGTTGGCCCGGTCGCGGACATCCACATTACCGATGATACCATAG
CACCTGACGGATTTAGTAGGGCTGCCGTACTGGCAGGTGGGACCTTCCCAGGGCCCC
TAATCACCGGGAACATGGGAGACGCCTTTAAGTTAAACGTCATCGACGAGTTGACG
GATGCCTCTATGTTGAAAAGTACCAGTATCCATTGGCACGGCTTCTTTCAGAAGGGA
ACGAACTGGGCTGATGGCCCAGCTTTCGTGAATCAATGCCCCATTACAACTGGAAAC
TCCTTCTTGTATGACTTCCAAGTGCCAGACCAAGCAGGTACTTATTGGTATCACTCCC
ACCTAAGCACTCAGTACTGTGACGGACTGAGAGGTGCCTTCGTGGTTTACGACCCAA
GTGATCCGCACAAAGATCTGTACGACGTGGACGATGAATCCACCGTCATAACCCTA
GCAGACTGGTACCACACGCTGGCCAGGCAGATTGTGGGAGTGGCGATTAGCGATAC
CACGCTTATCAATGGCCTGGGGCGTAATACAGACGGACCCGCAGATGCTGCCTTAG
CCGTGATCAATGTAGAAGCTGGCAAAAGATATAGATTTCGTTTAGTAAGCATCAGTT
GCGACCCGAATTGGGTGTTTAGTATTGACAATCATGACTTTACGGTTATTGAGGTAG
ACGGCGTGAACAGCCAGCCTCTGAATGTTGACAGCGTACAAATATTTGCAGGGCAG
AGGTATTCCCTAGTGTTGAACGCGAACCAGCCCGTCGATAACTATTGGATTAGGGCT
GATCCTAACCTTGGTACCACAGGGTTCGCGGGTGGAATAAATTCAGCAATTCTACGT
TATAAGGGTGCGGCCGTTGCCGAGCCGACTACATCCCAAACCACAAGCACCAAGCC
CTTATTGGAGACTGACTTGCACCCCTTGGTTAGTACACCAGTCCCAGGATTACCGCA
ACCTGGCGGCACGGATGTAGTCCAAAACCTTATTCTAGGCTTCAATGCTGGGCAGTT
CACAATCAATGGCGCATCCTTTGTGCCACCAACAGTTCCAGTTTTGTTACAAATCTTA
TCTGGAACAACGAACGCGCAAGACCTGCTACCGTCCGGTAGTGTATTTGAGTTACCG
TTGGGAAAAACGGTCGAATTAACCCTGGCAGCCGGCGTTTTAGGCGGACCACACCC
GTTTCACTTACACGGTCATAATTTCCATGTCGTCAGGTCCGCTGGACAGGACACGCC
CAATTACGACGATCCAATTGTCCGTGACGTTGTCTCAACCGGAGCGTCTGGGGACAA
TGTTACAATTAGGTTTACTACCGACAATCCTGGGCCCTGGTTCCTACACTGTCATATC
GATTGGCACCTAGAGGCAGGCTTTGCGGTTGTATTCGCAGAGGCAGTGAATGAGAC
TAAATCTGGCAATCCTACACCGGCAGCCTGGGATAACCTATGCACTCTTTACGATGC
TCTAGCTGATGGTGACAAG
>Oxid_8
Seq. ID NO: 46
ATGTCCTCCTGTTTGGCCGCTATATGGTCAAGGAAAAGAGCGGAGCATGCCGCGTCA
AGGCTTCCAGCTTTACAGGAGAAAAGGTCCACACTAAGCTACGCGTATGCTAGGTTA
GATGGCAGTCTTGCGAGTATGTTTCCAAATAGGTTTTGGTCAAGCGTTAGTCTTGGA
GCTAGGATTAAACCGGTGGATGGGAGTAGTGAAGAACCCACCGCAAGGCCCAGCAG
CTGTGCCAGGCCCTTCTTACACTCAGCATCATCTGAATCAGGGTTCGTCTCCTCCTCA
CGTCCGACCAGCTTTTGCGTTACGTGCTCCCGTCGTTGGAGATGCTGTAGTCTTTTGG
CAATGCTGGGATTCAGGTTCTTACACACAAGCGTCCTTGCTGCATTGACTCTTAGTCT
AAAGAGTTATGCGGCGATAGGACCGGTTACAGACTTGACCGTCGCTAATGCGAATA
TTTCACCCGATGGTTATGAAAGAGCTGCGGTGTTAGCCGGCGGTTCATTTCCCGGCC
CACTAATTACTGGCAGAAAGGGGGACCACTTTCAGATTAATGTAGTAGATCAGCTA
ACCAACCACACCATGCTTAAAAGCACCTCTATCCATTGGCACGGGCTGTTCCAGAAA
GGGACTAACTGGGCAGACGGGCCGGCGTTTGTTAACCAGTGTCCCATCTCCACTGGG
AACTCCTTCTTATACGACTTCCATGTTCCTGATCAAGCGGGGACTTTTTGGTATCATT
CCCATCTAAGCACACAGTACTGTGATGGTCTAAGGGGTGCCATGGTGGTGTATGACC
CCAATGACCCTCACAAGAACCTTTATGACGTAGATAATGACGATACCGTAATAACCC
TAGCAGATTGGTATCATGTAGCCTCTAAGCTGGGGCCTGCTGTCCCTTTTGGGGGGG
ACTCAACCTTGATAAATGGCAAGGGTCGTAGCACTGCAACACCAACCGCCGACCTT
GCTGTCATTAGTGTAACTCAAGGTAAAAGATATAGGTTCCGTCTGGTGTCACTTTCA
TGCGACCCGAATTTCACGTTTAGTATAGATGGTCATGCCCTGACCGTAATAGAGGCC
GATGCTGTTTCAACTCAGCCATTAACTGTCGACAGTATCCAAATATTCGCGGGTCAA
AGGTACTCCTTTGTGCTTAATGCCAATCAGTCCGTTGATTCATACTGGATTCGTGCCC
AGCCATCCCTTGGTAATGTGGGCTTTGATGGAGGGCTTAATTCTGCGATCCTTCGTTA
TGACGGGGCTGCGCCGACCGAACCATCCGCGCTAGCTGTTCCAGTCTCTACTAATCC
TTTGGTTGAGACGGCACTGAGGCCGCTTAATTCAATGCCCGTCCCCGGTAAGGCTGA
GGTGGGCGGTGTGGATAAAGCGATTAACCTTGCGTTTAGTTTCAATGGCACGAACTT
TTTCATCAATGGGGCAACGTTTGTGCCGCCCGCCGTGCCCGTTCTACTACAGATCAT
GAGTGGCGCCCAGAGTGCTAGTGATCTTCTTCCTAGCGGCTCAGTGTTTGTGCTACC
CAGTAACGCTACCATCGAATTAAGTTTTCCAGCAACTGCAAACGCCCCAGGCGCTCC
ACATCCCTTCCACCTACATGGACACACGTTCGCGGTAGTACGTTCTGCTGGTTCCGC
GGAGTATAATTATGAAAATCCTATATGGAGAGACGTTGTTTCAACCGGTTCTCCGGG
AGACAATGTCACCATACGTTTCAGGACCGATAATCCGGGCCCCTGGTTTCTGCATTG
TCATATCGATCCCCATCTGGAGGCCGGCTTTGCGGTGGTTATGGCGGAGGACACTAG
GGACGTCAAGGCCGACAATCCCGAACCTAAAGCCTGGGACGATCTTTGCCCCACAT
ACAATGCGCTAGCAGTGGATGACCAA
>Oxid_9
Seq. ID NO: 47
ATGTTTCCAGGGGCGCGTATTCTGGCCACCCTGACGTTGGCACTGCATCTGCTGCAC
GGTACCAATGCCGCAATAGGACCCACTGGAGACATGTACATAGTTAACGAAGACGT
GTCCCCTGACGGCTTCACCAGGTCCGCAGTAGTTGCTAGGAGTGATCCTACCACAAA
CGGGACATCCGAAACTCTAACGGGTGTCTTAGTTCAGGGGAACAAGGGAGACAACT
TCCAATTGAACGTGCTGAACCAGTTATCAGACACAACTATGCTAAAAACGACAAGC
ATTCATTGGCACGGCTTTTTTCAATCTGGATCCACCTGGGCCGACGGCCCAGCCTTTG
TAAACCAATGTCCAATAGCTAGCGGTAATTCTTTCCTTTACGATTTTAATGTACCAGA
CCAGGCCGGCACGTTCTGGTACCACAGTCATCTGTCAACCCAGTACTGTGACGGTTT
GAGGGGACCATTTATCGTTTACGACCCAAGCGATCCCCACCTGTCTCTATACGACGT
CGACAATGCGGACACTATCATAACGCTGGAAGACTGGTACCACGTAGTGGCACCTC
AGAACGCCGTATTGCCGACGGCCGATAGCACACTAATTAACGGCAAAGGCCGTTTC
GCCGGGGGTCCTACAAGCGCCTTAGCGGTCATCAATGTTGAGTCTAATAAACGTTAC
AGATTCCGTCTTATATCCATGTCCTGTGACCCTAATTTTACGTTTAGTATAGACGGAC
ATAGCCTACAAGTGATTGAGGCCGATGCCGTGAACATTGTACCGATAGTAGTGGATT
CCATCCAGATATTCGCCGGACAAAGGTACAGCTTTGTACTTAACGCTAACCAGACCG
TGGACAACTACTGGATCAGAGCTGATCCGAATTTGGGCAGCACTGGTTTCGACGGA
GGTATCAACTCTGCTATTTTAAGGTATGCTGGAGCAACGGAAGATGACCCCACGACG
ACAAGTTCCACCTCAACTCCATTAGAAGAAACAAACTTGGTACCCCTAGAGAATCCT
GGGGCGCCAGGACCGGCGGTACCCGGCGGTGCAGACATAAATATCAATCTTGCCAT
GGCTTTCGACGTGACGAATTTCGAATTAACGATTAACGGATCCCCTTTCAAAGCACC
TACCGCCCCCGTACTTTTACAGATATTGTCAGGAGCAACGACGGCCGCTTCCTTGCT
GCCATCAGGGTCCATTTACTCACTAGAAGCCAATAAAGTAGTGGAGATTTCCATACC
TGCTCTAGCTGTGGGGGGTCCGCATCCTTTCCACTTACACGGGCATACATTTGACGTT
ATACGTTCAGCGGGGAGTACGACTTACAATTTTGACACTCCCGCTCGTAGGGATGTC
GTTAATACAGGAACGGACGCTAATGATAATGTAACAATAAGATTTGTTACCGACAA
CCCAGGGCCGTGGTTTCTGCATTGCCACATAGACTGGCACTTAGAGATAGGGCTGGC
CGTCGTTTTCGCCGAGGACGTTACTAGCATAACCGCACCTCCAGCGGCGTGGGATGA
TTTGTGTCCCATCTATGACGCATTATCTGATAGCGACAAGGATAATCCTAGGTTTGG
ATTTGCACCAGCGACAGGAGGTAAAGCAACGGGCAGGAGAAATTGGTTCTCAAAGG
CTAGGCGTAGAGCAATTTTGGTTCCCTATTTAAAACTTTTAAAATTGGGGTTGGTGA
TGGTCTTCTATATAAGAGCAGAAAGGAACCATGGTAGACTTTCCCAATCTACACCTC
CGAATCGTAGGGTAGATCAGCGTGAATTAATTACTAACACATGGGTGGAGAGGTTTT
TCCTGCACCGTCTAATGTTTCTGAAGCTTTTTGTTGGAACCGCATGTTTCATGCACAT
CTTCAATTCAGTTAGCTCACTAGGGATGTGTACGTTGAGGACCAGCCATGGGTCCTC
CGAGTCATTAGCTTCTCCATCAGCGACCATGATGTTAGGAGGCGGCCTAACTCTTCT
TAGTGCTAACATAAGACTTTGGTGTTATGCCGAGATGAGAGATTTGTACGACTTCGA
AGTTAATATCAAAAAGGCCCACCGTCTTGTAACGACTGGGCCGTATAGTGTTTCTAT
GGTTATGTTTAGCAAGGATCATTGGTTGTATCAATGCGGTCTGCGTTCCATGGTTGG
CGTTGTGCTAAGTTGTATATGGTGCGCGGAAGTTGTACTGATCAACGGAATTATGGT
CCCGGCACGTATGAAGGTGGAAGACGACGGATTGAGAAGGCACTTCGGGCGTGAGT
GGGATGAATATGCATCACGTGTGGCCTATAGGTTGGTCCCCGAGATTTAT
>Oxid_10
Seq. ID NO: 48
ATGTCAAGTAAGAGCTTTATCTCTGCCGCGACCCTACTGGTCGGAATACTTACGCCG
AGTGTGGCGGCTGCTCCACCTAGCACCCCTGAGCAAAGGGACCTGTTGGTTCCCATT
ACAGAAAGGGAGGAAGCTGCTGTTAAAGCGCGTCAGCAGTCTTGCAACACTCCCTC
AAACCGTGCATGCTGGACGGATGGCTATGACATCAATACAGACTATGAAGTAGATT
CTCCTGATACGGGTGTTGTTCGTCCCTACACTTTGACGCTGACCGAGGTTGATAACT
GGACTGGGCCTGATGGTGTCGTCAAGGAAAAGGTTATGCTGGTAAACAATTCAATA
ATCGGACCCACAATTTTTGCCGATTGGGGTGATACCATCCAAGTCACGGTGATTAAT
AACCTTGAGACCAATGGAACGAGTATTCATTGGCACGGCCTACATCAGAAGGGTAC
GAACTTGCACGATGGAGCTAATGGGATTACTGAATGCCCCATCCCGCCCAAGGGGG
GCAGAAAGGTTTATAGATTCAAAGCACAGCAATATGGAACGAGTTGGTATCATAGT
CACTTTTCCGCGCAGTACGGCAACGGTGTGGTTGGCGCGATACAGATCAACGGGCC
GGCCAGTTTACCATACGATACGGACCTGGGCGTTTTTCCTATCAGCGATTATTATTAT
TCCTCAGCGGATGAGCTAGTTGAATTGACCAAAAACAGCGGTGCACCCTTTTCAGAT
AATGTCCTTTTTAACGGAACGGCAAAGCACCCAGAAACAGGCGAGGGCGAGTACGC
AAATGTAACGTTAACCCCAGGAAGGAGGCATCGTTTGCGTCTGATTAACACGAGTGT
TGAAAACCATTTCCAAGTCTCTCTAGTTAATCATACCATGACGATCATTGCCGCCGA
TATGGTTCCAGTAAATGCTATGACCGTTGATTCACTGTTCCTGGGCGTCGGACAAAG
GTACGACGTAGTAATAGAAGCTAGTAGAACTCCAGGGAATTATTGGTTCAATGTGA
CATTCGGGGGCGGCCTGTTGTGCGGAGGCAGTAGGAATCCTTACCCAGCTGCAATAT
TTCACTATGCAGGCGCCCCTGGTGGACCGCCGACTGATGAAGGAAAAGCGCCGGTG
GATCACAACTGCTTGGATCTGCCGAACCTTAAACCTGTTGTTGCTCGTGATGTGCCA
TTATCTGGTTTCGCCAAGAGGCCCGACAACACTTTAGACGTCACTTTGGACACGACT
GGAACTCCCCTTTTCGTCTGGAAGGTAAACGGTAGTGCTATTAACATAGACTGGGGC
CGTCCGGTCGTGGATTACGTACTAACACAAAACACTTCTTTCCCACCCGGTTACAAT
ATAGTCGAGGTCAACGGCGCAGATCAGTGGTCATACTGGTTGATTGAGAATGACCC
AGGTGCGCCATTCACGCTACCGCACCCGATGCACCTACATGGGCATGACTTTTATGT
ACTAGGTAGAAGTCCGGATGAATCACCTGCTAGCAATGAACGTCACGTATTTGATCC
CGCCCGTGATGCGGGATTACTGTCCGGGGCGAACCCAGTGAGGCGTGATGTTACTAT
GTTGCCTGCGTTTGGATGGGTTGTGCTGGCCTTCAGGGCTGACAACCCCGGGGCATG
GCTTTTTCATTGCCATATAGCATGGCACGTATCCGGCGGGCTAGGTGTTGTCTACCTA
GAGCGTGCAGACGACCTGAGGGGAGCGGTATCAGACGCGGACGCGGATGACTTGGA
TAGGCTTTGCGCTGATTGGAGGAGATACTGGCCGACAAATCCGTATCCCAAATCAGA
CTCTGGTCTT
>Oxid_11
Seq. ID NO: 49
ATGTCATCCCGTTTCCAGAGCCTATTTTTCTTTGTGCTGGTAAGCTTGACTGCGGTGG
CGAATGCGGCTATTGGGCCGGTGGCTGACCTTACACTTACAAACGCACAAGTTTCCC
CAGATGGCTTCGCTAGAGAAGCGGTCGTGGTTAACGGAATCACCCCAGCACCATTG
ATTACGGGGAACAAGGGGGACAGATTTCAGTTAAATGTGATCGACCAGCTTACTAA
CCACACGATGTTGAAGACGTCTTCTATACACTGGCATGGTTTTTTCCAGCAGGGTAC
TAACTGGGCAGATGGCCCTGCTTTCGTTAACCAGTGTCCGATTGCGTCCGGTCATAG
TTTTTTGTACGACTTTCAGGTCCCTGATCAAGCGGGGACGTTCTGGTATCACTCACAC
CTAAGTACCCAATACTGTGACGGACTGCGTGGACCGTTCGTGGTGTACGACCCTAAT
GATCCCCATGCGAGCCTTTATGACATCGACAATGACGATACTGTCATAACTCTGGCG
GACTGGTACCATGTAGCCGCGAAATTAGGTCCACGTTTCCCATTCGGTTCAGATAGC
ACCCTAATAAACGGCCTTGGCAGAACTACCGGAATTGCGCCGTCTGACCTTGCAGTC
ATCAAAGTGACACAGGGCAAGCGTTACCGTTTCCGTCTGGTCTCTTTGTCCTGTGAC
CCAAACCACACATTCTCCATTGACAATCACACCATGACGATCATCGAGGCCGACTCT
ATCAATACGCAGCCACTAGAGGTGGATAGCATCCAGATATTCGCTGCTCAGCGTTAT
TCTTTCGTGCTGGACGCTAGCCAACCGGTGGATAACTACTGGATAAGAGCAAATCCG
GCGTTCGGTAACACCGGGTTTGCTGGTGGGATAAACTCTGCCATACTTAGATACGAT
GGTGCACCAGAAATCGAGCCTACTTCTGTCCAAACAACCCCGACTAAGCCTCTGAAT
GAAGTGGATTTGCACCCTTTGTCACCGATGCCAGTACCAGGATCTCCAGAACCGGGA
GGAGTGGATAAGCCACTTAACCTAGTGTTCAATTTCAATGGGACAAACTTTTTCATT
AATGACCACACCTTTGTGCCACCCTCTGTGCCCGTACTTTTGCAAATATTGAGTGGTG
CTCAGGCGGCGCAAGACCTGGTCCCGGAGGGGTCCGTGTTCGTTCTTCCTAGTAATT
CTAGCATTGAGATCTCCTTTCCAGCAACCGCTAATGCTCCAGGTTTCCCGCATCCATT
CCATCTACACGGACACGCATTTGCGGTTGTAAGGAGTGCGGGGAGTTCAGTTTACAA
CTATGACAACCCCATATTCAGGGACGTAGTAAGCACAGGACAACCAGGTGACAATG
TGACTATAAGATTCGAGACCAATAACCCCGGTCCTTGGTTCTTACATTGCCACATAG
ACTTTCACTTAGACGCGGGTTTTGCAGTGGTCATGGCCGAGGATACTCCTGATACTA
AAGCCGCGAATCCAGTGCCTCAAGCCTGGTCTGATTTATGTCCGATCTATGATGCGC
TGGATCCTTCCGATTTA
>Oxid_12
Seq. ID NO: 50
ATGAGCTCCGGACTTCAACGTTTCAGTTTCTTCGTTACGTTAGCATTAGTGGCCCGTT
CACTTGCTGCAATCGGACCAGTGGCATCCCTGGTAGTTGCAAACGCTCCAGTGAGTC
CGGACGGTTTTCTTAGGGACGCCATTGTGGTAAACGGAGTGGTACCGAGTCCACTAA
TAACTGGCAAAAAAGGAGACCGTTTCCAGCTGAATGTCGATGATACCCTGACAAAT
CATAGTATGCTTAAGAGCACGAGCATACACTGGCACGGTTTTTTTCAAGCAGGAACA
AACTGGGCCGACGGACCGGCTTTCGTCAATCAATGTCCCATCGCTAGTGGGCACTCC
TTCCTATACGATTTTCATGTTCCAGACCAAGCGGGGACGTTTTGGTACCATAGTCATC
TAAGTACCCAATACTGCGATGGGCTTCGTGGGCCTTTCGTAGTGTACGACCCAAAGG
ATCCCCATGCGTCCAGGTACGATGTCGACAATGAAAGCACGGTGATTACGCTGACA
GATTGGTATCATACCGCTGCGAGGTTAGGACCGCGTTTTCCCCTTGGAGCAGACGCC
ACTCTAATCAATGGGTTGGGACGTTCTGCCAGTACACCGACCGCCGCGCTGGCTGTT
ATAAACGTACAACATGGGAAAAGATACAGGTTTAGGTTAGTATCTATCAGTTGTGAC
CCTAATTATACATTCTCTATAGATGGTCATAACCTTACGGTCATTGAAGTTGACGGC
ATCAATTCCCAGCCCTTACTGGTTGATAGTATCCAGATCTTCGCTGCCCAGAGATATT
CTTTTGTGCTGAATGCTAATCAGACAGTTGGTAACTATTGGGTCAGGGCCAACCCTA
ACTTTGGGACGGTTGGTTTTGCTGGGGGGATCAACTCAGCCATACTAAGGTACCAAG
GTGCGCCCGTAGCAGAACCTACTACCACGCAGACAACCAGTGTAATCCCTTTGATTG
AGACCAATCTGCATCCGCTTGCACGTATGCCCGTACCGGGCTCACCGACACCAGGA
GGAGTGGACAAAGCCTTAAATCTAGCTTTTAACTTCAATGGAACAAATTTCTTCATC
AACAACGCCACGTTTACACCACCAACGGTGCCTGTATTACTTCAGATCTTAAGCGGC
GCCCAGACGGCACAGGATTTGCTGCCAGCAGGATCAGTATATCCTCTACCCGCGCAC
TCAACCATAGAAATAACGCTTCCTGCCACAGCACTTGCTCCTGGGGCTCCACACCCT
TTCCACCTACATGGGCACGCATTCGCCGTAGTGAGATCTGCGGGATCCACGACTTAC
AATTACAATGACCCCATCTTCCGTGATGTGGTGAGCACAGGGACACCAGCAGCGGG
AGATAATGTTACTATTCGTTTCCAAACTGACAACCCGGGGCCATGGTTTCTGCACTG
CCACATAGATTTTCATCTTGACGCCGGCTTTGCGATCGTGTTCGCCGAGGATGTCGC
AGACGTGAAGGCCGCCAACCCCGTTCCAAAGGCGTGGTCAGATCTATGTCCGATAT
ATGACGGCTTATCTGAAGCCAATCAA
>p450_1
Seq. ID NO: 51
MAADSLVVLVLCLSCLLLLSLWRQSSGRGKLPPGPTPLPVIGNILQIGIKDISKSLTNLSK
VYGPVFTLYFGLKPIVVLHGYEAVKEALIDLGEEFSGRGIFPLAERANRGFGIVFSNGKK
WKEIRRFSLMTLRNFGMGKRSIEDRVQEEARCLVEELRKTKASPCDPTFILGCAPCNVIC
SIIFHKRFDYKDQQFLNLMEKLNENIKILSSPWIQICNNFSPIIDYFPGTHNKLLKNVAFMK
SYILEKVKEHQESMDMNNPQDFIDCFLMKMEKEKHNQPSEFTIESLENTAVDLFGAGTE
TTSTTLRYALLLLLKHPEVTAKVQEEIERVIGRNRSPCMQDRSHMPYTDAVVHEVQRYI
DLLPTSLPHAVTCDIKFRNYLIPKGTTILISLTSVLHDNKEFPNPEMFDPHHFLDEGGNFK
KSKYFMPFSAGKRICVGEALAGMELFLFLTSILQNFNLKSLVDPKNLDTTPVVNGFASVP
PFYQLCFIPV
>p450_2
Seq. ID NO: 52
MAADSLVVLVLCLSCLLLLSLWRQSSGRGKLPPGPTPLPVIGNILQIGIKDISKSLTNLSK
VYGPVFTLYFGLKPIVVLHGYEAVKEALIDLGEEFSGRGIFPLAERANRGFGIVFSNGKK
WKEIRRFSLMTLRNFGMGKRSIEDRVQEEARCLVEELRKTKASPCDPTFILGCAPCNVIC
SIIFHKRFDYKDQQFLNLMEKLNENVKILSSPWIQICNNFSPIIDYFPGTHNKLLKNVAFM
KSYILEKVKEHQESMDMNNPQDFIDCFLMKMEKEKHNQPSEFTIESLENTAVDLFGAGT
ETTSTTLRYALLLLLKHPEVTAKVQEEIERVIGRNRSPCMQDRSHMPYTDAVVHEVQRY
IDLLPTSLPHAVTCDIKFRNYLIPKGTTILISLTSVLHDNKEFPNPEMFDPHHFLDEGGNFK
KSNYFMPFSAGKRICVGEALARMELFLFLTSILQNFNLKSLVDPKNLDTTPVVNGFASVP
PFYQLCFIPV
>p450_3
Seq. ID NO: 53
MAADSFVVLVLCLSCLLLLSLWRQSSGRGKLPPGPTPLPVIGNILQIDIKDISKSLTNLSK
VYGPVFTLYFGLKPIVVLHGYEAVKEALIDLGEEFSGRGHFPLAERANRGFGIVFSNGKK
WKEIRRFSLMTLRNFGMGKRSIEDRVQEEARCLVEELRKTKASPCDPTFILGCAPCNVIC
SIIFRKRFDYKDQQFLNLMEKLNENVKILSSPWIQIYNNFSPIIDYFPGTHNKLLKNVAFM
KSYILEKVKEHQESMDMNNPQDFIDCFLMKMEKEKHNQPSEFTIESLENTAADLFGAGT
ETTSTTLRYALLLLLKHPEVTAKVQEEIERVIGRNRSPCMQDRSHMPYTDAVVHEVQRY
IDLLPTSLPHAVTCDIKFRNYLIPKGTTILISLTSVLHDNKEFPNPEMFDPHHFLDEGGNFK
KSNYFMPFSAGKRICVGEALARMELFLFLTSILQNFNLKSLVDPKNLDTTPVVNGFASVP
PFYQLCFIPV
>p450_4
Seq. ID NO: 54
MAADLVVFLALTLSCLILLSLWRQSSGRGKLPPGPTPLPIIGNFLQIDVKNISQSFTNFSKA
YGPVFTLYLGSKPTVILHGYEAVKEALIDRGEEFAGRGSFPMAEKIIKGFGVVFSNGNRW
KEMRRFTLMTLRNLGMGKRNIEDRVQEEAQCLVEELRKTKGSPCDPTFILSCAPCNVICS
IIFQNRFDYKDKEFLILMDKINENVKILSSPWLQVCNSFPSLIDYCPGSHHKIVKNFNYLK
SYLLEKIKEHKESLDVTNPRDFIDYYLIKQKQVNHIEQSEFSLENLASTINDLFGAGTETTS
TTLRYALLLLLKYPDVTAKVQEEIDRVVGRHRSPCMQDRSHMPYTDAMIHEVQRFIDLL
PTSLPHAVTCDIKFRKYLIPKGTTVITSLSSVLHDSKEFPNPEMFDPGHFLNANGNFKKSD
YFMPFSTGKRICAGEGLARMELFLILTTILQNFKLKSLVHPKEIDITPVMNGFASLPPPYQ
LCFIPL
>p450_5
Seq. ID NO: 55
MAAILGVFLGLFLTCLLLLSLWKQNFQRRNLPPGPTPLPIIGNILQIDLKDISKSLRNFSKV
YGPVFTLYLGRKPAVVLHGYEAVKEALIDHGEEFAGRGVFPVAQKFNKNCGVVFSSGR
TWKEMRRFSLMTLRNFGMGKRSIEDRVQEEARCLVDELRKTNGVPCDPTFILGCAPCN
VICSIVFQNRFDYKDQEFLALIDILNENVEILGSPWIQICNNFPAIIDYLPGRHRKLLKNFA
FAKHYFLAKVIQHQESLDINNPRDFIDCFLIKMEQEKHNPKTEFTCENLIFTASDLFAAGT
ETTSTTLRYSLLLLLKYPEVTAKVQEEIDHVIGRHRSPCMQDRHHMPYTDAVLHEIQRYI
DLLPTSLPHALTCDMKFRDYLIPKGTTVIASLTSVLYDDKEFPNPEKFDPSHFLDENGKF
KKSDYFFPFSTGKRICVGEGLARTELFLFLTTILQNFNLKSPVDLKELDTNPVANGFVSVP
PKFQICFIPI
>p450_6
Seq. ID NO: 56
MAAALIPDLAMETWLLLAVSLVLLYLYGTHSHGLFKKLGIPGPTPLPFLGNILSYHKGFC
MFDMECHKKYGKVWGFYDGQQPVLAITDPDMIKTVLVKECYSVFTNRRPFGPVGFMK
SAISIAEDEEWKRLRSLLSPTFTSGKLKEMVPIIAQYGDVLVRNLRREAETGKPVTLKDV
FGAYSMDVITSTSFGVNIDSLNNPQDPFVENTKKLLRFDFLDPFFLSITVFPFLIPILEVLNI
CVFPREVTNFLRKSVKRMKESRLEDTQKHRVDFLQLMIDSQNSKETESHKALSDLELVA
QSIIFIFAGYETTSSVLSFIMYELATHPDVQQKLQEEIDAVLPNKAPPTYDTVLQMEYLDM
VVNETLRLFPIAMRLERVCKKDVEINGMFIPKGVVVMIPSYALHRDPKYWTEPEKFLPE
RFSKKNKDNIDPYIYTPFGSGPRNCIGMRFALMNMKLALIRVLQNFSFKPCKETQIPLKLS
LGGLLQPEKPVVLKVESRDGTVSGA
>p450_7
Seq. ID NO: 57
MAADLIPNLAVETWLLLTKLEFGFYIFPFIYGTHSHGLFKKLGIPGPTPLPFLGNILSYRKG
FCMFDMECHKKYGKVWGFYDGRQPVLAITDPDMIKTVLVKECYSVFTNRRPFGPVGF
MKSAISIAEDEEWKRIRSLLSPTFTSGKLKEMVPIIAQYGDVLVRNLRREADTGKPVTLK
DVFGAYSMDVITSTSFGVNIDSLNNPQDPFVENTKKLLRFDFLDPFFLSIIVFPFLIPILEVL
NICVFPREVTNFLRKSVKRMKESRLEDTQKHRVDFLQLMIDSQNSKETESHKALSDLEL
VAQSIIFIFAGYETTSSVLSFIMYELATHPDVQQKLQEEIDAVLPNKAPPTYDTVLQMEYL
DMVVNETLRLFPVAMRLERVCKKDVEINGMFIPKGVVVMIPSYALHRDPKYWTEPEKF
LPERFSKKNKDNIDPYIYTPFGSGPRNCIGMRFALMNMKLALIRVLQNFSFKPCKETQIPL
KLRLGGLLQPEKPIVLKVESRDGTVSGA
>p450_8
Seq. ID NO: 58
MAAALIPDLAMETWLLLAVSLVLLYLYGTHSHGLFKKLGIPGPTPLPFLGNIWSYRKGF
CMFDMECHKKYGKVWGFYDGRQPVLAITDPDMIKTVLVKECYSVFTNRRPFGPVGFM
KSAISIAEDEEWKRLRSLLSPTFTSGKLKEMVPLIAQYGDVLVRNLRLEAETGKPVTMKV
ITSTSFGVNIDSLNNPQDPFVENTKKLLRFDFLDPFFLSIIVFPFLTPILEVLNISVFPRAVTS
FLRKSVKRMKESRLEDTQKHRVDFLQLMIDSQNSKETESHKALSDLELVAQSIIFIFAGY
ETTSSVLSFITYELATHPDVQQKLQEEIDAVLPNKAPPTYDTVLQMEYLDMVVNETLRLF
PIAMRLERVCKKDVEINGMFIPKGVVVMIPSYALHHDPKYWTEPEKFLPERFSKKNKDN
IDPYIYTPFGSGPRNCIGMRFALMNMKLALIRVLQNFSFKPCKETQIPLKLRLGGLLQPEK
PIVLKVESRDGTVSGA
>p450_9
Seq. ID NO: 59
MAAELIPSFSMETWVLLATSLVLLYIYGTYSYGLFKKLGIPGPRPVPYFGSTMAYHKGIP
EFDNQCFKKYGKMWGFYEGRQPMLAITDPDIIKTVLVKECYSVFTNRRIFGPMGIMKYA
ISLAWDEQWKRIRTLLSPAFTSGKLKEMFPIIGQYGDMLVRNLRKEAEKGNPVNMKDM
FGAYSMDVITGTAFGVNIDSLNNPHDPFVEHSKNLLRFRPFDPFILSIILFPFLNPVFEILNI
TLFPKSTVDFFTKSVKKIKESRLTDKQMNRVDLLQLMINSQNSKEIDNHKALSDIELVAQ
STIFIFGGYETTSSTLSFIIYELTTHPHVQQKVQEEIDATFPNKAPPTYDALVQMEYLDMV
VNETLRMFPIAGRLERVCKKDVEIHGVTIPKGTTVLVPLFVLHNNPELWPEPEEFRPERFS
KNNKDSINPYVYLPFGTGPRNCLGMRFAIMNIKLALVRILQNFSFKPCKETQIPLKLYTQ
GLTQPEQPVILKVVPRGLGPQVEPDFL
>p450_10
Seq. ID NO: 60
MAADSFPLLAALFFILAATWFISFRRPRNLPPGPFPYPIVGNMLQLGTQPHETFAKLSKKY
GPLMSIHLGSLYTVIVSSPEMAKEINIHKYGQVFSGRTVAQAVHACGHDKISMGFLPVGG
EWRDMRKICKEQMFSHQSMEDSQWLRKQKLQQLLEYAQKCSERGRAIDIREAAFITTL
NLMSATLFSMQATEFDSKVTMEFKEIIEGVASIVGVPNFADYFPILRPFDPQGVKRRADV
YFGRLLAIIEGFLNERVESRRTNPNAPKKDDFLETLVDTLQTNDNKLKTDHLTHLMLDLF
VGGSETSTTEIEWIMWELLANPEKMAKMKAELKSVMGEEKVVDESQMPRLPYLQAVV
KESMRLHPPGPLLLPRKAESDQVVNGYLIPKGAQVLINAWAIGRDHSIWKNPDSFEPERF
LDQKIDFKGTDYELIPFGSGRRVCPGMPLANRILHTVTATLVHNFDWKLERPEASDAHR
GVLFGFAVRRAVPLKIVPFKV
>p450_11
Seq. ID NO: 61
MAADPFPLVAAALFIAATWFITFKRRRNLPPGPFPYPIVGNMLQLGSQPHETFAKLSKKY
GPLMSIHLGSLYTVIISSPEMAKEIMHKYGQVFSGRTIAQAVHACDHDKISMGFLPVGAE
WRDMRKICKEQMFSHQSMEDSQNLRKQKLQQLLEYAQKCSEEGRGIDIREAAFITTLNL
MSATLFSMQATEFDSKVTMEFKEIIEGVASIVGVPNFADYFPILRPFDPQGVKRRADVYF
GRLLGLIEGYLNERIEFRKANPNAPKKDDFLETLVDALDAKDYKLKTEHLTHLMLDLFV
GGSETSTTEIEWIMWELLASPEKMAKVKAELKSVMGGEKVVDESMMPRLPYLQAVVK
ESMRLHPPGPLLLPRKAESDQVVNGYLIPKGAQVLINAWAMGRDPSLWKNPDSFEPERF
LDQKIDFKGTDYELIPFGSGRRVCPGMPLANRILHTVTATLVHNFDWKLERPEASDAHK
GVLFGFAVRRAVPLKIVPIKA
>p450_12
Seq. ID NO: 62
MAADSFPLLAALFFIAATITFLSFRRRRNLPPGPFPYPIVGNMLQLGANPHQVFAKLSKR
YGPLMSIHLGSLYTVIVSSPEMAKEILHRHGQVFSGRTIAQAVHACDHDKISMGFLPVAS
EWRDMRKICKEQMFSNQSMEASQGLRRQKLQQLLDHVQKCSDSGRAVDIREAAFITTL
NLMSATLFSSQATEFDSKATMEFKEIIEGVATIVGVPNFADYFPILRPFDPQGVKRRADVF
FGKLLAKIEGYLNERLESKRANPNAPKKDDFLEIVVDIIQANEFKLKTHHFTHLMLDLFV
GGSDTNTTSIEWAMSELVMNPDKMARLKAELKSVAGDEKIVDESAMPKLPYLQAVIKE
VMRIHPPGPLLLPRKAESDQEVNGYLIPKGTQILINAYAIGRDPSIWTDPETFDPERFLDN
KIDFKGQDYELLPFGSGRRVCPGMPLATRILHMATATLVHNFDWKLEDDSTAAADHAG
ELFGVAVRRAVPLRIIPIVKS
>CPR_1
Seq. ID NO: 63
MAAGDSHVDTSSTVSEAVAEEVSLFSMTDMILFSLIVGLLTYWFLFRKKKEEVPEFTKIQ
TLTSSVRESSFVEKMKKTGRNIIVFYGSQTGTAEEFANRLSKDAHRYGMRGMSADPEEY
DLADLSSLPEIDNALVVFCMATYGEGDPTDNAQDFYDWLQETDVDLSGVKFAVFGLGN
KTYEHFNAMGKYVDKRLEQLGAQRIFELGLGDDDGNLEEDFITWREQFWPAVCEHFGV
EATGEESSIRQYELVVHTDIDAAKVYMGEMGRLKSYENQKPPFDAKNPFLAAVTTNRK
LNQGTERHLMHLELDISDSKIRYESGDHVAVYPANDSALVNQLGKILGADLDVVMSLN
NLDEESNKKHPFPCPTSYRTALTYYLDITNPPRTNVLYELAQYASEPSEQELLRKMASSS
GEGKELYLSWVVEARRHILAILQDCPSLRPPIDHLCELLPRLQARYYSIASSSKVHPNSVH
ICAVVVEYETKAGRINKGVATNWLRAKEPAGENGGRALVPMFVRKSQFRLPFKATTPVI
MVGPGTGVAPFIGFIQERAWLRQQGKEVGETLLYYGCRRSDEDYLYREELAQFHRDGA
LTQLNVAFSREQSHKVYVQHLLKQDREHLWKLIEGGAHIYVCGDARNMARDVQNTFY
DIVAELGAMEHAQAVDYIKKLMTKGRYSLDVWS
>CPR_2
Seq. ID NO: 64
MAAPFGIDNTDFTVLAGLVLAVLLYVKRNSIKELLMSDDGDITAVSSGNRDIAQVVTEN
NKNYLVLYASQTGTAEDYAKKFSKELVAKFNLNVMCADVENYDFESLNDVPVIVSIFIS
TYGEGDFPDGAVNFEDFICNAEAGALSNLRYNMFGLGNSTYEFFNGAAKKAEKHLSAA
GAIRLGKLGEADDGAGTTDEDYMAWKDSILEVLKDELHLDEQEAKFTSQFQYTVLNEIT
DSMSLGEPSAHYLPSHQLNRNADGIQLGPFDLSQPYIAPIVKSRELFSSNDRNCIHSEFDL
SGSNIKYSTGDHLAVWPSNPLEKVEQFLSIFNLDPETIFDLKPLDPTVKVPFPTPTTIGAAI
KHYLEITGPVSRQLFSSLIQFAPNADVKEKLTLLSKDKDQFAVEITSKYFNIADALKYLSD
GAKWDTVPMQFLVESVPQMTPRYYSISSSSLSEKQTVHVTSIVENFPNPELPDAPPVVGV
TTNLLRNIQLAQNNVNIAETNLPVHYDLNGPRKLFANYKLPVHVRRSNFRLPSNPSTPVI
MIGPGTGVAPFRGFIRERVAFLESQKKGGNNVSLGKHILFYGSRNTDDFLYQDEWPEYA
KKLDGSFEMVVAHSRLPNTKKVYVQDKLKDYEDQVFEMINNGAFIYVCGDAKGMAK
GVSTALVGILSRGKSITTDEATELIKMLKTSGRYQEDVW
>CPR_3
Seq. ID NO: 65
MAAGDSHEDTSATVPEAVAEEVSLFSTTDIVLFSLIVGVLTYWFIFKKKKEEIPEFSKIQT
TAPPVKESSFVEKMKKTGRNIIVFYGSQTGTAEEFANRLSKDAHRYGMRGMSADPEEY
DLADLSSLPEIDKSLVVFCMATYGEGDPTDNAQDFYDWLQETDVDLTGVKFAVFGLGN
KTYEHFNAMGKYVDQRLEQLGAQRIFELGLGDDDGNLEEDFITWREQFWPAVCEFFGV
EATGEESSIRQYELVVHEDMDTAKVYTGEMGRLKSYENQKPPFDAKNPFLAAVTTNRK
LNQGTERHLMHLELDISDSKIRYESGDHVAVYPANDSTLVNQIGEILGADLDVIMSLNNL
DEESNKKHPFPCPTTYRTALTYYLDITNPPRTNVLYELAQYASEPSEQEHLHKMASSSGE
GKELYLSWVVEARRHILAILQDYPSLRPPIDHLCELLPRLQARYYSIASSSKVHPNSVHIC
AVAVEYEAKSGRVNKGVATSWLRTKEPAGENGRRALVPMFVRKSQFRLPFKPTTPVIM
VGPGTGVAPFMGFIQERAWLREQGKEVGETLLYYGCRRSDEDYLYREELARFHKDGAL
TQLNVAFSREQAHKVYVQHLLKRDKEHLWKLIHEGGAHIYVCGDARNMAKDVQNTFY
DIVAEFGPMEHTQAVDYVKKLMTKGRYSLDVWS
>CPR_4
Seq. ID NO: 66
MAAGDSHEDTSATMPEAVAEEVSLFSTTDMVLFSLIVGVLTYWFIFRKKKEEIPEFSKIQ
TTAPPVKESSFVEKMKKTGRNIIVFYGSQTGTAEEFANRLSKDAHRYGMRGMSADPEEY
DLADLSSLPEIDKSLVVFCMATYGEGDPTDNAQDFYDWLQETDVDLTGVKFAVFGLGN
KTYEHFNAMGKYVDQRLEQLGAQRIFELGLGDDDGNLEEDFITWREQFWPAVCEFFGV
EATGEESSIRQYELVVHEDMDAAKVYTGEMGRLKSYENQKPPFDAKNPFLAAVTANRK
LNQGTERHLMHLELDISDSKIRYESGDHVAVYPANDSALVNQIGEILGADLDVIMSLNN
LDEESNKKHPFPCPTTYRTALTYYLDITNPPRTNVLYELAQYASEPSEQEHLHKMASSSG
EGKELYLSWVVEARRHILAILQDYPSLRPPIDHLCELLPRLQARYYSIASSSKVHPNSVHI
CAVAVEYEAKSGRVNKGVATSWLRAKEPAGENGGRALVPMFVRKSQFRLPFKSTTPVI
MVGPGTGIAPFMGFIQERAWLREQGKEVGETLLYYGCRRSDEDYLYREELARFHKDGA
LTQLNVAFSREQAHKVYVQHLLKRDREHLWKLIHEGGAHIYVCGDARNMAKDVQNTF
YDIVAEFGPMEHTQAVDYVKKLMTKGRYSLDVWS
>CPR_5
Seq. ID NO: 67
MAAINMGDSHVDTSSTVSEAVAEEVSLFSMTDMILFSLIVGLLTYWFLFRKKKEEVPEFT
KIQTLTSSVRESSFVEKMKKTGRNIIVFYGSQTGTAEEFANRLSKDAHRYGMRGMSADP
EEYDLADLSSLPEIDNALVVFCMATYGEGDPTDNAQDFYDWLQETDVDLSGVKFAVFG
LGNKTYEHFNAMGKYVDKRLEQLGAQRIFELGLGDDDGNLEEDFITWREQFWPAVCEH
FGVEATGEESSIRQYELVVHTDIDAAKVYMGEMGRLKSYENQKPPFDAKNPFLAAVTT
NRKLNQGTERHLMHLELDISDSKIRYESGDHVAVYPANDSALVNQLGKILGADLDIVMS
LNNLDEESNKKHPFPCPTSYRTALTYYLDITNPPRTNVLYELAQYASEPSEQELLRKMAS
SSGEGKELYLSWVVEARRHILAILQDCPSLRPPIDHLCELLPRLQARYYSIASSSKVHPNS
VHICAVVVEYETKAGRINKGVATNWLRAKEPAGENGGRALVPMFVRKSQFRLPFKATT
PVIMVGPGTGVAPFIGFIQERAWLRQQGKEVGETLLYYGCRRSDEDYLYREELAQFHRD
GALTQLNVAFSREQSHKVYVQHLLKRDREHLWKLIEGGAHIYVCGDARNMARDVQNT
FYDIVAELGAMEHAQAVDYIKKLMTKGRYSLDVWS
>CPR_6
Seq. ID NO: 68
MAAINMGDSHMDTSSTVSEAVAEEVSLFSMTDMILFSLIVGLLTYWFLFRKKKEEVPEF
TKIQTLTSSVRESSFVEKMKKTGRNIIVFYGSQTGTAEEFANRLSKDAHRYGMRGMSAD
PEEYDLADLSSLPEIENALVVFCMATYGEGDPTDNAQDFYDWLQETDVDLSGVKFAVF
GLGNKTYEHFNAMGKYVDKRLEQLGAQRIFELGLGDDDGNLEEDFITWREQFWPAVCE
HFGVEATGEESSIRQYELVVHTDIDAAKVYMGEMGRLKSYENQKPPFDAKNPFLAAVT
TNRKLNQGTERHLMHLELDISDSKIRYESGDHVAVYPANDSALVNQLGKILGADLDVV
MSLNNLDEESNKKHPFPCPTSYRTALTYYLDITNPPRTNVLYELAQYASETSEQELLRK
MASSSGEGKELYLSWVVEARRHILAILQDCPSLRPPIDHLCELLPRLQARYYSIASSSKVH
PNSVHICAVVVEYETKAGRINKGVATNWLRAKEPAGENGGRALVPMFVRKSQFRLPFK
ATTPVIMVGPGTGVAPFIGFIQERAWLRQQGKEVGETLLYYGCRRSDEDYLYREELVQF
HRDGALTQLNVAFSREQSHKVYVQHLLKRDREHLWKLIEGGAHIYVCGDARNMARDV
QNTFYDIVAELGAMEHTQAVDYIKKLMTKGRYSLDVWS
>CPR_7
Seq. ID NO: 69
MAANMADSNMDAGTTTSEMVAEEVSLFSTTDVILFSLIVGVMTYWFLFRKKKEEVPEF
TKIQTTTSSVKDRSFVEKMKKTGRNIIVFYGSQTGTAEEFANRLSKDAHRYGMRGMAA
DPEEYDLADLSSLPEIEKALAIFCMATYGEGDPTDNAQDFYDWLQETDVDLSGVKYAVF
ALGNKTYEHFNAMGKYVDKRLEQLGAQRIFDLGLGDDDGNLEEDFITWREQFWPAVC
EHFGVEATGEESSIRQYELMVHTDMDMAKVYTGEMGRLKSYENQKPPFDAKNPFLAV
VTTNRKLNQGTERHLMHLELDISDSKIRYESGDHVAVYPANDSALVNQLGEILGADLDII
MSLNNLDEESNKKHPFPCPTSYRTALTYYLDITNPPRTNVLYELAQYASEPTEHEQLRK
MASSSGEGKELYLRWVLEARRHILAILQDYPSLRPPIDHLCELLPRLQARYYSIASSSKVH
PNSVHICAVAVEYETKTGRINKGVATSWLRAKEPAGENGGRALVPMYVRKSQFRLPFK
ATTPVIMVGPGTGVAPFIGFIQERAWLRQQGKEVGETLLYYGCRRSDEDYLYREELAGF
HKDGALTQLNVAFSREQPQKVYVQHLLKKDKEHLWKLIHEGGAHIYVCGDARNMARD
VQNTFYDIVAEQGAMEHAQAVDYVKKLMTKGRYSLDVWS
>CBNsyn_1
Seq. ID NO: 71
MAADFSGKNVWVTGAGKGIGYATALAFVEAGAKVTGFDQAFTQEQYPFATEVMDVA
DAAQVAQVCQRLLAETERLDALVNAAGILRMGATDQLSKEDWQQTFAVNVGGAFNLF
QQTMNQFRRQRGGAIVTVASDAAHTPRIGMSAYGASKAALKSLALSVGLELAGSGVRC
NVVSPGSTDTDMQRTLWVSDDAEEQRIRGFGEQFKLGIPLGKIARPQEIANTILFLASDL
ASHITLQDIVVDGGSTLGA
>CBNsyn_2
Seq. ID NO: 72
MAASDLHNESIFITGGGSGLGLALVERFIEEGAQVATLELSAAKVASLRQRFGEHILAVE
GNVTCYADYQRAVDQILTRSGKLDCFIGNAGIWDHNASLVNTPAETLETGFHELFNVNV
LGYLLGAKACAPALIASEGSMIFTLSNAAWYPGGGGPLYTASKHAATGLIRQLAYELAP
KVRVNGVGPCGMASDLRGPQALGQSETSIMQSLTPEKIAAILPLQFFPQPADFTGPYVML
TSRRNNRALSGVMINADAGLAIRGIRHVAAGLDL
>CBNsyn_3
Seq. ID NO: 73
MAATGWLAGKRALIVGAGSGIGRATVDAFLNEDARVAVLEYDSDKCATLRHQLPDVP
VIEGDGTTRTANDEAVQVAVDAFGGLDTLVNCVGIFDFYRRIQDIPAELIDQAFDEMFRI
NVLSHIHSVKAAVPALMGQDGASIVLTESASSFYPGRGGLLYVASKFAVRGVVTALAHE
LAPRIRVNGVAPGGTLNTDLRGLDSLDLGARRLDAAPDRARELAARTPLGVALSGEDH
AWSYVFLASHRSRGLTGETIHPDGGFSLGPPPQRN
>CBNsyn_4
Seq. ID NO: 74
MSSIETKIFPGRFDGRCLTITGAAQGIGLTVATRIAAEGGEVVLVDRADLVHEVAEQLRE
AGGKAHSVTADLETFEGAEEAISHAVRTTGRIDVLINVVGGTIWAKPYEHYAPEEIEKEI
RRSLFPTLWTCRAAAPHLIERRAGTIVNVSSVATRGVNRVPYSAAKGGVNAITASLALE
LAPYGVRVVATAPGGTVAPERRIARGPSPQSEQEKAWYQQIVDQTVDSSLLKRYGTLDE
QAAAICFLASEEASYITGTVLPVAGGDLG
>CBNsyn_5
Seq. ID NO: 75
MSSTGWLDGKRALVVGGGSGIGRAVVDAFLAEGACVAVLERDPNKCRVLREHLPQVP
VIEGDATRAADNDAAVAAAVAAFGGLDTLVNCVGIFDFYQGIEDIPADTLDVAFDEMF
RTNVLSHMHSVKAAVPELRKHRGSSIVLAESASSFYPGRGGVLYVSSKFAVRGLVTTLA
YELAPDIRVNGVAPGGTLNTDLRGLASLGRDADRLDDNPNRANELAARTPLNVALSGE
DHAWSFVFFASDRSRGITAGATHPDGGFGIGAPKPSTR
>CBNsyn_6
Seq. ID NO: 76
MSSGFLDGKVALVTGGGSGIGRAVVELYVQQGAKVGILEISPEKVKDLRNALPADSVV
VTEGDATSMADNERAVADVVDAFGPLTTLVCVVGVFDYFTEIPQLPKDKISEAFDQLFG
VNVKSNLLSVKAALDELIENEGDIILTLSNAAFYAGGGGPLYVSSKFAVRGLVTELAYEL
APKVRVNGVAPGGTITELRGIPALANEGQRLKDVPDIEGLIEGINPLGIVAQPEDHSWAY
ALLASRERTSAVTGTIINSDGGLGVRGMTRMAGLAQ
>CBNsyn_7
Seq. ID NO: 77
MSSSRSVTLVVGAAQGIGRATALTLATAGHRVVLADRDVDGLAETAALLHVAAPVHG
LDVCDAAGVAEAVARVEVEHGPVDALAHVAGVFTTGSVLDSDLAEWQRMFDVNVTG
LINVLRVVGHGMRERRRGAIVTVGSNSAGVPRVGMGAYGASKSAAHMLVRVLGLELA
RFGVRANVVAPGSTDTAMQRSLWPDPADDAGARTAIDGDAASFKVGIPLGRIADPADIA
DAVEFLLSDRARHITMQTLYVDGGATLRA
>CBNsyn_8
Seq. ID NO: 78
MSSQMLDDHVALILGGGSGLGLGIARHFLGEGAQVAIFEISESKLLDLKAEFGDDVLLLQ
GDVTSIDDLEAARAAVVDRFGRLDALIGAQGIFDGNIPLRDIPTERIEKVFDEVLHVDVL
GYILAARVFLEELEKTDGAIVFTSSTAAYAADGGGLFYTAAKGAVRSVINQLAFEFAPK
VRVNGVAPSGIANSQLQGPRALGLENNKQSDIPVEDFTNQFLSLTLTPTLPTPEEYAPLY
AYLASRNNTTMTGQTIIADQGLFNRAVISNGVADRVGK
>THCdeg_1
Seq. ID NO: 79
MSSSGPAHSNLEQVFANVASNYRGADVDLHAVYREMREKSPVLPENFMARLGVPSIAG
LDPNRPTFTLFKYDDVMAVMRDATNFTSGFIAEGLGSFFDGLILTAMDGEAHKNIRSLL
QPVFMPETVNRWKETKIDRVIREEYLRPMVASKRADIMEFALYFPIRVIYSLIGFPEDRPE
EIEQYAAWALAILAGPQVDPEKAAAARGAAMEAAQALYDVVKVVVAQRRAEGATGD
DLICRLIRAEYEGRSLDDHEITTFVRSLLPAASETTTRTFGTLMTLLLERPELLARIREDRS
LVGKAIDEAVRYEPVATFKVRQAAKDVEIRGVAIPKGAMVSCIVTSANRDEDAFENADT
FDIDRRAKPSFGFGFGPHMCIGQFVAKTEINCALNAILDLMPNIRLDPDKPAPEIIGAQLR
GPHHVHVIWD
>THCdeg_2
Seq. ID NO: 80
MSSRSTDLPDLKSAAFLADRYPTYRRLQSDFPHFEMNINGEECIVLTRYSDVDEVLRNPL
ATVQQAPGVFPERIGQGAGARFYRESLPNIDAPDHTRIRRIVTPAFNPKTVANMRGWVE
KVIVEHLDRLEGLDEIDFVSSFADPVPAEIACRLLHVPVSDAPELFARQHGLNAVLSVSDI
TPERLAEADASAAFYYEYMDDVLNTLKGKLPEDDFVGALMAAEARDSGLTRSELVTTL
IGFLVASYHTTKVAMTNTVLALLNHDGERARLVAQPDLARNAWEESLRYDSPVHFVHR
YASEPLTIGGQPVAQGKRLLLGLHAASRDENRFAQADHYLIDRPDNRHLAFAGGGHFCL
GSQLSRLEGDVLLRTIFQRFPAMRLTETRFERVPDLTFPMLLRMTVSLRAEQG
>THCdeg_3
Seq. ID NO: 81
MSSTSNSIRSPLSPPQPRRTPPPCTSSREPPIVRGTWLLGSTRDLLRDPLELGLRGYAEGGD
VVRYVVGLPGRRREFFTVNHPDGVGELLNAPRHLDYRKDSEFYRAMRDLYGNGLVTS
QDETWLRQRRFIQPLFTPQSVDGYVTPMVAEADRVAIRWHNCTSRLVDLDGEMRALTL
GVAARILFGVQAPRMLPILRTTLPVLGRAVLQQGASAIRFPSSWPTPGNRRIASAESRLD
GLCDAIIERRRTVAEPGTDLLGRLVAAREDGDTLSTEEIRDQVKVFLLAGHDTTATMLTF
ALYLLGKDAGVQDQARDEAERVLGAGTPTASDVHRLTYTTMVLEEAARLYPPSPYLTR
RAVEESEVCGYRIPAGADVNLAPWVIHHRADLWPDPFRFDPDRFTPDRVKERHKYAWF
PFGHGPRGCIGQRFAMLEAAVTLAILLREFEFRSPPGSVPLTVDLLLHPAGEVPCRVRRR
VPVHSAVHRTHQPS
>THCdeg_4
Seq. ID NO: 82
MSSAPDILSPEFLDNPYPLHRVLRDHYPALHHEGTDSYLISRYADCAEAFRSPKFSSRNY
EWQLEPIHGRTILQMEGREHSTHRALLNPFFRGNGLERFMPAITHNAAQLIGDIVARNAG
ELLGAVARQGEAELVSQFTSRFPINVMVDMLGLPKSDHERFRGWYFSIMAYLNNLAGD
PEINAAAERTHVELREYMLPIIRERRSGDGDDLLSRLCRAEVDGEQMSDEEIKAFVSLLL
VAGGETTDKAIASMIRNLIDHPDQMRAVREDRSLADRVIAETLRYSGPVHMIMRQTEDE
VQIEDSTIPAGATCIMMLAAANRDERHFSNPDEFDIFRTDLNVDRAFSGAANHVQFILGR
HFCVGSMLAKTEMTIALNLVLDTMDSIEYQDGFVPREEGLYTRSIPELRVKFEGKLG
>THCdeg_5
Seq. ID NO: 83
MSSSTPAAATSLESAFAGVADNYKGSDVDLHAIYRDMRRNSPVIAEDFMARLGVPNIAG
LDAKRPTFTLFKYKDVMSVLRDATNFTSGFIAEGLGAFFDGLILTGMDGEAHRRTRSLL
QPVFMPDVVNRWRETKMAPIVRNEYIEPMVPKRRADLMDFGLHFPIRLIYSLIGFPDNRP
EQIEQYAAWALAILAGPQVDAEKAAQARKAAMEAAQALYDAVKLEVTEVRKNGAQG
DDLICRLIRAEYEGRHLDDHEVTTFVRSLLPAAGETTTRTFGSLMVALLERPELLERVRA
DRSLVPKAIDEAVRFEPVATFKVRQAAQDTEIGGFSIPKGAMVQCIVSSANRDEEVFENS
ESFDIDRKLKPSFGFGFGPHMCIGQFIAKVELSVAVNTILDLLPNLRLDPDRPKPRIVGAQ
LRGPHALHVIWD
>THCdeg_6
Seq. ID NO: 84
MSSSPSVAELSQELGEAFRLSSMDDPYPMLAERRRETPVMKGDIMVALGAPSYMGQHA
GETHTVFRHDDVMAILRNHETFSSSIWEISQGPLIGRSILAMDGAEHRQWRGYLQSVFG
GKLLSSWDESIFRPLAAKYVADLASKRGADLIAMALEYPLRAIYEILGLEDFKDNYEEFH
ADVLTILLALWSTPDPAQADQFLLRFQKATEASARSWDRLLPIVQRKRAAGASRNDLIS
SLIRAEYEGGVLDDEQITSFLRSLLLAATDTTTRQFLNTLTLLLQRPDELDRIRRDRSRLR
LALAEGERLEPPALFIPRMITRDVVIRGTELTAGTPLLLAIGSANRDPEAYPPDPDEFRIDR
TGPHHATFGFGTHICSGMNTTRREIAALIDAMLDGLPGLRVDPDAPAPLISGIHFRGPSAL
PVVWD
>THCdeg_7
Seq. ID NO: 85
MSSDYSRTPESLRPADSYAALSYSTVNAALRNDRVFSSKMYDSTIGVFMGPTILAMSGT
KHRAHRNLVSAAFKPQSLRVWEPDIVRPICNALIDEFAGTGHADLVRDFTFEFPTRVIAR
LLGLPAEDLPFFRKAAVAIISYAGNVPRALEASEDLKNYFLGHIEQRRSQPTDDIISDLVT
AEVEGEQLTDEAIYSFLRLLLPAGLETTYRSSGNLLYLLLRHPRQFAAVQGNHGLIPQAV
EEGLRYETPLTFVQRFTTEDTELGGVPVPAGAVVDLVLGSANRDEDRWERPGEFDIFRK
PVPHISFTAGAHTCLGLHLARMETRVAVECLLTRLTNFRLQDEGDPHITGQPFRSPNLLP
VTFDVV
>THCdeg_8
Seq. ID NO: 86
MSSPTPRWRIPVLGDLLSVDPAKPVQKEMAMAAELGPLFERKIIGSRLTVVSGVDLVAE
VNDEKHWARALGRPILKLRDVAGDGLFTAFNSEPAWARAHSVLGPGFSQSALRTYHGS
MTRVLDDLVATWDDAAASGARVDVARDMTRLTFDVIGRAGFGRDFGSLRGDDLDPFA
AAMGRALGYVNQTSNDIPLLRMVFGRGAAKRYQTDVAFMRDTVDELVASRAGRAERS
DDLLDLMLHSADPDTGERLDMENIRNQVLTFLVAGNETTASTLAFALYFLAREPEVVER
ARAEIADVVGDGEIAFEQVAKLRYVRRVVDETLRLWPAAPGYFRKVRHDTVLGGRYP
MPKGSWVFVLLPQLHRDPVWGDDPERFDPDRFAPDAVRARPKDAYRPFGTGPRSCIGR
QFALHEAVLALATLLRRYDVAPDPAYRLDIVEAVTLKPRGFELTLQRR
>THCdeg_9
Seq. ID NO: 87
MSSSASSQSNLEQVFANVASNYRGADIDLHAVYREMREKSPVLPENFMARLGVPSIAGL
DPDRPAFTLFKYDDVMAVMRDATNFTSGFIAEGLGSFFDGLILTAMDGEAHKNIRSLLQ
PVFMPETVNRWKETKIDRVIREEYLQPMVASKGADIMEFALYFPIRVIYSLIGFPEDRPEE
IEQYAAWALAILAGPQVDPEKAVAARGAAMEAAQALYDVVKVVVAQRRSQGATGDD
LISRLIRAEYEGRSLDDHEITTFVRSLLPAASETTTRTFGTLMTLLLERPELLARIREDRSL
VPKAIDEAVRYEPVATFKVRQAAKDVEIRGVAIPQGAMVSCIVTSANRDEDAFENADTF
NIDRRAKPSFGFGFGPHMCIGQFVAKTEINCALNAILDLMPNIRLDPDKPAPEIIGAQLRG
PHHVHVIWD
>THCdeg_10
Seq. ID NO: 88
MSSTATELRDAPGSAPGLPRRSMLSLLPRMARDRLSVMTSVAARYGDAVTLPLGLSTLH
FFNHPDYAKHVLADNSSNYHKGIGLIHAKRALGDGLLTSEGELWRKQRKTIQPAFAVKR
LAGQAGAIAEEADRLVEHLLARQGRGPVDIRHEMTALTLGVLGRTLLDADLGAFGSVG
HWFEAVQDQAMFDMMSLGTVPLWSPLPKQLRFRRARRELESVVDRLVAQRGDRPRAD
GDDVVSRLVDSTGRERDPALRRKRMHDELVTLLLAGHETTASTLSWTFHLADEHPEVW
ERLHAEAVEVLGDRRPVFEDLHRLRYTNRVLNEVMRLYPPVWLLPRRAVADDVVGGY
RVPAGSDVLICPYTLHRHPEFWELPSRFDPDRFDPERSANRPRYAYIPFGAGPRFCVGNN
LGLMEAAFVIAAIARRMRLRKVPGGTVVPEPMLTLRVRSGLPMTVHALDR
>Oxid_1
Seq. ID NO: 89
MSSQRRDFLKYSVALGVASALPLWSRAVFAAERPTLPIPDLLTTDARNRIQLTIGAGQST
FGGKTATTWGYNGNLLGPAVKLQRGKAVTVDIYNQLTEETTLHWHGLEVPGEVDGGP
QGIIPPGGKRSVTLNVDQPAATCWFHPHQHGKTGRQVAMGLAGLVVIEDDEILKLMLP
KQWGIDDVPVIVQDKKFSADGQIDYQLDVMTAAVGWFGDTLLTNGAIYPQHAAPRGW
LRLRLLNGCNARSLNFATSDNRPLYVIASDGGLLPEPVKVSELPVLMGERFEVLVEVND
NKPFDLVTLPVSQMGMAIAPFDKPHPVMRIQPIAISASGALPDTLSSLPALPSLEGLTVRK
LQLSMDPMLDMMGMQMLMEKYGDQAMAGMDHSQMMGHMGHGNMNHMNHGGKF
DFHHANKINGQAFDMNKPMFAAAKGQYERWVISGVGDMMLHPFHIHGTQFRILSENG
KPPAAHRAGWKDTVKVEGNVSEVLVKFNHDAPKEHAYMAHCHLLEHEDTGMMLGFT
V
>Oxid_2
Seq. ID NO: 90
MSSRLSFLTSLVTLALVSSTYAGVGPVVDLTVSNAVISPDGFDRDAIVVNGVFPAPLITG
KKGDRFQLNVIDNMTNHTMLKSTSIHWHGFFQKGTNWADGGAFVNQCPIAPGHSFLYD
FRVPDQAGTFWYHSHLSTQYCDGLRGPIVVYDPNDPHADLYDVDNDSTVITLADWYH
VAARLGPRFPLGADSTVINGLGRSLSTPNADLAVISVTQGKRYRFRLISLSCDPFHTFSID
GHDLTIIEADSVNTEPLVVDAIPIFAGQRYSFVLSAVKDIDNYWIRADPNFGTTGFASGIN
SAILRYDGAAPIEPTAVLAPVSVNPLVETDLHPLEDMPVPGRPTKGGVDKAINLDFSFSFP
NFFINNATFTSPTVPILLQIMSGAQAAQDLLPSGSVIELPAQSTIELTLPATVNAPGVPHPF
HLHGHTFAVVRSAGSTAYNYDNPIWRDVVSTGTPAANDNVTIRFTTDNPGPWFLHCHI
DFHLEAGFAVVFAEGVPQTQVANPVPQAWEELCPIYDALPEDDQ
>Oxid_3
Seq. ID NO: 91
MSSFKVSCKVTNNNGDQNVETNSVDRRNVLLGLGGLYGVANAIPLAASAAPTPPPDLK
TCGKATISDGPLVGYTCCPPPMPTNFDNIPYYKFPSMTKLRIRSPAHAVDEEYIAKYNLAI
SRMKDLDKTEPLNPLGFKQQANIHCAYCNGAYVFGDKVLQVHNSWLFFPFHRWYLYF
YERILGKLIDDPTFALPYWNWDHPKGMRLPPMFDREGTSIYDERRNQQVRNGTVMDLG
SFGDKVETTQLQLMSNNLTLMYRQMVTNAPCPLLFFGAPYVLGNNVEAPGTIENIPHIP
VHIWAGTVRGSTFPNGDTSYGEDMGNFYSAGLDSVFYCHHGNVDRMWNEWKAIGGK
RRDLSEKDWLNSEFFFYDENKKPYRVKVRDCLDAKKMGYDYAPMPTPWRNFKPKTKV
SAGKVNTSSLPPVNEVFPLAKMDKVISFSINRPASSRTQQEKNEQEEMLTFDNIKYDNRG
YIRFDVFLNVDNNVNANELDKVEFAGSYTSLPHVHRVGENDHTATVTFQLAITELLEDI
GLEDEETIAVTLVPKKGGEGISIENVEIKLLDC
>Oxid_4
Seq. ID NO: 92
MSGQNKMGLILVFLFLDGLLVCLAADVDVHNYTFVLQEKNFTKWCSTKSMLVVNGSF
PGPTITARKGDTIFVNVINQGKYGLTIHWHGVKQPRNPWSDGPEYITQCPIKPGTNFIYEV
ILSTEEGTLWWHAHSDWTRATVHGALVILPANGTTYPFPPPYQEQTIVLASWFKGDVME
VITSSEETGVFPAAADGFTINGELGDLYNCSKETTYRLSVQPNKTYLLRIVNAVLNEEKF
FGIAKHTLTVVAQDASYIKPINTSYIMITPGQTMDVLFTTDQTPSHYYMVASPFHDALDT
FANFSTNAIIQYNGSYKAPKSPFVKPLPVYNDIKAADKFTGKLRSLANEKFPVNVPKVNV
RRIFMAVSLNIVKCANKSCNNNIGHSTSASLNNISFALPQTDVLQAYYRNISGVFGRDFP
TVQKKANFSLNTAQGTQVLMIEYGEAVEIVYQGTNLGAATSHPMHLHGFNFYLVGTGA
GTFNNVTDPPKYNLVDPPELNTINLPRIGWAAIRFVADNPGVWFLHCHFERHTTEGMAT
VVIVKDGGTTNTSMLPSPAYMPPCS
>Oxid_5
Seq. ID NO: 93
MSSRKICLGCSHSLSSQPFTYTTQKTVSSRRIGDSQWRLSRGYTRTLTSASASVATAPAK
LLTVNETQKCLRNMVRGGDVISYILSHSSRNADQNLKDLDSLILEPVCSATHEMFDVFEI
PEHILTPFCDNRNVPEEQVTRNPNLRTDCLTMKRFVLLQSLVAVASAGIGPVADLYVGN
RILAPDGFNRSTVLGGTSSSDFGFPAPLITGTKGDRFQLNVINQLTDTTMLRSTSIHWHGL
FQAGSSWADGPVGVNQCPIAPGNSFLYDFNVPDQAGTFWYHSHYSTQYCDGLRGAFV
VRDPNDPHASLYDVDNDDTVITLADWYHTSAKELSGSFPAEEATLINGLGRYSGGPTSP
LAIVNVEAGKRYRFRLVSISCDPFYTFSIDGHDLTIIEADGENTDPLVVDYLEIYAGQRYS
VVLNANQPVDNYWIRANSSNGPRDFVGGTNSAILRYAGASNSDPTTELGPRNNRLVEN
NLHALGSPGVPGTHTIGEADVNINLEILFTPPNVLTVNGAQFIPPTAPVLLQILSGTKQAT
DLLPPGSVYVLPRNAVVELTIPGGSGGSPHPMHLHGHVFDVVRSAGSDTINWDNPVRRD
VVNIGTSTSDNATIRFTTDNPGPWIFHCHIDWHLEVGLAVVFAEDPDTIENSTHPAAWDE
LCPIYDNLPSDEL
>Oxid_6
Seq. ID NO: 94
MSSTLEKFVDALPIPDTLKPVQQSKEKTYYEVTMEECTHQLHRDLPPTRLWGYNGLFPG
PTIEVKRNENVYVKWMNNLPSTHFLPIDHTIHHSDSQHEEPEVKTVVHLHGGVTPDDSD
GYPEAWFSKDFEQTGPYFKREVYHYPNQQRGAILWYHDHAMALTRLNVYAGLVGAYII
HDPKEKRLKLPSDEYDVPLLITDRTINEDGSLFYPSAPENPSPSLPNPSIVPAFCGETILVN
GKVWPYLEVEPRKYRFRVINASNTRTYNLSLDNGGDFIQIGSDGGLLPRSVKLNSFSLAP
AERYDIIIDFTAYEGESIILANSAGCGGDVNPETDANIMQFRVTKPLAQKDESRKPKYLAS
YPSVQHERIQNIRTLKLAGTQDEYGRPVLLLNNKRWHDPVTETPKVGTTEIWSIINPTRG
THPIHLHLVSFRVLDRRPFDIARYQESGELSYTGPAVPPPPSEKGWKDTIQAHAGEVLRIA
ATFGPYSGRYVWHCHILEHEDYDMMRPMDITDPHK
>Oxid_7
Seq. ID NO: 95
MSSVFSAAFSAFVALGLTLGAFAAVGPVADIHITDDTIAPDGFSRAAVLAGGTFPGPLIT
GNMGDAFKLNVIDELTDASMLKSTSIHWHGFFQKGTNWADGPAFVNQCPITTGNSFLY
DFQVPDQAGTYWYHSHLSTQYCDGLRGAFVVYDPSDPHKDLYDVDDESTVITLADWY
HTLARQIVGVAISDTTLINGLGRNTDGPADAALAVINVEAGKRYRFRLVSISCDPNWVFS
IDNHDFTVIEVDGVNSQPLNVDSVQIFAGQRYSLVLNANQPVDNYWIRADPNLGTTGFA
GGINSAILRYKGAAVAEPTTSQTTSTKPLLETDLHPLVSTPVPGLPQPGGTDVVQNLILGF
NAGQFTINGASFVPPTVPVLLQILSGTTNAQDLLPSGSVFELPLGKTVELTLAAGVLGGP
HPFHLHGHNFHVVRSAGQDTPNYDDPIVRDVVSTGASGDNVTIRFTTDNPGPWFLHCHI
DWHLEAGFAVVFAEAVNETKSGNPTPAAWDNLCTLYDALADGDK
>Oxid_8
Seq. ID NO: 96
MSSCLAAIWSRKRAEHAASRLPALQEKRSTLSYAYARLDGSLASMFPNRFWSSVSLGAR
IKPVDGSSEEPTARPSSCARPFLHSASSESGFVSSSRPTSFCVTCSRRWRCCSLLAMLGFR
FLHTSVLAALTLSLKSYAAIGPVTDLTVANANISPDGYERAAVLAGGSFPGPLITGRKGD
HFQINVVDQLTNHTMLKSTSIHWHGLFQKGTNWADGPAFVNQCPISTGNSFLYDFHVP
DQAGTFWYHSHLSTQYCDGLRGAMVVYDPNDPHKNLYDVDNDDTVITLADWYHVAS
KLGPAVPFGGDSTLINGKGRSTATPTADLAVISVTQGKRYRFRLVSLSCDPNFTFSIDGH
ALTVIEADAVSTQPLTVDSIQIFAGQRYSFVLNANQSVDSYWIRAQPSLGNVGFDGGLNS
AILRYDGAAPTEPSALAVPVSTNPLVETALRPLNSMPVPGKAEVGGVDKAINLAFSFNG
TNFFINGATFVPPAVPVLLQIMSGAQSASDLLPSGSVFVLPSNATIELSFPATANAPGAPH
PFHLHGHTFAVVRSAGSAEYNYENPIWRDVVSTGSPGDNVTIRFRTDNPGPWFLHCHID
PHLEAGFAVVMAEDTRDVKADNPEPKAWDDLCPTYNALAVDDQ
>Oxid_9
Seq. ID NO: 97
MFPGARILATLTLALHLLHGTNAAIGPTGDMYIVNEDVSPDGFTRSAVVARSDPTTNGT
SETLTGVLVQGNKGDNFQLNVLNQLSDTTMLKTTSIHWHGFFQSGSTWADGPAFVNQC
PIASGNSFLYDFNVPDQAGTFWYHSHLSTQYCDGLRGPFIVYDPSDPHLSLYDVDNADTI
ITLEDWYHVVAPQNAVLPTADSTLINGKGRFAGGPTSALAVINVESNKRYRFRLISMSC
DPNFTFSIDGHSLQVIEADAVNIVPIVVDSIQIFAGQRYSFVLNANQTVDNYWIRADPNLG
STGFDGGINSAILRYAGATEDDPTTTSSTSTPLEETNLVPLENPGAPGPAVPGGADININL
AMAFDVTNFELTINGSPFKAPTAPVLLQILSGATTAASLLPSGSIYSLEANKVVEISIPALA
VGGPHPFHLHGHTFDVIRSAGSTTYNFDTPARRDVVNTGTDANDNVTIRFVTDNPGPWF
LHCHIDWHLEIGLAVVFAEDVTSITAPPAAWDDLCPIYDALSDSDKDNPRFGFAPATGG
KATGRRNWFSKARRRAILVPYLKLLKLGLVMVFYIRAERNHGRLSQSTPPNRRVDQREL
ITNTWVERFFLHRLMFLKLFVGTACFMHIFNSVSSLGMCTLRTSHGSSESLASPSATMML
GGGLTLLSANIRLWCYAEMRDLYDFEVNIKKAHRLVTTGPYSVSMVMFSKDHWLYQC
GLRSMVGVVLSCIWCAEVVLINGIMVPARMKVEDDGLRRHFGREWDEYASRVAYRLV
PEIY
>Oxid_10
Seq. ID NO: 98
MSSKSFISAATLLVGILTPSVAAAPPSTPEQRDLLVPITEREEAAVKARQQSCNTPSNRAC
WTDGYDINTDYEVDSPDTGVVRPYTLTLTEVDNWTGPDGVVKEKVMLVNNSIIGPTIFA
DWGDTIQVTVINNLETNGTSIHWHGLHQKGTNLHDGANGITECPIPPKGGRKVYRFKAQ
QYGTSWYHSHFSAQYGNGVVGAIQINGPASLPYDTDLGVFPISDYYYSSADELVELTKN
SGAPFSDNVLFNGTAKHPETGEGEYANVTLTPGRRHRLRLINTSVENHFQVSLVNHTMT
IIAADMVPVNAMTVDSLFLGVGQRYDVVIEASRTPGNYWFNVTFGGGLLCGGSRNPYP
AAIFHYAGAPGGPPTDEGKAPVDHNCLDLPNLKPVVARDVPLSGFAKRPDNTLDVTLD
TTGTPLFVWKVNGSAINIDWGRPVVDYVLTQNTSFPPGYNIVEVNGADQWSYWLIEND
PGAPFTLPHPMHLHGHDFYVLGRSPDESPASNERHVFDPARDAGLLSGANPVRRDVTM
LPAFGWVVLAFRADNPGAWLFHCHIAWHVSGGLGVVYLERADDLRGAVSDADADDL
DRLCADWRRYWPTNPYPKSDSGL
>Oxid_11
Seq. ID NO: 99
MSSRFQSLFFFVLVSLTAVANAAIGPVADLTLTNAQVSPDGFAREAVVVNGITPAPLITG
NKGDRFQLNVIDQLTNHTMLKTSSIHWHGFFQQGTNWADGPAFVNQCPIASGHSFLYD
FQVPDQAGTFWYHSHLSTQYCDGLRGPFVVYDPNDPHASLYDIDNDDTVITLADWYHV
AAKLGPRFPFGSDSTLINGLGRTTGIAPSDLAVIKVTQGKRYRFRLVSLSCDPNHTFSIDN
HTMTIIEADSINTQPLEVDSIQIFAAQRYSFVLDASQPVDNYWIRANPAFGNTGFAGGINS
AILRYDGAPEIEPTSVQTTPTKPLNEVDLHPLSPMPVPGSPEPGGVDKPLNLVFNFNGTNF
FINDHTFVPPSVPVLLQILSGAQAAQDLVPEGSVFVLPSNSSIEISFPATANAPGFPHPFHL
HGHAFAVVRSAGSSVYNYDNPIFRDVVSTGQPGDNVTIRFETNNPGPWFLHCHIDFHLD
AGFAVVMAEDTPDTKAANPVPQAWSDLCPIYDALDPSDL
>Oxid_12
Seq. ID NO: 100
MSSGLQRFSFFVTLALVARSLAAIGPVASLVVANAPVSPDGFLRDAIVVNGVVPSPLITG
KKGDRFQLNVDDTLTNHSMLKSTSIHWHGFFQAGTNWADGPAFVNQCPIASGHSFLYD
FHVPDQAGTFWYHSHLSTQYCDGLRGPFVVYDPKDPHASRYDVDNESTVITLTDWYHT
AARLGPRFPLGADATLINGLGRSASTPTAALAVINVQHGKRYRFRLVSISCDPNYTFSID
GHNLTVIEVDGINSQPLLVDSIQIFAAQRYSFVLNANQTVGNYWVRANPNFGTVGFAGG
INSAILRYQGAPVAEPTTTQTTSVIPLIETNLHPLARMPVPGSPTPGGVDKALNLAFNFNG
TNFFINNATFTPPTVPVLLQILSGAQTAQDLLPAGSVYPLPAHSTIEITLPATALAPGAPHP
FHLHGHAFAVVRSAGSTTYNYNDPIFRDVVSTGTPAAGDNVTIRFQTDNPGPWFLHCHI
DFHLDAGFAIVFAEDVADVKAANPVPKAWSDLCPIYDGLSEANQ
>MBP
Seq. ID NO: 101
ATGAAGATTGAGGAGGGAAAACTTGTCATATGGATTAATGGCGACAAAGGCTATAA
TGGGTTAGCAGAAGTCGGTAAAAAGTTTGAGAAAGACACTGGGATTAAGGTAACGG
TCGAGCACCCAGATAAGCTGGAAGAGAAATTCCCACAGGTTGCCGCGACTGGGGAT
GGCCCCGACATCATATTCTGGGCGCACGACAGATTTGGCGGTTATGCACAAAGTGG
GTTACTAGCTGAAATTACCCCAGATAAGGCATTTCAAGACAAACTATATCCTTTCAC
TTGGGATGCGGTTAGATATAACGGAAAATTGATAGCCTATCCTATTGCCGTGGAGGC
TTTATCACTAATCTATAACAAGGACCTATTGCCGAACCCGCCCAAAACATGGGAAGA
AATCCCTGCCTTAGACAAAGAACTTAAAGCGAAAGGCAAGAGTGCTCTAATGTTCA
ATCTTCAAGAGCCTTATTTTACTTGGCCCTTGATAGCGGCCGATGGCGGCTACGCCTT
CAAGTACGAGAACGGGAAGTATGATATTAAAGACGTTGGAGTGGATAACGCGGGTG
CGAAGGCTGGCCTGACGTTCTTAGTGGACTTGATTAAAAATAAGCACATGAACGCG
GACACGGACTACAGCATCGCGGAGGCGGCTTTTAATAAGGGCGAAACTGCTATGAC
GATCAATGGACCTTGGGCTTGGTCAAATATAGATACAAGTAAGGTAAATTATGGAG
TAACTGTGCTGCCGACCTTTAAGGGCCAACCTAGTAAACCGTTTGTCGGCGTGTTGT
CCGCCGGGATAAACGCCGCCTCCCCCAACAAAGAATTAGCAAAGGAATTTTTGGAG
AATTACTTACTGACCGATGAGGGCTTGGAGGCAGTCAATAAGGATAAGCCCCTGGG
CGCTGTCGCATTGAAGTCATATGAAGAAGAACTTGCAAAAGATCCCCGTATTGCTGC
CACAATGGAGAATGCACAGAAAGGTGAAATAATGCCCAACATACCGCAGATGAGTG
CGTTCTGGTATGCGGTAAGAACAGCTGTTATCAACGCTGCGTCCGGGAGGCAAACA
GTTGATGAGGCTTTGAAAGACGCTCAGACCAATTCCTCCAGCAACAACAATAATAAT
AACAATAACAACAACTTAGGTATAGAAGGTAGATAA
>VEN
Seq. ID NO: 102
ATGGTTAGTAAAGGAGAAGAGTTATTCACTGGCGTTGTACCTATTCTGGTTGAGCTA
GACGGAGATGTTAATGGCCACAAATTCTCCGTATCCGGGGAGGGGGAGGGCGATGC
AACATATGGAAAACTTACGCTAAAACTAATCTGTACGACTGGGAAACTACCCGTTCC
GTGGCCCACATTGGTTACGACACTTGGCTATGGCCTACAGTGTTTCGCTAGATACCC
TGATCATATGAAGCAACATGATTTCTTTAAGAGTGCAATGCCGGAGGGTTACGTTCA
GGAAAGAACAATTTTCTTCAAGGATGACGGCAATTACAAGACGAGGGCCGAGGTAA
AATTCGAGGGGGATACGCTGGTTAACAGGATAGAATTAAAAGGTATAGATTTTAAA
GAAGACGGGAACATTCTAGGTCATAAACTTGAGTACAATTACAACTCCCATAATGTC
TACATAACAGCGGACAAGCAGAAGAATGGTATAAAGGCAAATTTTAAGATCAGACA
TAACATTGAAGACGGGGGAGTCCAGTTGGCTGACCACTATCAACAAAATACCCCCA
TTGGGGACGGTCCGGTGTTGCTTCCAGATAACCACTATCTTTCTTACCAGTCAGCCCT
ATCCAAAGACCCAAACGAGAAGAGGGATCATATGGTTCTTCTGGAGTTTGTCACCGC
AGCAGGGATTACTTTGGGGATGGACGAGCTATACAAGTAA
>MST
Seq. ID NO: 103
ATGGCTATGTTCTGCACTTTCTTCGAAAAACATCATCGTAAATGGGACATTTTACTA
GAGAAATCCACCGGTGTGATGGAGGCGATGAAAGTGACATCCGAAGAAAAGGAAC
AACTGAGCACAGCTATTGACCGTATGAACGAGGGCCTGGATGCTTTTATCCAGCTAT
ATAACGAGTCCGAAATAGACGAGCCCCTTATCCAGCTTGACGATGACACAGCCGAA
TTAATGAAACAAGCTAGAGATATGTACGGTCAGGAGAAGTTAAATGAAAAACTAAA
TACAATCATTAAGCAAATTTTGTCAATCTCTGTATCCGAGGAGGGAGAGAAAGAAG
GCAGCGGATCAGGATAA
>OSP
Seq. ID NO: 104
ATGTATCTTCTAGGGATTGGGCTTATTTTAGCACTGATTGCCTGCAAGCAAAACGTTT
CTTCACTAGACGAGAAAAACTCAGTGTCAGTAGACCTTCCTGGTGAGATGAAGGTTT
TGGTCAGCAAGGAAAAGAACAAAGATGGCAAGTACGATTTAATTGCTACCGTGGAT
AAGTTGGAGCTGAAAGGAACATCCGACAAGAACAACGGCTCTGGGGTACTTGAAGG
AGTCAAGGCCGATAAAAGCAAAGTCAAGCTAACAATTTCCGACGACGGGTCTGGAT
AA
>OLE
Seq. ID NO: 105
ATGGCAGACAGAGATAGGTCAGGTATCTATGGCGGTGCTCATGCGACGTATGGCCA
ACAGCAACAGCAAGGAGGAGGCGGGAGACCTATGGGCGAACAAGTCAAGGGCATG
CTGCATGACAAGGGCCCTACGGCGTCCCAGGCCTTGACAGTTGCTACCTTATTTCCT
TTAGGAGGGCTGCTTTTGGTGCTTAGTGGATTAGCCCTTACTGCTTCCGTAGTCGGTC
TAGCAGTCGCAACGCCGGTCTTTTTGATCTTCAGTCCGGTGCTAGTCCCAGCGGCAT
TGCTAATCGGCACTGCCGTCATGGGGTTCTTGACCTCCGGTGCTCTGGGTCTGGGTG
GTTTGTCCTCTCTGACCTGTCTAGCAAACACTGCGCGTCAGGCTTTTCAGCGTACCCC
TGACTACGTGGAAGAAGCTCATAGAAGGATGGCTGAGGCAGCAGCGCATGCCGGAC
ATAAGACGGCTCAGGCTGGGCAAGCTATTCAAGGCAGGGCTCAAGAGGCTGGCGCT
GGTGGTGGGGCCGGGTAA
>MBP
Seq. ID NO: 106
MSKIEEGKLVIWINGDKGYNGLAEVGKKFEKDTGIKVTVEHPDKLEEKFPQVAATGDGP
DIIFWAHDRFGGYAQSGLLAEITPDKAFQDKLYPFTWDAVRYNGKLIAYPIAVEALSLIY
NKDLLPNPPKTWEEIPALDKELKAKGKSALMFNLQEPYFTWPLIAADGGYAFKYENGK
YDIKDVGVDNAGAKAGLTFLVDLIKNKHMNADTDYSIAEAAFNKGETAMTINGPWAW
SNIDTSKVNYGVTVLPTFKGQPSKPFVGVLSAGINAASPNKELAKEFLENYLLTDEGLEA
VNKDKPLGAVALKSYEEELAKDPRIAATMENAQKGEIMPNIPQMSAFWYAVRTAVINA
ASGRQTVDEALKDAQTNSSSNNNNNNNNNNLGIEGR
>VEN
Seq. ID NO: 107
MVSKGEELFTGVVPILVELDGDVNGHKFSVSGEGEGDATYGKLTLKLICTTGKLPVPWP
TLVTTLGYGLQCFARYPDHMKQHDFFKSAMPEGYVQERTIFFKDDGNYKTRAEVKFEG
DTLVNRIELKGIDFKEDGNILGHKLEYNYNSHNVYITADKQKNGIKANFKIRHNIEDGGV
QLADHYQQNTPIGDGPVLLPDNHYLSYQSALSKDPNEKRDHMVLLEFVTAAGITLGMD
ELYK
>MST
Seq. ID NO: 108
MSAMFCTFFEKHHRKWDILLEKSTGVMEAMKVTSEEKEQLSTAIDRMNEGLDAFIQLY
NESEIDEPLIQLDDDTAELMKQARDMYGQEKLNEKLNTIIKQILSISVSEEGEKEGSGSG
>OSP
Seq. ID NO: 109
MSSYLLGIGLILALIACKQNVSSLDEKNSVSVDLPGEMKVLVSKEKNKDGKYDLIATVD
KLELKGTSDKNNGSGVLEGVKADKSKVKLTISDDGSG
>OLE
Seq. ID NO: 110
MSSADRDRSGIYGGAHATYGQQQQQGGGGRPMGEQVKGMLHDKGPTASQALTVATL
FPLGGLLLVLSGLALTASVVGLAVATPVFLIFSPVLVPAALLIGTAVMGFLTSGALGLGG
LSSLTCLANTARQAFQRTPDYVEEAHRRMAEAAAHAGHKTAQAGQAIQGRAQEAGAG
GGAG

What is claimed is:

1-76. (canceled)

77. A nucleic acid encoding an enzyme that can modify a first cannabinoid into a second cannabinoid or a non-cannabinoid, wherein the nucleic acid comprises any one of SEQ ID NOs:1-50.

78-88. (canceled)

89. An expression cassette comprising the nucleic acid of claim 77.

90-91. (canceled)

92. A cell comprising the expression cassette of claim 89, capable of expressing the enzyme that can modify a first cannabinoid into a second cannabinoid or a non-cannabinoid.

93. The cell of claim 92, which is a bacterial cell.

94. The cell of claim 92, which is a yeast cell.

95. The yeast cell of claim 95, which is a species of Saccharomyces, Candida, Pichia, Schizosaccharomyces, Scheffersomyces, Blakeslea, Rhodotorula, or Yarrowia.

96. The cell of claim 92, further comprising a THC biosynthetic pathway that allows the yeast cell to produce the first cannabinoid.

97. The cell of claim 96, wherein the cell can synthesize the first cannabinoid from a non-cannabinoid.

98. The cell of claim 96, wherein the cell comprises a recombinant geranyl pyrophosphate synthase and a cannabinoid synthase, wherein the cannabinoid synthase can combine a polyprenyl pyrophosphate with alkylresorcylic acid to create a cannabinoid.

99. The cell of claim 92, wherein the first cannabinoid is THC or THCA and the second cannabinoid is CBN or CBNA.

100-105. (canceled)

106. The cell of claim 92, wherein the first and/or second cannabinoid comprises the structure 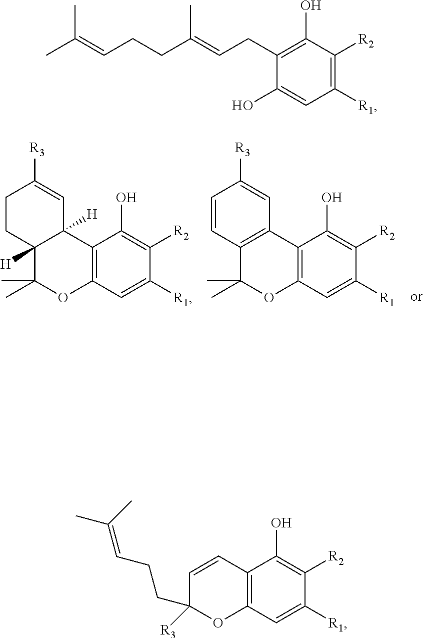
wherein R₁═CH₃, CH₂CH₃, (CH₂)₂CH₃, (CH₂)₃CH₃, (CH₂)₃CH₃, (CH₂)₄CH₃, (CH₂)₅CH₃, or (CH₂)₆CH₃; R₂═H or COOH; and R₃═CH₃ or CH₂OH.

107. The cell of claim 92, wherein the enzyme is an aromatase, a dehydrogenase, an oxidase or a desaturase.

108. The cell of claim 92, wherein the enzyme is an oxidase.

109. The cell of claim 108, wherein the oxidase is a laccase comprising an amino acid sequence comprising any one of SEQ ID NOs:92-100.

110. The cell of claim 108, wherein the oxidase is a cytochrome P450, expressed with a cytochrome P450 reductase (CPR).

111. The cell of claim 108, wherein the oxidase is selected from the group consisting of a flavin-dependent monooxygenase, a copper-dependent monooxygenase, a multicopper oxidase, a bacterial polysaccharide monooxygenase, a non-heme iron-dependent monooxygenase, a pterin-dependent monooxygenase, a diiron hydroxylase, an alpha-ketoglutarate-dependent hydroxylase, a cofactor-dependent monooxygenase, and a cofactor-independent monooxygenase.

112. The cell of claim 108, wherein the oxidase is a copper-dependent monooxygenase comprising an amino acid sequence having SEQ ID NO:89 or multicopper oxidase comprising an amino acid sequence having SEQ ID NO:90 or 91.

113. The cell of claim 92, wherein the first cannabinoid is converted into a second cannabinoid.

114. The cell of claim 113, wherein the first cannabinoid is tetrahydrocannabinol (THC) or tetrahydrocannabinolic acid (THCA) and the second cannabinoid is cannabinol (CBN) or cannabinolic acid (CBNA) and the enzyme is a desaturase, an aromatase, a dehydrogenase, or an oxidase.

115. The call of claim 113, wherein the first cannabinoid is tetrahydrocannabivarinic acid (THCVA), tetrahydrocannabiphorolic acid (TCHPA), tetrahydrocannabiorcinic acid (THCOA) or sesquiTHCA (THCFA) and the second cannabinoid is cannabinerolic acid (CBNA), cannabinerovarinic acid (CBNVA), cannabiphorolic acid (CBNPA), cannabinorcinic acid (CBNOA) or sesqui cannabinerolic acid (sesqui-CBNA), respectively.
